(19) United States
(12) Patent Application Publication
(10) Pub. No.: US 2004/0229779 A1
(43) Pub. Date: Nov. 18, 2004

(54) Therapeutic polypeptides, nucleic acids encoding same, and methods of use

(76) Inventors: Ramesh Kekuda, Stamford, CT (US); Uriel Malyankar, North Branford, CT (US); Li Li, Branford, CT (US); David Anderson, Plantsville, CT (US); Xiaojia (Sasha) Guo, Branford, CT (US); Mei Zhong, Branford, CT (US); Muralidhara Padigaru, Branford, CT (US); Stacie Casman, North Haven, CT (US); Ferenc Boldog, North Haven, CT (US); Charles Miller, Guilford, CT (US); Nikolai Khramtsov, Branford, CT (US); Corine Vernet, Chernex (CH); Meera Patturajan, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/637,313

(22) Filed: Aug. 8, 2003

Related U.S. Application Data
(63) Continuation-in-part of application No. 10/162,335, filed on Jun. 3, 2002.
(63) Continuation-in-part of application No. 10/211,689, filed on Aug. 1, 2002.
(63) Continuation-in-part of application No. 09/569,269, filed on May 11, 2000.
(63) Continuation-in-part of application No. 09/954,342, filed on Sep. 17, 2001, now abandoned.
(60) Provisional application No. 60/295,607, filed on Jun. 4, 2001.
(60) Provisional application No. 60/295,661, filed on Jun. 4, 2001.
(60) Provisional application No. 60/296,404, filed on Jun. 6, 2001.
(60) Provisional application No. 60/296,418, filed on Jun. 6, 2001.
(60) Provisional application No. 60/297,414, filed on Jun. 11, 2001.
(60) Provisional application No. 60/297,567, filed on Jun. 12, 2001.
(60) Provisional application No. 60/298,285, filed on Jun. 14, 2001.
(60) Provisional application No. 60/298,556, filed on Jun. 15, 2001.
(60) Provisional application No. 60/299,949, filed on Jun. 21, 2001.
(60) Provisional application No. 60/300,883, filed on Jun. 26, 2001.
(60) Provisional application No. 60/301,550, filed on Jun. 28, 2001.
(60) Provisional application No. 60/311,972, filed on Aug. 13, 2001.
(60) Provisional application No. 60/315,069, filed on Aug. 27, 2001.
(60) Provisional application No. 60/311,751, filed on Aug. 10, 2001.
(60) Provisional application No. 60/310,802, filed on Aug. 8, 2001.
(60) Provisional application No. 60/310,795, filed on Aug. 8, 2001.
(60) Provisional application No. 60/311,292, filed on Aug. 9, 2001.
(60) Provisional application No. 60/361,159, filed on Feb. 28, 2002.
(60) Provisional application No. 60/373,050, filed on Apr. 16, 2002.
(60) Provisional application No. 60/380,970, filed on May 15, 2002.
(60) Provisional application No. 60/311,979, filed on Aug. 13, 2001.
(60) Provisional application No. 60/381,030, filed on May 16, 2002.
(60) Provisional application No. 60/323,944, filed on Sep. 21, 2001.
(60) Provisional application No. 60/311,571, filed on Aug. 10, 2001.
(60) Provisional application No. 60/311,594, filed on Aug. 10, 2001.
(60) Provisional application No. 60/313,201, filed on Aug. 17, 2001.
(60) Provisional application No. 60/359,294, filed on Feb. 21, 2002.
(60) Provisional application No. 60/372,998, filed on Apr. 16, 2002.
(60) Provisional application No. 60/380,971, filed on May 15, 2002.
(60) Provisional application No. 60/312,892, filed on Aug. 16, 2001.
(60) Provisional application No. 60/322,716, filed on Sep. 17, 2001.
(60) Provisional application No. 60/360,890, filed on Feb. 28, 2002.
(60) Provisional application No. 60/314,031, filed on Aug. 21, 2001.
(60) Provisional application No. 60/315,853, filed on Aug. 29, 2001.
(60) Provisional application No. 60/134,315, filed on May 14, 1999.
(60) Provisional application No. 60/175,744, filed on Jan. 12, 2000.
(60) Provisional application No. 60/188,274, filed on Mar. 10, 2000.
(60) Provisional application No. 60/233,382, filed on Sep. 18, 2000.
(60) Provisional application No. 60/240,498, filed on Oct. 13, 2000.
(60) Provisional application No. 60/260,284, filed on Jan. 8, 2001.
(60) Provisional application No. 60/260,973, filed on Jan. 11, 2001.
(60) Provisional application No. 60/264,794, filed on Jan. 29, 2001.
(60) Provisional application No. 60/238,398, filed on Oct. 6, 2000.
(60) Provisional application No. 60/232,675, filed on Sep. 15, 2000.
(60) Provisional application No. 60/274,862, filed on Mar. 9, 2001.
(60) Provisional application No. 60/233,801, filed on Sep. 19, 2000.
(60) Provisional application No. 60/232,676, filed on Sep. 15, 2000.
(60) Provisional application No. 60/233,960, filed on Sep. 20, 2000.
(60) Provisional application No. 60/233,402, filed on Sep. 18, 2000.
(60) Provisional application No. 60/233,521, filed on Sep. 19, 2000.
(60) Provisional application No. 60/233,522, filed on Sep. 19, 2000.
(60) Provisional application No. 60/232,679, filed on Sep. 15, 2000.

(51) Int. Cl. A61K 38/17; C07K 14/47

(52) U.S. Cl. 514/12; 530/350

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. The invention further discloses therapeutic, diagnostic and research methods for diagnosis, treatment, and prevention of disorders involving any one of these novel human nucleic acids and proteins.

### RELATED APPLICATIONS

This application is a continuation in part of U.S. Ser. No. 10/162335, filed Jun. 3, 2002, which claims priority to U.S.S.N. 60/295607, filed Jun. 4, 2001; U.S.S.N. 60/295661, filed Jun. 4, 2001; U.S.S.N. 60/296404, filed Jun. 6, 2001; U.S.S.N. 60/296418, filed Jun. 6, 2001; U.S.S.N. 60/298285, filed Jun. 14, 2001; U.S.S.N. 60/298556, filed Jun. 15, 2001; U.S.S.N. 60/299949, filed Jun. 21, 2001; U.S.S.N. 60/300883, filed Jun. 26, 2001; U.S.S.N. 60/301550, filed Jun. 28, 2001; U.S.S.N. 60/311972, filed Aug. 13, 2001; U.S.S.N. 60/315071, filed Aug. 27, 2001; U.S.S.N. 60/315660, filed Aug. 29, 2001; U.S.S.N. 60/322293, filed Sep. 14, 2001; U.S.S.N. 60/322706, filed Sep. 17, 2001; U.S.S.N. 60/341186, filed Dec. 14, 2001; U.S.S.N. 60/361189, filed Feb. 28, 2001; U.S.S.N. 60/363673, filed Mar. 12, 2001; U.S.S.N. 60/363676, filed Mar. 12, 2002; U.S.S.N. 60/297414, filed Jun. 11, 2001; U.S.S.N. 60/297567, filed Jun. 12, 2001; and U.S.S.N. 60/315069, filed Aug. 27, 2001; a continuation in part of U.S. Ser. No. 10/211689, filed Aug. 1, 2002, which claims priority to U.S.S.N. 60/311751, filed Aug. 10, 2001; U.S.S.N. 60/310802, filed Aug. 8, 2001; U.S.S.N. 60/310795, filed Aug. 8, 2001; U.S.S.N. 60/311292, filed Aug. 9, 2001; U.S.S.N. 60/361159, filed Feb. 28, 2002; U.S.S.N. 60/373050, filed Apr. 16, 2002; U.S.S.N. 60/380970, filed May 15, 2002; U.S.S.N. 60/311979, filed Aug. 13, 2001; U.S.S.N. 60/381030, filed May 16, 2002; U.S.S.N. 60/323944, filed Sep. 21, 2001; U.S.S.N. 60/311571, filed Aug. 10, 2001; U.S.S.N. 60/311594, filed Aug. 10, 2001; U.S.S.N. 60/313201, filed Aug. 17, 2001; U.S.S.N. 60/359294, filed Feb. 21, 2002; U.S.S.N. 60/372998, filed Apr. 16, 2002; U.S.S.N. 60/380971, filed May 15, 2002; U.S.S.N. 60/312892, filed Aug. 16, 2001; U.S.S.N. 60/322716, filed Sep. 17, 2001; U.S.S.N. 60/360890, filed Feb. 28, 2002; U.S.S.N. 60/314031, filed Aug. 21, 2001; and U.S.S.N. 60/315853, filed Aug. 29, 2001; a continuation in part of U.S. Ser. No. 09/569269, filed May 11, 2000, which claims priority to U.S.S.N. 60/134315, filed May 14, 1999; U.S.S.N. 60/175744, filed Jan. 12, 2000; and U.S.S.N. 60/188274, filed Mar. 10, 2000; a continuation in part of U.S. Ser. No. 09/954342, filed Sep. 17, 2001, which claims priority to U.S.S.N. 60/233382, filed Sep. 18, 2000; U.S.S.N. 60/240,498, filed Oct. 13, 2000; U.S.S.N. 60/260,284, filed Jan. 8, 2001; U.S.S.N. 60/260,973, filed Jan. 11, 2001; U.S.S.N. 60/264,794, filed Jan. 29, 2001; U.S.S.N. 60/238,398, filed Oct. 6, 2000; U.S.S.N. 60/232,675, filed Sep. 15, 2000; U.S.S.N. 60/274,862, filed Mar. 9, 2001; U.S.S.N. 60/233,801, filed Sep. 19, 2000; U.S.S.N. 60/232,676, filed Sep. 15, 2000; U.S.S.N. 60/233,960, filed Sep. 20, 2000; U.S.S.N. 60/233,402, filed Sep. 18, 2000; U.S.S.N. 60/233,521, filed Sep. 19, 2000; U.S.S.N. 60/233,522, filed Sep. 19, 2000; and U.S.S.N. 60/232,679, filed Sep. 15, 2000 and a continuation in part of U.S. Ser. No. 10/379747, filed Mar. 5, 2003, which claims priority to U.S.S.N. 60/403485, filed Aug. 13, 2002; U.S.S.N. 60/365034 filed Mar. 15, 2002; U.S.S.N. 60/366420 filed Mar. 21, 2002; and U.S.S.N. 60/365477 filed Mar. 19, 2002; and this application also claims priority to U.S.S.N. 60/403485, filed Aug. 13, 2002; U.S.S.N. 60/414996, filed Sep. 30, 2002; U.S.S.N. 60/403815, filed Aug. 15, 2002; U.S.S.N. 60/425563, filed Nov. 12, 2002; U.S.S.N. 60/412995, filed Sep. 23, 2002; U.S.S.N. 60/403260, filed Aug. 14, 2002; U.S.S.N. 60/404190, filed Aug. 16, 2002; U.S.S.N. 60/402205, filed Aug. 9, 2002; U.S.S.N. 60/403398, filed Aug. 13, 2002; and U.S.S.N. 60/403517, filed Aug. 13, 2002. The contents of these applications are incorporated herein in their entirety.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes, which under normal conditions are exquisitely balanced to achieve the preservation and propagation of the cells. When such cells are components of multicellular organisms such as vertebrates or, more particularly, organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example, two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including, by way of nonlimiting example, induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 102. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO:2n, wherein n is an integer between 1 and 102, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 102. In another embodiment, the invention also comprises variants of the amino acid sequence selected from the group consisting of SEQ ID NO:2n, wherein n is an integer between 1 and 102, wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 102, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 102. These allelic variants include amino acid sequences that are the translations of nucleic acid sequences differing by a single nucleotide from nucleic acid sequences selected from the group consisting of SEQ ID NOS: 2n−1, wherein n is an integer between 1 and 102. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 102, and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 102, wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 102, in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 102, in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 102, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 102, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 102, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 102, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 102, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 102, or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 102, a variant of a mature form of the amino acid sequence selected from the group consisting of SEQ ID NO:2n, wherein n is an integer between 1 and 102, wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 102, a variant of the amino acid sequence selected from the group consisting of SEQ ID NO:2n, wherein n is an integer between 1 and 102, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 102, or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 102, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 102, that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 102, wherein the nucleic acid molecule differs by a single nucleotide from a nucleic acid sequence selected from the group consisting of SEQ ID NOS: 2n−1, wherein n is an integer between 1 and 102.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 102, wherein the nucleic acid molecule comprises a nucleotide sequence selected from the group consisting of the nucleotide sequence selected from the group consisting of SEQ ID NO:2n−1, wherein n is an integer between 1 and 102, a nucleotide sequence wherein one or more nucleotides in the nucleotide sequence selected from the group consisting of SEQ ID NO:2n−1, wherein n is an integer between 1 and 102, is changed from that selected from the group consisting of the chosen sequence to a different nucleotide provided that no more than 15% of the nucleotides are so changed; a nucleic acid fragment of the sequence selected from the group consisting of SEQ ID NO:2n−1, wherein n is an integer between 1 and 102, and a nucleic acid fragment wherein one or more nucleotides in the nucleotide sequence selected from the group consisting of SEQ ID NO:2n−1, wherein n is an integer between 1 and 102, is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 102, wherein the nucleic acid molecule hybridizes under stringent conditions to the nucleotide sequence selected from the group consisting of SEQ ID NO:2n−1, wherein n is an integer between 1 and 102, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 102, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 102. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 102, in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 102, in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| NOVX | Internal | SEQ ID NO | SEQ ID NO | |
|---|---|---|---|---|
| Assignment | Identification | (nucleic acid) | (amino acid) | Homology |
| NOV1a | CG104903-09 | 1 | 2 | Kininogen precursor (Alpha-2- |
| | | | | thiol proteinase inhibitor) |
| | | | | [Contains: Bradykinin] - Homo |
| | | | | sapiens |
| NOV1b | CG104903-03 | 3 | 4 | Kininogen precursor (Alpha-2- |
| | | | | thiol proteinase inhibitor) |
| | | | | [Contains: Bradykinin] - Homo |
| | | | | sapiens |
| NOV1c | 316514816 | 5 | 6 | Kininogen precursor (Alpha-2- |
| | | | | thiol proteinase inhibitor) |
| | | | | [Contains: Bradykinin] - Homo |
| | | | | sapiens |
| NOV1d | 308780224 | 7 | 8 | Kininogen precursor (Alpha-2- |
| | | | | thiol proteinase inhibitor) |
| | | | | [Contains: Bradykinin] - Homo |
| | | | | sapiens |
| NOV1e | 308900326 | 9 | 10 | Kininogen precursor (Alpha-2- |
| | | | | thiol proteinase inhibitor) |
| | | | | [Contains: Bradykinin] - Homo |
| | | | | sapiens |
| NOV1f | 308900357 | 11 | 12 | Kininogen precursor (Alpha-2- |
| | | | | thiol proteinase inhibitor) |
| | | | | [Contains: Bradykinin] - Homo |
| | | | | sapiens |
| NOV1g | 319687415 | 13 | 14 | Kininogen precursor (Alpha-2- |
| | | | | thiol proteinase inhibitor) |
| | | | | [Contains: Bradykinin] - Homo |
| | | | | sapiens |
| NOV1h | CG104903-10 | 15 | 16 | Kininogen precursor (Alpha-2- |
| | | | | thiol proteinase inhibitor) |
| | | | | [Contains: Bradykinin] - Homo |
| | | | | sapiens |
| NOV1i | 311750024 | 17 | 18 | Kininogen precursor (Alpha-2- |
| | | | | thiol proteinase inhibitor) |
| | | | | [Contains: Bradykinin] - Homo |
| | | | | sapiens |
| NOV1j | CG104903-01 | 19 | 20 | Kininogen precursor (Alpha-2- |
| | | | | thiol proteinase inhibitor) |
| | | | | [Contains: Bradykinin] - Homo |
| | | | | sapiens |
| NOV1k | CG104903-02 | 21 | 22 | Kininogen precursor (Alpha-2- |
| | | | | thiol proteinase inhibitor) |
| | | | | [Contains: Bradykinin] - Homo |
| | | | | sapiens |
| NOV1l | CG104903-04 | 23 | 24 | Kininogen precursor (Alpha-2- |
| | | | | thiol proteinase inhibitor) |
| | | | | [Contains: Bradykinin] - Homo |
| | | | | sapiens |
| NOV1m | CG104903-05 | 25 | 26 | Kininogen precursor (Alpha-2- |
| | | | | thiol proteinase inhibitor) |
| | | | | [Contains: Bradykinin] - Homo |
| | | | | sapiens |
| NOV1n | CG104903-06 | 27 | 28 | Kininogen precursor (Alpha-2- |
| | | | | thiol proteinase inhibitor) |
| | | | | [Contains: Bradykinin] - Homo |
| | | | | sapiens |
| NOV1o | CG104903-07 | 29 | 30 | Kininogen precursor (Alpha-2- |
| | | | | thiol proteinase inhibitor) |
| | | | | [Contains: Bradykinin] - Homo |
| | | | | sapiens |
| NOV1p | CG104903-08 | 31 | 32 | Kininogen precursor (Alpha-2- |
| | | | | thiol proteinase inhibitor) |
| | | | | [Contains: Bradykinin] - Homo |
| | | | | sapiens |
| NOV1q | CG104903-11 | 33 | 34 | Kininogen precursor (Alpha-2- |
| | | | | thiol proteinase inhibitor) |
| | | | | [Contains: Bradykinin] - Homo |
| | | | | sapiens |
| NOV1r | CG104903-12 | 35 | 36 | Kininogen precursor (Alpha-2- |
| | | | | thiol proteinase inhibitor) |
| | | | | [Contains: Bradykinin] - Homo |
| | | | | sapiens |
| NOV1s | CG104903-13 | 37 | 38 | Kininogen precursor (Alpha-2- |
| | | | | thiol proteinase inhibitor) |
| | | | | [Contains: Bradykinin] - Homo |
| | | | | sapiens |
| NOV1t | CG104903-14 | 39 | 40 | Kininogen precursor (Alpha-2- |
| | | | | thiol proteinase inhibitor) |
| | | | | [Contains: Bradykinin] - Homo |
| | | | | sapiens |
| NOV1u | CG104903-15 | 41 | 42 | Kininogen precursor (Alpha-2- |
| | | | | thiol proteinase inhibitor) |
| | | | | [Contains: Bradykinin] - Homo |
| | | | | sapiens |
| NOV1v | CG104903-16 | 43 | 44 | Kininogen precursor (Alpha-2- |
| | | | | thiol proteinase inhibitor) |
| | | | | [Contains: Bradykinin] - Homo |
| | | | | sapiens |
| NOV1w | CG104903-17 | 45 | 46 | Kininogen precursor (Alpha-2- |
| | | | | thiol proteinase inhibitor) |
| | | | | [Contains: Bradykinin] - Homo |
| | | | | sapiens |
| NOV1x | CG104903-18 | 47 | 48 | Kininogen precursor (Alpha-2- |
| | | | | thiol proteinase inhibitor) |
| | | | | [Contains: Bradykinin] - Homo |
| | | | | sapiens |
| NOV1y | CG104903-19 | 49 | 50 | Kininogen precursor (Alpha-2- |
| | | | | thiol proteinase inhibitor) |
| | | | | [Contains: Bradykinin] - Homo |
| | | | | sapiens |
| NOV1z | CG104903-20 | 51 | 52 | Kininogen precursor (Alpha-2- |
| | | | | thiol proteinase inhibitor) |
| | | | | [Contains: Bradykinin] - Homo |
| | | | | sapiens |
| NOV1aa | SNP13381566 | 53 | 54 | Kininogen precursor (Alpha-2- |
| | | | | thiol proteinase inhibitor) |
| | | | | [Contains: Bradykinin] - Homo |
| | | | | sapiens |
| NOV1ab | SNP13379157 | 55 | 56 | Kininogen precursor (Alpha-2- |
| | | | | thiol proteinase inhibitor) |
| | | | | [Contains: Bradykinin] - Homo |
| | | | | sapiens |
| NOV1ac | SNP13379158 | 57 | 58 | Kininogen precursor (Alpha-2- |
| | | | | thiol proteinase inhibitor) |
| | | | | [Contains: Bradykinin] - Homo |
| | | | | sapiens |
| NOV1ad | SNP13379159 | 59 | 60 | Kininogen precursor (Alpha-2- |
| | | | | thiol proteinase inhibitor) |
| | | | | [Contains: Bradykinin] - Homo |
| | | | | sapiens |
| NOV1ae | SNP13375447 | 61 | 62 | Kininogen precursor (Alpha-2- |
| | | | | thiol proteinase inhibitor) |
| | | | | [Contains: Bradykinin] - Homo |
| | | | | sapiens |
| NOV1af | SNP13380032 | 63 | 64 | Kininogen precursor (Alpha-2- |
| | | | | thiol proteinase inhibitor) |
| | | | | [Contains: Bradykinin] - Homo |
| | | | | sapiens |
| NOV1ag | SNP13380035 | 65 | 66 | Kininogen precursor (Alpha-2- |
| | | | | thiol proteinase inhibitor) |
| | | | | [Contains: Bradykinin] - Homo |
| | | | | sapiens |
| NOV1ah | SNP13375317 | 67 | 68 | Kininogen precursor (Alpha-2- |
| | | | | thiol proteinase inhibitor) |
| | | | | [Contains: Bradykinin] - Homo |
| | | | | sapiens |
| NOV1ai | SNP13375316 | 69 | 70 | Kininogen precursor (Alpha-2- |
| | | | | thiol proteinase inhibitor) |
| | | | | [Contains: Bradykinin] - Homo |
| | | | | sapiens |
| NOV1aj | SNP13379639 | 71 | 72 | Kininogen precursor (Alpha-2- |
| | | | | thiol proteinase inhibitor) |
| | | | | [Contains: Bradykinin] - Homo |
| | | | | sapiens |
| NOV1ak | SNP13376277 | 73 | 74 | Kininogen precursor (Alpha-2- |
| | | | | thiol proteinase inhibitor) |
| | | | | [Contains: Bradykinin] - Homo |
| | | | | sapiens |
| NOV1al | SNP13379163 | 75 | 76 | Kininogen precursor (Alpha-2- |
| | | | | thiol proteinase inhibitor) |
| | | | | [Contains: Bradykinin] - Homo |
| | | | | sapiens |
| NOV2a | CG120844-02 | 77 | 78 | Interleukin 1, beta - Homo |
| | | | | sapiens |
| NOV2b | 251426189 | 79 | 80 | Interleukin 1, beta - Homo |
| | | | | sapiens |
| NOV2c | CG120844-01 | 81 | 82 | Interleukin 1, beta - Homo |
| | | | | sapiens |
| NOV2d | SNP13377796 | 83 | 84 | Interleukin 1, beta - Homo |
| | | | | sapiens |
| NOV2e | SNP13377795 | 85 | 86 | Interleukin 1, beta - Homo |
| | | | | sapiens |
| NOV2f | SNP13377794 | 87 | 88 | Interleukin 1, beta - Homo |
| | | | | sapiens |
| NOV2g | SNP13377793 | 89 | 90 | Interleukin 1, beta - Homo |
| | | | | sapiens |
| NOV2h | SNP13377781 | 91 | 92 | Interleukin 1, beta - Homo |
| | | | | sapiens |
| NOV2i | SNP13377792 | 93 | 94 | Interleukin 1, beta - Homo |
| | | | | sapiens |
| NOV3a | CG127616-01 | 95 | 96 | Erythropoietin precursor |
| | | | | (Epoetin) - Homo sapiens |
| NOV3b | CG127616-02 | 97 | 98 | Erythropoietin precursor |
| | | | | (Epoetin) - Homo sapiens |
| NOV3c | 227803412 | 99 | 100 | Erythropoietin precursor |
| | | | | (Epoetin) - Homo sapiens |
| NOV3d | CG127616-03 | 101 | 102 | Erythropoietin precursor |
| | | | | (Epoetin) - Homo sapiens |
| NOV3e | CG127616-04 | 103 | 104 | Erythropoietin precursor |
| | | | | (Epoetin) - Homo sapiens |
| NOV3f | CG127616-05 | 105 | 106 | Erythropoietin precursor |
| | | | | (Epoetin) - Homo sapiens |
| NOV3g | CG127616-06 | 107 | 108 | Erythropoietin precursor |
| | | | | (Epoetin) - Homo sapiens |
| NOV3h | CG127616-07 | 109 | 110 | Erythropoietin precursor |
| | | | | (Epoetin) - Homo sapiens |
| NOV3i | CG127616-08 | 111 | 112 | Erythropoietin precursor |
| | | | | (Epoetin) - Homo sapiens |
| NOV3j | CG127616-09 | 113 | 114 | Erythropoietin precursor |
| | | | | (Epoetin) - Homo sapiens |
| NOV4a | CG54455-03 | 115 | 116 | Fibroblast growth factor-22 |
| | | | | precursor (FGF-22) - Homo |
| | | | | sapiens |
| NOV4b | 260403849 | 117 | 118 | Fibroblast growth factor-22 |
| | | | | precursor (FGF-22) - Homo |
| | | | | sapiens |
| NOV4c | CG54455-07 | 119 | 120 | Fibroblast growth factor-22 |
| | | | | precursor (FGF-22) - Homo |
| | | | | sapiens |
| NOV4d | 306448506 | 121 | 122 | Fibroblast growth factor-22 |
| | | | | precursor (FGF-22) - Homo |
| | | | | sapiens |
| NOV4e | CG54455-01 | 123 | 124 | Fibroblast growth factor-22 |
| | | | | precursor (FGF-22) - Homo |
| | | | | sapiens |
| NOV4f | CG54455-02 | 125 | 126 | Fibroblast growth factor-22 |
| | | | | precursor (FGF-22) - Homo |
| | | | | sapiens |
| NOV4g | CG54455-04 | 127 | 128 | Fibroblast growth factor-22 |
| | | | | precursor (FGF-22) - Homo |
| | | | | sapiens |
| NOV4h | CG54455-05 | 129 | 130 | Fibroblast growth factor-22 |
| | | | | precursor (FGF-22) - Homo |
| | | | | sapiens |
| NOV4i | CG54455-06 | 131 | 132 | Fibroblast growth factor-22 |
| | | | | precursor (FGF-22) - Homo |
| | | | | sapiens |
| NOV4j | CG54455-08 | 133 | 134 | Fibroblast growth factor-22 |
| | | | | precursor (FGF-22) - Homo |
| | | | | sapiens |
| NOV4k | CG54455-09 | 135 | 136 | Fibroblast growth factor-22 |
| | | | | precursor (FGF-22) - Homo |
| | | | | sapiens |
| NOV4l | SNP13379002 | 137 | 138 | Fibroblast growth factor-22 |
| | | | | precursor (FGF-22) - Homo |
| | | | | sapiens |
| NOV5a | CG54611-06 | 139 | 140 | Wnt-3a protein precursor - |
| | | | | Homo sapiens |
| NOV5b | 283841210 | 141 | 142 | Wnt-3a protein precursor - |
| | | | | Homo sapiens |
| NOV5c | CG54611-01 | 143 | 144 | Wnt-3a protein precursor - |
| | | | | Homo sapiens |
| NOV5d | CG54611-02 | 145 | 146 | Wnt-3a protein precursor - |
| | | | | Homo sapiens |
| NOV5e | CG54611-03 | 147 | 148 | Wnt-3a protein precursor - |
| | | | | Homo sapiens |
| NOV5f | CG54611-04 | 149 | 150 | Wnt-3a protein precursor - |
| | | | | Homo sapiens |
| NOV5g | CG54611-05 | 151 | 152 | Wnt-3a protein precursor - |
| | | | | Homo sapiens |
| NOV5h | CG54611-07 | 153 | 154 | Wnt-3a protein precursor - |
| | | | | Homo sapiens |
| NOV5i | CG54611-08 | 155 | 156 | Wnt-3a protein precursor - |
| | | | | Homo sapiens |
| NOV5j | CG54611-09 | 157 | 158 | Wnt-3a protein precursor - |
| | | | | Homo sapiens |
| NOV5k | CG54611-10 | 159 | 160 | Wnt-3a protein precursor - |
| | | | | Homo sapiens |
| NOV5l | CG54611-11 | 161 | 162 | Wnt-3a protein precursor - |
| | | | | Homo sapiens |
| NOV5m | CG54611-12 | 163 | 164 | Wnt-3a protein precursor - |
| | | | | Homo sapiens |
| NOV5n | CG54611-13 | 165 | 166 | Wnt-3a protein precursor - |
| | | | | Homo sapiens |
| NOV5o | CG54611-14 | 167 | 168 | Wnt-3a protein precursor - |
| | | | | Homo sapiens |
| NOV5p | CG54611-15 | 169 | 170 | Wnt-3a protein precursor - |
| | | | | Homo sapiens |
| NOV5q | CG54611-16 | 171 | 172 | Wnt-3a protein precursor - |
| | | | | Homo sapiens |
| NOV5r | CG54611-17 | 173 | 174 | Wnt-3a protein precursor - |
| | | | | Homo sapiens |
| NOV5s | CG54611-18 | 175 | 176 | Wnt-3a protein precursor - |
| | | | | Homo sapiens |
| NOV5t | SNP13378438 | 177 | 178 | Wnt-3a protein precursor - |
| | | | | Homo sapiens |
| NOV5u | SNP13378437 | 179 | 180 | Wnt-3a protein precursor - |
| | | | | Homo sapiens |
| NOV5v | SNP13381548 | 181 | 182 | Wnt-3a protein precursor - |
| | | | | Homo sapiens |
| NOV5w | SNP13381645 | 183 | 184 | Wnt-3a protein precursor - |
| | | | | Homo sapiens |
| NOV5x | SNP13381646 | 185 | 186 | Wnt-3a protein precursor - |
| | | | | Homo sapiens |
| NOV5y | SNP13381647 | 187 | 188 | Wnt-3a protein precursor - |
| | | | | Homo sapiens |
| NOV5z | SNP13381648 | 189 | 190 | Wnt-3a protein precursor - |
| | | | | Homo sapiens |
| NOV5aa | SNP13381649 | 191 | 192 | Wnt-3a protein precursor - |
| | | | | Homo sapiens |
| NOV6a | CG92035-02 | 193 | 194 | C1q-related factor precursor - |
| | | | | Homo sapiens |
| NOV6b | 214458541 | 195 | 196 | C1q-related factor precursor - |
| | | | | Homo sapiens |
| NOV6c | 214458545 | 197 | 198 | C1q-related factor precursor - |
| | | | | Homo sapiens |
| NOV6d | 214458564 | 199 | 200 | C1q-related factor precursor - |
| | | | | Homo sapiens |
| NOV6e | CG92035-01 | 201 | 202 | C1q-related factor precursor - |
| | | | | Homo sapiens |
| NOV6f | CG92035-03 | 203 | 204 | C1q-related factor precursor - |
| | | | | Homo sapiens |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders, conditions and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., detection of a variety of cancers.

Additional utilities for NOVX nucleic acids and polypeptides according to the invention are disclosed herein.

### NOVX clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO:2n, wherein n is an integer between 1 and 102, (b) a variant of a mature form of the amino acid sequence selected from the group consisting of SEQ ID NO:2n, wherein n is an integer between 1 and 102, wherein any amino acid in the mature form is changed to a different amino acid, provided that no more than 15% of the amino acid residues in the sequence of the mature form are so changed; (c) an amino acid sequence selected from the group consisting of SEQ ID NO:2n, wherein n is an integer between 1 and 102, (d) a variant of the amino acid sequence selected from the group consisting of SEQ ID NO:2n, wherein n is an integer between 1 and 102, wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO:2n, wherein n is an integer between 1 and 102; (b) a variant of a mature form of the amino acid sequence selected from the group consisting of SEQ ID NO:2n, wherein n is an integer between 1 and 102, wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO:2n, wherein n is an integer between 1 and 102; (d) a variant of the amino acid sequence selected from the group consisting of SEQ ID NO:2n, wherein n is an integer between 1 and 102, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO:2n, wherein n is an integer between 1 and 102, or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO:2n−1, wherein n is an integer between 1 and 102; (b) a nucleotide sequence wherein one or more nucleotides in the nucleotide sequence selected from the group consisting of SEQ ID NO:2n−1, wherein n is an integer between 1 and 102, is changed from that selected from the group consisting of the chosen sequence to a different nucleotide provided that no more than 15% of the nucleotides are so changed; (c) a nucleic acid fragment of the sequence selected from the group consisting of SEQ ID NO:2n−1, wherein n is an integer between 1 and 102; and (d) a nucleic acid fragment wherein one or more nucleotides in the nucleotide sequence selected from the group consisting of SEQ ID NO:2n−1, wherein n is an integer between 1 and 102, is changed from that selected from the group consisting of the chosen sequence to a different nucleotide provided that no more than 15% of the nucleotides are so changed.

### NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide, precursor form, or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a post-translational modification step other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, about 4 kb, about 3 kb, about 2 kb, about 1 kb, about 0.5 kb, or about 0.1 kb, of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NOS: 2n−1, wherein n is an integer between 1 and 102, or a complement of this nucleotide sequence, can be isolated using standard molecular biology techniques and the sequence information provided herein. Using all or a portion of the nucleic acid sequence of SEQ ID NOS:2n−1, wherein n is an integer between 1 and 102, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993).

A nucleic acid of the invention can be amplified using cDNA, mRNA or, alternatively, genomic DNA as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:2n−1, wherein n is an integer between 1 and 102, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:2n−1, wherein n is an integer between 1 and 102, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NOS:2n−1, wherein n is an integer between 1 and 102, is one that is sufficiently complementary to the nucleotide sequence of SEQ ID NOS:2n−1, wherein n is an integer between 1 and 102, that it can hydrogen bond with few or no mismatches to a nucleotide sequence of SEQ ID NOS:2n−1, wherein n is an integer between 1 and 102, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 102, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 102; or an anti-sense strand nucleotide sequence of SEQ ID NO:2n−1, wherein n is an integer between 1 and 102; or of a naturally occurring mutant of SEQ ID NO:2n−1, wherein n is an integer between 1 and 102.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted. “A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 102, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

### NOVX Nucleic Acid and Polypeptide Variants

The invention further encompasses nucleic acid molecules that differ from the nucleotide sequences of SEQ ID NO:2n−1, wherein n is an integer between 1 and 102, due to degeneracy of the genetic code and thus encode the same NOVX proteins as that encoded by the nucleotide sequences of SEQ ID NO:2n−1, wherein n is an integer between 1 and 102. In another embodiment, an isolated nucleic acid molecule of the invention has a nucleotide sequence encoding a protein having an amino acid sequence of SEQ ID NO:2n, wherein n is an integer between 1 and 102.

In addition to the human NOVX nucleotide sequences of SEQ ID NO:2n−1, wherein n is an integer between 1 and 102, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 102, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 102. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 102, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 102, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 102, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

### Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 102, thereby leading to changes in the amino acid sequences of the encoded NOVX protein, without altering the functional ability of that NOVX protein. For example, nucleotide substitutions leading to amino acid substitutions at “inon-essential” amino acid residues can be made in the sequence of SEQ ID NO:2n, wherein n is an integer between 1 and 102. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 102, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 102. Preferably, the protein encoded by the nucleic acid molecule is at least about 60% homologous to SEQ ID NO:2n, wherein n is an integer between 1 and 102; more preferably at least about 70% homologous to SEQ ID NO:2n, wherein n is an integer between 1 and 102; still more preferably at least about 80% homologous to SEQ ID NO:2n, wherein n is an integer between 1 and 102; even more preferably at least about 90% homologous to SEQ ID NO:2n, wherein n is an integer between 1 and 102; and most preferably at least about 95% homologous to SEQ ID NO:2n, wherein n is an integer between 1 and 102.

An isolated nucleic acid molecule encoding a NOVX protein homologous to the protein of SEQ ID NO:2n, wherein n is an integer between 1 and 102, can be created by introducing one or more nucleotide substitutions, additions or deletions into the nucleotide sequence of SEQ ID NO:2n−1, wherein n is an integer between 1 and 102, such that one or more amino acid substitutions, additions or deletions are introduced into the encoded protein.

Mutations can be introduced any one of SEQ ID NO:2n−1, wherein n is an integer between 1 and 102, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 102, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

### Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01n75164, WO01/92513, WO01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX-) phenotype in the treated subject sample. The NOVX- phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

### Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

### Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

### RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

### Cell Culture

A cell culture known in the art to regularly express NOVX is propagated using standard conditions. 24 hours before transfection, at approximately 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

### Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 102, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 102, or antisense nucleic acids complementary to a NOVX nucleic acid sequence of SEQ ID NO:2n−1, wherein n is an integer between 1 and 102, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

### Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 102). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996. supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. W088/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

### NOVX Polypeptides

A polypeptide according to the invention includes a polypeptide including the amino acid sequence of NOVX polypeptides whose sequences are provided in any one of SEQ ID NO:2n, wherein n is an integer between 1 and 102. The invention also includes a mutant or variant protein any of whose residues may be changed from the corresponding residues shown in any one of SEQ ID NO:2n, wherein n is an integer between 1 and 102, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 102) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 102. In other embodiments, the NOVX protein is substantially homologous to SEQ ID NO:2n, wherein n is an integer between 1 and 102, and retains the functional activity of the protein of SEQ ID NO:2n, wherein n is an integer between 1 and 102, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 102, and retains the functional activity of the NOVX proteins of SEQ ID NO:2n, wherein n is an integer between 1 and 102.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 102.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 102, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

### NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

### Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with Si nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

### NOVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 102, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 μM to about 1 μM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

### Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

### Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

### Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

### Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication W094/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

### F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F_{(ab′)2} fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD 16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design. 3: 219-230 (1989).

### Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, 131I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

### Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

### Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or ³H.

### Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

### Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

### ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

### NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, useful expression vectors in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pV_{L} series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

### Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NOS:2n−1, wherein n is an integer between 1 and 102, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NOS:2n−1, wherein n is an integer between 1 and 102), but more preferably, is a non-human homologue of a human NOVX gene. For example, a mouse homologue of human NOVX gene of SEQ ID NOS:2n−1, wherein n is an integer between 1 and 102, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

### Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

### Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with 125I, 35S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule a NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-1 14, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

### Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

### Chromosome Mapping

Once the sequence (or a portion of the sequence) of a gene has been isolated, this sequence can be used to map the location of the gene on a chromosome. This process is called chromosome mapping. Accordingly, portions or fragments of a NOVX sequence, i.e., of SEQ ID NOS:2n−1, wherein n is an integer between 1 and 102, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NOS:2n−1, wherein n is an integer between 1 and 102, are used, a more appropriate number of primers for positive individual identification would be 500-2,000.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials. These and other agents are described in further detail in the following sections.

### Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:2n−1, wherein n is an integer between 1 and 102, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., F_{ab} or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

### Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing sample and control nucleic acids, e.g., DNA or RNA to high-density arrays containing hundreds or thousands of oligonucleotide probes. See, e.g., Cronin, et al., 1996. Human Mutation 7: 244-255; Kozal, et al., 1996. Nat. Med. 2: 753-759. For example, genetic mutations in NOVX can be identified in two-dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome Pregnancy Zone Protein Precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

### Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

### Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

### Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

### Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

### Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide And Polypeptide Sequences, And Homology Data

The NOV1 clone was analyzed, and the nucleotide and encoded polypeptide sequences are shown in Table 1A.

TABLE 1A
NOV1 Sequence Analysis

| | | |
|---|---|---|
| NOV1a, CG104903-09 | SEQ ID NO: 1 | 1984 bp |
| DNA Sequence | ORF Start: ATG at 50 | ORF Stop: TAA at 1982 |

AATTCCGGTTGAAACCATCCCTCAGCTCCTAGAGGGAGATTCTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGCATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCACAGCTGG
AGGAGACACTGACTCACACCATCACAAAGCTTAATGCAGAGAATAACGCAACTTTCTATTTCAAGATT
GACAATGTGAAAAAAGGAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATG
TTGAACACCAAGGGGGCCATGTCCTTGACCATGGACATAAGCATAAGCATGGTCATGGCCACGGAAAA
CATAAAAATAAAGGCAAAAAGAATGGAAACCACAATGGTTGGAAAACAGAGCATTTGGCAAGCTCTTC
TGAAGACAGTACTACACCTTCTGCACAGACACAAGAGAAGACACAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACCATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCTTAA

| | | | |
|---|---|---|---|
| NOV1a, CG104903-09 | | | |
| Protein Sequence | SEQ ID NO: 2 | 644 aa | MW at 71956.8 kD |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFODSDLIATMMPPISPAPIOSDDDWIPDIOIDPNGLSFNPISDFPDTTSP
KCPGRPWKSVSEINPTTQMKESYYFDLTDGLS

| | | |
|---|---|---|
| NOV1b, CG104903-03 | SEQ ID NO: 3 | 1981 bp |
| DNA Sequence | ORF Start: ATG at 50 | ORF Stop: end of sequence |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGACCTGG
AGGAGACACTGACTCACACCATCACAAAGCTTAATGCAGAGAATAACGCAACTTTCTATTTGAAGATT
GACAATGTGAAAAAAGCAAGAGTACAGGTGGTGGCTGGCAAGAAATATTTTATTGACTTCGTGGGCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTCATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATCATG
CCTCCTATATCACCAGCTCCCATACAGAGTGATGACGATTGGATCCCTGATATCCAGATACACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1b, CG104903-03 | | | |
| Protein Sequence | SEQ ID NO: 4 | 644 aa | MW at 71956.8 kD |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLCHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNCLSFNPISDFPDTTSP
KCPGRPWKSVSEINPTTQMKESYYFDLTDGLS

| | | |
|---|---|---|
| NOV1c, 316514816 | SEQ ID NO: 5 | 1935 bp |
| DNA Sequence | ORF Start: at 1 | ORF Stop: TAG at 1933 |

ATGAAACTAATTACCATCCTTTTCCTCTGCTCCAGGCTACTACTAAGTTTAAC
CCAGGAATCACAGTCCGAGGAAATTGACTGCAATGACAAGGATTTATTTAAAGCTGTGGATGCTGCTC
TGAAGAAATATAACAGTCAAAACCAAAGTAACAACCAGTTTGTATTGTACCGCATAACTGAAGCCACT
AAGACGGTTGGCTCTGACACGTTTTATTCCTTCAAGTACGAAATCAAGGAGGGGGACTGTCCTGTTCA
AAGTGGCAAAACCTGGCAGGACTGTGAGTACAAGGATGCAGCAAAAGCAGCCACTGGAGAATGCACGG
CAACCGTGGGGAAGAGGAGCAGTACCAAATTCTCCGTGGCTACCCAGACCTGCCAGATTACTCCAGCC
GACGGCCCTGTGGTGACAGCCCAGTACGACTGCCTCGGCTGTGTGCATCCTATATCAACGCAGAGCCC
AGACCTGGAGCCCATTCTGAGACACGGCATTCAGTACTTTAACAACAACACTCAACATTCCTCCCTCT
TCACGCTTAATGAAGTAAAACGGGCCCAAAGACAGGTGGTGGCTGGATTGAACTTTCGAATTACCTAC
TCAATTGTGCAAACGAATTGTTCCAAAGAGAATTTTCTGTTCTTAACTCCAGACTGCAAGTCCCTTTG
GAATGGTGATACCGGTGAATGTACAGATAATGCATACATCGATATTCAGCTACGAATTGCTTCCTTCT
CACAGAACTGTGACATTTATCCAGGGAAGGATTTTGTACAACCACCTACCAAGATTTGCGTGGGCTGC
CCCAGAGATATACCCACCAACAGCCCAGAGCTGGAGGAGACACTGACTCACACCATCACAAAGCTTAA
TGCAGAGAATAACGCAACTTTCTATTTCAAGATTGACAATGTGAAAAAAGCAAGAGTACAGGTGGTGG
CTGGCAAGAAATATTTTATTGACTTCGTGGCCAGGGAAACCACATGTTCCAAGGAAAGTAATGAAGAG
TTGACCGAAAGCTGTGAGACCAAAAAACTTGGCCAAAGCCTAGATTGCAACGCTGAAGTTTATGTGGT
ACCCTGGGAGAAAAAAATTTACCCTACTGTCAACTGTCAACCACTGGGAATGATCTCACTGATGAAAA
GGCCTCCAGGTTTTTCACCTTTCCGATCATCACGAATAGGGGAAATAAAAGAAGAAACAACTGTAAGT
CCACCCCACACTTCCATGGCACCTGCACAAGATGAAGAGCGGGATTCAGGAAAAGAACAAGGGCATAC
TCGTAGACATGACTGGGGCCATGAAAAACAAAGAAAACATAATCTTGGCCATGGCCATAAACATGAAC
GTGACCAAGGGCATGGGCACCAAAGAGGACATGGCCTTGGCCATGGACACGAACAACAGCATGGTCTT
GGTCATGGACATAAGTTCAAACTTGATGATGATCTTGAACACCAAGGGGGCCATGTCCTTGACCATGG
ACATAAGCATAAGCATGGTCATGGCCACGGAAAACATAAAAATAAAGGCAAAAAGAATGGAAAGCACA
ATGGTTGGAAAACAGAGCATTTGGCAAGCTCTTCTGAAGACAGTACTACACCTTCTGCACAGACACAA
GACAAGACAGAAGGGCCAACACCCATCCCTTCCCTAGCCAAGCCAGGTGTAACAGTTACCTTTTCTGA
CTTTCAGGACTCTCATCTCATTCCAACTATGATGCCTCCTATATCACCAGCTCCCATACAGAGTGATG
ACGATTGGATCCCTGATATCCAGATAGACCCAAATGGCCTTTCATTTAACCCAATATCAGATTTTCCA
GACACGACCTCCCCAAAATGTCCTGGACGCCCCTGGAAGTCAGTTAGTGAAATTAATCCAACCACACA
AATGAAAGAATCTTATTATTTCGATCTCACTGATGGCCTTTCTTAG

| | | | |
|---|---|---|---|
| NOV1c, 316514816 | | | |
| Protein Sequence | SEQ ID NO: 6 | 644 aa | MW at 71927.7 kD |

MKLITILFLCSRLLLSLTQESQSEEIDCNDKDLFKAVDAALKKYNSQNQSNNQFVLYRITEAT
KTVGSDTFYSFKYEIKEGDCPVQSGKTWQDCEYKDAAKAATGECTATVGKRSSTKFSVATQTCQITPA
EGPVVTAQYDCLGCVHPISTQSPDLEPILRHGIQYFNNNTQHSSLFTLNEVKRAQRQVVAGLNFRITY
SIVQTNCSKENFLFLTPDCKSLWNGDTGECTDNAYIDIQLRIASFSQNCDIYPGKDFVQPPTKICVGC
PRDIPTNSPELEETLTHTITKLNAENNATFYFKIDNVKKARVQVVAGKKYFIDFVARETTCSKESNEE
LTESCETKKLGQSLDCNAEVYVVPWEKKIYPTVNCQPLGMISLMKRPPGFSPFRSSRIGEIKEETTVS
PPHTSMAPAQDEERDSGKEQGHTRRHDWGHEKQRKHNLGHGHKHERDQGHGHQRGHGLGHGHEQQHGL
GHGHKFKLDDDLEHQGGHVLDHGHKHKHGHGHGKHKNKGKKNGKHNGWKTEHLASSSEDSTTPSAQTQ
EKTEGPTPIPSLAKPGVTVTFSDFQDSDLIATMMPPISPAPIQSDDDWIPDIQIDPNGLSFNPISDFP
DTTSPKCPGRPWKSVSEINPTTQMKESYYFDLTDGLS

| | | |
|---|---|---|
| NOV1d, 308780224 | SEQ ID NO: 7 | 327 bp |
| DNA Sequence | ORF Start: at 1 | ORF Stop: end of sequence |

TGCACCTGCACAAGATGAAGAGCGGGATTCAGGAAAAGAACAAGGGCATACTCGTAGAC
ATGACTGGGGCCATGAAAAACAAAGAAAACATAATCTTGGCCATGGCCATAAACATGAACGTGACCAA
GCGCATGGGCACCAAAGAGGACATGGCCTTGGCCATGGACACGAACAACAGCATGGTCTTGGTCATGG
ACATAAGTTCAAACTTCATGATGATCTTGAACACCAAGGGGGCCATGTCCTTGACCATGGACATAAGC
ATAAGCATGGTCATGGCCACGGAAAAGATAAAAATAAAGGCAAAAAGAATGGAAAGCACAATGG

| | | | |
|---|---|---|---|
| NOV1d, 308780224 | | | |
| Protein Sequence | SEQ ID NO: 8 | 109 aa | MW at 12335.1 kD |

APAQDEERDSGKEQGHTRRHDWGHEKQRKHNLGHGHKHERDQGHGHQRGHGLGHGHEQQHGLGHG
HKFKLDDDLEHQGGHVLDHGHKHKHGHGHGKHKNKGKKNGKHNG

| | | |
|---|---|---|
| NOV1e, 308900326 | SEQ ID NO: 9 | 1071 bp |
| DNA Sequence | ORF Start: at 1 | ORF Stop: end of sequence |

ATGAAACTAATTACCATCCTTTTCCTCTGCTCCAGGCTACTACTAAGTTTAA
CCCAGGAATCACAGTCCGAGGAAATTGACTGCAATGACAAGGATTTATTTAAAGCTGTGGATGCTGCT
CTGAAGAAATATAACAGTCAAAACCAAAGTAACAACCAGTTTGTATTGTACCGCATAACTCAAGCCAC
TAAGACGGTTGGCTCTGACACGTTTTATTCCTTCAAGTACGAAATCAAGGAGGGCGATTGTCCTGTTC
AAAGTGGCAAAACCTGGCAGGACTGTGAGTACAAGGATGCTGCAAAAGCAGCCACTGGAGAATGCACG
GCAACCGTGGGGAAGAGGAGCAGTACGAAATTCTCCGTGGCTACCCAGACCTGCCAGATTACTCCACC
CGAGGGCCCTGTGGTGACAGCCCAGTACGACTGCCTCGGCTGTGTGCATCCTATATCAACGCAGAGCC
CAGACCTGGAGCCCATTCTGAGACACGGCATTCAGTACTTTAACAACAACACTCAACATTCCTCCCTC
TTCATGCTTAATGAAGTAAAACGGGCCCAAAGACAGGTGGTGGCTGGATTGAACTTTCGAATTACCTA
CTCAATTGTGCAAACGAATTGTTCCAAAGAGAATTTTCTGTTCTTAACTCCAGACTGCAAGTCCCTTT
GGAATGGTGATACCGGTGAATGTACAGATAATGCATACATCGATATTCAGCTACGAATTGCTTCCTTC
TCACAGAACTGTGACATTTATCCAGGGAAGGATTTTGTACAACCACCTACCAAGATTTGCGTGGGCTG
CCCCAGGGATATACCCACCAACAGCCCAGAGCTGGAGGAGACACTGACTCACACCATCACAAAGCTTA
ATGCAGAGAATAACGCAACTTTCTATTTCAAGATTGACAATGTGAAAAAAGCAAGAGTACAGGTGGTG
GCTGGCAAGAAATATTTTATTGACTTCGTGGCCAGGGAAACCACATGTTCCAAGCAAAGTAATGAAGA
GTTGACCGAAAGCTGTGAGACCAAAAAACTTGGCCAAAGCCTAGATTGCAACGCTGAAGTTTATGTG

| | | | |
|---|---|---|---|
| NOV1e, 308900326 | | | |
| Protein Sequence | SEQ ID NO: 10 | 357 aa | MW at 40027.4 kD |

MKLITILFLCSRLLLSLTOESOSEEIDCNDKDLFKAVDAALKKYNSONOSNNOFVLYRITEAT
KTVGSDTFYSFKYEIKEGDCPVQSGKTWQDCEYKDAAKAATGECTATVGKRSSTKFSVATQTCQITPA
EGPVVTAQYDCLGCVHPISTQSPDLEPILRHGIQYFNNNTQHSSLFMLNEVKBAQRQVVAGLNFRITY
SIVQTNCSKENFLFLTPDCKSLWNGDTGECTDNAYIDIQLRIASFSQNCDIYPGKDFVQPPTKICVGC
PRDIPTNSPELEETLTHTITKLNAENNATFYFKIDNVKKARVQVVAGKKYFIDFVARETTCSKESNEE
LTESCETKKLGQSLDCNAEVYV

| | | |
|---|---|---|
| NOV1f, 308900357 | SEQ ID NO: 11 | 729 bp |
| DNA Sequence | ORF Start: at 1 | ORF Stop: end of sequence |

GGTTTTTCACCTTTCCGATCATCACGAATAGGGGAAATAAAAGAAGAAACAACTGTAA
GTCCACCCCACACTTCCATGGCACCTGCACAAGATGAAGAGCGGGATTCAGGAAAAGAACAAGGGCAT
ACTCGTAGACATGACTGGGGCCATGAAAAACAAAGAAAACATAATCTTGGCCATGGCCATAAACATGA
ACGTGACCAAGGGCATGGGCACCAAAGAGGACATGGCCTTGGCCATGGACACGAACAACAGCATGGTC
TTGGTCATGGACATAAGTTCAAACTTCATGATGATCTTGAACACCAAGGGGGCCATGTCCTTGACCAT
GGACATAAGCATAAGCATGGTCATGGCCACGGAAAACATAAAAATAAAGGCAAAAAGAATGGAAAGCA
CAATGGTTGGAAAACAGAGCATTTGGCAAGCTCTTCTGAAGACAGTACTACACCTTCTGCACAGACAC
AAGAGAAGACAGAAGGGCCAACACCCATCCCTTCCCTAGCCAAGCCAGGTGTAACAGTTACCTTTTCT
GACTTTCAGGACTCTGATCTCATTGCAACTATGATGCCTCCTATATCACCAGCTCCCATACAGAGTGA
TGACGATTGGATCCCTGATATCCAGATAGACCCAAATGGCCTTTCATTTAACCCAATATCAGATTTTC
CAGACACGACCTCCCCAAAATGTCCTGGACGCCCCTGGAAGTCAGTTAGTGAAATTAAT

| | | | |
|---|---|---|---|
| NOV1f, 308900357 | | | |
| Protein Sequence | SEQ ID NO: 12 | 243 aa | MW at 26861.3 kD |

GFSPFRSSRIGEIKEETTVSPPHTSMAPAQDEERDSGKEQGHTRRHDWGHEKQRKHNLGHGHKHE
RDQGHGHQRGHGLGHGHEQQHGLGHGHKFKLDDDLEHQGGHVLDHGHKHKHGHGHGKHKNKGKKNGKH
NGWKTEHLASSSEDSTTPSAQTQEKTEGPTPIPSLAKPGVTVTFSDFQDSDLIATMMPPISPAPIQSD
DDWIPDIQIDPNGLSFNPISDFPDTTSPKCPGRPWKSVSEIN

| | | |
|---|---|---|
| NOV1g, 319687415 | SEQ ID NO: 13 | 1848 bp |
| DNA Sequence | ORF Start: at 1 | ORF Stop: end of sequence |

CCCACCGACTGCAATCACAAGGATTTATTTAAAGCTGTGGATGCTGCTCTGAAGAAAT
ATAACAGTCAAAACCAAAGTAACAACCAGTTTGTATTGTACCGCATAACTGAACCCACTAAGACGGTT
GGCTCTGACACGTTTTATTCCTTCAAGTACGAAATCAAGGAGGGGGATTGTCCTGTTCAAAGTGGCAA
AACCTGGCAGGACTGTGAGTACAAGGATGCTGCAAAAGCAGCCACTGGAGAATGCACGGCAACCGTGG
GGAAGAGGAGCAGTACGAAATTCTCCGTGGCTACCCAGACCTGCCAGATTACTCCAGCCGAGGGCCCT
GTGGTGACAGCCCAGTACGACTGCCTCGGCTGTGTGCATCCTATATCAACGCAGAGCCCAGACCTGGA
GCCCATTCTGAGACACGGCATTCAGTACTTTAACAACAACACTCAACATTCCTCCCTCTTCATGCTTA
ATGAAGTAAAACGGGCCCAAAGACAGGTGGTGGCTGGATTGAACTTTCGAATTACCTACTCAATTGTG
CAAACGAATTGTTCCAAAGAGAATTTTCTGTTCTTAACTCCAGACTGCAAGTCCCTTTGGAATGGTGA
TACCGGTGAATGTACAGATAATGCATACATCGATATTCAGCTACGAATTGCTTCCTTCTCACAGAACT
GTGACATTTATCCAGGGAAGGATTTTGTACAACCACCTACCAAGATTTGCGTGGGCTGCCCCAGGGAT
ATACCCACCAACAGCCCAGAGCTGGAGGAGACACTGACTCACACCATCACAAAGCTTAATGCAGAGAA
TAACGCAACTTTCTATTTCAAGATTGACAATGTGAAAAAAGCAAGAGTACAGGTGGTGGCTGGCAAGA
AATATTTTATTGACTTCGTGGCCAGGGAAACCACATGTTCCAAGGAAAGTAATGAAGAGTTGACCGAA
AGCTGTGAGACCAAAAAACTTGGCCAAAGCCTAGATTGCAACGCTGAAGTTTATGTGGTACCCTGGGA
GAAAAAAATTTACCCTACTGTCAACTGTCAACCACTGGGAATGATCTCACTGATGAAAAGGCCTCCAG
GTTTTTCACCTTTCCGATCATCACGAATAGGGGAAATAAAAGAAGAAACAACTGTAAGTCCACCCCAC
ACTTCCATGGCACCTGCACAAGATGAAGAGCGGGATTCAGGAAAAGAACAAGGGCATACTCGTAGACA
TCACTGGGGCCATGAAAAACAAAGAAAACATAATCTTGGCCATGGCCATAAACATGAACGTGACCAAG
GGCATGGGCACCAAACAGGACATGGCCTTGGCCATGGACACGAACAACAGCATGGTCTTGGTCATGGA
CATAAGTTCAAACTTGATGATGATCTTGAACACCAAGGGGGCCATGTCCTTGACCATGGACATAAGCA
TAAGCATGGTCATGGCCACGGAAAACATAAAAATAAAGGCAAAAAGAATGGAAAGCACAATGGTTGGA
AAACAGAGCATTTGGCAAGCTCTTCTGAAGACAGTACTACACCTTCTGCACAGACACAAGAGAAGACA
GAAGGGCCAACACCCATCCCTTCCCTAGCCAAGCCAGGTGTAACAGTTACCTTTTCTGACTTTCAGCA
CTCTGATCTCATTGCAACTATGATGCCTCCTATATCACCAGCTCCCATACAGAGTGATGACGATTGGA
TCCCTGATATCCAGATAGACCCAAATGGCCTTTCATTTAACCCAATATCAGATTTTCCAGACACGACC
TCCCCAAAATGTCCTGGACGCCCCTGGAAGTCAGTTAGTGAAATTAATCCAACCACACAAATGAAAGA
ATCTTATTATTTCGATCTCACT

| | | | |
|---|---|---|---|
| NOV1g, 319687415 | | | |
| Protein Sequence | SEQ ID NO: 14 | 616 aa | MW at 68791.8 kD |

PTDCNDKDLFKAVDAALKKYNSQNQSNNQFVLYRITEATKTVGSDTFYSFKYEIKEGDCPVQSGK
TWQDCEYKDAAKAATGECTATVGKRSSTKFSVATQTCQITPAEGPVVTAQYDCLGCVHPISTQSPDLE
PILRHGIQYFNNNTQHSSLFMLNEVKRAQRQVVAGLNFRITYSIVQTNCSKENFLFLTPDCKSLWNGD
TGECTDNAYIDIQLRIASFSQNCDIYPGKDFVQPPTKICVGCPRDIPTNSPELEETLTHTITKLNAEN
NATFYFKIDNVKKARVOVVAGKKYFIDFVARETTCSKESNEELTESCETKKLGOSLDCNAEVYVVPWE
KKIYPTVNCQPLGMISLMKRPPGFSPFRSSRIGEIKEETTVSPPHTSMAPAQDEERDSGKEQGHTRRH
DWGHEKQRKHNLGHGHKHERDQGHGHQRGHGLGHGHEQQHGLGHGHKFKLDDDLEHQGGHVLDHGHKH
KHGHGHGKHKNKGKKNGKHNGWKTEHLASSSEDSTTPSAQTQEKTEGPTPIPSLAKPGVTVTFSDFQD
SDLIATMMPPISPAPIQSDDDWIPDIQIDPNGLSFNPISDFPDTTSPKCPGRPWKSVSEINPTTQMKE
SYYFDLT

| | | |
|---|---|---|
| NOV1h, CG104903-10 | SEQ ID NO: 15 | 1863 bp |
| DNA Sequence | ORF Start: at 1 | ORF Stop: at 1863 |

GAGGAAATTGACTGCAATGACAAGGATTTATTTAAAGCTGTGGATGCTGCTCTGAAGAAATA
TAACAGTCAAAACCAAAGTAACAACCAGTTTGTATTGTACCGCATAACTGAAGCCACTAAGACGGTTG
GCTCTGACACGTTTTATTCCTTCAAGTACGAAATCAAGGAGGGGGATTGTCCTGTTCAAAGTGGCAAA
ACCTGCCAGGACTGTGAGTACAAGGATGCTGCAAAAGCAGCCACTGGAGAATGCACGGCAACCGTGGG
GAAGAGGAGCAGTACGAAATTCTCCGTGGCTACCCAGACCTGCCAGATTACTCCAGCCGAGGGCCCTG
TGGTGACAGCCCAGTACGACTGCCTCGGCTGTGTGCATCCTATATCAACGCAGAGCCCAGACCTGGAG
CCCATTCTGAGACACGGCATTCAGTACTTTAACAACAACACTCAACATTCCTCCCTCTTCATGCTTAA
TGAAGTAAAACGGGCCCAAAGACAGGTGGTGGCTGGATTGAACTTTCGAATTACCTACTCAATTGTGC
AAACGAATTGTTCCAAAGAGAATTTTCTGTTCTTAACTCCAGACTGCAAGTCCCTTTGGAATGGTGAT
ACCGGTGAATGTACAGATAATGCATACATCCATATTCAGCTACGAATTGCTTCCTTCTCACAGAACTG
TGACATTTATCCAGGGAAGGATTTTGTACAACCACCTACCAAGATTTGCCTGGGCTGCCCCAGGGATA
TACCCACCAACAGCCCAGAGCTGGAGGAGACACTGACTCACACCATCACAAAGCTTAATGCAGAGAAT
AACGCAACTTTCTATTTCAAGATTGACAATGTGAAAAAAGCAAGAGTACAGGTGGTGGCTGGCAAGAA
ATATTTTATTGACTTCGTGGCCAGGGAAACCACATGTTCCAAGGAAAGTAATGAAGAGTTGACCGAAA
GCTGTGAGACCAAAAAACTTGGCCAAAGCCTAGATTGCAACGCTGAAGTTTATGTGGTACCCTGGGAG
AAAAAAATTTACCCTACTGTCAACTCTCAACCACTGGGAATGATCTCACTGATGAAAAGGCCTCCAGG
TTTTTCACCTTTCCGATCATCACGAATAGGGGAAATAAAAGAAGAAACAACTGTAAGTCCACCCCACA
CTTCCATGGCACCTGCACAAGATGAAGAGCGGGATTCAGGAAAAGAACAAGGGCATACTCGTAGACAT
GACTGGGGCCATGAAAAACAAAGAAAACATAATCTTCGCCATGGCCATAAACATGAACGTGACCAAGC
GCATGGGCACCAAAGAGGACATGGCCTTGGCCATGGACACGAACAACAGCATGGTCTTGGTCATGGAC
ATAAGTTCAAACTTGATGATGATCTTGAACACCAAGGGGGCCATGTCCTTGACCATGGACATAAGCAT
AAGCATGGTCATGGCCACGGAAAACATAAAAATAAAGGCAAAAAGAATGGAAAGCACAATGGTTGGAA
AACAGAGCATTTGGCAAGCTCTTCTGAAGACAGTACTACACCTTCTGCACAGACACAAGAGAAGACAG
AAGCGCCAACACCCATCCCTTCCCTAGCCAAGCCAGGTGTAACAGTTACCTTTTCTGACTTTCAGGAC
TCTGATCTCATTGCAACTATGATGCCTCCTATATCACCAGCTCCCATACAGAGTGATGACGATTGGAT
CCCTGATATCCAGATAGACCCAAATGGCCTTTCATTTAACCCAATATCAGATTTTCCAGACACGACCT
CCCCAAAATGTCCTGGACGCCCCTGGAAGTCAGTTAGTGAAATTAATCCAACCACACAAATGAAAGAA
TCTTATTATTTCGATCTCACTGATGGCCTTTCT

| | | | |
|---|---|---|---|
| NOV1h, CG104903-10 | | | |
| Protein Sequence | SEQ ID NO: 16 | 621 aa | MW at 69336.6 kD |

EEIDCNDKDLFKAVDAALKKYNSQNQSNNQFVLYRITEATKTVGSDTFYSFKYEIKEGDCPVQSGKTW
QDCEYKDAAKAATGECTATVGKRSSTKFSVATQTCQITPAEGPVVTAQYDCLGCVHPISTQSPDLEPI
LRHGIQYFNNNTQHSSLFMLNEVKRAQRQVVAGLNFRITYSIVQTNCSKENFLFLTPDCKSLWNGDTG
ECTDNAYIDIQLRIASFSQNCDIYPGKDFVQPPTKICVGCPRDIPTNSPELEETLTHTTTKLNAENNA
TFYFKIDNVKKARVQVVAGKKYFIDFVARETTCSKESNEELTESCETKKLGQSLDCNAEVYVVPWEKK
IYPTVNCQPLGMISLMKRPPGFSPFRSSRIGEIKEETTVSPPHTSMAPAQDEERDSGKEQGHTRRHDW
GHEKQRKHNLCHGHKHERDQGHGHQRGHGLGHGHEQQHGLGHGHKFKLDDDLEHQGGHVLDHGHKHKH
GHGHGKHKNKGKKNGKHNGWKTEHLASSSEDSTTPSAQTQEKTEGPTPIPSLAKPGVTVTFSDFQDSD
LIATMMPPISPAPIQSDDDWIPDIQIDPNGLSFNPISDFPDTTSPKCPGRPWKSVSEINPTTQMKESY
YFDLTDGLS

| | | |
|---|---|---|
| NOV1i, 311750024 | SEQ ID NO: 17 | 1866 bp |
| DNA Sequence | ORF Start: at 1 | ORF Stop: end of sequence |

TCTGAGGAAATTGACTGCAATGACAAGGATTTATTTAAAGCTGTGGATGCTGCTCTGAAGA
AATATAACAGTCAAAACCAAAGTAACAACCAGTTTGTATTGTACCGCATAACTGAAGCCACTAAGACC
GTTGGCTCTGACACGTTTTATTCCTTCAAGTACGAAATCAAGGAGGGGGATTGTCCTGTTCAAAGTGG
CAAAACCTGGCAGGACTGTGAGTACAAGGATGCTGCAAAAGCAGCCACTGGAGAATGCACGGCAACCG
TGGGGAAGAGGAGCAGTACGAAATTCTCCGTGGCTACCCAGACCTGCCAGATTACTCCAGCCGAGGGC
CCTGTCGTGACAGCCCAGTACGACTGCCTCGGCTGTGTGCATCCTATATCAACGCAGAGCCCAGACCT
GGAGCCCATTCTGAGACACGGCATTCAGTACTTTAACAACAACACTCAACATTCCTCCCTCTTCATGC
TTAATGAAGTAAAACGGGCCCAAAGACAGGTGGTGGCTGGATTGAACTTTCGAATTACCTACTCAATT
GTGCAAACCAATTGTTCCAAAGAGAATTTTCTGTTCTTAACTCCAGACTGCAAGTCCCTTTGGAATGG
TGATACCGGTGAATGTACAGATAATGCATACATCGATATTCAGCTACGAATTGCTTCCTTCTCACAGA
ACTGTGACATTTATCCAGGGAAGGATTTTGTACAACCACCTACCAAGATTTGCGTGGGCTGCCCCAGG
GATATACCCACCAACAGCCCAGAGCTGGAGGAGACACTGACTCACACCATCACAAAGCTTAATGCAGA
GAATAACGCAACTTTCTATTTCAAGATTGACAATGTGAAAAAAGCAAGAGTACAGGTGGTGGCTGGCA
AGAAATATTTTATTGACTTCGTGGCCAGGGAAACCACATGTTCCAAGGAAAGTAATGAAGAGTTGACC
GAAAGCTGTGAGACCAAAAAACTTGGCCAAAGCCTAGATTGCAACGCTGAAGTTTATGTGGTACCCTG
GGAGAAAAAAATTTACCCTACTGTCAACTGTCAACCACTGGGAATGATCTCACTGATGAAAAGGCCTC
CAGGTTTTTCACCTTTCCGATCATCACGAATAGGGGAAATAAAAGAAGAAACAACTGTAAGTCCACCC
CACACTTCCATGGCACCTGCACAAGATGAAGAGCGGGATTCAGGAAAAGAACAAGGGCATACTCGTAG
ACATGACTGGGGCCATGAAAAACAAAGAAAACATAATCTTGGCCATGGCCATAAACATGAACGTGACC
AAGGGCATGGGCACCAAAGAGGACATGGCCTTGGCCATGGACACGAACAACAGCATGGTCTTGGTCAT
GGACATAAGTTCAAACTTGATGATGATCTTGAACACCAAGGGGGCCATGTCCTTGACCATGGACATAA
GCATAAGCATGGTCATGGCCACGGAAAACATAAAAATAAAGGCAAAAAGAATGGAAAGCACAATGGTT
GGAAAACAGAGCATTTGGCAAGCTCTTCTGAAGACAGTACTACACCTTCTGCACAGACACAAGAGAAG
ACAGAAGGGCCAACACCCATCCCTTCCCTAGCCAAGCCAGGTGTAACAGTTACCTTTTCTGACTTTCA
GGACTCTGATCTCATTGCAACTATGATGCCTCCTATATCACCAGCTCCCATACAGAGTGATGACGATT
GGATCCCTGATATCCAGATAGACCCAAATGGCCTTTCATTTAACCCAATATCAGATTTTCCAGACACG
ACCTCCCCAAAATGTCCTGGACGCCCCTGGAAGTCAGTTAGTGAAATTAATCCAACCACACAAATGAA
AGAATCTTATTATTTCGATCTCACTGATGGCCTTTCT

| | | | |
|---|---|---|---|
| NOV1i, 311750024 | | | |
| Protein Sequence | SEQ ID NO: 18 | 622 aa | MW at 69424.4 kD |

SEEIDCNDKDLFKAVDAALKKYNSQNQSNNQFVLYRITEATKTVGSDTFYSFKYEIKEGDCPVQSG
KTWQDCEYKDAAKAATGECTATVGKRSSTKFSVATQTCQITPAEGPVVTAQYDCLGCVHPISTQSPDL
EPILRHGIQYFNNNTQHSSLFMLNEVKRAQRQVVAGLNFRITYSIVQTNCSKENFLFLTPDCKSLWNG
DTGECTDNAYIDIQLRIASFSQNCDIYPGKDFVQPPTKICVGCPRDIPTNSPELEETLTHTITKLNAE
NNATFYFKIDNVKKARVQVVAGKKYFIDFVARETTCSKESNEELTESCETKKLGQSLDCNAEVYVVPW
EKKIYPTVNCQPLGMISLMKRPPGFSPFRSSRIGEIKEETTVSPPHTSMAPAQDEERDSGKEQGHTRR
HDWGHEKQRKHNLGHGHKHERDQGHGHQRGHGLGHGHEQQHGLGHGHKFKLDDDLEHQGGHVLDHGHK
HKHGHGHGKHKNKGKKNGKHNGWKTEHLASSSEDSTTPSAQTQEKTEGPTPIPSLAKPGVTVTFSDFQ
DSDLIATMMPPISPAPIQSDDDWIPDIQIDPNGLSFNPISDFPDTTSPKCPGRPWKSVSEINPTTQMK
ESYYFDLTDGLS

| | | |
|---|---|---|
| NOV1j, CG104903-01 | SEQ ID NO: 19 | 1357 bp |
| DNA Sequence | ORF Start: ATG at 1 | ORF Stop: TGA at 1195 |

ATGAAACTAATTACCATCCTTTTCCTCTGCTCCAGGCTACTACTAAGTTTAACCCAGGAATCACAGTC
CGAGGAAATTGATGACTGCAATGACAAGGATTTATTTAAAGCTGTGGATGCTGCTCTGAAGAAATATA
ACAGTCAAAACCAAAGTAACAACCAGTTTGTATTGTACCGCAAAACCTGGCAGGACTGTGAGTACAAG
GATGCTGCAAAAGCAGCCACTGGAGAATGCACAGCAACCGTGGGGAAGAGGAGCAGTACGAAATTCTC
CGTGGCTACCCAGACCTGGCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCACGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATCAAAAGGCCTCCAGGTTTTTCACCTTTCCGATCATCACG
AATAGGGGAAATAAAAGAAGAAACAACTAGTCACCTAAGGTCCTGCGAGTACAAGGGTCGACCCCCAA
AGGCAGGGGCAGAGCCAGCATCTGAGAGGGAGGTCTCTTGACCAATGGGCAGAATCTTCACTCCAGGC
ACATAGCCCGAACCACCTCTGCCAGCAACCTTGAGAGGAAGGACAAGAAGAAAGATGCGATAGAATTT
AAATAGAGAAGAATGCCATTTTATCACTCTGCCTCTGGGTGAAATAAAGATCAGTCTTGATGTTC

| | | | |
|---|---|---|---|
| NOV1j, CG104903-01 | | | |
| Protein Sequence | SEQ ID NO: 20 | 398 aa | MW at 44684.1 kD |

MKLITILFLCSRLLLSLTQESQSEEIDDCNDKDLFKAVDAALKKYNSQNQSNNQFVLYRKTWQDCEYK
DAAKAATGECTATVGKRSSTKFSVATQTCQITPAEGPVVTAQYDCLGCVHPISTQSPDLEPILRHGIQ
YFNNNTQHSSLFTLNEVKRAQRQVVAGLNFRITYSIVQTNCSKENFLFLTPDCKSLWNGDTGECTDNA
YIDIQLRIASFSQNCDIYPGKDFVQPPTKICVGCPRDIPTNSPELEETLTHTITKLNAENNATFYFKI
DNVKKARVQVVAGKKYFIDFVARETTCSKESNEELTESCETKKLGQSLDCNAEVYVVPWEKKIYPTVN
CQPLGMISLMKRPPGFSPFRSSRIGEIKEETTSHLRSCEYKCRPPKAGAEPASEREVS

| | | |
|---|---|---|
| NOV1k, CG104903-02 | SEQ ID NO: 21 | 1848 bp |
| DNA Sequence | ORF Start: ATG at 1 | ORF Stop: TAA at 1846 |

ATGAAACTAATTACCATCCTTTTCCTCTGCTCCAGGCTACTACTAAGTTTAACCCAGGAATCACAGTC
CGAGGAAATTGATGACTGCAATGACAAGGATTTATTTAAAGCTGTCGATGCTGCTCTGAAGAAATATA
ACAGTCAAAACCAAAGTAACAACCAGTTTGTATTGTACCGCAAAACCTGGCAGGACTGTGAGTACAAG
GATGCTGCAAAAGCACCCACTGGAGAATGCACAGCAACCGTGGGGAAGAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCACGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTCACATTTATCCAGGGAAGGATTT
TGTACAACCACCTAGCAAGATTTGCGTGGGCTGCCCCAGAGATATACCCACCAACAGCCCAGAGCTGG
AGGAGACACTGACTCACACCATCAACAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAACAATGGAAAGCACAATGGTTGGAAAACAGAGCATTTGGCAACCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTCGATCCCTGATATCCAGACAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCTTAA

| | | | |
|---|---|---|---|
| NOV1k, CG104903-02 | | | |
| Protein Sequence | SEQ ID NO: 22 | 615 aa | MW at 68746.1 kD |

MKLITILFLCSRLLLSLTQESQSEEIDDCNDKDLFKAVDAALKKYNSQNQSNNQFVLYRKTWQDCEYK
DAAKAATGECTATVGKRSSTKFSVATQTCQITPAEGPVVTAQYDCLGCVHPISTQSPDLEPILRHGIQ
YFNNNTQHSSLFTLNEVKRAQRQVVAGLNFRITYSIVQTNCSKENFLFLTPDCKSLWNGDTGECTDNA
YIDIQLRIASFSQNCDIYPGKDFVQPPTKICVGCPRDIPTNSPELEETLTHTITKLNAENNATFYFKI
DNVKKARVQVVAGKKYFIDFVARETTCSKESNEELTESCETKKLGQSLDCNAEVYVVPWEKKIYPTVN
CQPLGMISLMKRPPGFSPFRSSRIGEIKEETTVSPPHTSMAPAQDEERDSGKEQGHTRRHDWGHEKQR
KHNLGHGHKHERDQGHGHQRGHGLGHGHEQQHGLGHGHKFKLDDDLEHQGGHVLDHGHKHKHGHGHGK
HKNKGKKNGKHNGWKTEHLASSSEDSTTPSAQTQEKTEGPTPIPSLAKPGVTVTFSDFQDSDLIATMM
PPISPAPIQSDDDWIPDIQTDPNGLSFNPISDFPDTTSPKCPGRPWKSVSEINPTTQMKESYYFDLTD
GLS

| | | |
|---|---|---|
| NOV1l, CG104903-04 | SEQ ID NO: 23 | 1380 bp |
| DNA Sequence | ORF Start: ATG at 3 | ORF Stop: TGA at 1284 |

TCATGAAACTAATTACCATCCTTTTCCTCTGCTCCAGGCTGCTACTAAGTTTAACCCAGGAATCACAG
TCCGAGGAAATTGACTGCAATGACAAGGATTTATTTAAAGCTGTGGATGCTGCTCTGAAGAAATATAA
CAGTCAAAACCAAAGTAACAACCAGTTTGTATTGTACCGCATAACTGAAGCCACTAAGACGGTTGGCT
CTGACACGTTTTATTCCTTCAAGTACGAAATCAAGGAGGGGGATTGTCCTGTTCAAAGTGGCAAAACC
TGGCAGGACTGTGAGTACAAGGATGCTGCAAAAGCAGCCACTGGAGAATGCACGGCAACCGTGGGGAA
GAGGAGCAGTACGAAATTCTCCGTGGCTACCCAGACCTGCCAGATTACTCCAGCCGAGGGCCCTGTGG
TGACAGCCCAGTACGACTGCCTCCGCTGTGTGCATCCTATATCAACGCAGAGCCCAGACCTGGAGCCC
ATTCTGAGACACGGCATTCAGTACTTTAACAACAACACTCAACATTCCTCCCTCTTCATGCTTAATGA
AGTAAAACGGGCCCAAAGACAGGTGGTGGCTGGATTGAACTTTCGAATTACCTACTCAATTGTGCAAA
CGAATTGTTCCAAAGAGAATTTTCTGTTCTTAACTCCAGACTGCAAGTCCCTTTGGAATGGTGATACC
GGTGAATGTACAGATAATGCATACATCGATATTCAGCTACGAATTGCTTCCTTCTCACAGAACTGTGA
CATTTATCCAGGGAAGGATTTTGTACAACCACCTACCAAGATTTGCGTGGGCTGCCCCAGAGATATAC
CCACCAACAGCCCAGAGCTGGAGGAGACACTGACTCACACCATCACAAAGCTTAATGCAGAGAATAAC
GCAACTTTCTATTTCAAGATTGACAATGTGAAAAAAGCAAGAGTACAGGTGGTGGCTGGCAAGAAATA
TTTTATTGACTTCGTGGCCAGGGAAACCACATGTTCCAAGGAAAGTAATGAAGAGTTGACCGAAAGCT
GTGAGACCAAAAAACTTGGCCAAAGCCTAGATTGCAACGCTGAAGTTTATGTGGTACCCTGGGAGAAA
AAAATTTACCCTACTGTCAACTGTCAACCACTGGGAATGATCTCACTGATGAAAAGGCCTCCAGGTTT
TTCACCTTTCCGATCATCACGAATAGGGGAAATAAAAGAAGAAACAACTAGTCACCTAAGGTCCTGCG
AGTACAAGGGTCGACCCCCAAAGGCAGGGGCAGAGCCAGCATCTGAGAGGGAGGTCTCTTGACCAATC
GGCAGAATCTTCACTCCAGGCACATAGCCCCAACCACCTCTGCCAGCAACCTTGAGAGGAAGGACAAG
AAGAAAGATGGGATAGAATT

| | | | |
|---|---|---|---|
| NOV1l, CG104903-04 | | | |
| Protein Sequence | SEQ ID NO: 24 | 427 aa | MW at 47882.7 kD |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPCKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTSHLRSCE
YKGRPPKAGAEPASEREVS

| | | |
|---|---|---|
| NOV1m, CG104903-05 | SEQ ID NO: 25 | 1297 bp |
| DNA Sequence | ORF Start: ATG at 50 | ORF Stop: TAA at 1295 |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTAGCGCATAACTGAAGCCACTAAGACGGTTGGCTCTGACACGTTTTATTGCTTCA
AGTACGAAATCAAGGAGGGGGATTGTCCTGTTCAAAGTCGCAAAACCTGGCAGGACTGTGAGTACAAG
GATGCTGCAAAAGCAGCCACTGGAGAATGCACAGCAACCGTGGGGAAGAGGAGCACTACGAAATTCTC
CGTGGCTACCCAGACCTGCCAGATTACTCCAGCCGAGGGCCCTCTGGTGACAGCCCAGTACGACTGCC
TCGGCTGTGTGCATCCTATATCAACGCAGAGCCCAGGTTTTTCACCTTTCCGATCATCACGAATAGGG
GAAATAAAAGAAGAAACAACTGTAAGTCCACCCCACACTTCCATGGCACCTGCACAAGATGAAGAGCG
GGATTCAGGAAAAGAACAAGGGCATACTCGTAGACATGACTGGGGCCATGAAAAACAAAGAAAACATA
ATCTTGGCCATGGCCATAAACATGAACGTGACCAAGGGCATGGGCACCAAAGAGGACATGGCCTTGGC
CATGGACACGAACAACAGCATGGTCTTGGTCATGGACATAAGTTCAAACTTGATGATGATCTTGAACA
CCAAGGGGGCCATGTCCTTGACCATGGACATAAGCATAAGCATGGTCATGGCCACGGAAAACATAAAA
ATAAAGGCAAAAAGAATGGAAAGCACAATGGTTGGAAAACAGAGCATTTGGCAAGCTCTTCTGAAGAC
AGTACTACACCTTCTGCACAGACACAAGAGAAGACAGAAGCGCCAACACCCATCCCTTCCCTAGCCAA
GCCAGGTGTAACAGTTACCTTTTCTGACTTTCAGGACTCTGATCTCATTGCAACTATGATGCCTCCTA
TATCACCAGCTCCCATACAGAGTGATGACGATTGGATCCCTGATATCCAGATAGACCCAAATCGCCTT
TCATTTAACCCAATATCAGATTTTCCAGACACGACCTCCCCAAAATGTCCTGGACGCCCCTGGAAGTC
AGTTAGTGAAATTAATCCAACCACACAAATGAAAGAATCTTATTATTTCGATCTCACTGATGGCCTTT
CTTAA

| | | | |
|---|---|---|---|
| NOV1m, CG104903-05 | | | |
| Protein Sequence | SEQ ID NO: 26 | 415 aa | MW at 45897.3 kD |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPGFSPFRSSRIGEIKEETTVSPPHTSMAPAQDEERDSGKEQGHTRRHDWGH
EKQRKHNLGHGHKHERDQGHGHQRGHGLGHGHEQQHGLGHGHKFKLDDDLEHQGGHVLDHGHKHKHGH
GHGKHKNKGKKNGKHNGWKTEHLASSSEDSTTPSAQTQEKTEGPTPIPSLAKPGVTVTFSDFQDSDLI
ATMMPPISPAPIQSDDDWTPDIQIDPNGLSFNPISDFPDTTSPKCPGRPWKSVSEINPTTQMKESYYF
DLTDGLS

| | | |
|---|---|---|
| NOV1n, CG104903-06 | SEQ ID NO: 27 | 1892 bp |
| DNA Sequence | ORF Start: ATG at 50 | ORF Stop: TAA at 458 |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAC
GATGCTGCAAAAGCAGCCACTGGAGAATGCACAGCAACCGTGGGAAGAGGAGCAGTACGAAATTCTCC
GTGGCTACCCAGACCTGGAGCCCATTCTGAGACACGGCATTCAGTACTTTAACAACAACACTCAACAT
TCCTCCCTCTTCACGCTTAATGAAGTAAAACGGGCCCAAAGACAGGTGGTGGCTGGATTGAACTTTCG
AATTACCTACTCAATTGTGCAAACGAATTGTTCCAAAGAGAATTTTCTGTTCTTAACTCCAGACTGCA
AGTCCCTTTGGAATGGTGATACCGGTGAATGTACAGATAATGCATACATCGATATTCAGCTACGAATT
GCTTCCTTCTCACAGAACTGTGACATTTATCCAGGGAAGGATTTTGTACAACCACCTACCAAGATTTG
CGTGGGCTGCCCCAGAGATATACCCACCAACAGCCCAGAGCTGGAGGAGACACTGACTCACACCATCA
CAAAGCTTAATGCAGAGAATAACGCAACTTTCTATTTCAAGATTGACAATGTGAAAAAAGCAAGACTA
CAGGTGGTGGCTGGCAAGAAATATTTTATTGACTTCGTGGCCAGGGAAACCACATGTTCCAAGGAAAG
TAATGAACAGTTGACCCAAAGCTGTGAGACCAAAAAACTTGGCCAAAGCCTAGATTGCAACGCTGAAG
TTTATGTGGTACCCTGGGAGAAAAAAATTTACCCTACTGTCAACTGTCAACCACTGGGAATGATCTCA
CTGATGAAAAGGCCTCCAGGTTTTTCACCTTTCCGATCATCACGAATAGGGGAAATAAAAGAAGAAAC
AACTGTAAGTCCACCCCACACTTCCATGGCACCTGCACAAGATGAAGAGCGGGATTCAGGAAAAGAAC
AAGGGCATACTCGTAGACATGACTGGGGCCATGAAAAACAAAGAAAACATAATCTTGGCCATGGCCAT
AAACATGAACGTCACCAAGGGCATGGGCACCAAAGAGGACATGGCCTTGGCCATGGACACGAACAACA
GCATGGTCTTGGTCATGGACATAAGTTCAAACTTGATGATGATCTTGAACACCAAGGGGGCCATGTCC
TTGACCATGGACATAAGCATAAGCATGGTCATGGCCACGGAAAACATAAAAATAAAGGCAAAAAGAAT
GGAAAGCACAATGGTTGGAAAACAGAGCATTTGGCAAGCTCTTCTGAAGACAGTACTACACCTTCTGC
ACAGACACAAGAGAAGACAGAAGGGCCAACACCCATCCCTTCCCTAGCCAAGCCAGGTGTAACAGTTA
CCTTTTCTGACTTTCAGGACTCTGATCTCATTGCAACTATGATGCCTCCTATATCACCAGCTCCCATA
CAGAGTGATGACGATTGGATCCCTGATATCCAGATAGACCCAAATGGCCTTTCATTTAACCCAATATC
AGATTTTCCAGACACGACCTCCCCAAAATGTCCTGGACGCCCCTGGAAGTCACTTAGTGAAATTAATC
CAACCACACAAATGAAAGAATCTTATTATTTCGATCTCACTGATGGCCTTTCTTAA

| | | | |
|---|---|---|---|
| NOV1n, CG104903-06 | | | |
| Protein Sequence | SEQ ID NO: 28 | 136 aa | MW at 15218.9 kD |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGRGAVRNSPWLPRPGAHSETRHSVL

| | | |
|---|---|---|
| NOV1o, CG104903-07 | SEQ ID NO: 29 | 670 bp |
| DNA Sequence | ORF Start: ATG at 1 | ORF Stop: TGA at 508 |

ATGAAACTAATTACCATCCTTTTCCTCTGCTCCAGGCTACTACTAAGTTTAACCCAGGAATCACAGTC
CGAGGAAATTGATGACTGCAATGACAAGCATTTATTTAAAGCTGTGGATGCTGCTCTGAAGAAATATA
ACAGTCAAAACCAAAGTAACAACCAGTTTGTATTGTACCGCAAAACCTGGCAGGACTGTGAGTACAAG
GATGCTGCAAAAGCAGCCACTGGAGAATGCACAGCAACCGTGGGGAACAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGGTTTTTCACCTTTCCGATCATCACGAATAGGG
GAAATAAAAGAAGAAACAACTAGTCACCTAAGGTCCTGCGAGTACAAGGGTCGACCCCCAAAGGCAGG
GGCAGAGCCAGCATCTGAGAGGCAGGTCTCTTGACCAATGGGCAGAATCTTCACTCCAGGCACATAGC
CCCAACCACCTCTGCCAGCAACCTTGAGAGGAAGGACAAGAAGAAAGATGGGATAGAATTTAAATAGA
GAAGAATGCCATTTTATCACTCTGCCTCTGGGTGAAATAAAGATCAGTCTTGATGTTC

| | | | |
|---|---|---|---|
| NOV1o, CG104903-07 | | | |
| Protein Sequence | SEQ ID NO: 30 | 169 aa | MW at 18654.7 kD |

MKLITILFLCSRLLLSLTQESQSEEIDDCNDKDLFKAVDAALKKYNSQNQSNNQFVLYRKTWQDCEYK
DAAKAATGECTATVGKRSSTKFSVATQTCQITPAEGPVVTAQYDCLGCVHPISTQSPGFSPFRSSRIG
EIKEETTSHLRSCEYKGRPPKAGAEPASEREVS

| | | |
|---|---|---|
| NOV1p, CG104903-08 | SEQ ID NO: 31 | 1193 bp |
| DNA Sequence | ORF Start: ATG at 1 | ORF Stop: TGA at 1171 |

ATGAAACTAATTACCATCCTTTTCCTCTGCTCCAGGCTACTACTAAGTTTAACCCAGGAATCACAGTC
CGAGGAAATTGACTGCAATGACAAGGATTTATTTAAAGCTGTGGATGCTGCTCTGAAGAAATATAACA
GTCAAAACCAAAGTAACAACCAGTTTGTATTGTACCGCATAACTGAAGCCACTAAGACGGCCACTGGA
GAATGCACGGCAACCGTGGGGAAGAGGAGCAGTACGAAATTCTCCGTGGCTACCCAGACCTGCCAGAT
TACTCCAGCCGAGGGCCCTGTGGTGACAGCCCAGTACGACTGCCTCGGCTGTGTGCATCCTATATCAA
CGCAGAGCCCAGACCTGCAGCCCATTCTGAGACACGGCATTCAGTACTTTAACAACAACACTCAACAT
TCCTCCCTCTTCACGCTTAATGAAGTAAAACGGGCCCAAAGACACGTGGTGGCTGGATTGAACTTTCG
AATTACCTACTCAATTGTGCAAACGAATTGTTCCAAAGAGAATTTTCTGTTCTTAACTCCAGACTGCG
AGTCCCTTTGGAATGGTGATACCGGTGAATGTACAGATAATGCATACATCGATATTCAGCTACGAATT
GCTTCCTTCTCACAGAACTGTGACATTTATCCAGGGAAGGATTTTGTACAACCACCTACCAAGATTTG
CGTGGGCTGCCCCAGAGATATACCCACCAACAGCCCAGAGCTGGAGGAGACACTGACTCACACCATCA
CAAAGCTTAATGCAGAGAATAACGCAACTTTCTATTTCAAGATTGACAATGTGAAAAAAGCAAGAGTA
CAGGTGGTGGCTCGCAAGAAATATTTTATTGACTTCGTGGCCAGGGAAACCACATGTTCCAAGGAAAG
TAATGAAGAGTTGACCGAAAGCTGTGAGACCAAAAAACTTGGCCAAAGCCTAGATTGCAACGCTGAAG
TTTATGTGGTACCCTGGGAGAAAAAAATTTACCCTACTGTCAACTGTCAACCACTGGGAATGATCTCA
CTGATGAAAAGGCCTCCAGGTTTTTCACCTTTCCGATCATCACGAATAGGGGAAATAAAAGAAGAAAC
AACTAGTCACCTAAGGTCCTGCGAGTACAAGGGTCGACCCCCAAAGGCAGGCGCAGAGCCAGTATCTG
AGAGGGAGGTCTCTTGACCAATGGGCAGAATCTTCAC

| | | | |
|---|---|---|---|
| NOV1p, CG104903-08 | | | |
| Protein Sequence | SEQ ID NO: 32 | 390 aa | MW at 43704.0 kD |

MKLITILFLCSRLLLSLTQESQSEEIDCNDKDLFKAVDAALKKYNSQNQSNNQFVLYRITEATKTATG
ECTATVGKRSSTKFSVATQTCQITPAEGPVVTAQYDCLGCVHPISTQSPDLEPILRHGIQYFNNNTQH
SSLFTLNEVKRAQRQVVAGLNFRITYSIVQTNCSKENFLFLTPDCESLWNGDTGECTDNAYIDIQLRI
ASFSQNCDIYPGKDFVQPPTKICVGCPRDTPTNSPELEETLTHTITKLNAENNATFYFKIDNVKKARV
QVVAGKKYFIDFVARETTCSKESNEELTESCETKKLGQSLDCNAEVYVVPWEKKIYPTVNCQPLGMIS
LMKRPPGFSPFRSSRIGEIKEETTSHLRSCEYKGRPPKAGAEPVSEREVS

| | | |
|---|---|---|
| NOV1q, CG104903-11 | SEQ ID NO: 33 | 48 bp |
| DNA Sequence | ORF Start: at 1 | ORF Stop: end of sequence |

CATAAAAATAAAGGCAAAAAGAATGGAAAGCACAATGGTTGGAAAACA

| | | | |
|---|---|---|---|
| NOV1q, CG104903-11 | | | |
| Protein Sequence | SEQ ID NO: 34 | 16 aa | MW at 1862.2 kD |

| | |
|---|---|
| HKNKGKKNGKHNGWKT | +TZ,46 |

| | | |
|---|---|---|
| NOV1r, CG104903-12 | SEQ ID NO: 35 | 48 bp |
| DNA Sequence | ORF Start: at 1 | ORF Stop: end of sequence |

AAGCATGGTCATGGCCACGGAAAACATAAAAATAAAGGCAAAAAGAAT

| | | | |
|---|---|---|---|
| NOV1r, CG104903-12 | | | |
| Protein Sequence | SEQ ID NO: 36 | 16 aa | MW at 1792.1 kD |

KHGHGHGKHKNKGKKN

| | | |
|---|---|---|
| NOV1s, CG104903-13 | SEQ ID NO: 37 | 48 bp |
| DNA Sequence | ORF Start: at 1 | ORF Stop: end of sequence |

GTCCTTGACCATGGACATAAGCATAAGCATGGTCATGGCCACGGAAAA

| NOV1s, CG104903-13 | | | |
|---|---|---|---|
| Protein Sequence | SEQ ID NO: 38 | 16 aa | MW at 1781.0 kD |
| VLDHGHKHKHGHGHGK | | | |

| | | |
|---|---|---|
| NOV1t, CG104903-14 | SEQ ID NO: 39 | 48 bp |
| DNA Sequence | ORF Start: at 1 | ORF Stop: end of sequence |

GGACATGGCCTTGGCCATGGACACGAACAACAGCATGGTCTTGGTCAT

| | | | |
|---|---|---|---|
| NOV1t, CG104903-14 | | | |
| Protein Sequence | SEQ ID NO: 40 | 16 aa | MW at 1657.8 kD |

GHGLGHGHEQQHGLGH

| | | |
|---|---|---|
| NOV1u, CG104903-15 | SEQ ID NO: 41 | 48 bp |
| DNA Sequence | ORF Start: at 1 | ORF Stop: end of sequence |

GACCAAGGGCATGGGCACCAAAGAGGACATGGCCTTGGCCATGGACAC

| | | | |
|---|---|---|---|
| NOV1u, CG104903-15 | | | |
| Protein Sequence | SEQ ID NO: 42 | 16 aa | MW at 1686.8 kD |

DQGHGHQRGHGLGHGH

| | | |
|---|---|---|
| NOV1v, CG104903-16 | SEQ ID NO: 43 | 1863 bp |
| DNA Sequence | ORF Start: at 1 | ORF Stop: at 1863 |

GAGGAATTGACTGCAATGACAAGGATTTATTTAAAGCTGTGGATGCTGCTCTGAAGA
AATATAACAGTCAAAACCAAAGTAACAACCAGTTTGTATTGTACCGCATAACTGAAGCCACTAAGACG
GTTGGCTCTGACACGTTTTATTCCTTCAAGTACGAAATCAAGGAGGGGGATTGTCCTGTTCAAAGTGG
CAAAACCTGGCAGGACTGTGAGTACAAGGATGCTGCAAAAGCAGCCACTGGAGAATGCACGGCAACCG
TGGGGAAGAGGAGCAGTACGAAATTCTCCGTGGCTACCCAGACCTGCCAGATTACTCCAGCCGAGGGC
CCTGTGGTGACAGCCCAGTACGACTGCCTCGGCTGTGTGCATCCTATATCAACGCAGAGCCCAGACCT
GGAGCCCATTCTGAGACACGGCATTCAGTACTTTAACAACAACACTCAACATTCCTCCCTCTTCATGC
TTAATGAAGTAAAACGGGCCCAAAGACAGGTGGTGGCTGGATTGAACTTTCGAATTACCTACTCAATT
GTGCAAACGAATTGTTCCAAAGAGAATTTTCTGTTCTTAACTCCAGACTGCAAGTCCCTTTGGAATGG
TGATACCGGTGAATGTACAGATAATGCATACATCGATATTCAGCTACGAATTGCTTCCTTCTCACAGA
ACTCTGACATTTATCCAGGGAAGGATTTTGTACAACCACCTACCAAGATTTGCGTGGGCTGCCCCAGG
GATATACCCACCAACAGCCCAGAGCTGGAGGAGACACTGACTCACACCATCACAAAGCTTAATGCAGA
GAATAACGCAACTTTCTATTTCAAGATTGACAATGTGAAAAAAGCAAGAGTACAGCTGGTGGCTGGCA
AGAAATATTTTATTGACTTCGTGGCCAGGGAAACCACATGTTCCAAGGAAAGTAATGAAGACTTGACC
GAAAGCTGTGAGACCAAAAAACTTGGCCAAAGCCTAGATTGCAACGCTGAAGTTTATGTGGTACCCTG
GGAGAAAAAAATTTACCCTACTGTCAACTGTCAACCACTGGCAATGATCTCACTGATGAAAAGGCCTC
CAGGTTTTTCACCTTTCCCATCATCACGAATAGCGGAAATAAAAGAAGAAACAACTGTAAGTCCACCC
CACACTTCCATGGCACCTGCACAACATGAAGAGCGGGATTCAGGAAAAGAACAAGGGCATACTCGTAG
ACATGACTGGGGCCATGAAAAACAAAGAAAACATAATCTTGGCCATGGCCATAAACATGAACGTGACC
AAGGGCATGGGCACCAAAGAGGACATGGCCTTGGCCATGGACACGAACAACAGCATGGTCTTGGTCAT
GGACATAAGTTCAAACTTGATGATGATCTTGAACACCAAGGGGGCCATGTCCTTGACCATGGACATAA
GCATAAGCATGGTCATGGCCACGGAAAACATAAAAATAAAGGCAAAAAGAATGGAAAGCACAATGGTT
GGAAAACAGAGCATTTGGCAAGCTCTTCTGAAGACAGTACTACACCTTCTGCACAGACACAAGAGAAG
ACAGAAGGGCCAACACCCATCCCTTCCCTAGCCAAGCCAGGTGTAACAGTTACCTTTTCTGACTTTCA
GGACTCTGATCTCATTGCAACTATGATGCCTCCTATATCACCAGCTCCCATACAGAGTGATGACGATT
GGATCCCTGATATCCAGATAGACCCAAATGGCCTTTCATTTAACCCAATATCAGATTTTCCAGACACG
ACCTCCCCAAAATGTCCTGGACGCCCCTGGAAGTCAGTTAGTGAAATTAATCCAACCACACAAATGAA
AGAATCTTATTATTTCGATCTCACTGATGGCCTTTCT

| | | | |
|---|---|---|---|
| NOV1v, CG104903-16 | | | |
| Protein Sequence | SEQ ID NO: 44 | 621 aa | MW at 69336.6 kD |

EEIDCNDKDLFKAVDAALKKYNSQNQSNNQFVLYRITEATKTVGSDTFYSFKYETKEGDCPVQSGKTW
QDCEYKDAAKAATGECTATVGKRSSTKFSVATQTCQITPAEGPVVTAQYDCLGCVHPISTQSPDLEPI
LRHGIQYFNNNTQHSSLFMLNEVKRAQRQVVAGLNFRITYSIVQTNCSKENFLFLTPDCKSLWNGDTG
ECTDNAYIDIQLRIASFSQNCDIYPGKDFVQPPTKICVGCPRDIPTNSPELEETLTHTITKLNAENNA
TFYFKIDNVKKARVQWAGKKYFIDFVARETTCSKESNEELTESCETKKLGQSLDCNAEVYVVNPWEKK
IYPTVNCQPLGMISLMKRPPGFSPFRSSRIGEIKEETTVSPPHTSMAPAQDEERDSGKEQGHTRRHDW
GHEKQRKHNLGHGHKHERDQGHGHQRGHGLGHGHEQQHGLGHCHKFKLDDDLEHQGGHVLDHGHKHKH
GHGHGKHKNKGKKNGKHNGWKTEHLASSSEDSTTPSAQTQEKTEGPTPIPSLAKPGVTVTFSDFQDSD
LIATMMPPISPAPIQSDDDWIPDIQIDPNGLSFNPISDFPDTTSPKCPGRPWKSVSETNPTTQMKESY
YFDLTDGLS

| | | |
|---|---|---|
| NOV1w, CG104903-17 | SEQ ID NO: 45 | 1071 bp |
| DNA Sequence | ORF Start: ATG at 1 | ORF Stop: at 1071 |

ATGAAACTAATTACCATCCTTTTCCTCTGCTCCAGGCTACTACTAAGTTTAA
CCCAGGAATCACAGTCCGAGGAAATTGACTGCAATGACAAGGATTTATTTAAAGCTGTGGATGCTGCT
CTGAAGAAATATAACAGTCAAAACCAAAGTAACAACCAGTTTGTATTGTACCGCATAACTGAAGCCAC
TAAGACGGTTGGCTCTGACACGTTTTATTCCTTCAAGTACGAAATCAAGGAGGGGGATTCTCCTGTTC
AAAGTGGCAAAACCTGGCAGGACTGTGAGTACAAGGATGCTGCAAAAGCAGCCACTGGAGAATGCACG
GCAACCGTGCGGAAGAGGAGCAGTACGAAATTCTCCGTGGCTACCCAGACCTGCCAGATTACTCCAGC
CGAGGGCCCTGTGGTGACAGCCCAGTACGACTGCCTCGGCTGTGTGCATCCTATATCAACGCAGAGCC
CAGACCTGGAGCCCATTCTGAGACACGGCATTCAGTACTTTAACAACAACACTCAACATTCCTCCCTC
TTCATGCTTAATGAAGTAAAACGGGCCCAAAGACAGGTGGTGGCTGGATTGAACTTTCGAATTACCTA
CTCAATTGTGCAAACGAATTGTTCCAAAGAGAATTTTCTGTTCTTAACTCCAGACTGCAAGTCCCTTT
GGAATGGTGATACCGGTGAATGTACAGATAATGCATACATCGATATTCAGCTACGAATTGCTTCCTTC
TCACAGAACTGTGACATTTATCCAGGGAAGGATTTTGTACAACCACCTACCAAGATTTGCGTCGGCTG
CCCCAGGGATATACCCACCAACAGCCCAGAGCTGGAGGAGACACTGACTCACACCATCACAAAGCTTA
ATGCAGAGAATAACGCAACTTTCTATTTCAAGATTGACAATGTGAAAAAAGCAAGAGTACAGGTGGTG
GCTGGCAAGAAATATTTTATTGACTTCGTGGCCAGGGAAACCACATGTTCCAAGGAAAGTAATGAAGA
GTTGACCGAAAGCTGTGACACCAAAAAACTTGGCCAAAGCCTAGATTGCAACGCTGAAGTTTATGTG

| | | | |
|---|---|---|---|
| NOV1w, CG104903-17 | | | |
| Protein Sequence | SEQ ID NO: 46 | 357 aa | MW at 40026.7 kD |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRTASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYV

| | | |
|---|---|---|
| NOV1x, CG104903-18 | SEQ ID NO: 47 | 327 bp |
| DNA Sequence | ORF Start: at 1 | ORF Stop: at 327 |

GCACCTGCACAAGATGAAGAGCGGGATTCAGGAAAAGAACAAGGGCATACTCGTAGAC
ATGACTGGGGCCATGAAAAACAAAGAAAACATAATCTTGGCCATGGCCATAAACATGAACGTCACCAA
GGGCATGGGCACCAAAGAGGACATGGCCTTGGCCATGGACACGAACAACAGCATGGTCTTGGTCATGG
ACATAAGTTCAAACTTGATGATGATCTTGAACACCAAGGGGGCCATGTCCTTGACCATGGACATAAGC
ATAAGCATGGTCATGGCCACGGAAAACATAAAAATAAAGGCAAAAAGAATGGAAAGCACAATGGT

| | | | |
|---|---|---|---|
| NOV1x, CG104903-18 | | | |
| Protein Sequence | SEQ ID NO: 48 | 109 aa | MW at 12335.3 kD |

APAQDEERDSGKEQGHTRRHDWGHEKQRKHNLGHGHKHERDQGHGHQRGHGLGHGHEQQHGLCHGHKF
KLDDDLEHQGGHVLDHGHKHKHGHGHGKHKNKGKKNGKHNG

| | | |
|---|---|---|
| NOV1y, CG104903-19 | SEQ ID NO: 49 | 729 bp |
| DNA Sequence | ORF Start: at 1 | ORF Stop: at 729 |

GGTTTTTCACCTTTCCGATCATCACGAATAGGGGAAATAAAAGAAGAAACAACTGTAA
GTCCACCCCACACTTCCATGGCACCTGCACAAGATGAAGAGCGGGATTCAGGAAAAGAACAAGGGCAT
ACTCGTAGACATGACTGGGGCCATGAAAAACAAAGAAAACATAATCTTGGCCATGGCCATAAACATGA
ACGTGACCAAGGGCATGGGCACCAAAGAGGACATGGCCTTGGCCATGGACACGAACAACAGCATGGTC
TTGGTCATGGACATAAGTTCAAACTTGATGATGATCTTGAACACCAAGGGGGCCATGTCCTTGACCAT
GGACATAAGCATAAGCATGGTCATGGCCACGGAAAACATAAAAATAAAGGCAAAAAGAATGGAAAGCA
CAATGGTTGGAAAACAGAGCATTTGGCAAGCTCTTCTGAAGACAGTACTACACCTTCTGCACAGACAC
AACAGAAGACAGAAGGGCCAACACCCATCCCTTCCCTAGCCAAGCCAGGTGTAACAGTTACCTTTTCT
GACTTTCAGGACTCTGATCTCATTGCAACTATGATGCCTCCTATATCACCAGCTCCCATACAGAGTGA
TGACGATTGGATCCCTGATATCCAGATAGACCCAAATGGCCTTTCATTTAACCCAATATCAGATTTTC
CAGACACGACCTCCCCAAAATGTCCTGGACGCCCCTGGAAGTCACTTAGTGAAATTAAT

| | | | |
|---|---|---|---|
| NOV1y, CG104903-19 | | | |
| Protein Sequence | SEQ ID NO: 50 | 243 aa | MW at 26861.2 kD |

GFSPFRSSRIGEIKEETTVSPPHTSMAPAQDEERDSGKEQGHTRRHDWGHEKQRKHNLGHGHKHERDQ
GHGHQRGHGLGHGHEQQHGLGHGHKFKLDDDLEHQGGHVLDHGHKHKHGHGHGKHKNKGKKNGKHNGW
KTEHLASSSEDSTTPSAQTQEKTEGPTPIPSLAKPGVTVTFSDFQDSDLIATMMPPISPAPIQSDDDW
IPDIQIDPNGLSFNPISDFPDTTSPKCPGRPWKSVSEIN

| | | |
|---|---|---|
| NOV1z, CG104903-20 | SEQ ID NO: 51 | 1935 bp |
| DNA Sequence | ORF Start: ATG at 1 | ORF Stop: TAG at 1935 |

ATGAAACTAATTACCATCCTTTTCCTCTGCTCCAGGCTACTACTAAGTTTAAC
CCAGGAATCACAGTCCGAGGAAATTGACTCCAATGACAAGGATTTATTTAAAGCTGTGGATCCTGCTC
TGAAGAAATATAACAGTCAAAACCAAAGTAACAACCAGTTTGTATTGTACCGCATAACTGAAGCCACT
AAGACGGTTGGCTCTGACACGTTTTATTCCTTCAAGTACGAAATCAAGGAGGGGGACTGTCCTGTTCA
AAGTGGCAAAACCTGGCAGGACTGTGAGTACAAGGATGCAGCAAAAGCAGCCACTGGAGAATGCACGG
CAACCGTGGGGAAGAGGAGCAGTACGAAATTCTCCGTGGCTACCCAGACCTGCCAGATTACTCCAGCC
GAGGGCCCTGTGGTGACAGCCCAGTACGACTGCCTCGGCTGTGTGCATCCTATATCAACGCAGAGCCC
AGACCTGGAGCCCATTCTGAGACACGGCATTCAGTACTTTAACAACAACACTCAACATTCCTCCCTCT
TCACGCTTAATGAAGTAAAACGGGCCCAAAGACAGGTGGTGGCTGGATTGAACTTTCGAATTACCTAC
TCAATTGTGCAAACGAATTGTTCCAAAGAGAATTTTGTGTTCTTAACTCCAGACTGCAAGTCCCTTTG
GAATGGTGATACCGGTGAATGTACAGATAATGCATACATCGATATTCAGCTACGAATTGCTTCCTTCT
CACAGAACTGTGACATTTATCCAGGGAAGGATTTTGTACAACCACCTACCAAGATTTGCGTGGGCTGC
CCCAGAGATATACCCACCAACAGCCCAGAGCTGGAGGAGACACTGACTCACACCATCACAAAGCTTAA
TCCAGAGAATAACGCAACTTTCTATTTCAAGATTGACAATGTGAAAAAAGCAAGAGTACAGGTGGTGG
CTGGCAAGAAATATTTTATTGACTTCGTGGCCAGGGAAACCACATGTTCCAAGGAAAGTAATGAAGAG
TTGACCGAAAGCTGTGAGACCAAAAAACTTGGCCAAAGCCTAGATTGCAACGCTGAAGTTTATGTCGT
ACCCTGGGAGAAAAAAATTTACCCTACTGTCAACTGTCAACCACTGGGAATGATCTCACTGATGAAAA
GGCCTCCAGGTTTTTCACCTTTCCGATCATCACGAATAGGGGAAATAAAAGAAGAAACAACTGTAAGT
CCACCCCACACTTCCATGGCACCTGCACAAGATGAAGAGCGGGATTCAGGAAAAGAACAAGGGCATAC
TCGTAGACATGACTGGGGCCATGAAAAACAAAGAAAACATAATCTTGGCCATGGCCATAAACATGAAC
GTGACCAAGGGCATGGGCACCAAAGAGGACATGGCCTTGGCCATGGACACGAACAACAGCATGGTCTT
GGTCATGGACATAAGTTCAAACTTGATGATGATCTTGAACACCAAGGCGGCCATGTCCTTGACCATGG
ACATAAGCATAAGCATGGTCATGGCCACGGAAAACATAAAAATAAAGGCAAAAAGAATGGAAAGCACA
ATGGTTGGAAAACAGAGCATTTGGCAAGCTCTTCTGAAGACAGTACTACACCTTCTGCACAGACACAA
GAGAAGACAGAAGGGCCAACACCCATCCCTTCCCTAGCCAAGCCAGGTGTAACAGTTACCTTTTCTGA
CTTTCAGGACTCTGATCTCATTGCAACTATGATGCCTCCTATATCACCAGCTCCCATACAGAGTGATG
ACGATTGGATCCCTGATATCCAGATAGACCCAAATGGCCTTTCATTTAACCCAATATCAGATTTTCCA
GACACGACCTCCCCAAAATGTCCTGGACGCCCCTGGAAGTCAGTTAGTGAAATTAATCCAACCACACA
AATGAAAGAATCTTATTATTTCGATCTCACTGATGGCCTTTCTTAG

| | | | |
|---|---|---|---|
| NOV1z, CG104903-20 | | | |
| Protein Sequence | SEQ ID NO: 52 | 644 aa | MW at 71926.7 kD |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFTLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSEINPTTQMKESYYFDLTDGLS

| | | |
|---|---|---|
| | SEQ ID NO: 53 | 1981 bp |
| NOV1aa, SNP13381566 of | ORF Start: ATG at 50 | ORF Stop: end of sequence |
| CG104903-03, DNA Sequence | SNP Pos: 105 | SNP Change: A to G |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCGGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTCTC
CGTGGCTACCCAGACCTGCCACATTACTCCAGCCGACGGCCCTGTGGTGACACCCCAGTACGACTGCC
TCGGCTGTGTGCATCCTATATCAACCCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAACAGTACAGGTGGTGGCTGGCAAGAAATATTTTATTGACTTCGTGGCCAG
GGAAACCACATGTTCCAAGGAAAGTAATGAAGAGTTGACCGAAAGCTGTCAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAACGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGCCCACGGAAAA
CATAAAAATAAAGGCAAAAAGAATGGAAAGCACAATCGTTGGAAAACAGAGCATTTGGCAAGCTCTTC
TGAAGACAGTACTACACCTTCTGCACAGACACAAGAGAACACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACAGGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1aa, SNP13381566 of | SEQ ID NO: 54 | | MW at 71984.8 kD |
| CG104903-03, Protein Sequence | SNP Pos: 19 | 644 aa | SNP Change: Gln to Arg |

MKLITILFLCSRLLLSLTRESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPTLRHCIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFTDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWCHEKQRKHNLGHGHKHERDQGHGHQRGHGLGHGHEQQHGLGHGHK
FKLDDDLEHQGCHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQTDPNGLSFNPISDFPDTTSP
KCPGRPWKSVSEINPTTQMKESYYFDLTDGLS

| | | |
|---|---|---|
| | SEQ ID NO: 55 | 1981 bp |
| NOV1ab, SNP13379157 of | ORF Start: ATG at 50 | ORF Stop: end of sequence |
| CG104903-03, DNA Sequence | SNP Pos: 253 | SNP Change: T to C |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCCGACACGTTTTATTCCTTCA
ACTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTGTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGCGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ab, SNP13379157 of | SEQ ID NO: 56 | | MW at 71956.8 kD |
| CG104903-03, Protein Sequence | SNP Pos: 68 | 644 aa | SNP Change: Ser to Ser |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHCIQYFNNNTQHSSLFMLNEVKEAQRQVVAGLNFRITYSIVQT
NCSKENFLFLTPDCKSLWNGDTGECTDNAYIDIQLRIASFSQNCDIYPGKDFVQPFTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSEINPTTQMKESYYFDLTDGLS

| | | |
|---|---|---|
| | SEQ ID NO: 57 | 1981 bp |
| NOV1ac, SNP13379158 of | ORF Start: ATG at 50 | ORF Stop: end of sequence |
| CG104903-03, DNA Sequence | SNP Pos: 295 | SNP Change: T to C |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGACTGTCCTGTTCAAAGTGGCAAAACCTGGCAGGACTGTCAGTACAAG
GATGCTGCAAAAGCAGCCACTGGAGAATGCACGGCAACCGTGGGGAAGAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCCGTGAATGTACAGATAATGCA
TACATCGATATTCAGCTACGAATTGCTTCCTTCTCACAGAACTGTGACATTTATCCAGGGAAGCATTT
TGTACAACCACCTACCAAGATTTGCGTGGGCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGCCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATCGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTGTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ac, SNP13379158 of | SEQ ID NO: 58 | | MW at 71956.8 kD |
| CG104903-03, Protein Sequence | SNP Pos: 82 | 644 aa | SNP Change: Asp to Asp |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSEINPTTQMKESYYFDLTDGLS

| | | |
|---|---|---|
| | SEQ ID NO: 59 | 1981 bp |
| NOV1ad, SNP13379159 of | ORF Start: ATG at 50 | ORF Stop: end of sequence |
| CG104903-03, DNA Sequence | SNP Pos: 356 | SNP Change: G to A |

AATTCCGGTTGAAACCATCCCTCAGCTCCTACAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAACCACTGGAGAATGCACGGCAACCGTGGGGAACAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGACCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACACCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACGTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ad, SNP13379159 of | SEQ ID NO: 60 | | MW at 71986.8 kD |
| CG104903-03, Protein Sequence | SNP Pos: 103 | 644 aa | SNP Change: Ala to Thr |

MKLITILFLCSRLLLSLTQESQSEEIDCNDKDLFKAVDAALKKYNSQNQSNNQFVLYRITEATKTVGS
DTFYSFKYEIKEGDCPVQSCKTWQDCEYKDAAKATTGECTATVGKRSSTKFSVATQTCQITPAEGPVV
TAQYDCLGCVHPISTQSPDLEPILRHGIQYFNNNTQHSSLFMLNEVKPAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSEINPTTQMKESYYFDLTDGLS

| | | |
|---|---|---|
| | SEQ ID NO: 61 | 1981 bp |
| NOV1ae, SNP13375447 of | ORF Start: ATG at 50 | ORF Stop: end of sequence |
| CG104903-03, DNA Sequence | SNP Pos: 392 | SNP Change: A to U |

AATTCCGGTTGAAACCATCCCTCAGCTCCTAGAGGCAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGCCTCTGACACGTTTTATTCCTTCA
AGTACGAAATCAAGGAGGGGGATTGTCCTGTTCAAAGTGGCAAAACCTCGCAGGACTGTGAGTACAAG
GATGCTGCAAAAGCAGCCACTGGAGAATGCACGGCAACCGTGGGGAAGAGGGGCAGTACGAAATTCTC
CGTGGCTACCCAGACCTGCCAGATTACTCCAGCCGAGGGCCCTCTGGTGACAGCCCAGTACGACTGCC
TCGCCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACACAACTGTGACATTTATCCAGGGAAGGATTT
TGTACAACCACCTACCAAGATTTGCGTGGCCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAC
GGAAACCACATGTTCCAAGGAAAGTAATGAAGAGTTGACCGAAAGCTGTGAGACCAAAAAACTTGGCC
AAAGCCTAGATTGCAACGCTCAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
ATACGGGAAATAAAAGAAGAAACAACTGTAACTCCACCCCACACTTCCATGGCACCTGCACAAAGATG
AAGAGCCCGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTCATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ae, SNP13375447 of | SEQ ID NO: 62 | | MW at 71926.8 kD |
| CG104903-03, Protein Sequence | SNP Pos: 115 | 644 aa | SNP Change: Ser to Gly |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRGSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSEINPTTQMKESYYFDLTDGLS

| | | |
|---|---|---|
| | SEQ ID NO: 63 | 1981 bp |
| NOV1af, SNP13380032 of | Start: ATG at 50 | ORF Stop: end of sequence |
| CG104903-03, DNA Sequence | SNP Pos: 456 | SNP Change: T to C |

AATTCCGGTTGAAACCATCCCTCAGCTCCTAGAGGGACATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTCTC
CGTGGCTACCCAGACCTGCCACATTACTCCAGCCCAGGGCCCTGTGGCGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGACAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TCTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1af, SNP13380032 of | SEQ ID NO: 64 | | MW at 71928.7 kD |
| CG104903-03, Protein Sequence | SNP Pos: 136 | 644 aa | SNP Change: Val to Ala |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVA
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDTYPGKDFVQPPTKICVGCPRDIP
TNSPELEETLTHTITKLNAENNATFYFKIDNVKKARVQVVAGKKYFIDFVARETTCSKESNEELTESC
ETKKLGQSLDCNAEVYVVPWEKKIYPTVNCQPLGMISLMKRPPGFSPFRSSRTGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHQHGKHKNKGKKNGKHNGWKTEHLASSSEDSTTPSAQTQEKTEG
PTPIPSLAKPGVTVTFSDFQDSDLIATMMPPISPAPIQSDDDWTPDIQIDPNGLSFNPISDFPDTTSP
KCPGRPWKSVSEINPTTQMKESYYFDLTDGLS

| | | |
|---|---|---|
| | SEQ ID NO: 65 | 1981 bp |
| NOV1ag, SNP13380035 of | ORF Start: ATG at 50 | ORF Stop: end of sequence |
| CG104903-03, DNA Sequence | SNP Pos: 978 | SNP Change: A to G |

AATTCCGGTTGAAACCATCCCTCAGCTCCTAGAGGGAGATTGTTACATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATCCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACGGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ag, SNP13380035 of | SEQ ID NO: 66 | | MW at 71984.8 kD |
| CG104903-03, Protein Sequence | SNP Pos: 310 | 644 aa | SNP Change: Gln to Arg |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHCIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVCCPRDIP
TNSPELEETLTHTITKLNAENNATFYFKIDNVKKARVRVVAGKKYFIDFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KGPGRPWKSVSEINPTTQMKESYYFDLTDGLS

| | | |
|---|---|---|
| | SEQ ID NO: 67 | 1981 bp |
| NOV1ah, SNP13375317 of | ORF Start: ATG at 50 | ORF Stop: end of sequence |
| CG104903-03, DNA Sequence | SNP Pos: 1008 | SNP Change: A to G |

AATTCCGGTTCAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGAGGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAACCAGCCACTGGAGAATGCACGGCAACCGTGGGGAAGAGGACCAGTACGAAATTCTC
CGTGGCTACCGAGACCTGCCAGATTACTCGAGCCGAGGGCCCTGTGGTGAGAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTGGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCGAGGGAAGGATTT
TGTACAACCACCTACCAAGATTTGCGTGGGCTGCCCCAGAGATATACCCACCAACACCCCAGAGCTGG
AGGAGACACTGACTCACACCATGACAAAGCTTAATGCAGAGAATAACGCAACTTTCTATTTCAAGATT
GACAATGTGAAAAAAGCAAGAGTACAGGTGGTGGCTGGCAAGAAATATTTTATTGGCTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGACAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGCACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAACGCAAAAAGAATGGAAAGCACAATGGTTGGAAAACAGAGCATTTGGCAAGCTCTTC
TGAAGACAGTACTACACCTTCTGCACACACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ah, SNP13375317 of | SEQ ID NO: 68 | | MW at 71898.8 kD |
| CG104903-03, Protein Sequence | SNP Pos: 320 | 644 aa | SNP Change: Asp to Gly |

MKLITILFLCSRLLLSLTQESQSEEIDCNDKDLFKAVDAALKKYNSQNQSNNQFVLYRITEATKTVGS
DTFYSFKYEIKEGDCPVOSGKTWODCEYKDAAKAATGECTATVGKRSSTKFSVATOTCOITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTCECTDNAYIDIQLRIASFSQNCDIYPGKDFVQPPTKICVGCPRDIP
TNSPELEETLTHTITKLNAENNATFYFKIDNVKKARVQVVAGKKYFIGFVARETTCSKESNEELTESC
ETKKLGQSLDCNAEVYVVPWEKKIYPTVNCQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSEINPTTQMKESYYFDLTDGLS

| | | |
|---|---|---|
| | SEQ ID NO: 69 | 1981 bp |
| NOV1ai, SNP13375316 of | ORF Start: ATG at 50 | ORF Stop: end of sequence |
| CG104903-03, DNA Sequence | SNP Pos: 1023 | SNP Change: A to G |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGAGATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGGAACCACATGTTCCAAGGAAAGTAATGAAGAGTTGACCGAAAGCTGTGAGACCAAAAAACTTGGCC
AAAGCCTAGATTGGAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTCACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ai, SNP13375316 of | SEQ ID NO: 70 | | MW at 71884.7 kD |
| CG104903-03, Protein Sequence | SNP Pos: 325 | 644 aa | SNP Change: Glu to Gly |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARGTTCSKESNEELTESC
ETKKLGQSLDCNAEVYVVPWEKKIYPTVNCQPLGMISLMKRPPGFSPFRSSRIGEIKEETTVSPPHTS
MAPAQDEERDSCKEQGHTRRHDWGHEKQRKHNLGHGHKHERDQGHGHQRGHGLGHGHEQQHGLGHGHK
FKLDDDLEHQGGHVLDHCHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSEINPTTQMKESYYFDLTDGLS

| | | |
|---|---|---|
| | SEQ ID NO: 71 | 1981 bp |
| NOV1aj, SNP13379639 of | ORF Start: ATG at 50 | ORF Stop: end of sequence |
| CG104903-03, DNA Sequence | SNP Pos: 1041 | SNP Change: A to G |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAACAGGAGCAGTACGAAATTCTC
CGTGGCTACCCAGACCTGCCACATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTGTTCATGCTTAATGAAGTAAAACGGGCCCAAAGACA
GGTGGTGGCTGGATTGAACTTTCGAATTACCTACTCAATTGTGCAAACGAATTGTTCCAAAGACAATT
TTCTGTTCTTAACTCCAGACTGCAAGTCCCTTTGGAATGGTGATACCGGTGAATGTACAGATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGACATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGGAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
TGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGC
CCTTGGCCATGGACACGAACAACAGCATCGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1aj, SNP13379639 of | SEQ ID NO: 72 | | MW at 71884.7 kD |
| CG104903-03, Protein Sequence | SNP Pos: 331 | 644 aa | SNP Change: Glu to Gly |

MKLITILFLCSRLLLSLTQESQSEEIDCNDKDLFKAVDAALKKYNSQNQSNNQFVLYRITEATKTVGS
DTFYSFKYEIKECDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKGSNEELTESC
ETKKLCQSLDCNAEVYVVPWEKKIYPTVNCQPLGMISLMKRPPCFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNCWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KCPGRPWKSVSETNPTTQMKESYYFDLTDGLS

| | | |
|---|---|---|
| | SEQ ID NO: 73 | 1981 bp |
| NOV1ak, SNP13376277 of | ORF Start: ATG at 50 | ORF Stop: end of sequence |
| CG104903-03, DNA Sequence | SNP Pos: 1157 | SNP Change: T to C |

AATTCCGGTTGAAACCATCCCTCAGGTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGCAGCCACTGGAGAATGCACGGCAACCGTGGGGAAGAGGAGCAGTACGAAATTCTC
CGTGGCTACCCAGACCTGCCAGATTACTCCAGCCGAGGGCCCTGTGGTGACACCCCAGTACGACTGCC
TCGGCTGTGTGCATCCTATATCAACGCACAGCCCAGACCTGGAGCCCATTCTGAGACACGGCATTCAG
TACTTTAACAACAACACTCAACATTCCTCCCTCTTCATGCTTAATGAAGTAAAACGGCCCCAAAGACA
GGTGGTGGCTGGATTCAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACACATAATGCA
TACATCGATATTCAGCTACGAATTGCTTCCTTCTCACAGAACTGTGACATTTATCCAGGGAAGGATTT
TGTACAACCACCTACCAAGATTTGCGTGGGCTGCCCCAGACATATACCCACCAACAGCCCAGAGCTGG
AGGAGACACTGACTCACACCATCACAAAGCTTAATGCAGAGAATAACGCAACTTTCTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTAGATTGCAACGCTGAAGTTTATGTGGTACCCTGGGAGAAAAAAATTTACCCTACTGTCAAC
CGTCAACCACTGGGAATGATCTCACTGATGAAAAGGCCTCCAGGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAACCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCATACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1ak, SNP13376277 of | SEQ ID NO: 74 | | MW at 72009.8 kD |
| CG104903-03, Protein Sequence | SNP Pos: 370 | 644 aa | SNP Change: Cys to Arg |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFTDFVARETTCSKESNEELTESC
ETKKLGQSLDCNAEVYVVPWEKKIYPTVNRQPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPIQSDDDWIPDIQIDPNGLSFNPISDFPDTTSP
KGPGRPWKSVSEINPTTQMKESYYFDLTDGLS

| | | |
|---|---|---|
| | SEQ ID NO: 75 | 1981 bp |
| NOV1al, SNP13379163 of | ORF Start: ATG at 50 | ORF Stop: end of sequence |
| CG104903-03, DNA Sequence | SNP Pos: 1791 | SNP Change: T to C |

AATTCCGGTTGAAACCATCCCTCAGCTCCTAGAGGGAGATTGTTAGATCATGAAACTAATTACCATCC
TTTTCCTCTGCTCCAGGCTACTACTAAGTTTAACCCAGGAATCACAGTCCGAGGAAATTGACTGCAAT
GACAAGGATTTATTTAAAGCTGTGGATGCTGCTCTGAAGAAATATAACAGTCAAAACCAAAGTAACAA
CCAGTTTGTATTGTACCGCATAACTGAAGCCACTAAGACGGTTGGCTCTGACACGTTTTATTCCTTCA
AGTACGAAATCAAGGAGGGGGATTGTCCTGTTCAAAGTGGCAAAACCTGGCAGGACTGTGAGTACAAG
GATGCTGCAAAAGGAGCCACTGGAGAATGCAGGGCAACCGTCGGGAAGAGGAGCAGTACGAAATTCTC
CGTGGCTACCCAGACCTGCCAGATTACTCCAGCCGAGGGCCCTGTGGTGACAGCCCAGTACGACTGCC
TCGGCTGTGTGCATCCTATATCAACGCAGAGCCCAGACCTGGAGCCCATTCTGAGACACGGCATTCAG
TACTTTAACAACAACACTCAACATTCCTCCCTCTTCATGCTTAATGAAGTAAAACGGGCCCAAAGACA
GGTGGTGGCTGGATTGAACTTTCGAATTACCTACTCAATTGTGCAAACGAATTGTTCCAAAGAGAATT
TTCTGTTCTTAACTCCAGACTGCAAGTCCCTTTGGAATGGTGATACCGGTGAATGTACAGATAATGCA
TACATCGATATTCACCTACGAATTGCTTCCTTCTCACAGAACTGTGACATTTATCCAGGGAAGGATTT
TGTACAACCACCTACCAAGATTTGCGTGGGCTGCGCCAGAGATATACCCACCAACAGCCCAGAGCTGG
AGGAGACACTGACTCACACCATCACAAAGCTTAATGCAGAGAATAACGCAACTTTGTATTTCAAGATT
GACAATGTGAAAAAAGCAAGAGTACAGGTGGTGGCTGGCAAGAAATATTTTATTGACTTCGTGGCCAG
GGAAACCACATGTTCCAAGGAAAGTAATGAAGAGTTGACCGAAAGCTGTGAGACCAAAAAACTTGGCC
AAAGCCTACATTGCAACGCTGAAGTTTATGTGGTACCCTGGGAGAAAAAAATTTACCCTACTGTCAAC
TGTCAACCACTGGGAATGATCTCACTGATGAAAAGGCCTCCACGTTTTTCACCTTTCCGATCATCACG
AATAGGGGAAATAAAAGAAGAAACAACTGTAAGTCCACCCCACACTTCCATGGCACCTGCACAAGATG
AAGAGCGGGATTCAGGAAAAGAACAAGGGCATACTCGTAGACATGACTGGGGCCATGAAAAACAAAGA
AAACATAATCTTGGCCATGGCCATAAACATGAACGTGACCAAGGGCATGGGCACCAAAGAGGACATGG
CCTTGGCCATGGACACGAACAACAGCATGGTCTTGGTCATGGACATAAGTTCAAACTTGATGATGATC
TTGAACACCAAGGGGGCCATGTCCTTGACCATGGACATAAGCATAAGCATGGTCATGGCCACGGAAAA
CATAAAAATAAAGGCAAAAAGAATGGAAAGCACAATGGTTGGAAAACAGAGCATTTGGCAAGCTCTTC
TGAAGACAGTACTACACCTTCTGCACAGACACAAGAGAAGACAGAAGGGCCAACACCCATCCCTTCCC
TAGCCAAGCCAGGTGTAACAGTTACCTTTTCTGACTTTCAGGACTCTGATCTCATTGCAACTATGATG
CCTCCTATATCACCAGCTCCCACACAGAGTGATGACGATTGGATCCCTGATATCCAGATAGACCCAAA
TGGCCTTTCATTTAACCCAATATCAGATTTTCCAGACACGACCTCCCCAAAATGTCCTGGACGCCCCT
GGAAGTCAGTTAGTGAAATTAATCCAACCACACAAATGAAAGAATCTTATTATTTCGATCTCACTGAT
GGCCTTTCT

| | | | |
|---|---|---|---|
| NOV1al, SNP13379163 of | SEQ ID NO: 76 | | MW at 71944.7 kD |
| CG104903-03, Protein Sequence | SNP Pos: 581 | 644 aa | SNP Change: Ile to Thr |

MKLITILFLCSRLLLSLTQESQSEEIDCNDKDLFKAVDAALKKYNSQNQSNNQFVLYRITEATKTVGS
DTFYSFKYEIKEGDCPVQSGKTWQDCEYKDAAKAATGECTATVGKRSSTKFSVATQTCQITPAEGPVV
TAQYDCLGCVHPISTQSPDLEPILRHGIQYFNNNTQHSSLFMLNEVKRAQRQVVAGLNFRITYSIVQT
NCSKENFLFLTPDCKSLWNGDTGECTDNAYIDIQLRIASFSQNCDIYPGKDFVQPPTKICVGCPRDIP
TNSPELEETLTHTITKLNAENNATFYFKIDNVKKARVQVVAGKKYFIDFVARETTCSKESNEELTESC
ETKKLGOSLDCNAEVYVVPWEKKIYPTVNCOPLGMISLMKRPPGFSPFRSSRIGEIKEETTVSPPHTS
MAPAQDEERDSGKEQGHTRRHDWGHEKQRKHNLGHGHKHERDQGHGHQRGHGLGHGHEQQHGLGHGHK
FKLDDDLEHQGGHVLDHGHKHKHGHGHGKHKNKGKKNGKHNGWKTEHLASSSEDSTTPSAQTQEKTEG
PTPIPSLAKPGVTVTFSDFQDSDLIATMMPPISPAPTQSDDDWIPDIQIDPNGLSFNPISDFPDTTSP
KGPGRPWKSVSEINPTTQMKESYYFDLTDGLS

A ClustalW comparison of the above protein sequences yields the following sequence alignment shown in Table 1B.

TABLE 1B
Comparison of the NOV1 protein sequences.

| | |
|---|---|
| NOV1a | -----MKLITILFLCSRLLLSLTQESQSEEIDCNDKDLFKAVDAALKKYNSQNQSNNQFV |
| NOV1b | ------------------------------------------------------------ |
| NOV1c | HRGRTMKLITILFLCSRLLLSLTQESQSEEIDCNDKDLFKAVDAALKKYNSQNQSNNQFV |
| NOV1d | ------------------------------------------------------------ |
| NOV1e | ------------------------------------------------------------ |
| NOV1f | ------------------------------------------------------------ |
| NOV1g | ----------------------------------TRSPTDCNDKDLFKAVDAALKKYNSQ |
| NOV1h | ----------------------------EEIDCNDKDLFKAVDAALKKYNSQNQSNNQFV |
| NOV1i | -----------------------------------------TRSEEIDCNDKDLFKAVDA |
| NOV1j | ------------------------------------------------------------ |
| NOV1k | ------------------------------------------------------------ |
| NOV1l | ------------------------------------------------------------ |
| NOV1m | ------------------------------------------------------------ |
| NOV1n | ------------------------------------------------------------ |
| NOV1o | ------------------------------------------------------------ |
| NOV1p | ------------------------------------------------------------ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | ----------------------------EEIDCNDKDLFKAVDAALKKYNSQNQSNNQFV |
| NOV1w | ------------------------------------------------------------ |
| NOV1x | ------------------------------------------------------------ |
| NOV1y | ------------------------------------------------------------ |
| NOV1z | -----MKLITILFLCSRLLLSLTQESQSEEIDCNDKDLFKAVDAALKKYNSQNQSNNQFV |
| NOV1a | LYRITEATKTVGSDTFYSFKYEIKEGDCPVQSGKTWQDCEYKDAAKAATGECTATVGKRS |
| NOV1b | -MKLITILFLCSRLLLSLTQESQSEEIDCNDKDLFKAVDAALKKYNSQNQSNNQFVLYRI |
| NOV1c | LYRITEATKTVGSDTFYSFKYEIKEGDCPVQSGKTWQDCEYKDAAKAATGECTATVGKRS |
| NOV1d | ------------------------------------------------------------ |
| NOV1e | ------------------------------------------------------------ |
| NOV1f | ------------------------------------------------------------ |
| NOV1g | NQSNNQFVLYRITEATKTVGSDTFYSFKYEIKEGDCPVQSGKTWQDCEYKDAAKAATGEC |
| NOV1h | LYRITEATKTVGSDTFYSFKYEIKEGDCPVQSGKTWQDCEYKDAAKAATGECTATVGKRS |
| NOV1i | ALKKYNSQNQSNNQFVLYRITEATKTVGSDTFYSFKYEIKEGDCPVQSGKTWQDCEYKDA |
| NOV1j | ------------------------------------------------------------ |
| NOV1k | ----------------------MKLITILFLCSRLLLSLTQESQSEEIDDCNDKDLFKAV |
| NOV1l | ------------------------------------------------------------ |
| NOV1m | ------------------------------------------------------------ |
| NOV1n | ------------------------------------------------------------ |
| NOV1o | ------------------------------------------------------------ |
| NOV1p | ------------------------------------------------------------ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | LYRITEATKTVGSDTFYSFKYEIKEGDCPVQSGKTWQDCEYKDAAKAATGECTATVGKRS |
| NOV1w | ------------------------------------------------------------ |
| NOV1x | ------------------------------------------------------------ |
| NOV1y | ------------------------------------------------------------ |
| NOV1z | LYRITEATKTVGSDTFYSFKYEIKEGDCPVQSGKTWQDCEYKDAAKAATGECTATVGKRS |
| NOV1a | STKFSVATQTCQITPAEGPVVTAQYDCLGCVHPISTQSPDLEPILRHGIQYFNNNTQHSS |
| NOV1b | TEATKTVGSDTFYSFKYEIKEGDCPVQSGKTWQDCEYKDAAKAATGECTATVGKRSSTKF |
| NOV1c | STKFSVATQTCQITPAEGPVVTAQYDCLGCVHPISTQSPDLEPILRHGIQYFNNNTQHSS |
| NOV1d | ------------------------------------------------------------ |
| NOV1e | ------------------------------------------------------------ |
| NOV1f | ------------------------------------------------------------ |
| NOV1g | TATVGKRSSTKFSVATQTCQITPAEGPVVTAQYDCLGCVHPISTQSPDLEPILRHGIQYF |
| NOV1h | STKFSVATQTCQITPAEGPVVTAQYDCLGCVHPISTQSPDLEPILRHGIQYFNNNTQHSS |
| NOV1i | AKAATGECTATVGKRSSTKFSVATQTCQITPAEGPVVTAQYDCLGCVHPISTQSPDLEPI |
| NOV1j | ------------------------------------------------------------ |
| NOV1k | DAALKKYNSQNQSNNQFVLYRKTWQDCEYKDAAKAATGECTATVGKRSSTKFSVATQTCQ |
| NOV1l | ------------------------------------------------------------ |
| NOV1m | ------------------------------------------------------------ |
| NOV1n | ------------------------------------------------------------ |
| NOV1o | ------------------------------------------------------------ |
| NOV1p | ------------------------------------------------------------ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | STKFSVATQTCQITPAEGPVVTAQYDCLGCVHPISTQSPDLEPILRHGIQYFNNNTQHSS |
| NOV1w | ------------------------------------------------------------ |
| NOV1x | ------------------------------------------------------------ |
| NOV1y | ------------------------------------------------------------ |
| NOV1z | STKFSVATQTCQITPAEGPVVTAQYDCLGCVHPISTQSPDLEPILRHGIQYFNNNTQHSS |
| NOV1a | LFMLNEVKRAQRQVVAGLNFRITYSIVQTNCSKENFLFLTPDCKSLWNGDTGECTDNAYI |
| NOV1b | SVATQTCQITPAEGPVVTAQYDCLGCVHPISTQSPDLEPILRHGIQYFNNNTQHSSLFML |
| NOV1c | LFTLNEVKEAQRQVVAGLNFRITYSIVQTNCSKENFLFLTPDCKSLWNGDTGECTDNAYI |
| NOV1d | ------------------------------------------------------------ |
| NOV1e | ------------------------------------------------------------ |
| NOV1f | ------------------------------------------------------------ |
| NOV1g | NNNTQHSSLFMLNEVKRAQRQVVAGLNFRITYSIVQTNCSKENFLFLTPDCKSLWNGDTG |
| NOV1h | LFMLNEVKRAQRQVVAGLNFRITYSIVQTNCSKENFLFLTPDCKSLWNGDTGECTDNAYI |
| NOV1i | LRHGIQYFNNNTQHSSLFMLNEVKRAQRQVVAGLNFRITYSIVQTNCSKENFLFLTPDCK |
| NOV1j | ------------------------------------------------------------ |
| NOV1k | ITPAEGPVVTAQYDCLGCVHPISTQSPDLEPILRHGIQYFNNNTQHSSLFTLNEVKRAQR |
| NOV1l | ------------------------------------------------------------ |
| NOV1m | ------------------------------------------------------------ |
| NOV1n | ------------------------------------------------------------ |
| NOV1o | ------------------------------------------------------------ |
| NOV1p | ------------------------------------------------------------ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | LFMLNEVKRAQRQVVAGLNFRITYSIVQTNCSKENFLFLTPDCKSLWNGDTGECTDNAYI |
| NOV1w | ------------------------------------------------------------ |
| NOV1x | ------------------------------------------------------------ |
| NOV1y | ------------------------------------------------------------ |
| NOV1z | LFTLNEVKRAQRQVVAGLNFRITYSIVQTNCSKENFLFLTPDCKSLWNGDTGECTDNAYI |
| NOV1a | DIQLRIASFSQNCDIYPGKDFVQPPTKICVCCPRDIPTNSPELEETLTHTITKLNAENNA |
| NOV1b | NEVKRAQRQVVAGLNFRITYSIVQTNCSKENFLFLTPDCKSLWNGDTGECTDNAYIDIQL |
| NOV1c | DIQLRIASFSQNCDIYPGKDFVQPPTKICVGCPRDIPTNSPELEETLTHTITKLNAENNA |
| NOV1d | ------------------------------------------------------------ |
| NOV1e | ------------------------------------------------------------ |
| NOV1f | ------------------------------------------------------------ |
| NOV1g | ECTDNAYIDIQLRIASFSQNCDIYPGKDFVQPPTKICVGCPRDIPTNSPELEETLTHTIT |
| NOV1h | DIQLRIASFSQNCDIYPGKDFVQPPTKICVGCPRDIPTNSPELEETLTHTITKLNAENNA |
| NOV1i | SLWNGDTGECTDNAYIDIQLRIASFSQNCDIYPGKDFVQPPTKICVGCPRDIPTNSPELE |
| NOV1j | ------------------------------------------------------------ |
| NOV1k | QVVAGLNFRITYSIVQTNCSKENFLFLTPDCKSLWNGDTGECTDNAYIDIQLRIASFSQN |
| NOV1l | ------------------------------------------------------------ |
| NOV1m | --------------------------------------------------MKLITILFLC |
| NOV1n | ------------------------------------------------------------ |
| NOV1o | ------------------------------------------------------------ |
| NOV1p | ------------------------------------------------------------ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | DIQLRIASFSQNCDIYPGKDFVQPPTKICVGCPRDIPTNSPELEETLTHTITKLNAENNA |
| NOV1w | ------------------------------------------------------------ |
| NOV1x | ------------------------------------------------------------ |
| NOV1y | ------------------------------------------------------------ |
| NOV1z | DIQLRIASFSQNCDIYPGKDFVQPPTKICVGCPRDIPTNSPELEETLTHTITKLNAENNA |
| NOV1a | TFYFKIDNVKKARVQVVAGKKYFIDFVARETTCSKESNEELTESCETKKLGQSLDCNAEV |
| NOV1b | RIASFSQNCDIYPGKDFVQPPTKICVGCPRDIPTNSPELEETLTHTITKLNAENNATFYF |
| NOV1c | TFYFKIDNVKKARVQVVAGKKYFIDFVARETTCSKESNEELTESCETKKLGQSLDCNAEV |
| NOV1d | ------------------------------------------------------------ |
| NOV1e | ------------------------------------------------------------ |
| NOV1f | ------------------------------------------------------------ |
| NOV1g | KLNAENNATFYFKIDNVKKARVQVVAGKKYFIDFVARETTCSKESNEELTESCETKKLGQ |
| NOV1h | TFYFKIDNVKKARVQVVAGKKYFIDFVARETTCSKESNEELTESCETKKLGQSLDCNAEV |
| NOV1i | ETLTHTITKLNAENNATFYFKIDNVKKARVQVVAGKKYFIDFVARETTCSKESNEELTES |
| NOV1j | ------------------------------------------------------------ |
| NOV1k | CDIYPGKDFVQPPTKICVGCPRDIPTNSPELEETLTHTITKLNAENNATFYFKIDNVKKA |
| NOV1l | ------------------------------------------------------------ |
| NOV1m | SRLLLSLTQESQSEEIDCNDKDLFKAVDAALKKYNSQNQSNNQFVLYRITEATKTVGSDT |
| NOV1n | ------------------------------------------------------------ |
| NOV1o | ------------------------------------------------------------ |
| NOV1p | ------------------------------------------------------------ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | TFYFKIDNVKKARVQVVAGKKYFIDFVARETTCSKESNEELTESCETKKLGQSLDCNAEV |
| NOV1w | ------------------------------------------------------------ |
| NOV1x | ------------------------------------------------------------ |
| NOV1y | ------------------------------------------------------------ |
| NOV1z | TFYFKIDNVKKARVQVVAGKKYFIDFVARETTCSKESNEELTESCETKKLGQSLDCNAEV |
| NOV1a | YVVPWEKKIYPTVNCQPLGMISLMKRPPGFSPFRSSRIGEIKEETTVSPPHTSMAPAQDE |
| NOV1b | KIDNVKKARVQVVAGKKYFIDFVARETTCSKESNEELTESCETKKLGQSLDCNAEVYVVP |
| NOV1c | YVVPWEKKIYPTVNCQPLGMISLMKRPPGFSPFRSSRIGEIKEETTVSPPHTSMAPAQDE |
| NOV1d | -----------------------------------------------------------T |
| NOV1e | ------------------------------------------------------------ |
| NOV1f | -------------------------TRSGFSPFRSSRIGEIKEETTVSPPHTSMAPAQDE |
| NOV1g | SLDCNAEVYVVPWEKKIYPTVNCQPLGMISLMKRPPGFSPFRSSRIGEIKEETTVSPPHT |
| NOV1h | YVVPWEKKIYPTVNCQPLGMISLMKRPPGFSPFRSSRIGEIKEETTVSPPHTSMAPAQDE |
| NOV1i | CETKKLGQSLDCNAEVYVVFWEKKIYPTVNCQPLGMISLMKRPPGFSPFRSSRIGEIKEE |
| NOV1j | ------------------------------------------------------------ |
| NOV1k | RVQVVAGKKYFIDFVARETTCSKESNEELTESCETKKLGQSLDCNAEVYVVPWEKKIYPT |
| NOV1l | ------------------------------------------------------------ |
| NOV1m | FYSFKYEIKEGDCPVQSGKTWQDCEYKDAAKAATGECTATVCKRSSTKFSVATQTCQITP |
| NOV1n | ------------------------------------------------------------ |
| NOV1o | ------------------------------------------------------------ |
| NOV1p | ------------------------------------------------------------ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | YVVPWEKKIYPTVNCQPLGMISLMKRPPGFSPFRSSRIGEIKEETTVSPPHTSMAPAQDE |
| NOV1w | ------------------------------------------------------------ |
| NOV1x | ------------------------------------------------------------ |
| NOV1y | ----------------------------GFSPFRSSRIGEIKEETTVSPPHTSMAPAQDE |
| NOV1z | YVVPWEKKIYPTVNCQPLGMISLMKRPPGFSPFRSSRIGEIKEETTVSPPHTSMAPAQDE |
| NOV1a | ERDSGKEQGHTRRHDWGHEKQRKHNLGHGHKHERDQGHGHQRGHGLGHGHEQQHGLGHGH |
| NOV1b | WEKKIYPTVNCQPLGMISLMKRPPGFSPFRSSRIGEIKEETTVSPPHTSMAFAQDEERDS |
| NOV1c | ERDSGKEQGHTRRHDWGHEKQRKHNLGHGHKHERDQGHGHQRGHGLGHGHEQQHGLGHGH |
| NOV1d | RSAPAQDEERDSGKEQGHTRRHDWGHEKQRKHNLGHGHKHERDQGHGHQRGHGLGHGHEQ |
| NOV1e | -----------------------------------------------TRSPTMKLITILF |
| NOV1f | ERDSGKEQGHTRRHDWGHEKQRKHNLGHGHKHERDQGHCHQRGHGLGHGHEQQHGLGHGH |
| NOV1g | SMAPAQDEERDSGKEQGHTRRHDWGHEKQRKHNLGHGHKHERDQGHGHQRGHGLGHGHEQ |
| NOV1h | ERDSGKEQGHTRRHDWGHEKQRKHNLGHGHKHERDQGHGHQRGHGLGHGHEQQHGLGHGH |
| NOV1i | TTVSPPHTSMAPAQDEERDSGKEQGHTRRHDWGHEKQRKHNLGHGHKHERDQGHGHQRGH |
| NOV1j | ----------------------------------------------------MKLITILF |
| NOV1k | VNCQPLGMISLMKRPPCFSPFRSSRIGEIKEETTVSPPHTSMAPAQDEERDSGKEQGHTR |
| NOV1l | ----------------------------------------------------MKLITILF |
| NOV1m | AEGPVVTAQYDCLGCVHPISTQSPGFSPFRSSRIGEIKEETTVSPPHTSMAPAQDEERDS |
| NOV1n | ----------------------------------------------------MKLITILF |
| NOV1o | ----------------------------------------------------MKLITILF |
| NOV1p | ----------------------------------------------------MKLITILF |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | ERDSGKEQHHTRRHDWGHEKQRKHNLGHGHKHERDQGHGHQRGHGLGHGHEQQHGLGHGH |
| NOV1w | ----------------------------------------------------MKLITILF |
| NOV1x | ----------APAQDEERDSGKEQGHTRRHDWGHEKQRKHNLGHGHKHERDQGHGHQRGH |
| NOV1y | ERDSGKEQGHTRRHDWGHEKQRKHNLGHGHKHERDQGHGHQRGHGLGHGHEQQHGLGHGH |
| NOV1z | ERDSGKEQGHTRRHDWGHEKQRKHNLGHGHKHERDQGHGHQRGHGLGHGHEQQHOLGHGH |
| NOV1a | KFKLDDDLEHQGGHVLDHGHKHKHGHCHGKHKNKGKKNGKHNGWKTEHLASSSEDSTTPS |
| NOV1b | GKEQGHTRRHDWGHEKQRKHNLGHGHKHERDQGHGHQRGHGLGHGHEQQHGLGHGHKFKL |
| NOV1c | KFKLDDDLEHQGGHVLDHGHKHKHGHGHOKHKNKGKKNGKHNGWKTEHLASSSEDSTTPS |
| NOV1d | QHGLGHGHKFKLDDDLEHQGGHVLDHGNKHKHGHGHGKHKNKGKKNGKHNGVDG------ |
| NOV1e | LCSRLLLSLTQESQSE-EIDCNDKDLFKAVDAALKKYNSQNQSNNQFVLYRITEATKTVG |
| NOV1f | KFKLDDDLEHQGGHVLDHGHKHKHGHGHGKHKNKGKKNGKHNGWKTEHLASSSEDSTTPS |
| NOV1g | QHGLGHGHKFKLDDDLEHQGGHVLDHGHKHKHGHGHGKHKNKGKKNGKHNGWKTEHLASS |
| NOV1h | KFKLDDDLEHQGGHVLDHGHKHKHGHGHGKHKNKGKKNGKHNGWKTEHLASSSEDSTTPS |
| NOV1i | GLGHGHEQQHGLGHGHKFKLDDDLEHQGGHVLDHGHKHKHGHGHGKHKNKGKKNGKHNGW |
| NOV1j | LCSRLLLSLTQESQSEEIDDCNDKDLFKAVDAALKKYNSQNQSNNQFVLYR--------- |
| NOV1k | RHDWGHEKQRKHNLGHGHKHERDQGHGHQRGHGLGHGHEQQHGLGHGHKFKLDDDLEHQG |
| NOV1l | LCSRLLLSLTQESQSE-EIDCNDKDLFKAVDAALKKYNSQNQSNNQFVLYRITEATKTVG |
| NOV1m | GKEQGHTRRHDWGHEKQRKHNLGHGHKHERDQGHGHQRGHGLGHGHEQQHGLGHGHKFKL |
| NOV1n | LCSRLLLSLTQESQSE-EIDCNDKDLFKAVDAALKKYNSQNQSNNQFVLYRITEATKTVG |
| NOV1o | LCSRLLLSLTQESQSEEIDDCNDKDLFKAVDAALKKYNSQNQSNNQFVLYR--------- |
| NOV1p | LCSRLLLSLTQESQSE-EIDCNDKDLFKAVDAALKKYNSQNQSNNQFVLYR--------- |
| NOV1q | ------------------------------HKNKGKKNGKHNGWKT-------------- |
| NOV1r | ------------------------------KHGHGHGKHKNKGKKN-------------- |
| NOV1s | ------------------------------VLDHGHKHKHGHGHGK-------------- |
| NOV1t | ------------------------------GHGLGHGHEQQHGLGH-------------- |
| NOV1u | ------------------------------DQGHGHQRGHGLGHGH-------------- |
| NOV1v | KFKLDDDLEHQGGHVLDHGHKHKHGHGHGKHKNKGKKNGKHNGWKTEHLASSSEDSTTPS |
| NOV1w | LCSRLLLSLTQESQSE-EIDCNDKDLFKAVDAALKKYNSQNQSNNQFVLYRITEATKTVG |
| NOV1x | GLGHGHEQQHGLGHGHKFKLDDDLEHQGGHVLDHGHKHKHGHGHGKHKNKGKKNCKHNG- |
| NOV1y | KFKLDDDLEHQGGHVLDHGHKHKHCHGHGKHKNKGKKNGKHNGWKTEHLASSSEDSTTPS |
| NOV1z | KFKLDDDLEHQGGHVLDHGHKHKHGHGHGKHKNKGKKNGKHNGWKTEHLASSSEDSTTPS |
| NOV1a | AQTQEKTEGPTPIPSLAKPGVTVTFSDFQDSDLIATMMPPISPAPIQSDDDWIPDIQIDP |
| NOV1b | DDDLEHQGGHVLDHGHKHKHGHGHGKHKNKGKKNGKHNGWKTEHLASSSEDSTTPSAQTQ |
| NOV1c | AQTQEKTEGPTPIPSLAKPGVTVTFSDFQDSDLIATMMPPISPAPIQSDDDWIPDIQIDP |
| NOV1d | ------------------------------------------------------------ |
| NOV1e | SDTFYSFKYEIKEGDCPVQSGKTWQDCEYKDAAKAATGECTATVGKRSSTKFSVATQTCQ |
| NOV1f | AQTQEKTEGPTPIPSLAKPGVTVTFSDFQDSDLIATMMPPISPAPIQSDDDWIPDIQIDP |
| NOV1g | SEDSTTPSAQTQEKTEGPTPIPSLAKPGVTVTFSDFQDSDLIATMMPPISPAPIQSDDDW |
| NOV1h | AQTQEKTEGPTPIPSLAKPGVTVTFSDFQDSDLIATMMPPISPAPIQSDDDWIPDIQIDP |
| NOV1i | KTEHLASSSEDSTTPSAQTQEKTEGPTPIPSLAKPGVTVTFSDFQDSDLIATMMPPISPA |
| NOV1j | ---------------------KTWQDCEYKDAAKAATGECTATVGKRSSTKFSVATQTCQ |
| NOV1k | GHVLDHGHKHKHGHGHGKHKNKGKKNGKHNGWKTEHLASSSEDSTTPSAQTQEKTEGPTP |
| NOV1l | SDTFYSFKYEIKEGDCPVQSGKTWQDCEYKDAAKAATGECTATVGKRSSTKFSVATQTCQ |
| NOV1m | DDDLEHQGGHVLDHGHKHKHGHGHGKHKNKGKKNGKHNGWKTEHLASSSEDSTTPSAQTQ |
| NOV1n | SDTFYSFKYEIKEGDCPVQSGKTWQDCEYKDAAKAATGECTATVGRG-------AVRNSP |
| NOV1o | ---------------------KTWQDCEYKDAAKAATGECTATVGKRSSTKFSVATQTCQ |
| NOV1p | ---------------------------IT-EATKTATGECTATVGKRSSTKFSVATQTCQ |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | AQTQEKTEGPTPIPSLAKPGVTVTFSDFQDSDLIATMMPPISPAPIQSDDDWIPDIQIDP |
| NOV1w | SDTFYSFKYEIKEGDCPVQSGKTWQDCEYKDAAKAATGECTATVGKRSSTKFSVATQTCQ |
| NOV1x | ------------------------------------------------------------ |
| NOV1y | AQTQEKTEGPTPIPSLAKPGVTVTFSDFQDSDLIATMMPPISPAPIQSDDDWIPDIQIDP |
| NOV1z | AQTQEKTEGPTPIPSLAKPGVTVTFSDFQDSDLIATMMPPISPAPIQSDDDWIPDIQIDP |
| NOV1a | NGLSFNPISDFPDTTSPKCPGRPWKSVSEINPTTQMKESYYFDLTDGLS----------- |
| NOV1b | EKTEGPTPIPSLAKPGVTVTFSDFQDSDLIATMMPPISPAPIQSDDDWIPDIQIDPNGLS |
| NOV1c | NGLSFNPISDFPDTTSPKCPGRPWKSVSEINPTTQMKESYYFDLTDGLS----------- |
| NOV1d | ------------------------------------------------------------ |
| NOV1e | ITPAEGPVVTAQYDCLGCVHPISTQSPDLEPILRHCIQYFNNNTQHSSLFMLNEVKRAQR |
| NOV1f | NGLSFNPISDFPDTTSPKCPGRPWKSVSEINLEG-------------------------- |
| NOV1g | IPDIQIDPNGLSFNPISDFPDTTSPKCPGRPWKSVSEINPTTQMKESYYFDLTLEG---- |
| NOV1h | NGLSFNPISDFPDTTSPKCPGRPWKSVSEINPTTQMKESYYFDLTDCLS----------- |
| NOV1i | PIQSDDDWIPDIQIDPNGLSFNPISDFPDTTSPKCPGRPWKSVSEINPTTQMKESYYFDL |
| NOV1j | ITPAEGPVVTAQYDCLGCVHPISTQSPDLEPILRHGIQYFNNNTQHSSLFTLNEVKRAQR |
| NOV1k | IPSLAKPGVTVTFSDFQDSDLIATMMPPISPAPIQSDDDWIPDIQTDPNGLSFNPISDFP |
| NOV1l | ITPAEGPVVTAQYDCLGCVHPISTQSPDLEPILRHGIQYFNNNTQHSSLFMLNEVKRAQR |
| NOV1m | EKTEGPTPIPSLAKPGVTVTFSDFQDSDLIATMMPPISPAPIQSDDDWIPDIQIDPNGLS |
| NOV1n | WLPRPGAHSETRHSVL-------------------------------------------- |
| NOV1o | ITPAEGPVVTAQYDCLGCVHPISTQS---------P------------------------ |
| NOV1p | ITPAEGPVVTAQYDCLGCVHPISTQSPDLEPILRHGIQYFNNNTQHSSLFTLNEVKRAQR |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | NGLSFNPISDFPDTTSPKCPGRPWKSVSEINPTTQMKESYYFDLTDGLS----------- |
| NOV1w | ITPAEGPVVTAQYDCLGCVHPISTQSPDLEPILRHGIQYFNNNTQHSSLFMLNEVKRAQR |
| NOV1x | ------------------------------------------------------------ |
| NOV1y | NGLSFNPISDFPDTTSPKCPGRPWKSVSEIN----------------------------- |
| NOV1z | NGLSFNPISDFPDTTSPKCPGRPWKSVSEINPTTQMKESYYFDLTDGLS----------- |
| NOV1a | ------------------------------------------------------------ |
| NOV1b | FNPISDFPDTTSPKCPGRPWKSVSEINPTTQMKESYYFDLTDGLS--------------- |
| NOV1c | ------------------------------------------------------------ |
| NOV1d | ------------------------------------------------------------ |
| NOV1e | QVVAGLNFRITYSIVQTNCSKENFLFLTPDCKSLWNGDTGECTDNAYIDIQLRIASFSQN |
| NOV1f | ------------------------------------------------------------ |
| NOV1g | ------------------------------------------------------------ |
| NOV1h | ------------------------------------------------------------ |
| NOV1i | TDGLSVDG---------------------------------------------------- |
| NOV1j | QVVAGLNFRITYSIVQTNCSKENFLFLTPDCKSLWNGDTGECTDNAYIDIQLRIASFSQN |
| NOV1k | DTTSPKCPGRPWKSVSEINPTTQMKESYYFDLTDGLS----------------------- |
| NOV1l | QVVAGLNFRITYSIVQTNCSKENFLFLTPDCKSLWNGDTGECTDNAYIDIQLRIASFSQN |
| NOV1m | FNPISDFPDTTSPKCPGRPWKSVSEINPTTQMKESYYFDLTDGLS--------------- |
| NOV1n | ------------------------------------------------------------ |
| NOV1o | ------------------------------------------------------------ |
| NOV1p | QVVAGLNFRITYSIVQTNCSKENFLFLTPDCESLWNGDTGECTDNAYIDIQLRIASFSQN |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | ------------------------------------------------------------ |
| NOV1w | QVVAGLNFRITYSIVQTNCSKENFLFLTPDCKSLWNGDTGECTDNAYIDIQLRIASFSQN |
| NOV1x | ------------------------------------------------------------ |
| NOV1y | ------------------------------------------------------------ |
| NOV1z | ------------------------------------------------------------ |
| NOV1a | ------------------------------------------------------------ |
| NOV1b | ------------------------------------------------------------ |
| NOV1c | ------------------------------------------------------------ |
| NOV1d | ------------------------------------------------------------ |
| NOV1e | CDIYPGKDFVQPPTKICVGCPRDIPTNSPELEETLTHTITKLNAENNATFYFKIDNVKKA |
| NOV1f | ------------------------------------------------------------ |
| NOV1g | ------------------------------------------------------------ |
| NOV1h | ------------------------------------------------------------ |
| NOV1i | ------------------------------------------------------------ |
| NOV1j | CDIYPGKDFVQPPTKICVGCPRDIPTNSPELEETLTHTITKLNAENNATFYFKIDNVKKA |
| NOV1k | ------------------------------------------------------------ |
| NOV1l | CDIYPGKDFVQPPTKICVGCPRDIPTNSPELEETLTHTITKLNAENNATFYFKIDNVKKA |
| NOV1m | ------------------------------------------------------------ |
| NOV1n | ------------------------------------------------------------ |
| NOV1o | ------------------------------------------------------------ |
| NOV1p | CDIYPGKDFVQPPTKICVGCPRDIPTNSPELEETLTHTITKLNAENNATFYFKIDNVKKA |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | ------------------------------------------------------------ |
| NOV1w | CDIYPGKDFVQPPTKICVGCPRDIPTNSPELEETLTHTITKLNAENNATFYFKIDNVKKA |
| NOV1x | ------------------------------------------------------------ |
| NOV1y | ------------------------------------------------------------ |
| NOV1z | ------------------------------------------------------------ |
| NOV1a | ------------------------------------------------------------ |
| NOV1b | ------------------------------------------------------------ |
| NOV1c | ------------------------------------------------------------ |
| NOV1d | ------------------------------------------------------------ |
| NOV1e | RVQVVAGKKYFIDFVARETTCSKESNEELTESCETKKLGQSLDCNAEVYVLEG------- |
| NOV1f | ------------------------------------------------------------ |
| NOV1g | ------------------------------------------------------------ |
| NOV1h | ------------------------------------------------------------ |
| NOV1i | ------------------------------------------------------------ |
| NOV1j | RVQVVAGKKYFIDFVARETTCSKESNEELTESCETKKLGQSLDCNAEVYVVPWEKKIYPT |
| NOV1k | ------------------------------------------------------------ |
| NOV1l | RVQVVAGKKYFIDFVARETTCSKESNEELTESCETKKLGQSLDCNAEVYVVPWEKKIYPT |
| NOV1m | ------------------------------------------------------------ |
| NOV1n | ------------------------------------------------------------ |
| NOV1o | ------------------------------------------------------------ |
| NOV1p | RVQVVAGKKYFIDFVARETTCSKESNEELTESCETKKLGQSLDCNAEVYVVPWEKKIYPT |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | ------------------------------------------------------------ |
| NOV1w | RVQVVAGKKYFIDFVARETTCSKESNEELTESCETKKLGQSLDCNAEVYV---------- |
| NOV1x | ------------------------------------------------------------ |
| NOV1y | ------------------------------------------------------------ |
| NOV1z | ------------------------------------------------------------ |
| NOV1a | ------------------------------------------------------------ |
| NOV1b | ------------------------------------------------------------ |
| NOV1c | ------------------------------------------------------------ |
| NOV1d | ------------------------------------------------------------ |
| NOV1e | ------------------------------------------------------------ |
| NOV1f | ------------------------------------------------------------ |
| NOV1g | ------------------------------------------------------------ |
| NOV1h | ------------------------------------------------------------ |
| NOV1i | ------------------------------------------------------------ |
| NOV1j | VNCQPLGMISLMKRPPGFSPFRSSRIGEIKEETTSHLRSCEYKGRPPKAGAEPASEREVS |
| NOV1k | ------------------------------------------------------------ |
| NOV1l | VNCQPLGMISLMKRPPGFSPFRSSRIGEIKEETTSHLRSCEYKGRPPKAGAEPASEREVS |
| NOV1m | ------------------------------------------------------------ |
| NOV1n | ------------------------------------------------------------ |
| NOV1o | ----------------GFSPFRSSRIGEIKEETTSHLRSCEYKGRPPKAGAEPASEREVS |
| NOV1p | VNCQPLGMISLMKRPPGFSPFRSSRIGEIKEETTSHLRSCEYKGRPPKAGAEPVSEREVS |
| NOV1q | ------------------------------------------------------------ |
| NOV1r | ------------------------------------------------------------ |
| NOV1s | ------------------------------------------------------------ |
| NOV1t | ------------------------------------------------------------ |
| NOV1u | ------------------------------------------------------------ |
| NOV1v | ------------------------------------------------------------ |
| NOV1w | ------------------------------------------------------------ |
| NOV1x | ------------------------------------------------------------ |
| NOV1y | ------------------------------------------------------------ |
| NOV1z | ------------------------------------------------------------ |
| NOV1a | (SEQ ID NO: 2) |
| NOV1b | (SEQ ID NO: 4) |
| NOV1c | (SEQ ID NO: 6) |
| NOV1d | (SEQ ID NO: 8) |
| NOV1e | (SEQ ID NO: 10) |
| NOV1f | (SEQ ID NO: 12) |
| NOV1g | (SEQ ID NO: 14) |
| NOV1h | (SEQ ID NO: 16) |
| NOV1i | (SEQ ID NO: 18) |
| NOV1j | (SEQ ID NO: 20) |
| NOV1k | (SEQ ID NO: 22) |
| NOV1l | (SEQ ID NO: 24) |
| NOV1m | (SEQ ID NO: 26) |
| NOV1n | (SEQ ID NO: 28) |
| NOV1o | (SEQ ID NO: 30) |
| NOV1p | (SEQ ID NO: 32) |
| NOV1q | (SEQ ID NO: 34) |
| NOV1r | (SEQ ID NO: 36) |
| NOV1s | (SEQ ID NO: 38) |
| NOV1t | (SEQ ID NO: 40) |
| NOV1u | (SEQ ID NO: 42) |
| NOV1v | (SEQ ID NO: 44) |
| NOV1w | (SEQ ID NO: 46) |
| NOV1x | (SEQ ID NO: 48) |
| NOV1y | (SEQ ID NO: 50) |
| NOV1z | (SEQ ID NO: 52) |

Further analysis of the NOV1a protein yielded the following properties shown in Table 1C.

TABLE 1C
Protein Sequence Properties NOV1a

| SignalP analysis: | Cleavage site between residues 24 and 25 | | | |
|---|---|---|---|---|
| PSORT II | PSG: a new signal peptide prediction method | | | |
| analysis: | N-region: length 2; pos.chg 1; neg.chg 0 | | | |
| | H-region: length 9; peak value 11.25 | | | |
| | PSG score: 6.85 | | | |
| | GvH: von Heijne's method for signal seq. recognition | | | |
| | GvH score (threshold: −2.1): 0.04 | | | |
| | possible cleavage site: between 18 and 19 | | | |
| | >>> Seems to have a cleavable signal peptide (1 to 18) | | | |
| | ALOM: Klein et al's method for TM region allocation | | | |
| | Init position for calculation: 19 | | | |
| | Tentative number of TMS(s) for the threshold 0.5: 0 | | | |
| | number of TMS(s) . . . fixed | | | |
| | PERIPHERAL Likelihood = 7.90 (at 135) | | | |
| | ALOM score: 7.90 (number of TMSs: 0) | | | |
| | MTOP: Prediction of membrane topology (Hartmann et al.) | | | |
| | Center position for calculation: 9 | | | |
| | Charge difference: −4.0 C(−2.0)-N(2.0) | | | |
| | N >= C: N-terminal side will be inside | | | |
| | MITDISC: discrimination of mitochondrial targeting seq | | | |
| | R content: | 1 | Hyd Moment(75): | 3.52 |
| | Hyd Moment (95): | 7.10 | G content: | 0 |
| | D/E content: | 1 | S/T content: | 4 |

Score: −3.12
Gavel: prediction of cleavage sites for mitochondrial preseq
R-2 motif at 22 SRL|LL
NUCDISC: discrimination of nuclear localization signals
pat4: none
pat7: none
bipartite: none
content of basic residues: 11.2%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 89
COIL: Lupas's algorithm to detect coiled-coil regions
total: 0 residues
Final Results (k = 9/23):
66.7%: extracellular, including cell wall
22.2%: mitochondrial
11.1%: nuclear
>> prediction for CG104903-09 is exc (k = 9)

A search of the NOV1a protein against the Geneseq database, a proprietary database that contains sequences published in patents and patent publication, yielded several homologous proteins shown in Table 1D.

TABLE 1D
Geneseq Results for NOV1a

| | | | Identities/ | |
|---|---|---|---|---|
| | | | Similarities for | |
| Geneseq | Protein/Organism/Length | NOV1a Residues/ | the Matched | Expect |
| Identifier | [Patent #, Date] | Match Residues | Region | Value |
| ABG21101 | Novel human diagnostic protein | 1 . . . 644 | 644/644 (100%) | 0.0 |
| | #21092 - Homo sapiens, 644 aa. | 1 . . . 644 | 644/644 (100%) | |
| | [WO200175067-A2, 11 OCT. | | | |
| | 2001] | | | |
| ABB78710 | Human high molecular weight | 1 . . . 644 | 643/644 (99%) | 0.0 |
| | kininogen (HK) protein - Homo | 1 . . . 644 | 643/644 (99%) | |
| | sapiens, 644 aa. [WO200214369- | | | |
| | A2, 21 FEB. 2002] | | | |
| ABB78707 | Human high molecular weight | 19 . . . 644 | 625/626 (99%) | 0.0 |
| | kininogen (HK) mature protein | 1 . . . 626 | 625/626 (99%) | |
| | SEQ ID NO: 1 - Homo sapiens, 626 | | | |
| | aa. [WO200214369-A2, 21 FEB. | | | |
| | 2002] | | | |
| ABR41202 | Human DITHP extracellular | 288 . . . 644 | 355/357 (99%) | 0.0 |
| | signalling protein - Homo sapiens, | 1 . . . 357 | 355/357 (99%) | |
| | 357 aa. [WO200297031-A2, 05 | | | |
| | DEC. 2002] | | | |
| ABG21105 | Novel human diagnostic protein | 1 . . . 416 | 385/432 (89%) | 0.0 |
| | #21096 - Homo sapiens, 435 aa. | 2 . . . 433 | 389/432 (89%) | |
| | [WO200175067-A2, 11 OCT. | | | |
| | 2001] | | | |

In a BLAST search of public sequence databases, the NOV1a protein was found to have homology to the proteins shown in the BLASTP data in Table 1E.

TABLE 1E
Public BLASTP Results for NOV1a

| | | | Identities/ | |
|---|---|---|---|---|
| Protein | | | Similarities for | |
| Accession | | NOV1a Residues/ | the Matched | Expect |
| Number | Protein/Organism/Length | Match Residues | Portion | Value |
| P01042 | Kininogen precursor (Alpha-2- | 1 . . . 644 | 643/644 (99%) | 0.0 |
| | thiol proteinase inhibitor) | 1 . . . 644 | 643/644 (99%) | |
| | [Contains: Bradykinin] - Homo | | | |
| | sapiens (Human), 644 aa. | | | |
| P01044 | Kininogen, HMW I precursor | 1 . . . 643 | 454/644 (70%) | 0.0 |
| | (Thiol proteinase inhibitor) | 1 . . . 620 | 517/644 (79%) | |
| | [Contains: Bradykinin] - Bos | | | |
| | taurus (Bovine), 621 aa. | | | |
| P01045 | Kininogen, HMW II precursor | 1 . . . 643 | 450/644 (69%) | 0.0 |
| | (Thiol proteinase inhibitor) | 1 . . . 618 | 515/644 (79%) | |
| | [Contains: Bradykinin] - Bos | | | |
| | taurus (Bovine), 619 aa. | | | |
| AAO61092 | Kininogen - Homo sapiens | 1 . . . 416 | 406/425 (95%) | 0.0 |
| | (Human), 427 aa. | 1 . . . 425 | 407/425 (95%) | |
| O08677 | Kininogen precursor [Contains: | 1 . . . 644 | 386/668 (57%) | 0.0 |
| | Bradykinin] - Mus musculus | 1 . . . 661 | 461/668 (68%) | |
| | (Mouse), 661 aa. | | | |

PFam analysis predicts that the NOV1a protein contains the domains shown in the Table 1F.

TABLE 1F
Domain Analysis of NOV1a

| | | Identities/ | |
|---|---|---|---|
| | | Similarities | |
| | NOV1a Match | for the | Expect |
| Pfam Domain | Region | Matched Region | Value |
| Cathelicidins | 30 . . . 95 | 16/70 (23%) | 0.57 |
| | | 34/70 (49%) | |
| cystatin | 23 . . . 126 | 30/112 (27%) | 1.2e−30 |
| | | 91/112 (81%) | |
| cystatin | 144 . . . 248 | 28/113 (25%) | 2.2e−34 |
| | | 93/113 (82%) | |
| cystatin | 266 . . . 370 | 32/113 (28%) | 2.7e−38 |
| | | 94/113 (83%) | |

### Example 2

The NOV2 clone was analyzed, and the nucleotide and encoded polypeptide sequences are shown in Table 2A.

TABLE 2A
NOV2 Sequence Analysis

| | | |
|---|---|---|
| NOV2a, CG120844-02 | SEQ ID NO: 77 | 689 bp |
| DNA Sequence | ORF Start: at 2 | ORF Stop: TAA at 659 |

CTCCTTCCAGGACCTGGACCTCTGCCCTCTGGATGGCGGCATCCAGCTACGAATCTCCGACCACCACT
ACAGCAAGGGGTTCAGGCAGGCCGCGTCAGTTGTTGTGGCCATGGACAAGCTGAGGAAGATGCTGGTT
CCCTGCCCACAGACCTTCCAGGAGAATGACCTGAGCACCTTCTTTCCCTTCATCTTTGAAGAAGAACC
TATCTTCTTCGACACATGGGATAACGAGGCTTATGTGCACGATGCACCTGTACGATCACTGAACTGCA
CGCTCCGGGACTCACAGCAAAAAAGCTTGGTGATGTCTGGTCCATATGAACTGAAAGCTCTCCACCTC
CAAGGAGAAGAAAGTAATGACAAAATACCTGTGGCCTTGGGCCTCAAGGAAAAGAATCTGTACCTGTC
CTGCGTGTTGAAAGATGATAAGCCCACTCTACAGCTGGAGAGTGTAGATCCCAAAAATTACCCAAAGA
AGAAGATGGAAAAGCGATTTGTCTTCAACAAGATAGAAATCAATAACAAGCTGGAATTTGAGTCTGCC
CAGTTCCCCAACTGGTACATCAGCACCTCTCAAGCAGAAAACATGCCCGTCTTCCTGGGAGGGACCAA
AGGCGGCCAGGATATAACTGACTTCACCATGCAATTTGTGTCTTCCTAAAGAGAGCTGTACCCAGAGA
GTCCTGTGC

| | | | |
|---|---|---|---|
| NOV2a, CG120844-02 | | | |
| Protein Sequence | SEQ ID NO: 78 | 219 aa | MW at 25075.3 kD |

SFQDLDLCPLDGGIQLRISDHHYSKGFRQAASVVVAMDKLRKMLVPCPQTFQENDLSTFFPFIFEEEP
IFFDTWDNEAYVHDAPVRSLNCTLRDSQQKSLVMSGPYELKALHLQGEESNDKIPVALGLKEKNLYLS
CVLKDDKPTLQLESVDPKNYPKKKMEKRFVFNKIEINNKLEFESAQFPNWYISTSQAENMPVFLGGTK
GGQDITDFTMQFVSS

| | | |
|---|---|---|
| NOV2b, 251426189 | SEQ ID NO: 79 | 400 bp |
| DNA Sequence | ORF Start: at 2 | ORF Stop: end of sequence |

CACCGGATCCCTCCGGGACTCACAGCAAAAAAGCTTGGTGATGTCTGGTCCATATGAACTGAAAGCTC
TCCACCTCCAAGGAGAAGAAAGTAATGACAAAATACCTGTGGCCTTGGGCCTCAAGGAAAAGAATCTG
TACCTGTCCTGCGTGTTGAAAGATGATAAGCCCACTCTACAGCTGGAGAGTGTAGATCCCAAAAATTA
CCCAAAGAAGAAGATGGAAAAGCGATTTGTCTTCAACAAGATAGAAATCAATAACAAGCTGGAATTTG
AGTCTGCCCAGTTCCCCAACTGGTACATCAGCACCTCTCAAGCAGAAAACATGCCCGTCTTCCTCGGA
GGGACCAAAGGCGGCCAGGATATAACTGACTTCACCATGGAATTTGTGTCTCTCGAGGGC

| | | | |
|---|---|---|---|
| NOV2b, 251426189 | | | |
| Protein Sequence | SEQ ID NO: 80 | 133 aa | MW at 15050.1 kD |

TGSLRDSQQKSLVMSGPYELKALHLQGEESNDKTPVALGLKEKNLYLSCVLKDDKPTLQLESVDPKNY
PKKKMEKRFVFNKIEINNKLEFESAQFPNWYISTSQAENMPVFLGGTKGCQDITDFTMQFVSLEG

| | | |
|---|---|---|
| NOV2c, CG120844-01 | SEQ ID NO: 81 | 820 bp |
| DNA Sequence | ORF Start: ATG at 3 | ORF Stop: TAA at 810 |

CCATGGCAGAAGTACCTGAGCTCGCCAGTGAAATGATGGCTTATTACAGTGGCAATGAGGATGACTTG
TTCTTTGAAGCTGATGGCCCTAAACAGATGAAGTGCTCCTTCCAGGACCTGGACCTCTGCCCTCTCCA
TGGCGGCATCCAGCTACGAATCTCCGACCACCACTACAGCAAGGGCTTCAGGCAGGCCGCGTCAGTTG
TTGTGGCCATGGACAAGCTGAGGAAGATGCTGGTTCCCTGCCCACAGACCTTCCAGGAGAATGACCTG
AGCACCTTCTTTCCCTTCATCTTTGAAGAAGAACCTATCTTCTTCGACACATGGGATAACGAGGCTTA
TGTGCACGATGCACCTCTACGATCACTGAACTGCACGCTCCGGGACTCACAGCAAAAAAGCTTGGTGA
TGTCTGGTCCATATGAACTGAAAGCTCTCCACCTCCAGGGACAGGATATGGAGCAACAAGTGGTGTTC
TCCATGTCCTTTGTACAAGGAGAAGAAAGTAATGACAAAATACCTGTGGCCTTGGGCCTCAAGGAAAA
GAATCTGTACCTGTCCTGCGTGTTGAAAGATGATAAGCCCACTCTACAGCTGGAGAGTGTAGATCCCA
AAAATTACCCAAAGAAGAAGATGGAAAAGCGATTTGTCTTCAACAAGATAGAAATCAATAACAAGCTG
GAATTTGAGTCTGCCCAGTTCCCCAACTGGTACATCAGCACCTCTCAAGCAGAAAACATGCCCGTCTT
CCTGGGAGGGACCAAAGGCGGCCAGGATATAACTGACTTCACCATGCAATTTGTGTCTTCTTAAAGAG
AGCT

| | | | |
|---|---|---|---|
| NOV2c, CG120844-01 | | | |
| Protein Sequence | SEQ ID NO: 82 | 269 aa | MW at 30747.6 kD |

MAEVPELASEMMAYYSGNEDDLFFEADGPKOMKCSFODLDLCPLDGGIOLRISDHHYSKGFROAASVV
VAMDKLRKMLVPCPQTFQENDLSTFFPFIFEEEPIFFDTWDNEAYVHDAPVRSLNCTLRDSQQKSLVM
SGPYELKALHLQGQDMEQQVVFSMSFVQGEESNDKIPVALGLKEKNLYLSCVLKDDKPTLQLESVDPK
NYPKKKMEKRFVFNKIEINNKLEFESAQFPNWYISTSQAENNPVFLGGTKGGQDITDFTMQFVSS

| | | |
|---|---|---|
| | SEQ ID NO: 83 | 689 bp |
| NOV2d, SNP13377796 of | ORF Start: at 2 | ORF Stop: TAA at 659 |
| CG120844-02, DNA Sequence | SNP Pos: 231 | SNP Change: A to G |

CTCCTTCCAGGACCTGGACCTCTGCCCTCTGGATGGCGGCATCCACCTACGAATCTCCCACCACCACT
ACAGCAAGGGCTTCAGGCAGGCCGCGTCAGTTGTTGTCGCCATGGACAAGCTGAGGAAGATGCTGGTT
CCCTGCCCACAGACCTTCCAGGAGAATGACCTGAGCACCTTCTTTCCCTTCATCTTTGAAGAAGAACC
TATCTTCTTCGACACATGGGATAACGGGGCTTATGTCCACGATGCACCTGTACGATCACTGAACTGCA
CGCTCCGGGACTCACAGCAAAAAAGCTTGGTGATGTCTGGTCCATATGAACTGAAAGCTCTCCACCTC
CAAGGAGAAGAAAGTAATGACAAAATACCTGTCGCCTTGGGCCTCAAGGAAAAGAATCTGTACCTGTC
CTGCGTGTTGAAAGATGATAAGCCCACTCTACAGCTGGAGAGTGTAGATCCCAAAAATTACCCAAAGA
AGAAGATGGAAAAGCGATTTGTCTTCAACAAGATAGAAATCAATAACAACCTGGAATTTGAGTCTGGC
CAGTTCCCCAACTGGTACATCAGCACCTCTCAAGCAGAAAACATGCCCGTCTTCCTGGCAGGGACCAA
AGGCGGCCAGGATATAACTGACTTCACCATGCAATTTGTGTCTTCCTAAAGAGAGCTGTACCCAGAGA
GTCCTGTGC

| | | | |
|---|---|---|---|
| NOV2d, SNP13377796 of | SEQ ID NO: 84 | | MW at 25003.2 kD |
| CG120844-02, Protein Sequence | SNP Pos: 77 | 219 aa | SNP Change: Glu to Gly |

SFQDLDLCPLDGGIQLRISDHHYSKGFRQAASVVVAMDKLRKNLVPCPQTFQENDLSTFFPFIFEEEP
IFFDTWDNGAYVHDAPVRSLNCTLRDSQQKSLVMSGPYELKALHLQGEESNDKTPVALGLKEKNLYLS
CVLKDDKPTLQLESVDPKNYPKKKMEKRFVFNKIEINNKLEFESAQFPNWYISTSQAENMPVFLGGTK
GGQDITDFTMQFVSS

| | | |
|---|---|---|
| | SEQ ID NO: 85 | 689 bp |
| NOV2e, SNP13377795 of | ORF Start: at 2 | ORF Stop: TAA at 659 |
| CG120844-02, DNA Sequence | SNP Pos: 272 | SNP Change: A to G |

CTCCTTCCAGGACCTGGACCTCTGCCCTCTGGATGGCGGCATCCAGCTACGAATCTCCGACCACCACT
ACAGCAAGGGCTTCAGGCAGGCCGCGTCAGTTGTTGTGGCCATGGACAAGCTGAGCAAGATGCTGGTT
CCCTGCCCACACACCTTCCAGGAGAATGACCTGAGCACCTTCTTTCCCTTCATCTTTGAAGAAGAACC
TATCTTCTTCGACACATGGGATAACGAGGCTTATGTGCACGATGCACCTGTACGATCACTGAACTGCG
CGCTCCGGGACTCACAGCAAAAAAGCTTGGTGATGTCTGGTCCATATGAACTGAAAGCTCTCCACCTC
CAAGGAGAAGAAAGTAATGACAAAATACCTGTGGCCTTGGGCCTCAAGGAAAAGAATCTGTACCTGTC
CTGCGTGTTGAAAGATGATAAGCCCACTCTACAGCTGGAGAGTGTAGATCCCAAAAATTACCCAAAGA
AGAAGATGGAAAAGCGATTTGTCTTCAACAAGATAGAAATCAATAACAAGCTGGAATTTGAGTCTGCC
CAGTTCCCCAACTGGTACATCAGCACCTCTCAAGCAGAAAACATGCCCGTCTTCCTGGGAGGGACCAA
AGGCGGCCAGGATATAACTGACTTCACCATGCAATTTGTGTCTTCCTAAAGAGAGCTGTACCCAGAGA
GTCCTGTGC

| | | | |
|---|---|---|---|
| NOV2e, SNP13377795 of | SEQ ID NO: 86 | | MW at 25045.2 kD |
| CG120844-02, Protein Sequence | SNP Pos: 91 | 219 aa | SNP Change: Thr to Ala |

SFQDLDLCPLDGGIQLRISDHHYSKGFRQAASVVVAMDKLRKMLVPCPQTFQENDLSTFFPFIFEEEP
IFFDTWDNEAYVHDAPVRSLNCALRDSQQKSLVMSGPYELKALHLQGEESNDKIPVALGLKEKNLYLS
CVLKDDKPTLQLESVDPKNYPKKKMEKRFVFNKIEINNKLEFESAQFPNWYISTSQAENMPVFLGGTK
GGQDITDFTMQFVSS

| | | |
|---|---|---|
| | SEQ ID NO: 87 | 689 bp |
| NOV2f, SNP13377794 of | ORF Start: at 2 | ORF Stop: TAA at 659 |
| CG120844-02, DNA Sequence | SNP Pos: 374 | SNP Change: G to A |

CTCCTTCCAGGACCTGGACCTCTGCCCTCTCGATGGCGGCATCCAGCTACGAATCTCCGACCACCACT
ACAGCAAGGGCTTCAGGCAGGCCGCGTCAGTTGTTGTGGCCATGGACAAGCTGAGGAAGATGCTGGTT
CCCTGCCCACAGACCTTCCAGGAGAATGACCTGAGCACCTTCTTTCCCTTCATCTTTGAAGAACAACC
TATCTTCTTCGACACATGGGATAACGAGGCTTATGTGCACGATGCACCTGTACGATCACTGAACTGCA
CGCTCCGGGACTCACAGCAAAAAAGCTTGGTGATGTCTGGTCCATATGAACTGAAAGCTCTCCACCTC
CAAGCAGAAGAAAGTAATGACAAAATACCTGTGACCTTGGGCCTCAAGGAAAAGAATCTGTACCTGTC
CTGCGTGTTGAAAGATGATAAGCCCACTCTACAGCTGGAGAGTGTAGATCCCAAAAATTACCCAAAGA
AGAAGATGGAAAAGCGATTTGTCTTCAACAAGATAGAAATCAATAACAAGCTGGAATTTGAGTCTGCC
CAGTTCCCCAACTGGTACATCAGCACCTCTCAAGCAGAAAACATGCCCGTCTTCCTGGGAGGGACCAA
AGGCGGCCAGGATATAACTGACTTCACCATGCAATTTGTGTCTTCCTAAAGAGAGCTGTACCCAGAGA
GTCCTCTGC

| | | | |
|---|---|---|---|
| NOV2f, SNP13377794 of | SEQ ID NO: 88 | | MW at 25105.3 kD |
| CG120844-02, Protein Sequence | SNP Pos: 125 | 219 aa | SNP Change: Ala to Thr |

SFQDLDLCPLDGGIQLRISDHHYSKGFRQAASVVVAMDKLRKMLVPCPQTFQENDLSTFFPFIFEEEP
IFFDTWDNEAYVHDAPVRSLNCTLRDSQQKSLVMSGPYELKALHLQGEESNDKIPVTLGLKEKNLYLS
CVLKDDKPTLQLESVDPKNYPKKKMEKRFVFNKIEINNKLEFESAQFPNWYISTSQAENMPVFLGGTK
GGQDITDFTMQFVSS

| | | |
|---|---|---|
| | SEQ ID NO: 89 | 689 bp |
| NOV2g, SNP13377793 of | ORF Start: at 2 | ORF Stop: TAA at 659 |
| CG120844-02, DNA Sequence | SNP Pos: 522 | SNP Change: A to G |

CTCCTTCCAGGACCTGGACCTCTGCCCTCTGGATGGCGGCATCCAGCTACGAATCTCCGACCACCACT
ACAGCAAGGGCTTCAGGCAGGCCGCGTCAGTTGTTGTGGCCATGGACAAGCTGAGGAAGATGCTGGTT
CCCTGCCCACAGACCTTCCAGGAGAATGACCTGAGCACCTTCTTTCCCTTCATCTTTGAAGAAGAACC
TATCTTCTTCGACACATGGGATAACGAGGCTTATGTGCACGATGCACCTGTACCATCACTGAACTGCA
CGCTCCGGGACTCACAGCAAAAAAGCTTGGTGATGTCTGGTCCATATGAACTGAAAGCTCTCCACCTC
CAACGAGAAGAAAGTAATGACAAAATACCTGTGGCCTTGGGCCTCAAGGAAAACAATCTGTACCTGTC
CTGCGTGTTGAAAGATGATAAGCCCACTCTACAGCTGGAGAGTGTAGATCCCAAAAATTACCCAAAGA
AGAAGATGGAAAAGCGATTTGTCTTCAACAAGATAGAAATCAATAGCAAGCTGGAATTTGAGTCTGCC
CAGTTCCCCAACTGGTACATCAGCACCTCTCAAGCAGAAAACATGCGCGTCTTCCTGGGAGGGACCAA
AGGCGGCCAGGATATAACTGACTTCACCATGCAATTTGTGTCTTCCTAAAGAGAGCTGTACCCAGAGA
GTCCTGTGC

| | | | |
|---|---|---|---|
| NOV2g, SNP13377793 of | SEQ ID NO: 90 | | MW at 25048.3 kD |
| CG120844-02, Protein Sequence | SNP Pos: 174 | 219 aa | SNP Change: Asn to Ser |

SFQDLDLCPLDGGIQLRISDHHYSKGFRQAASVVVAMDKLRKMLVPCPQTFQENDLSTFFPFIFEEEP
IFFDTWDNEAYVHDAPVRSLNCTLRDSQQKSLVMSGPYELKALHLQGEESNDKIPVALGLKEKNLYLS
CVLKDDKPTLQLESVDPKNYPKKKMEKRFVFNKIEINSKLEFESAQFPNWYISTSQAENMPVFLGGTK
GGQDITDFTMQFVSS

| | | |
|---|---|---|
| | SEQ ID NO: 91 | 689 bp |
| NOV2h, SNP13377781 of | ORF Start: at 2 | ORF Stop: TAA at 659 |
| CG120844-02, DNA Sequence | Pos: 566 | SNP Change: A to G |

CTCCTTCCAGGACCTGCACCTCTGCCCTCTGGATGGCGGCATCCAGCTACGAATCTCCGACCACCACT
ACAGCAAGGGCTTCAGGCAGGCCGCGTCAGTTGTTGTGGCCATGGACAAGCTGAGGAAGATGCTGGTT
CCCTGCCCACAGACCTTCCAGGAGAATGACCTGAGCACCTTCTTTCCCTTCATCTTTGAAGAAGAACC
TATCTTCTTCGACACATGGGATAACGAGGCTTATGTGCACGATGCACCTGTACGATCACTGAACTGCA
CGCTCCGGGACTCACAGCAAAAAAGCTTGGTGATGTCTCGTCCATATGAACTGAAAGCTCTCCACCTC
CAAGGAGAAGAAAGTAATGACAAAATACCTGTGGCCTTGGGCCTCAAGGAAAAGAATCTGTACCTGTC
CTGCGTGTTGAAAGATGATAACCCCACTCTACAGCTGGAGAGTGTAGATCCCAAAAATTACCCAAAGA
ACAAGATGGAAAAGCGATTTGTCTTCAACAAGATAGAAATCAATAACAAGCTGGAATTTGAGTCTGCC
CAGTTCCCCAACTGGTACATCGGCACCTCTCAAGCAGAAAACATGCCCGTCTTCCTGGGAGGGACCAA
AGGCGGCCAGGATATAACTGACTTCACCATGCAATTTGTGTCTTCCTAAAGAGAGCTGTACCCAGAGA
GTCCTGTCC

| | | | |
|---|---|---|---|
| NOV2h, SNP13377781 of | SEQ ID NO: 92 | | MW at 25045.3 kD |
| CG120844-02, Protein Sequence | SNP Pos: 189 | 219 aa | SNP Change: Ser to Gly |

SFQDLDLCPLDGGIQLRISDHHYSKGFRQAASVVVAMDKLRKMLVPCPQTFQENDLSTFFPFIFEEEP
IFFDTWDNEAYVHDAPVRSLNCTLRDSQQKSLVMSGPYELKALHLQGEESNDKIPVALCLKEKNLYLS
CVLKDDKPTLQLESVDPKNYPKKKMEKRFVFNKIEINNKLEFESAQFPNWYIGTSQAENMPVFLGGTK
GGQDITDFTMQFVSS

| | | |
|---|---|---|
| | SEQ ID NO: 93 | 689 bp |
| NOV2i, SNP13377792 of | ORF Start: at 2 | ORF Stop: TAA at 659 |
| CG120844-02, DNA Sequence | SNP Pos: 608 | SNP Change: A to G |

CTCCTTCCAGGACCTGGACCTCTGCCCTCTGGATGGCGGCATCCAGCTACGAATCTCCGACCACCACT
ACAGCAAGGGCTTCAGGCAGGCCGCGTCAGTTGTTGTGGCCATGGACAAGCTGAGGAAGATGCTGGTT
CCCTGCCCACAGACCTTCCAGGAGAATGACCTGAGCACCTTCTTTCCCTTCATCTTTGAAGAAGAACC
TATCTTCTTCGACACATGGGATAACGAGGCTTATGTGCACGATGCACCTGTACGATCACTGAACTGCA
CGCTCCGGCACTCACAGCAAAAAAGCTTGGTGATGTCTGGTCCATATGAACTGAAAGCTCTCCACCTC
CAAGGAGAAGAAAGTAATGACAAAATACCTGTGGCCTTGGGCCTCAAGGAAAAGAATCTGTACCTGTC
CTGCGTGTTGAAAGATGATAAGCCCACTCTACAGCTGGAGAGTGTAGATCCCAAAAATTACCCAAAGA
AGAAGATGGAAAAGCGATTTGTCTTCAACAAGATAGAAATCAATAACAAGCTGGAATTTGAGTCTGCC
CAGTTCCCCAACTGGTACATCAGCACCTCTCAAGCAGAAAACATGCCCGTCTTCCTGGGAGGGGCCAA
AGGCGGCCAGGATATAACTGACTTCACCATGCAATTTGTGTCTTCCTAAAGAGAGCTGTACCCAGAGA
GTCCTGTGC

| | | | |
|---|---|---|---|
| NOV2i, SNP13377792 of | SEQ ID NO: 94 | | MW at 25045.2 kD |
| CG120844-02, Protein Sequence | SNP Pos: 203 | 219 aa | SNP Change: Thr to Ala |

SFQDLDLCPLDGGIQLRISDHHYSKGFRQAASVVVAMDKLRKMLVPCPQTFQENDLSTFFPFIFEEEP
IFFDTWDNEAYVHDAPVRSLNCTLRDSQQKSLVMSGPYELKALHLQGEESNDKIPVALGLKEKNLYLS
CVLKDDKPTLQLESVDPKNYPKKKMEKRFVFNKIEINNKLEFESAQFPNWYISTSQAENMPVFLGGAK
GGQDITDFTMQFVSS

A ClustalW comparison of the above protein sequences yields the following sequence alignment shown in Table 2B.

TABLE 2B
Comparison of the NOV2 protein sequences.

| | |
|---|---|
| NOV2a | ----------------------------------SFQDLDLCPLDGGIQLRISDHHYSKG |
| NOV2b | ------------------------------------------------------------ |
| NOV2c | MAEVPELASEMMAYYSGNEDDLFFEADGPKQMKCSFQDLDLCPLDGGIQLRISDHHYSKG |
| NOV2a | FRQAASVVVAMDKLRKMLVPCPQTFQENDLSTFFPFIFEEEPIFFDTWDNEAYVHDAPVR |
| NOV2b | ------------------------------------------------------------ |
| NOV2c | FRQAASVVVAMDKLRKMLVPCPQTFQENDLSTFFPFIFEEEPIFFDTWDNEAYVHDAPVR |
| NOV2a | SLNCTLRDSQQKSLVMSGPYELKALHLQG----------------EESNDKIPVALGLKE |
| NOV2b | --TGSLRDSQQKSLVMSGPYELKALHLQG----------------EESNDKIPVALGLKE |
| NOV2c | SLNCTLRDSQQKSLVMSGPYELKALHLQGQDMEQQVVFSMSFVQGEESNDKIPVALGLKE |
| NOV2a | KNLYLSCVLKDDKPTLQLESVDPKNYPKKKMEKRFVFNKIEINNKLEFESAQFPNWYIST |
| NOV2b | KNLYLSCVLKDDKPTLQLESVDPKNYPKKKMEKRFVFNKIEINNKLEFESAQFPNWYIST |
| NOV2c | KNLYLSCVLKDDKPTLQLESVDPKNYPKKKMEKRFVFNKIEINNKLEFESAQFPNWYIST |
| NOV2a | SQAENMPVFLGGTKGGQDITDFTMQFVSS-- |
| NOV2b | SQAENMPVFLGGTKGGQDITDFTMQFVSLEG |
| NOV2c | SQAENMPVFLGGTKGGQDITDFTMQFVSS-- |
| NOV2a | (SEQ ID NO: 78) |
| NOV2b | (SEQ ID NO: 80) |
| NOV2c | (SEQ ID NO: 82) |

Further analysis of the NOV2a protein yielded the following properties shown in Table 2C.

TABLE 2C
Protein Sequence Properties NOV2a

| SignalP analysis: | No Known Signal Sequence Predicted | | | |
|---|---|---|---|---|
| PSORT II | PSG: a new signal peptide prediction method | | | |
| analysis: | N-region: length 11; pos.chg 0; neg.chg 3 | | | |
| | H-region: length 5; peak value 0.00 | | | |
| | PSG score: −4.40 | | | |
| | GvH: von Heijne's method for signal seg. recognition | | | |
| | GvH score (threshold: −2.1): −6.91 | | | |
| | possible cleavage site: between 52 and 53 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 5.73 (at 31) | | | |
| | ALOM score: 5.73 (number of TMSs: 0) | | | |
| | MITDISC: discrimination of mitochondrial targeting seq | | | |
| | R content: | 0 | Hyd Moment(75): | 2.74 |
| | Hyd Moment(95): | 5.40 | G content: | 0 |
| | D/E content: | 2 | S/T content: | 1 |

Score: −7.29
Gavel: prediction of cleavage sites for mitochondrial preseq
cleavage site motif not found
NUCDISC: discrimination of nuclear localization signals
pat4: PKKK (4) at 157
pat7: PKKKMEK (5) at 157
bipartite: none
content of basic residues: 11.0%
NLS Score: 0.21
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 70.6
COIL: Lupas's algorithm to detect coiled-coil regions
total: 0 residues
Final Results (k = 9/23):
43.5%: cytoplasmic
34.8%: nuclear
17.4%: mitochondrial
4.3%: Golgi
>> prediction for CG120844-02 is cyt (k = 23)

A search of the NOV2a protein against the Geneseq database, a proprietary database that contains sequences published in patents and patent publication, yielded several homologous proteins shown in Table 2D.

TABLE 2D
Geneseq Results for NOV2a

| | | | Identities/ | |
|---|---|---|---|---|
| | | | Similarities for | |
| Geneseq | Protein/Organism/Length | NOV2a Residues/ | the Matched | Expect |
| Identifier | [Patent #, Date] | Match Residues | Region | Value |
| ABU08119 | Recombinant interleukin 1 beta | 1 . . . 219 | 219/235 (93%) | e−124 |
| | protein - Homo sapiens, 269 aa. | 35 . . . 269 | 219/235 (93%) | |
| | [GB2375604-A, 20 NOV. 2002] | | | |
| AAE33564 | Human interleukin-1B (IL-1B) - | 1 . . . 219 | 219/235 (93%) | e−124 |
| | Homo sapiens, 269 aa. | 35 . . . 269 | 219/235 (93%) | |
| | [WO2002101015-A2, 19 DEC. | | | |
| | 2002] | | | |
| AAU11115 | Human Interleukin-1beta precursor | 1 . . . 219 | 219/235 (93%) | e−124 |
| | protein - Homo sapiens, 269 aa. | 35 . . . 269 | 219/235 (93%) | |
| | [US6306613-B1, 23 OCT. 2001] | | | |
| AAU80166 | Human interleukin 1 beta - Homo | 1 . . . 219 | 219/235 (93%) | e−124 |
| | sapiens, 269 aa. [WO200224951- | 35 . . . 269 | 219/235 (93%) | |
| | A1, 28 MAR. 2002] | | | |
| AAB35251 | Human pre-interleukin-1 beta - | 1 . . . 219 | 219/235 (93%) | e−124 |
| | Homo sapiens, 269 aa. | 35 . . . 269 | 219/235 (93%) | |
| | [US6187550-B1, 13 FEB. 2001] | | | |

In a BLAST search of public sequence databases, the NOV2a protein was found to have homology to the proteins shown in the BLASTP data in Table 2E.

TABLE 2E
Public BLASTP Results for NOV2a

| | | | Identities/ | |
|---|---|---|---|---|
| Protein | | | Similarities for | |
| Accession | | NOV2a Residues/ | the Matched | Expect |
| Number | Protein/Organism/Length | Match Residues | Portion | Value |
| AAP35877 | Interleukin 1, beta - Homo sapiens | 1 . . . 219 | 219/235 (93%) | e−123 |
| | (Human), 269 aa. | 35 . . . 269 | 219/235 (93%) | |
| AAP36922 | Homo sapiens interleukin 1, beta - | 1 . . . 219 | 219/235 (93%) | e−123 |
| | synthetic construct, 270 aa | 35 . . . 269 | 219/235 (93%) | |
| | (fragment). | | | |
| E975886 | PROTEIN SEQUENCE 6 FROM | 1 . . . 219 | 219/235 (93%) | e−123 |
| | PATENT NUMBER AU601173 - | 4 . . . 238 | 219/235 (93%) | |
| | unidentified, 238 aa. | | | |
| P01584 | Interleukin-1 beta precursor (IL-1 | 1 . . . 219 | 219/235 (93%) | e−123 |
| | beta) (Catabolin) - Homo sapiens | 35 . . . 269 | 219/235 (93%) | |
| | (Human), 269 aa. | | | |
| Q8MKH3 | Interleukin-1 beta - Saimiri | 1 . . . 219 | 198/235 (84%) | e−110 |
| | sciureus (Common squirrel | 35 . . . 269 | 207/235 (87%) | |
| | monkey), 269 aa. | | | |

PFam analysis predicts that the NOV2a protein contains the domains shown in the Table 2F.

TABLE 2F
Domain Analysis of NOV2a

| | | Identities/ | |
|---|---|---|---|
| | | Similarities | |
| | NOV2a Match | for the | Expect |
| Pfam Domain | Region | Matched Region | Value |
| IL1_propep | 1 . . . 69 | 42/111 (38%) | 5e−21 |
| | | 67/111 (60%) | |
| IL1 | 92 . . . 218 | 77/147 (52%) | 3.2e−69 |
| | | 123/147 (84%) | |

### Example 3

The NOV3 clone was analyzed, and the nucleotide and encoded polypeptide sequences are shown in Table 4A.

TABLE 3A
NOV3 Sequence Analysis

| | | |
|---|---|---|
| NOV3a, CG127616-01 | SEQ ID NO: 95 | 500 bp |
| DNA Sequence | ORF Start: ATG at 182 | ORF Stop: TGA at 494 |

CCCGGACCCGGACCGGGGCCACCGCGCCCGCTCTGCTCCGACACCGCGCCCCCTGGACAGCCGCCCTC
TCCTCCAGGCCCGTGGGGCTGGCCCTGCACCGCCGAGCTTCCCGGGATGAGGGCCCCCGGTGTGGTCA
CCCGGCGCGCCCCAGGTCGCTGAGGGACCCCGGCCAGGCGCGGAGATGGGGGTGCACGAATGTCCTGC
CTGGCTGTGGCTTCTCCTGTCCCTGCTGTCGCTCCCTCTGGGCCTCCCAGTCCTGGGCGCCCCACCAC
GCCTCATCTGTGACAGCCGAGTCCTGGAGAGGTACCTCTTGGAGGCCAAGGAGGCCGAGAATATCACG
AAGGAAGCCATCTCCCCTCCAGATGCGGCCTCAGCTGCTCCACTCCGAACAATCACTGCTGACACTTT
CCGCAAACTCTTCCGAGTCTACTCCAATTTCCTCCGGGGAAAGCTGAAGCTGTACACAGGGGAGGCCT
GCAGGACAGGGGACAGATGACCAG

| | | | |
|---|---|---|---|
| NOV3a, CG127616-01 | | | |
| Protein Sequence | SEQ ID NO: 96 | 104 aa | MW at 11567.4 kD |

MGVHECPAWLWLLLSLLSLPLGLPVLGAPPRLICDSRVLERYLLEAKEAENITKEAISPPDAASAAPL
RTITADTFRKLFRVYSNFLRGKLKLYTGEACRTGDR

| | | |
|---|---|---|
| NOV3b, CG127616-02 | SEQ ID NO: 97 | 324 bp |
| DNA Sequence | ORF Start: at 3 | ORF Stop: TGA at 318 |

CCTGGCTATCTGTTCTAGAATGTCCTGCCTGGCTGTGGCTTCTGCTGTCCCTGCTGTCGCTCCCTCTG
GGCCTCCCAGTCCTGGGCGCCCCACCACGCCTCATCTGTGACAGCCGAGTCCTGGAGAGGTACCTCTT
GGAGGCCAAGGAGGCCGAGAATATCACGAAGGAAGCCATCTCCCCTCCACATGCGGCCTCAGCTGCTC
CACTCCGAACAATCACTGCTGACACTTTCCGCAAACTCTTCCCAGTCTACTCCAATTTCCTCCGGGGA
AAGCTGAAGCTGTACACACGGGAGGCCTGCAGGACAGGGGACAGATGACCAG

| | | | |
|---|---|---|---|
| NOV3b, CG127616-02 | | | |
| Protein Sequence | SEQ ID NO: 98 | 105 aa | MW at 11741.6 kD |

WLSVLECPAWLWLLLSLLSLPLGLPVLGAPPRLICDSRVLERYLLEAKEAENITKEAISPPDAASAAP
LRTITADTFRKLFRVYSNFLRGKLKLYTGEACRTGDR

| | | |
|---|---|---|
| NOV3c, 227803412 | SEQ ID NO: 99 | 336 bp |
| DNA Sequence | ORF Start: at 1 | ORF Stop: TGA at 325 |

CGCGGATCCACCATGGGGGTGCACGAATGTCCTGCCTGGCTGTGGCTTCTCCTGTCCCTGCTGTCGCT
CCCTCTGGGCCTCCCAGTCCTGGGCGCCCCACCACGCCTCATCTGTGACACCCGAGTCCTGGAGAGGT
ACCTCTTGGAGGCCAAGGAGGCCGAGAATATCACGAAGGAAGCCATCTCCCCTCCAGATGCGGCCTCA
GCTGCTCCACTCCGAACAATCACTGCTGACACTTTCCGCAAACTCTTCCCAGTCTACTCCAATTTCCT
CCGGGGAAAGCTGAAGCTGTACACAGGGGAGGCCTGCAGGACAGGGGACAGATGACTCGAGCGG

| | | | |
|---|---|---|---|
| NOV3c, 227803412 | | | |
| Protein Sequence | SEQ ID NO: 100 | 108 aa | MW at 11968.8 kD |

RGSTMGVHECPAWLWLLLSLLSLPLGLPVLGAPPRLICDSRVLERYLLEAKEAENITKEAISPPDAAS
AAPLRTITADTFRKLFRVYSNFLRGKLKLYTGEACRTGDR

| | | |
|---|---|---|
| NOV3d, CG127616-03 | SEQ ID NO: 101 | 591 bp |
| DNA Sequence | ORF Start: at 3 | ORF Stop: TGA at 585 |

CCTGGCTATCTGTTCTAGAATGTCCTGCCTGGCTGTGGCTTCTCCTGTCCCTGCTGTCGCTCCCTCTG
GGCCTCCCAGTCCTGGGCGCCCCACCACGCCTCATCTGTGACAGCCGAGTCCTGGAGAGGTACCTCTT
GGAGGCCAAGGAGGCCGAGAATATCACCACGGGCTGTGCTGAACACTGCAGCTTGAATGAGAATATCA
CTGTCCCAGACACCAAAGTTAATTTCTATGCCTGGAAGAGGATGGAGGTCGGGCACCAGGCCGTAGAA
GTCTGGCAGGGCCTGGCCCTGCTGTCGGAAGCTGTCCTGCGGGGCCAGGCCCTGTTGATCAACTCTTC
CCAGCCGTGGGAGCCCCTGCAGCTGCATGTGGATAAAGCCGTCAGTGGCCTTCGCAGCCTCACCACTC
TGCTTCGGGCTCTGGGAGCCCAGAAGGAAGCCATCTCCCCTCCAGATGCGGCCTCAGCTGCTCCACTC
CGAACAATCACTGCTGACACTTTCCGCAAACTCTTCCGAGTCTACTCCAATTTCCTCCGGGGAAAGCT
GAAGCTGTACACAGGGGAGGCCTGCAGGACAGGGGACAGATGACCAG

| | | | |
|---|---|---|---|
| NOV3d, CG127616-03 | | | |
| Protein Sequence | SEQ ID NO: 102 | 194 aa | MW at 21494.7 kD |

WLSVLECPAWLWLLLSLLSLPLGLPVLGAPPRLICDSRVLERYLLEAKEAENITTGCAEHCSLNENIT
VPDTKVNFYAWKRMEVGQQAVEVWQGLALLSEAVLRGQALLINSSQPWEPLQLHVDKAVSGLRSLTTL
LRALGAQKEAISPPDAASAAPLRTITADTFRKLFRVYSNFLRGKLKLYTGEACRTGDR

| | | |
|---|---|---|
| NOV3e, CG127616-04 | SEQ ID NO: 103 | 282 bp |
| DNA Sequence | ORF Start: at 7 | ORF Stop: at 277 |

GGATCCTGGCTTCTCCTGTCCCTGCTGTCGCTCCCTCTGGCCCTCCCAGTCCTGGGCGCCCCACCACG
CCTCATCTGTGACAGCCGAGTCCTGGACAGGTACCTCTTGGAGGCCAAGCAGGCCGAGAATATCACGA
AGGAAGCCATCTCCCCTCCAGATGCGGCCTCAGCTGCTCCACTCCGAACAATCACTGCTGACACTTTC
CGCAAACTCTTCCGAGTCTACTCCAATTTCCTCCGGGGAAAGCTGAAGCTGTACACAGGGGAGGCCTG
CAGGCTCGAG

| | | | |
|---|---|---|---|
| NOV3e, CG127616-04 | | | |
| Protein Sequence | SEQ ID NO: 104 | 90 aa | MW at 10013.6 kD |

WLLLSLLSLPLGLPVLGAPPRLICDSRVLERYLLEAKEAENITKEAISPPDAASAAPLRTITADTFRK
LFRVYSNFLRGKLKLYTGEACR

| | | |
|---|---|---|
| NOV3f,CG127616-05 | SEQ ID NO: 105 | 1012 bp |
| DNA Sequence | ORF Start: ATG at 476 | ORF Stop: TGA at 815 |

CTCGCTGTGGCTTCTCCTGTCCCTGCTGTCGCTCCCTCTGGGCCTCCCAGTCCTGGGCGCCCCACCAC
GCCTCATCTGTGACAGCCGAGTCCTGGAGAGGTACCTCTTGGAGGCCAAGGAGGCCGAGAATATCACG
GTGAGACCCCTTCCCCACCACATTCCACAGAACTCACGCTCAGGGCTTCAGGGAACTCCTCCCAGATC
CAGGAACCTGGCACTTGGTTTGGGGTGGAGTTGGGAAGCTAGACACTGCCCCCCTACATAAGAATAAG
TCTGGTGGCCCCAAACCATACCTGGAAACTAGGCAAGGAGCAAAGCCAGCAGATCCTACAGCCTGTGG
GCCAGGGCCAGAGCCTTCAGGGACCCTTGAGTCCCCGGGCTGTGTGCATTTCAGACGGGCTGTGCTGA
ACACTGCAGCTTGAATGAGAATATCACTGTCCCAGACACCAAAGTTAATTTCTATGCCTGGAAGAGGA
TGGAGGTCGGGCAGCAGGCCGTAGAAGTCTGGCAGGGCCTGGCCCTGCTGTCGGAAGCTGTCCTGCGG
GGCCAGGCCCTGTTGGTCAACTCTTCCCAGCCGTGGGAGCCCCTGCAGCTGCATGTGGATAAAGCCGT
CAGTGGCCTTCGCAGCCTCACCACTCTGCTTCGGGCTCTGGGAGCCCAGAAGGAAGCCATCTCCCCTC
CAGATGCGGCCTCAGCTGCTCCACTCCCAACAATCACTGCTGACACTTTCCGCAAACTCTTCCGAGTC
TACTCCAATTTCCTCCGGGGAAAGCTCAAGCTGTACACAGGGGAGGCCTGCAGGACAGGGGACAGATG
ACCAGGTGTGTCCACCTGGGCATATCCACCACTTCCCTCACCAACATTGCTTGTGCCACACCCTCCCC
CGCCACTCCTGAACCCCTTGACTCCCGGGTGGTGGGAACCATGAATACAGGATGGGGGCTGCCTCTTG
CTCTCTTGGGGTCCAAGTTTTGTCTATTCTTCAACCTCATTGTCATGAATTCAAACCACC

| | | | |
|---|---|---|---|
| NOV3f, CG127616-05 | | | |
| Protein Sequence | SEQ ID NO: 106 | 113 aa | MW at 12334.1 kD |

MEVGQQAVEVWQGLALLSEAVLRGQALLVNSSQPWEPLQLHVDKAVSGLRSLTTLLRALGAQKEAISP
PDAASAAPLRTITADTFRKLFRVYSNFLRGKLKLYTGEACRTGDR

| | | |
|---|---|---|
| NOV3g, CG127616-06 | SEQ ID NO: 107 | 1012 bp |
| DNA Sequence | ORF Start: ATG at 476 | ORF Stop: TGA at 815 |

CTGGCTGTGGCTTCTCCTGTCCCTGCTGTCCCTCCCTCTGGGCCTCCCAGTCCTGGGCGCCCCACCAC
GCCTCATCTGTGACAGCCGAGTCCTGGAGAGGTACCTCTTGGAGGCCAAGGAGGCCGAGAATATCACG
GTGAGACCCCTTCCCCAGCACATTCCACAGAACTCACGCTCAGGGCTTCAGGGAACTCCTCCCAGATC
CAGGAACCTGGCACTTGGTTTGGGGTGCAGTTGGGAAGCTAGACACTGCCCCCCTACATAAGAATAAG
TCTGGTGGCCCCAAACCATACCTGGAAACTAGGCAAGGAGCAAAGCCAGCAGATCCTACAGCCTGTGG
GCCAGGGCCAGAGCCTTCAGGGACCCTTGACTCCCCGGGCTGTGTGCATTTCAGACGGGCTGTGCTCA
ACACTGCAGCTTGAATGAGAATATCACTGTCCCAGACACCAAAGTTAATTTCTATGCCTGGAAGAGGA
TGGAGGTCGGGCAGCAGGCCGTAGAAGTCTGGCAGGGCCTGGCCCTGCTGTCGGAAGCTGTCCTGCGG
GGCCAGGCCCTGTTGGTCAACTCTTCCCAGCCGTGGGAGCCCCTGCAGCTGCATGTGGATAAAGCCGT
CAGTGGCCTTCGCAGCCTCACCACTCTGCTTCGGGCTCTGGGAGCCCAGAAGGAAGCCATCTCCCCTC
CAGATGCGGCCTCAGCTGCTCCACTCCGAACAATCACTGCTGACACTTTCCGCAAACTCTTCCGAGTC
TACTCCAATTTCCTCCGGGGAAAGCTGAAGCTGTACACAGGGGAGGCCTGCAGGACAGGGGACAGATG
ACCAGGTGTGTCCACCTGGGCATATCCACCACTTCCCTCACCAACATTGCTTGTGCCACACCCTCCCC
CGCCAGTCCTGAACCCCTTGACTCCGGGGTGGTGGGAACCATGAATACAGGATGGGGGCTGCCTCTTG
CTCTCTTGGGGTCCAAGTTTTGTGTATTCTTCAACCTCATTCTCATGAATTGAAACCACC

| | | | |
|---|---|---|---|
| NOV3g, CG127616-06 | | | |
| Protein Sequence | SEQ ID NO: 108 | 113 aa | MW at 12334.1 kD |

MEVGQQAVEVWQGLALLSEAVLRGQALLVNSSQPWEPLQLHVDKAVSGLRSLTTLLRALGAQKEAISP
PDAASAAPLRTITADTFRKLFRVYSNFLRGKLKLYTGEACRTGDR

| | | |
|---|---|---|
| NOV3h, CG127616-07 | SEQ ID NO: 109 | 1012 bp |
| DNA Sequence | ORF Start: ATG at 476 | ORF Stop: TGA at 815 |

CTGGCTGTGGCTTCTCCTGTCCCTGCTGTCCCTCCCTCTGGGCCTCCCAGTCCTGGGCGCCCCACCAC
GCCTCATCTGTGACAGCCGAGTCCTGGACAGGTACCTCTTGGAGGCCAAGGAGGCCCAGAATATCACG
GTGAGACCCCTTCCCCAGCACATTCCACAGAACTCACGCTCAGGGCTTCACGGAACTCCTCCCAGATC
CAGGAACCTGGCACTTGGTTTGGGGTGGAGTTGGGAAGCTAGACACTGCCCCCCTACATAACAATAAG
TCTGGTGCCCCCAAACCATACCTGGAAACTAGGCAAGGAGCAAAGCCAGCAGATCCTACAGCCTGTGG
GCCACGGCCAGAGCCTTCAGCGACCCTTGACTCCCCGGGCTGTGTGCATTTCAGACGGGCTGTGCTGA
ACACTGCAGCTTGAATGAGAATATCACTGTCCCAGACACCAAAGTTAATTTCTATGCCTGGAAGAGGA
TGGAGGTCGGGCAGCAGGCCGTAGAAGTCTGGCAGGGCCTGCCCCTGCTGTCGGAAGCTGTCCTGCGG
GGCCAGGCCCTGTTGGTCAACTCTTCCCAGCCGTGGGAGCCCCTGCAGCTGCATGTGGATAAAGCCGT
CAGTGGCCTTCGCAGCCTCACCACTCTGCTTCGGGCTCTGGGAGCCCAGAAGGAAGCCATCTCCCCTC
CAGATGCGGCCTCAGCTGCTCCACTCCGAACAATCACTGCTGACACTTTCCGCAAACTCTTCCGAGTC
TACTCCAATTTCCTCCGGGGAAAGCTGAAGCTGTACACAGCGGAGGCCTGCAGGACAGGCGACAGATG
ACCAGGTCTGTCCACCTGGGCATATCCACCACTTCCCTCACCAACATTGCTTGTGCCACACCCTCCCC
CGCCACTCCTGAACCCCTTGACTCCGGGGTGGTGGGAACCATGAATACAGGATGGGGGCTGCCTCTTG
CTCTCTTGGGGTCCAAGTTTTGTGTATTCTTCAACCTCATTGTCATGAATTCAAACCACC

| | | | |
|---|---|---|---|
| NOV3h, CG127616-07 | | | |
| Protein Sequence | SEQ ID NO: 110 | 113 aa | MW at 12334.1 kD |

MEVGQQAVEVWQGLALLSEAVLRGQALLVNSSQPWEPLQLHVDKAVSGLRSLTTLLRALGAQKEAISP
PDAASAAPLRTITADTFRKLFRVYSNFLRGKLKLYTGEACRTGDR

| | | |
|---|---|---|
| NOV3i,CG127616-08 | SEQ ID NO: 111 | 333 bp |
| DNA Sequence | ORF Start: ATG at 10 | ORF Stop: TGA at 322 |

CGCGGATCCATGGGGGTGCACGAATGTCCTGCCTGGCTGTGGCTTCTCCTGTCCCTGCTGTCGCTCCC
TCTGGGCCTCCCAGTCCTGGGCGCCCCACCACGCCTCATCTGTGACAGCCGAGTCCTGGAGAGGTACC
TCTTGGAGGCCAAGGAGGCCGAGAATATCACGAAGGAAGCCATCTCCCCTCCAGATGCGGCCTCAGCT
GCTCCACTCCGAACAATCACTGCTGACACTTTCCGCAAACTCTTCCGAGTCTACTCCAATTTCCTCCG
GGGAAAGCTGAAGCTGTACACAGGGGAGGCCTGCAGGACAGGGGACAGATGACTCGAGCGG

| | | | |
|---|---|---|---|
| NOV3i, CG127616-08 | | | |
| Protein Sequence | SEQ ID NO: 112 | 104 aa | MW at 11567.4 kD |

MGVHECPAWLWLLLSLLSLPLGLPVLGAPPRLICDSRVLERYLLEAKEAENITKEAISPPDAASAAPL
RTITADTFRKLFRVYSNFLRGKLKLYTGEACRTGDR

| | | |
|---|---|---|
| NOV3j, CG127616-09 | SEQ ID NO: 113 | 330 bp |
| DNA Sequence | ORF Start: at 22 | ORF Stop: at 322 |

CGCGGATCCATGGGGGTGCACGAATGTCCTGCCTGGCTGTGGCTTCTCCTGTCCCTGCTGTCGCTCCC
TCTGGGCCTCCCAGTCCTGGGCGCCCCACCACGCCTCATCTGTGACAGCCGAGTCCTGGAGAGGTACC
TCTTGGAGGCCAAGGAGGCCGAGAATATCACGAAGGAAGCCATCTCCCCTCCAGATGCGGCCTCAGCT
GCTCCACTCCGAACAATCACTGCTGACACTTTCCGCAAACTCTTCCGAGTCTACTCCAATTTCCTCCG
GGGAAAGCTGAAGCTGTACACAGCGGAGGCCTCCACGACAGGGGACAGACTCGAGCGG

| | | | |
|---|---|---|---|
| NOV3j, CG127616-09 | | | |
| Protein Sequence | SEQ ID NO: 114 | 100 aa | MW at 11142.8 kD |

ECPAWLWLLLSLLSLPLGLPVLGAPPRLICDSRVLERYLLEAKEAENITKEAISPPDAASAAPLRTIT
ADTFRKLFRVYSNFLRGKLKLYTGEACRTGDR

A ClustalW comparison of the above protein sequences yields the following sequence alignment shown in Table 3B.

TABLE 3B
Comparison of the NOV3 protein sequences.

| | |
|---|---|
| NOV3a | ----MGVHECPAWLWLLLSLLSLPLGLPVLGAPPRLICDSRVLERYLLEAKEAENITK-- |
| NOV3b | ---WLSVLECPAWLWLLLSLLSLPLGLPVLGAPPRLICDSRVLERYLLEAKEAENITK-- |
| NOV3c | RGSTMGVHECPAWLWLLLSLLSLPLGLPVLGAPPRLICDSRVLERYLLEAKEAENITK-- |
| NOV3d | ---WLSVLECPAWLWLLLSLLSLPLGLPVLGAPPRLICDSRVLERYLLEAKEAENITTGC |
| NOV3e | --------------WLLLSLLSLPLGLPVLGAPPRLICDSRVLERYLLEAKEAENITK-- |
| NOV3f | ----MEVGQQAVEVWQGLALLSEAV----LRGQALLVNSSQPWEPLQLHVDKAVSGLRSL |
| NOV3g | ----MEVGQQAVEVWQGLALLSEAV----LRGQALLVNSSQPWEPLQLHVDKAVSGLRSL |
| NOV3h | ----MEVGQQAVEVWQGLALLSEAV----LRGQALLVNSSQPWEPLQLHVDKAVSGLRSL |
| NOV3i | ----MGVHECPAWLWLLLSLLSLPLGLPVLGAPPRLICDSRVLERYLLEAKEAENITK-- |
| NOV3j | --------ECPAWLWLLLSLLSLPLGLPVLGAPPRLICDSRVLERYLLEAKEAENITK-- |
| NOV3a | ------------------------------------------------------------ |
| NOV3b | ------------------------------------------------------------ |
| NOV3c | ------------------------------------------------------------ |
| NOV3d | AEHCSLNENITVPDTKVNFYAWKRMEVGQQAVEVWQGLALLSEAVLRGQALLINSSQPWE |
| NOV3e | ------------------------------------------------------------ |
| NOV3f | TTLLRALG---------------------------------------------------- |
| NOV3g | TTLLRALG---------------------------------------------------- |
| NOV3h | TTLLRALG---------------------------------------------------- |
| NOV3i | ------------------------------------------------------------ |
| NOV3j | ------------------------------------------------------------ |
| NOV3a | ---------------------------EAISPPDAASAAPLRTITADTFRKLFRVYSNFL |
| NOV3b | ---------------------------EAISPPDAASAAPLRTITADTFRKLFRVYSNFL |
| NOV3c | ---------------------------EAISPPDAASAAPLRTITADTFRKLFRVYSNFL |
| NOV3d | PLQLHVDKAVSCLRSLTTLLPALGAQKEAISPPDAASAAPLRTITADTFRKLFRVYSNFL |
| NOV3e | ---------------------------EAISPPDAASAAPLRTITADTFRKLFRVYSNFL |
| NOV3f | ------------------------AQKEAISPPDAASAAPLRTITADTFRKLFRVYSNFL |
| NOV3g | ------------------------AQKEAISPPDAASAAPLRTITADTFRKLFRVYSNFL |
| NOV3h | ------------------------AQKEAISPPDAASAAPLRTITADTFRKLFRVYSNFL |
| NOV3i | ---------------------------EAISPPDAASAAPLRTITADTFRKLFRVYSNFL |
| NOV3j | ---------------------------EAISPPDAASAAPLRTITADTFRKLFRVYSNFL |
| NOV3a | RGKLKLYTGEACRTGDR |
| NOV3b | RGKLKLYTGEACRTGDR |
| NOV3c | RGKLKLYTGEACRTGDR |
| NOV3d | RGKLKLYTGEACRTGDR |
| NOV3e | RGKLKLYTGEACR---- |
| NOV3f | RGKLKLYTGEACRTGDR |
| NOV3g | RGKLKLYTGEACRTGDR |
| NOV3h | RGKLKLYTGEACRTGDR |
| NOV3i | RGKLKLYTGEACRTGDR |
| NOV3j | RGKLKLYTGEACRTGDR |
| NOV3a | (SEQ ID NO: 102) |
| NOV3b | (SEQ ID NO: 104) |
| NOV3c | (SEQ ID NO: 106) |
| NOV3d | (SEQ ID NO: 108) |
| NOV3e | (SEQ ID NO: 110) |
| NOV3f | (SEQ ID NO: 112) |
| NOV3g | (SEQ ID NO: 114) |
| NOV3h | (SEQ ID NO: 116) |
| NOV3i | (SEQ ID NO: 118) |
| NOV3j | (SEQ ID NO: 120) |

Further analysis of the NOV3a protein yielded the following properties shown in Table 3C.

TABLE 3C
Protein Sequence Properties NOV3a

| SignalP analysis: | Cleavage site between residues 28 and 29 | | | |
|---|---|---|---|---|
| PSORT II | PSG: a new signal peptide prediction method | | | |
| analysis: | N-region: length 5; pos.chg 0; neg.chg 1 | | | |
| | H-region: length 25; peak value 0.00 | | | |
| | PSG score: −4.40 | | | |
| | GvH: von Heijne's method for signal seq. recognition | | | |
| | GvH score (threshold: −2.1): 1.97 | | | |
| | possible cleavage site: between 22 and 23 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL | Likelihood = −4.88 Transmembrane 10-26 | | |
| | PERIPHERAL | Likelihood = 11.09 (at 56) | | |
| | ALOM score: −4.88 (number of TMSs: 1) | | | |
| | MTOP: Prediction of membrane topology (Hartmann et al.) | | | |
| | Center position for calculation: 17 | | | |
| | Charge difference: −0.5 C(0.0)-N(0.5) | | | |
| | N >= C: N-terminal side will be inside | | | |
| | >>> membrane topology: type 2 (cytoplasmic tail 1 to 10) | | | |
| | MITDISC: discrimination of mitochondrial targeting seq | | | |
| | R content: | 1 | Hyd Moment (75): | 6.40 |
| | Hyd Moment (95): | 3.98 | G content: | 3 |
| | D/E content: | 2 | S/T content: | 2 |

```
Score: −7.17
Gavel: prediction of cleavage sites for mitochondrial preseq
R-2 motif at 41 PRL|IC
NUCDISC: discrimination of nuclear localization signals
pat4: none
pat7: none
bipartite: none
content of basic residues: 13.5%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
30.4%: mitochondrial
26.1%: cytoplasmic
13.0%: Golgi
  8.7%: vacuolar
  8.7%: endoplasmic reticulum
  4.3%: extracellular, including cell wall
  4.3%: nuclear
  4.3%: vesicles of secretory system
>> prediction for CG127616-01 is mit (k = 23)
```

A search of the NOV3a protein against the Geneseq database, a proprietary database that contains sequences published in patents and patent publication, yielded several homologous proteins shown in Table 3D.

TABLE 3D
Geneseq Results for NOV3a

| | | | Identities/ | |
|---|---|---|---|---|
| | | NOV3a | Similarities | |
| | | Residues/ | for the | |
| Geneseq | Protein/Organism/Length [Patent | Match | Matched | Expect |
| Identifier | #, Date] | Residues | Region | Value |
| AAW14143 | Erythropoietin variant JM - Homo | 1 . . . 54 | 54/54 (100%) | 2e−25 |
| | sapiens, 193 aa. [WO9708307-A1, | 1 . . . 54 | 54/54 (100%) | |
| | 06 MAR. 1997] | | | |
| AAE32131 | Human erythropoietin protein - | 1 . . . 53 | 53/53 (100%) | 6e−25 |
| | Homo sapiens, 193 aa. | 1 . . . 53 | 53/53 (100%) | |
| | [WO200285940-A2, 31 OCT. 2002] | | | |
| AAE15348 | Human erythropoietin (Epo) N47-Fc | 1 . . . 53 | 53/53 (100%) | 6e−25 |
| | fusion protein - Homo sapiens, 420 | 1 . . . 53 | 53/53 (100%) | |
| | aa. [WO200181405-A2, 01 NOV. | | | |
| | 2001] | | | |
| AAE15341 | Human erythropoietin (Epo) | 1 . . . 53 | 53/53 (100%) | 6e−25 |
| | protein - Homo sapiens, 193 aa. | 1 . . . 53 | 53/53 (100%) | |
| | [WO200181405-A2, 01 NOV. 2001] | | | |
| ABB79939 | Human erythropoietin-HCG C- | 1 . . . 53 | 53/53 (100%) | 6e−25 |
| | terminal peptide fusion protein | 1 . . . 53 | 53/53 (100%) | |
| | ECTP - Homo sapiens, 220 aa. | | | |
| | [WO200248194-A1, 20 JUN. 2002] | | | |

In a BLAST search of public sequence databases, the NOV3a protein was found to have homology to the proteins shown in the BLASTP data in Table 3E.

TABLE 3E
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P01588 | Erythropoietin precursor (Epoetin) - | 1 . . . 53 | 53/53 (100%) | 2e−24 |
| | Homo sapiens (Human), 193 aa. | 1 . . . 53 | 53/53 (100%) | |
| P07865 | Erythropoietin precursor - Macaca | 1 . . . 53 | 50/53 (94%) | 3e−23 |
| | fascicularis (Crab eating macaque) | 1 . . . 53 | 52/53 (97%) | |
| | (Cynomolgus monkey), 192 aa. | | | |
| Q28513 | Erythropoietin precursor - Macaca | 1 . . . 53 | 49/53 (92%) | 4e−23 |
| | mulatta (Rhesus macaque), 192 aa. | 1 . . . 53 | 52/53 (97%) | |
| CAC41224 | Sequence 1 from Patent | 41 . . . 104 | 54/64 (84%) | 2e−22 |
| | WO0136489 - Homo sapiens | 103 . . . 166 | 54/64 (84%) | |
| | (Human), 166 aa (fragment). | | | |
| Q867B1 | Erythropoietin - Equus caballus | 41 . . . 104 | 47/64 (73%) | 1e−18 |
| | (Horse), 192 aa. | 129 . . . 192 | 48/64 (74%) | |

PFam analysis predicts that the NOV3a protein contains the domains shown in the Table 3F.

TABLE 3F
Domain Analysis of NOV3a

| | | Identities/ | |
|---|---|---|---|
| | | Similarities | |
| | NOV3a Match | for the | Expect |
| Pfam Domain | Region | Matched Region | Value |
| EPO_TPO | 11 . . . 104 | 77/185 (42%) | 6e−13 |
| | | 92/185 (50%) | |

### Example 4

The NOV4 clone was analyzed, and the nucleotide and encoded polypeptide sequences are shown in Table 4A.

TABLE 4A
NOV4 Sequence Analysis

| | | |
|---|---|---|
| NOV4a, CG54455-03 | SEQ ID NO: 115 | 510 bp |
| DNA Sequence | ORF Start: ATG at 1 | ORF Stop: end of sequence |

ATGCGCCGCCGCCTGTGGCTGGGCCTGGCCTGGCTGCTGCTGGCGCGGGCGCCGGACGCCGCGGGAAC
CCCGAGCGCGTCGCGGGGACCGCGCAGCTACCCGCACCTGGAGGGCGACGTGCGCTGGCGGCGCCTCT
TCTCCTCCACTCACTTCTTCCTGCCCGTGGATCCCGGCGGCCGCGTGCAGGGCACCCGCTGGCGCCAC
GGCCAGGACAGCATCCTGCAGATCCGCTCTGTACACGTGGGCGTCGTGGTCATCAAAGCAGTGTCCTC
AGGCTTCTACGTGGCCATGAACCGCCGGGGCCGCCTCTACGGGTCGCGACTCTACACCGTGGACTGCA
GGTTCCGGGAGCGCATCGAAGAGAACGGCCACAACACCTACGCCTCACAGCGCTGGCGCCGCCGCGGC
CAGCCCATGTTCCTGGCGCTGGACAGGAGGGGGGGGCCCCGGCCAGGCGGCCGGACGCGGCGGTAGCA
CCTGTCCGCCCACTTCCTGCCCGTCCTGGTCTCC

| | | | |
|---|---|---|---|
| NOV4a, CG54455-03 | | | |
| Protein Sequence | SEQ ID NO: 116 | 170 aa | MW at 19662.4 kD |

MRRRLWLGLAWLLLARAPDAAGTPSASRGPRSYPHLEGDVRWRRLFSSTHFFLRVDPGGRVQGTRWRH
GQDSILEIRSVHVGVVVIKAVSSGFYVAMNRRGRLYGSRLYTVDCRFRERIEENGHNTYASQRWRRRG
QPMFLALDRRGGPRPGGRTRRYHLSAHFLPVLVS

| | | |
|---|---|---|
| NOV4b, 260403849 | SEQ ID NO: 117 | 527 bp |
| DNA Sequence | ORF Start: at 3 | ORF Stop: end of sequence |

AGATCTCCACCATGCGCCGCCGCCTGTGGCTGGGCCTGGCCTGGCTGCTGCTGGCGCGGGCGCCGGAC
GCCGCGGGAACCCCGAGCGCGTCGCGGGGACCGCGCAGCTACCCGCACCTCGAGGGCGACGTGCGCTG
GCGGCGTCTCTTCTCCTCCACTCACTTCTTCCTGCGCGTGGATCCCGGCCGCCGCGTGCAGGGCACCC
GCTGGCGCCACGGCCAGGACAGCATCCTGGAGATCCGCTCTGTACACGTGGGCGTCGTGGTCATCAAA
GCAGTGTCCTCAGGCTTCTACGTGGCCATGAACCGCCGGGGCCGCCTCTACGGGTCGCGACTCTACAC
CGTGGACTGCAGGTTCCGGGAGCGCATCGAAGAGAACGGCCACAACACCTACGCCTCACAGCGCTGGC
GCCGCCCCGGCCAGCCCATGTTCCTGGCGCTGGACAGGAGGGGGGGGCCCCGGCCAGGCGGCCGGACG
CGGCGGTACCACCTGTCCGCCCACTTCCTGCCCGTCCTGGTCTCCCTCGAG

| | | | |
|---|---|---|---|
| NOV4b, 260403849 | | | |
| Protein Sequence | SEQ ID NO: 118 | 175 aa | MW at 20206.0 kD |

ISTMRRRLWLGLAWLLLARAPDAAGTPSASRGPRSYPHLECDVRWRRLFSSTHFFLRVDPGGRVQGTR
WRHGQDSILEIRSVHVGVVVIKAVSSGFYVAMNRRGRLYGSRLYTVDCRFRERIEENCHNTYASQRWR
RRGQPMFLALDRRGGPRPGGRTRRYHLSAHFLPVLVSLE

| | | |
|---|---|---|
| NOV4c, CG54455-07 | SEQ ID NO: 119 | 527 bp |
| DNA Sequence | ORF Start: ATG at 12 | ORF Stop: at 522 |

AGATCTCCACCATGCGCCGCCGCCTGTGGCTGGGCCTGGCCTGGCTGCTGCTGGCGCGGGCGCGGGAC
GCCGCGGCAACCCCGAGCGCGTCGCGGGGACCGCGCAGCTACCCGCACCTGGAGGGCGACGTGGGCTG
GCGGCGTCTCTTCTCCTCCACTCACTTCTTCCTGCGCGTGGATCCCGGCGGCCGCGTGCAGGGCACCC
GCTGGCGCCACGGCCAGGACAGCATCCTGGAGATCCGCTCTGTACACGTGGGCGTCGTGGTCATCAAA
GCAGTGTCCTCAGGCTTCTACGTGGCCATGAACCGCCGGGGCCGCCTCTACGGGTCGCGACTCTACAC
CGTGGACTGCAGGTTCCGGGAGCGCATCGAAGAGAACGGCCACAACACCTACGCCTCACAGCGCTGGC
GCCGCCGCGGCCAGCCCATGTTCCTGGCGCTGGACAGGAGGGGGGGGCCCCGGCCAGGCGGCCGGACG
CGGCGGTACCACCTGTCCGCCCACTTCCTGCCCGTCCTGGTCTCCCTCGAG

| | | | |
|---|---|---|---|
| NOV4c, CG54455-07 | | | |
| Protein Sequence | SEQ ID NO: 120 | 170 aa | MW at 19662.4 kD |

MRRRLWLGLAWLLLARAPDAAGTPSASRGPRSYPHLEGDVRWRRLFSSTHFFLRVDPGGRVQGTRWRH
GQDSILEIRSVHVGVVVIKAVSSGFYVAMNRRGRLYGSRLYTVDCRFRERIEENGHNTYASQRWRRRG
QPMFLALDRRGGPRPGGRTRRYHLSAHFLPVLVS

| | | |
|---|---|---|
| NOV4d, 306448506 | SEQ ID NO: 121 | 529 bp |
| DNA Sequence | ORF Start: at 2 | ORF Stop: TAG at 518 |

CACCCATATGCGCCGCCGCCTGTGGCTGGGCCTGGCCTGGCTGCTGCTGGCGCGGGCGCCGGACGCCG
CGGGAACCCCGAGCGCGTCGCGGGGACCGCGCAGCTACCCGCACCTGGAGGGCGACGTGCGCTGGCGG
CGTCTCTTCTCCTCCACTCACTTCTTCCTGCGCGTGGATCCCGGCGCCCGCGTGCAGGGCACCCGCTG
GCGCCACGGCCAGGACAGCATCCTGGAGATCCGCTCTGTACACGTGGGCGTCGTGGTCATCAAAGCAG
TGTCCTCAGGCTTCTACGTGGCCATGAACCGCCGGGGCCGCCTCTACGGGTCGCGACTCTACACCGTG
GACTGCAGGTTCCGGGAGCGCATCGAAGAGAACGGCCACAACACCTACGCCTCACAGCGCTGGCGCCG
CCGCGGCCAGCCCATGTTCCTGGCGCTGGACAGGAGGGGGGGGCCCCGGCCAGGCGGCCGGACGCGGC
GGTACCACCTGTCCGCCCACTTCCTGCCCGTCCTGGTCTCCTAGCTCGAGGGC

| | | | |
|---|---|---|---|
| NOV4d, 306448506 | | | |
| Protein Sequence | SEQ ID NO: 122 | 172 aa | MW at 19900.6 kD |

THMRRRLWLGLAWLLLARAPDAAGTPSASRGPRSYPHLEGDVRWRRLFSSTHFFLRVDPGGRVQGTRW
RHGQDSILEIRSVHVGVVVIKAVSSGFYVAMNRRGRLYGSRLYTVDCRFRERIEENGHNTYASQRWRR
RGQPMFLALDRRGGPRPGGRTRRYHLSAHFLPVLVS

| | | |
|---|---|---|
| NOV4e, CG54455-01 | SEQ ID NO: 123 | 1340 bp |
| DNA Sequence | ORF Start: ATG at 130 | ORF Stop: TGA at 640 |

CCATTGGCCGGCGTCCCCGCCCCAGCGAACCCGGCCCCGCCCCCGAGGCGCCCCATTGGCCCCGCCGC
GCGAAGGCAGAGCCGCGGACGCCCGGGAGCGACGAGCGCGCAGCCAACCGGGTGCCGGGTCATGCGCC
GCCGCCTGTGGCTGGGCCTGGCCTGGCTGCTGCTGGCGCGGGCGCCGGACGCCGCGGGAACCCCGAGC
GCGTCGCGGGGACCGCGCAGCTACCCGCACCTGGAGGGCGACGTGCGCTGGCGGCGCCTCTTCTCCTC
CACTCACTTCTTCCTGCGCGTGGATCCCGGCGGCCGCGTGCAGGGCACCCGCTGGCGCCACGGCCAGG
ACAGCATCCTGGAGATCCGCTCTGTACACGTGGGCGTCGTGGTCATCAAAGCAGTGTCCTCAGGCTTC
TACGTGGCCATGAACCGCCGGGGCCGCGTCTACGGGTCGCGACTCTACACCGTGGACTGCAGGTTCCG
GGAGCGCATCGAAGAGAACGGCCACAACACCTACGCCTCACAGCGCTGGCGCCGCCGCGGCCAGCCCA
TGTTCCTGGCGCTGGACAGGAGGGGGGGGCCCCGGCCAGGCGGCCGGACGCGGCGGTACCACCTGTCC
GCCCACTTCCTGCCCGTCCTGGTCTCCTGAGGCCCTGAGAGGCCGGCGGCTCCCCAAGCCATTGGCCG
GCGTCCCCGCCCCAGCCAACCCGGCCCCGCCCCCCAGGCGCCCCATTGCCCCCGCCGCGCGAAGGCAG
AGCCGCGGACGCCCGGGAGCGACGAGCGCGCAGCGAACCGGGTGCCGGGTCATGCCCCGCCGCCTGTG
GCTGGGCCTGGCCTGGCTGCTGCTGGCGCGGGCGCCGGACGCCGCGGGAACCCCGAGCGCGTCGCGGC
GACCGCGCAGCTACCCGCACCTGGAGGGCGACGTGCGCTGGCGGCGCCTCTTCTCCTCCACTCACTTC
TTCCTGCGCGTGGATCCCGGCGGCCGCGTGCAGGGCACCCGCTGGCGCCACGGCCAGGACAGCATCCT
GGAGATCCGCTCTGTACACGTGGGCGTCGTGGTCATCAAAGCAGTGTCCTCAGGCTTCTACGTGGCCA
TGAACCGCCGGGGCCGCCTCTACGGGTCGCGACTCTACACCGTGGACTGCAGGTTCCGGGAGCGCATC
GAAGAGAACGGCCACAACACCTACGCCTCACAGCGCTGGCGCCGCCGCGGCCAGCCCATGTTCCTGGC
GCTGGACAGGAGGGGCGGGCCCCGGCCAGGCGGCCGGACGCGGCGGTACCACCTGTCCGCCCACTTCC
TGCCCGTCCTGGTCTCCTGAGGCCCTGAGAGGCCGGCGGCTCCCCAAG

| | | | |
|---|---|---|---|
| NOV4e, CG54455-01 | | | |
| Protein Sequence | SEQ ID NO: 124 | 170 aa | MW at 19662.4 kD |

MRRRLWLGLAWLLLARAPDAAGTPSASRGPRSYPHLEGDVRWRRLFSSTHFFLRVDPGGRVQGTRWRH
GQDSILEIRSVHVGVVVIKAVSSGFYVAMNRRGRLYGSRLYTVDCRFRERIEENGHNTYASQRWRRRG
QPMFLALDRRGGPRPGGRTRRYHLSAHFLPVLVS

| | | |
|---|---|---|
| NOV4f, CG54455-02 | SEQ ID NO: 125 | 943 bp |
| DNA Sequence | ORF Start: at 3 | ORF Stop: TGA at 483 |

TAGGCCGCCTCTGGCTGGGCCTAGCCTGGCTGCTGTTGACGCGGGCACCGGGCGCTCCGGGAGGGTAC
CCGCATCTGGAGGGCGACGTGCGCTGGCGCCGCCTCTTCTCCTCCACTCACTTTTTCCTGCGTGTGGA
CCTTGGTGGTCGGGTGCAGGGGAGGCGTTGGCGGCACGGCCAGGACAGTATAGTGGAGATCCGTTCTG
TCCGTGTGGGCACTGTGGTGATCAAAGCTGTGTACTCAGGCTTCTATGTGGCCATGAATCGCAGGGGC
CGCCTCTATGGGTCGCGGGTCTACTCTGTGGACTGTAGGTTCCGGGAGCGCATCGAGGAGAACGGCTA
CAACACATACGCCTCGCGACGTTGGAGGCACCGCGGCCGACCCATGTTCCTGGCACTTGACAGCCAAG
GCATTCCCAGGCAAGGCAGACGGACACGACGGCACCAACTGTCCACACACTTCCTGCCAGTCTTGGTC
TCGTCTTGAAGGGCCTGCCAATGGTTCACGAGGCATGGGCGGCACACAGGCCCTGGAAGATCCGGACC
TGAACAACCAAGGGCCAGGCCAGAGACCCTGGGCCAACACGAGTCTTTATGTCACAAGCCGGGCCCCC
GCTGGCTGCCGGGCATGGAGACATGGCAGGGTCCCTGCAAGTGAACCCAGCGCTCAGGGGGATACACA
GAACTGGCAGTTTGCATCCATCTAGTTTGCAGATGAGAACACTCTGGGCACACCACGGAGAGGTTTGC
AGGTGGGAACACACACCAGGTGGATGAGGAAAGCAGGCAGGGAGGACCGGGGAGGGTGGACATGTCAC
GGAGGGCAGGGCCCGCGTCAGTGGGGACAGAGACATGTTCGCCCATGTGGCCGAAGCTTGGGGCTGGA
GTTAAGAGCACTTCTTGCTCTTCCAAGGGGCCTGAGTTTAATTCTTCCACATCAGGCTG

| | | | |
|---|---|---|---|
| NOV4f, CG54455-02 | | | |
| Protein Sequence | SEQ ID NO: 126 | 160 aa | MW at 18639.2 kD |

GRLWLGLAWLLLTRAPGAPGGYPHLEGDVRWRRLFSSTHFFLRVDLGGRVQGTRWRHGQDSIVEIRSV
RVGTVVIKAVYSGFYVAMNRRGRLYGSRVYSVDCRFRERIEENGYNTYASRRWRHRGRPMFLALDSQG
IPRQGRRTRRHQLSTHFLPVLVSS

| | | |
|---|---|---|
| NOV4g, CG54455-04 | SEQ ID NO: 127 | 444 bp |
| DNA Sequence | ORF Start: at 1 | ORF Stop: end of sequence |

ACCCCGAGCGCGTCGCGGCGACCGCGCAGCTACCCGCACCTGGACGGCGACGTGCGCTGGCGGCGCCT
CTTCTCCTCCACTCACTTCTTCCTGCGCCTGGATCCCGGCGGCCGCGTGCAGGGCACCCGCTGGCGCC
ACGGCCAGGACAGCATCCTGGAGATCCGCTCTGTACACGTGGGCGTCGTGGTCATCAAAGCAGTGTCC
TCAGGCTTCTACGTGGCCATGAACCGCCGGGGCCGCCTCTACGGGTCGCGACTCTACACCGTGGACTG
CAGGTTCCGGGAGCGCATCGAAGAGAACGGCCACAACACCTACGCCTCACAGCGCTGGCGCCGCCGCG
GCCAGCCCATGTTCCTGCCGCTGGACAGGAGGGGGGGGCCCCGGCCAGGCCGCCGGACGCGGCGGTAC
CACCTGTCCGCCCACTTCCTGCCCGTCCTGGTCTCC

| | | | |
|---|---|---|---|
| NOV4g, CG54455-04 | | | |
| Protein Sequence | SEQ ID NO: 128 | 148 aa | MW at 17173.4 kD |

TPSASRGPRSYPHLEGDVRWRRLFSSTHFFLRVDPGGRVQGTRWRHGQDSILEIRSVHVGVVVIKAVS
SGFYVAMNRRCRLYGSRLYTVDCRFRERIEENGHNTYASQRWRRRGQPMFLALDRRGGPRPGGRTRRY
HLSAHFLPVLVS

| | | |
|---|---|---|
| NOV4h, CG54455-05 | SEQ ID NO: 129 | 550 bp |
| DNA Sequence | ORF Start: ATG at 19 | ORF Stop: TGA at 529 |

CGAACCGGGTGCCGGGTCATGCGCCGCCGCCTGTGGCTGGGCCTGGCCTGGCTGCTGCTGGCGCGGGC
GCCGGACGCCCCGGGAACCCCGAGCGCGTCGCCGGGACCGCGCAGCTACCCGCACCTGGAGGGCGACG
TGCGCTGGCGGCGCCTCTTCTCCTCCACTCACTTCTTCCTGCGCGTGGATCCCGGCGGCCGCGTGCAG
GGCACCCGCTGGCGCCACGGCCAGGACAGCATCCTGGAGATCCGCTCTGTACACGTGGGCGTCGTGGT
CATCAAAGCAGTGTCCTCAGGCTTCTACGTGGCCATGAACCGCCGGGGCCGCCTCTACGGGTCGCGAC
TCTACACCGTGGACTGCAGGTTCCGGGAGCCCATCGAAGAGAACGGCCACAACACCTACGCCTCACAG
CGCTGGCGCCGCCGCGGCCAGCCCATGTTCCTGGCGCTGGACAGGAGGGGGGGGCCCCGGCCAGGCGG
CCGGACGCGGCGGTACCACCTGTCCGCCCACTTCCTGCCCGTCCTGGTCTCCTGAGGCCCTGAGAGGC
CGGCGG

| | | | |
|---|---|---|---|
| NOV4h, CG54455-05 | | | |
| Protein Sequence | SEQ ID NO: 130 | 170 aa | MW at 19662.4 kD |

MRRRLWLGLAWLLLARAPDAAGTPSASRGPRSYPHLEGDVRWRRLFSSTHFFLRVDPGGRVQGTRWRH
GQDSILEIRSVHVGVVVIKAVSSGFYVAMNRRGRLYGSRLYTVDCRFRERIEENGHNTYASQRWRRRG
QPMFLALDRRGGPRPGGRTRRYHLSAHFLPVLVS

| | | |
|---|---|---|
| NOV4i, CG54455-06 | SEQ ID NO: 131 | 456 bp |
| DNA Sequence | ORF Start: at 7 | ORF Stop: at 451 |

AGATCTACCCCGAGCGCGTCGCGGGGACCGCGCAGCTACCCGCACCTGGAGGGCGACGTGCGCTGGCG
GCGTCTCTTCTCCTCCACTCACTTCTTCCTGCGCGTGGATCCCGGCGGCCGCGTGCAGGGCACCCGCT
GGCGCCACGGCCAGGACAGCATCCTGGAGATCCGCTCTGTACACGTGGGCGTCGTGGTCATCAAAGCA
GTGTCCTCAGGCTTCTACGTGGCCATGAACCGCCGGGGCCGCCTCTACGGGTCGCGACTCTACACCGT
GGACTGCAGGTTCCGGGAGCGCATCGAAGAGAACGGCCACAACACCTACGCCTCACAGCGCTGGCGCC
GCCGCGGCCAGCCCATGTTCCTGGCGCTGGACAGGAGGGGGGGGCCCCGGCCAGGCGGCCGGACGCGG
CCGTACCACCTGTCCGCCCACTTCCTGCCCGTCCTGGTCTCCCTCGAG

| | | | |
|---|---|---|---|
| NOV4i, CG54455-06 | | | |
| Protein Sequence | SEQ ID NO: 132 | 148 aa | MW at 17173.4 kD |

TPSASRGPRSYPHLEGDVRWRRLFSSTHFFLRVDPGGRVQGTRWRHGQDSILEIRSVHVGVVVIKAVS
SGFYVAMNRRGRLYGSRLYTVDCRFRERTEENGHNTYASQRWRRRGQPMFLALDRRGGPRPGGRTRRY
HLSAHFLPVLVS

| | | |
|---|---|---|
| NOV4j, CG54455-08 | SEQ ID NO: 133 | 538 bp |
| DNA Sequence | ORF Start: ATG at 17 | ORF Stop: TAG at 527 |

CACCGAGCTCCCCACCATGCGCCGCCGCCTGTGGCTGGGCCTGGCCTGGCTGCTGCTGGCGCGGGCGC
CGGACGCCGCGGGAACCCCGAGCGCGTCGCGGGGACCGCGCAGCTACCCGCACCTGGAGGGCGACGTG
CGCTGGCGGCGTCTCTTCTCCTCCACTCACTTCTTCCTGCGCGTGGATCCCGGCGGCCGCGTGCAGGG
CACCCGCTGGCGCCACGGCCAGGACAGCATCCTGGAGATCCGCTCTGTACACGTGGGCGTCGTGCTCA
TCAAAGCAGTGTCCTCAGGCTTCTACGTGGCCATGAACCGCCGGGGCCGCCTCTACGGGTCGCGACTC
TACACCGTGGACTGCAGGTTCCCGGAGCGCATCGAACAGAACGGCCACAACACCTACGCCTCACAGCG
CTGGCGCCGCCGCGGCCAGCCCATGTTCCTGGCCCTGGACAGGAGGGGGGGGCCCCGGCCAGGCGGCC
GGACGCGGCGGTACCACCTGTCCGCCCACTTCCTGCCCGTCCTGGTCTCCTAGGTCGACGGC

| | | | |
|---|---|---|---|
| NOV4j, CG54455-08 | | | |
| Protein Sequence | SEQ ID NO: 134 | 170 aa | MW at 19662.4 kD |

MRRRLWLGLAWLLLARAPDAAGTPSASRGPRSYPHLEGDVRWRRLFSSTHFFLRVDPGGRVQGTRWRH
GQDSILEIRSVHVGVVVIKAVSSGFYVAMNRRGRLYGSRLYTVDCRFRERIEENGHNTYASQRWRRRG
QPMFLALDRRGGPRPGGRTRRYHLSAHFLPVLVS

| | | |
|---|---|---|
| NOV4k, CG54455-09 | SEQ ID NO: 135 | 530 bp |
| DNA Sequence | ORF Start: ATG at 12 | ORF Stop: TAG at 528 |

AGATCTCCACCATGCGCCCCCGCCTGTGGCTGGGCCTGGCCTGGCTGCTGCTGGCGCGGGCGCCCGAC
GCCGCGGGAACCCCGAGCGCGTCGCGGGGACCGCGCAGCTACCCGCACCTGGAGGGCGACGTGCGCTG
GCGGCGTCTCTTCTCCTCCACTCACTTCTTCCTGCCCGTGGATCCCGGCGGCCGCGTGCAGGGCACCC
GCTGCCGCCACGGCCAGGACAGCATCCTGGAGATCCGCTCTGTACACGTGGGCGTCGTGGTCATCAAA
GCAGTGTCCTCAGGCTTCTACGTGGCCATGAACCGCCGGGGCCGCCTCTACGGGTCGCGACTCTACAC
CGTGGACTGCAGGTTCCGGGAGCGCATCGAAGAGAACGGCTACAACACCTACGCCTCACAGCCCTGGC
GCCGCCGCGGCCAGCCCATGTTCCTGGCGCTGGACAGGAGGGGGGGGCCCCGGCCAGGCGGCCGGACG
CGGCGGTACCACCTGTCCGCCCACTTCCTGCCCGTCCTGGTCTCCCTCGAGTAG

| | | | |
|---|---|---|---|
| NOV4k, CG54455-09 | | | |
| Protein Sequence | SEQ ID NO: 136 | 172 aa | MW at 19930.7 kD |

MRRRLWLGLAWLLLARAPDAAGTPSASRGPRSYPHLEGDVRWRRLFSSTHFFLRVDPGGRVQGTRWRH
GQDSILEIRSVHVGVVVIKAVSSGFYVAMNRRGRLYGSRLYTVDCRFRERIEENGYNTYASQRWRRRG
QPMFLALDRRGGPRPGGRTRRYHLSAHFLPVLVSLE

| | | |
|---|---|---|
| | SEQ ID NO: 137 | 510 bp |
| NOV4l, SNP13379002 of | ORF Start: ATG at 1 | ORF Stop: end of sequence |
| CG54455-03, DNA Sequence | SNP Pos: 358 | SNP Change: G to A |

ATGCGCCGCCGCCTGTGGCTGGGCCTGGCCTGGCTGCTGCTGGCGCGGGCGCCGGACGCCGCGGGAAC
CCCCAGCGCGTCGCGGGGACCCCGCAGCTACCCGCACCTGGAGGGCGACGTGCGCTGGCGGCGCCTCT
TCTCCTCCACTCACTTCTTCCTGCGCGTGGATCCCGGCGGCCGCGTGCAGGGCACCCGCTGGCGCCAC
GGCCAGGACAGCATCCTGGAGATCCGCTCTGTACACGTGGGCGTCGTGGTCATCAAAGCAGTGTCCTC
AGGCTTCTACGTGGCCATGAACCGCCGGGGCCGCCTCTACGGGTCGCGACTCTACACCGTGGACTGCA
GGTTCCCGGAGCGCATCAAAGAGAACGGCCACAACACCTACGCCTCACAGCGCTGGCGCCGCCGCGGC
CAGCCCATGTTCCTGGCGCTGGACAGGAGGGGGGGGCCCCGGCCAGCCGGCCGGACGCGGCGGTACCA
CCTGTCCGCCCACTTCCTGCCCGTCCTGGTCTCC

| | | | |
|---|---|---|---|
| NOV4l, SNP13379002 of | SEQ ID NO: 138 | | MW at 19661.4 kD |
| CG54455-03, Protein Sequence | SNP Pos: 120 | 170 aa | SNP Change: Glu to Lys |

MRRRLWLGLAWLLLARAPDAAGTPSASRGPRSYPHLEGDVRWRRLFSSTHFFLRVDPGGRVQGTRWRH
GQDSILEIRSVHVGVVVIKAVSSGFYVAMNRRGRLYGSRLYTVDCRFRERIKENGHNTYASQRWRRRG
QPMFLALDRRGGPRPGGRTRRYHLSAHFLPVLVS

A ClustalW comparison of the above protein sequences yields the following sequence alignment shown in Table 4B.

TABLE 4B
Comparison of the NOV4 protein sequences.

| | |
|---|---|
| NOV4a | ---MRRRLWLGLAWLLLARAPDAAGTPSASRGPRSYPHLEGDVRWRRLFSSTHFFLRVDP |
| NOV4b | ISTMRRRLWLGLAWLLLARAPDAAGTPSASRGPRSYPHLEGDVRWRRLFSSTHFFLRVDP |
| NOV4c | ---MRRRLWLGLAWLLLARAPDAAGTPSASRGPRSYPHLEGDVRWRRLFSSTHFFLRVDP |
| NOV4d | -THMRRRLWLGLAWLLLARAPDAAGTPSASRGPRSYPHLEGDVRWRRLFSSTHFFLRVDP |
| NOV4e | ---MRRRLWLGLAWLLLARAPDAAGTPSASRGPRSYPHLEGDVRWRRLFSSTHFFLRVDP |
| NOV4f | -----GRLWLGLAWLLLTRAP---GAPGG------YPHLEGDVRWRRLFSSTHFFLRVDL |
| NOV4g | -------------------------TPSASRGPRSYPHLEGDVRWRRLFSSTHFFLRVDP |
| NOV4h | ---MRRRLWLGLAWLLLARAPDAAGTPSASRGPRSYPHLEGDVRWRRLFSSTHFFLRVDP |
| NOV4i | -------------------------TPSASRGPRSYPHLEGDVRWRRLFSSTHFFLRVDP |
| NOV4j | ---MRRRLWLGLAWLLLARAPDAAGTPSASRGPRSYPHLEGDVRWRRLFSSTHFFLRVDP |
| NOV4k | ---MRRRLWLGLAWLLLARAPDAAGTPSASRGPRSYPHLEGDVRWRRLFSSTHFFLRVDP |
| NOV4a | GGRVQGTRWRHGQDSILEIRSVHVGVVVIKAVSSGFYVAMNRRGRLYGSRLYTVDCRFRE |
| NOV4b | GGRVQGTRWRHGQDSILEIRSVHVGVVVIKAVSSGFYVAMNRRGRLYGSRLYTVDCRFRE |
| NOV4c | GGRVQGTRWRHGQDSILEIRSVHVGVVVIKAVSSGFYVAMNRRGRLYGSRLYTVDCRFRE |
| NOV4d | GGRVQGTRWRHGQDSILEIRSVHVGVVVIKAVSSGFYVAMNRRGRLYGSRLYTVDCRFRE |
| NOV4e | GGRVQGTRWRHGQDSILEIRSVHVGVVVIKAVSSGFYVAMNRRGRLYGSRLYTVDCRFRE |
| NOV4f | GGRVQGTRWRHGQDSIVEIRSVRVGTVVIKAVYSGFYVAMNRRGRLYGSRVYSVDCRFRE |
| NOV4g | GGRVQGTRWRHGQDSILEIRSVHVGVVVIKAVSSGFYVAMNRRGRLYGSRLYTVDCRFRE |
| NOV4h | GGRVQGTRWRHGQDSILEIRSVHVGVVVIKAVSSGFYVAMNRRCRLYGSRLYTVDCRFRE |
| NOV4i | GGRVQGTRWRHGQDSILEIRSVHVGVVVIKAVSSGFYVAMNRRGRLYGSRLYTVDCRFRE |
| NOV4j | GGRVQGTRWRHGQDSILEIRSVHVGVVVIKAVSSGFYVAMNRRGRLYGSRLYTVDCRFRE |
| NOV4k | GGRVQGTRWRHGQDSILEIRSVHVGVVVIKAVSSGFYVAMNRRGRLYGSRLYTVDCRFRE |
| NOV4a | RIEENGHNTYASQRWRRRGQPMFLALDRRGGPRPGGRTRRYHLSAHFLPVLVS-- |
| NOV4b | RIEENGHNTYASQRWRRRGQPMFLALDRRGGPRPGGRTRRYHLSAHFLPVLVSLE |
| NOV4c | RIEENGHNTYASQRWRRRGQPMFLALDRRGGPRPGGRTRRYHLSAHFLPVLVS-- |
| NOV4d | RIEENGHNTYASQRWRRRGQPMFLALDRRGGPRPGGRTRRYHLSAHFLPVLVS-- |
| NOV4e | RIEENGHNTYASQRWRRRGQPMFLALDRRGGPRPGGRTRRYHLSAHFLPVLVS-- |
| NOV4f | RIEENGYNTYASRRWRHRGRPMFLALDSQGIPRQGRRTRRHQLSTHFLPVLVSS- |
| NOV4g | RIEENGHNTYASQRWRRRGQPMFLALDRRGGPRPGGRTRRYHLSAHFLPVLVS-- |
| NOV4h | RIEENGHNTYASQRWRRRGQPMFLALDRRGGPRPGGRTRRYHLSAHFLPVLVS-- |
| NOV4i | RIEENGHNTYASQRWRRRGQPMFLALDRRGGPRPGGRTRRYHLSAHFLPVLVS-- |
| NOV4j | RIEENGHNTYASQRWRRRGQPMFLALDRRGGPRPGGRTRRYHLSAHFLPVLVS-- |
| NOV4k | RIEENGYNTYASQRWRRRGQPMFLALDRRGGPRPGGRTRRYHLSAHFLPVLVSLE |
| NOV4a | (SEQ ID NO: 116) |
| NOV4b | (SEQ ID NO: 118) |
| NOV4c | (SEQ ID NO: 120) |
| NOV4d | (SEQ ID NO: 122) |
| NOV4e | (SEQ ID NO: 124) |
| NOV4f | (SEQ ID NO: 126) |
| NOV4g | (SEQ ID NO: 128) |
| NOV4h | (SEQ ID NO: 130) |
| NOV4i | (SEQ ID NO: 132) |
| NOV4j | (SEQ ID NO: 134) |
| NOV4k | (SEQ ID NO: 136) |

Further analysis of the NOV4a protein yielded the following properties shown in Table 4C.

TABLE 4C
Protein Sequence Properties NOV4a

| SignalP analysis: | Cleavage site between residues 23 and 24 | | | |
|---|---|---|---|---|
| PSORT II | PSG: a new signal peptide prediction method | | | |
| analysis: | N-region: length 4; pos.chg 3; neg.chg 0 | | | |
| | H-region: length 11; peak value 9.66 | | | |
| | PSG score: 5.26 | | | |
| | GvH: von Heijne's method for signal seq. recognition | | | |
| | GvH score (threshold: −2.1): 2.77 | | | |
| | possible cleavage site: between 17 and 18 | | | |
| | >>> Seems to have a cleavable signal peptide (1 to 17) | | | |
| | ALOM: Klein et al's method for TM region allocation | | | |
| | Init position for calculation: 18 | | | |
| | Tentative number of TMS(s) for the threshold 0.5: 1 | | | |
| | Number of TMS(s) for threshold 0.5: 0 | | | |
| | PERIPHERAL Likelihood = 12.15 (at 45) | | | |
| | ALOM score: −1.86 (number of TMSs: 0) | | | |
| | MTOP: Prediction of membrane topology (Hartmann et al.) | | | |
| | Center position for calculation: 8 | | | |
| | Charge difference: −3.0 C(1.0)-N(4.0) | | | |
| | N >= C: N-terminal side will be inside | | | |
| | MITDISC: discrimination of mitochondrial targeting seq | | | |
| | R content: | 4 | Hyd Moment(75): | 12.20 |
| | Hyd Moment(95): | 7.10 | G content: | 1 |
| | D/E content: | 1 | S/T content: | 0 |

```
Score: −0.86
Gavel: prediction of cleavage sites for mitochondrial preseq
R-10 motif at 54 RRL FS
NUCDISC: discrimination of nuclear localization signals
pat4: none
pat7: PGGRTRR (3) at 151
bipartite: none
content of basic residues: 18.8%
NLS Score: −0.22
KDEL: ER retention motif in the C-terminus: none
ER Membrane Retention Signals:
XXRR-like motif in the N-terminus: RRRL
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupas's algorithm to detect coiled-coil regions
total: 0 residues
Final Results (k = 9/23):
47.8%: mitochondrial
17.4%: endoplasmic reticulum
  8.7%: extracellular, including cell wall
  8.7%: Golgi
  8.7%: cytoplasmic
  4.3%: vacuolar
  4.3%: nuclear
>> prediction for CG54455-03 is mit (k = 23)
```

A search of the NOV4a protein against the Geneseq database, a proprietary database that contains sequences published in patents and patent publication, yielded several homologous proteins shown in Table 4D.

TABLE 4D
Geneseq Results for NOV4a

| | | | Identities/ | |
|---|---|---|---|---|
| | | | Similarities for | |
| Geneseq | Protein/Organism/Length | NOV4a Residues/ | the Matched | Expect |
| Identifier | [Patent #, Date] | Match Residues | Region | Value |
| AAE18804 | Human FGF-like precursor | 1 . . . 170 | 170/170 (100%) | 1e−98 |
| | protein - Homo sapiens, 170 aa. | 1 . . . 170 | 170/170 (100%) | |
| | [US2002001825-A1, 03 JAN. | | | |
| | 2002] | | | |
| ABB75884 | Human FGF homologue zFGF10 - | 1 . . . 170 | 170/170 (100%) | 1e−98 |
| | Homo sapiens, 170 aa. | 1 . . . 170 | 170/170 (100%) | |
| | [US2002031805-A1, 14 MAR. | | | |
| | 2002] | | | |
| ABG34060 | Human Pro peptide #31 - Homo | 1 . . . 170 | 170/170 (100%) | 1e−98 |
| | sapiens, 170 aa. [WO200224888- | 1 . . . 170 | 170/170 (100%) | |
| | A2, 28 MAR. 2002] | | | |
| AAU98086 | Human fibroblast growth factor | 1 . . . 170 | 170/170 (100%) | 1e−98 |
| | FGF-23 protein sequence - Homo | 1 . . . 170 | 170/170 (100%) | |
| | sapiens, 170 aa. [WO200246424- | | | |
| | A2, 13 JUN. 2002] | | | |
| AAB49649 | Human SEC1 protein sequence | 1 . . . 170 | 170/170 (100%) | 1e−98 |
| | SEQ ID 2 - Homo sapiens, 170 aa. | 1 . . . 170 | 170/170 (100%) | |
| | [WO200070046-A2, 23 NOV. | | | |
| | 2000] | | | |

In a BLAST search of public sequence databases, the NOV4a protein was found to have homology to the proteins shown in the BLASTP data in Table 4E.

TABLE 4E
Public BLASTP Results for NOV4a

| | | | Identities/ | |
|---|---|---|---|---|
| Protein | | | Similarities for | |
| Accession | | NOV4a Residues/ | the Matched | Expect |
| Number | Protein/Organism/Length | Match Residues | Portion | Value |
| Q9HCT0 | Fibroblast growth factor-22 | 1 . . . 170 | 170/170 (100%) | 3e−98 |
| | precursor (FGF-22) - Homo | 1 . . . 170 | 170/170 (100%) | |
| | sapiens (Human), 170 aa. | | | |
| Q8VI79 | Fibroblast growth factor 22 - | 1 . . . 170 | 139/170 (81%) | 3e−76 |
| | Rattus norvegicus (Rat), 162 aa. | 1 . . . 161 | 147/170 (85%) | |
| Q9ESS2 | Fibroblast growth factor-22 | 1 . . . 170 | 138/170 (81%) | 6e−75 |
| | precursor (FGF-22) - Mus | 1 . . . 161 | 146/170 (85%) | |
| | musculus (Mouse), 162 aa. | | | |
| O60371 | R33683_2 - Homo sapiens | 73 . . . 170 | 98/98 (100%) | 8e−52 |
| | (Human), 129 aa. | 32 . . . 129 | 98/98 (100%) | |
| CAD61991 | Sequence 41 from Patent | 25 . . . 169 | 79/145 (54%) | 5e−43 |
| | EP1247530 - Homo sapiens | 28 . . . 172 | 112/145 (76%) | |
| | (Human), 174 aa. | | | |

PFam analysis predicts that the NOV4a protein contains the domains shown in the Table 4F.

TABLE 4F
Domain Analysis of NOV4a

| | | Identities/ | |
|---|---|---|---|
| | | Similarities | |
| | NOV4a Match | for the | Expect |
| Pfam Domain | Region | Matched Region | Value |
| FGF | 39 . . . 168 | 63/147 (43%) | 1.8e−53 |
| | | 105/147 (71%) | |

### Example 5

The NOV5 clone was analyzed, and the nucleotide and encoded polypeptide sequences are shown in Table 5A.

TABLE 5A
NOV5 Sequence Analysis

| | | |
|---|---|---|
| NOV5a, CG54611-06 | SEQ ID NO: 139 | 1081 bp |
| DNA Sequence | ORF Start: ATG at 19 | ORF Stop: TAG at 1075 |

GCGCCCTCTCGCGCGGCGATGGCCCCACTCGGATACTTCTTACTCCTCTGCAGCCTGAAGCAGCCTCT
GGGCAGCTACCCGATCTGGTGGTCGCTGGCTGTTGGGCCACAGTATTCCTCCCTGGGCTCGCAGCCCA
TCCTGTGTGCCAGCATCCCGGGCCTGGTCCCCAAGCAGCTCCGCTTCTGCAGGAACTACGTGGAGATC
ATGCCCAGCGTGGCCGAGGGCATCAAGATTGGCATCCAGGAGTGCCAGCACCAGTTCCGCGGCCGCCG
GTGGAACTGCACCACCGTCCACGACAGCCTGGCCATCTTCGGGCCCGTGCTGGACAAAGCTACCAGGG
AGTCGGCCTTTGTCCACGCCATTGCCTCAGCCGGTGTGGCCTTTGCAGTGACACGCTCATGTGCAGAA
GGCACGGCCGCCATCTGTGGCTGCAGCAGCCGCCACCAGGCCTCACCAGGCAAGGCCTGGAAGTGGGG
TGGCTGTAGCGAGGACATCGAGTTTGGTGGGATGGTGTCTCGGGAGTTCGCCGACGCCCGGGAGAACC
GGCCAGATGCCCGCTCAGCCATGAACCGCCACAACAACGAGGCTGGGCGCCAGGCCATCGCCAGCCAC
ATGCACCTCAAGTGCAAGTGCCACGGGCTGTCGGGCAGCTGCGAGGTGAAGACATGCTGGTGGTCGCA
ACCCGACTTCCGCGCCATCGGTGACTTCCTCAAGGACAAGTACGACAGCGCCTCGGAGATGGTGGTGG
AGAAGCACCGGGAGTCCCGCGGCTGGGTGGAGACCCTGCGGCCGCGCTACACCTACTTCAAGGTGCCC
ACGGAGCGCGACCTGGTCTACTACGAGGCCTCGCCCAACTTCTGCGAGCCCAACCCTCAGACGGGCTC
CTTCGGCACGCGCGACCGCACCTGCAACGTCAGCTCGCACGGCATCGACGGCTGCGACCTGCTGTGCT
GCGGCCGCGGCCACAACGCGCGAGCGGAGCGGCGCCGGGAGAAGTGCCGCTGCGTGTTCCACTGGTGC
TGCTACGTCAGCTGCCAGGAGTGCACGCGCGTCTACGACGTGCACACCTGCAAGTAGGCAC

| | | | |
|---|---|---|---|
| NOV5a, CG54611-06 | | | |
| Protein Sequence | SEQ ID NO: 140 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCCRGHNARAERRREKCRCVFHWCCYVSCQ
ECTRVYDVHTCK

| | | |
|---|---|---|
| NOV5b, 283841210 | SEQ ID NO: 141 | 1014 bp |
| DNA Sequence | ORF Start: at 1 | ORF Stop: end of sequence |

GGATCCAGCTACCCGATCTGGTGGTCGCTGGCTGTTGGGCCACAGTATTCCTCCCTGGGCTCGCAGCC
CATCCTGTGTGCCAGCATCCCGGGCCTGGTCCCCAAGCAGCTCCGCTTCTGCAGGAACTACGTGGAGA
TCATGCCCAGCGTGGCCGAGGGCATCAAGATTGGCATCCAGGAGTGCCAGCACCAGTTCCGCGGCCGC
CGGTGGAACTGCACCACCGTCCACCACAGCCTGGCCATCTTCGGGCCCGTGCTGGACAAAGCTACCAG
GGAGTCGGCCTTTGTCCACGCCATTGCCTCAGCCGGTGTGGCCTTTGCAGTGACACGCTCATGTGCAG
AAGGCACGGCCGCCATCTGTGGCTGCAGCAGCCGCCACCAGGGCTCACCAGGCAAGGGCTGGAAGTGG
GGTGGCTGTAGCGAGGACATCGAGTTTGGTGGGATGGTGTCTCGGGAGTTCGCCGACGCCCGGGAGAA
CCGGCCAGATGCCCGCTCAGCCATGAACCGCCACAACAACGAGGCTGGGCGCCAGGCCATCGCCAGCC
ACATGCACCTCAAGTGCAACTGCCACGGGCTGTCGGGCAGCTGCGAGGTGAAGACATGCTGGTGGTCG
CAACCCGACTTCCGCGCCATCGGTGACTTCCTCAAGGACAAGTACGACAGCGCCTCGGAGATGGTGGT
GGAGAAGCACCGGGAGTCCCGCGGCTGGGTGGAGACCCTGCGGCCGCGCTACACCTACTTCAAGGTGC
CCACGGAGCGCGACCTCGTCTACTACGAGGCCTCGCCCAACTTCTGCGAGCCCAACCCTGAGACGGGC
TCCTTCGGCACGCGCGACCGCACCTGCAACGTCAGCTCGCACGGCATCGACGGCTGCGACCTGCTGTG
CTGCGGCCGCGGCCACAACGCGCGAGCGGAGCGGCGCCGGGAGAAGTGCCGCTGCGTGTTCCACTGGT
GCTGCTACGTCAGCTGCCAGGAGTGCACGCGCGTCTACGACGTCCACACCTGCAAGCTCGAG

| | | | |
|---|---|---|---|
| NOV5b, 283841210 | | | |
| Protein Sequence | SEQ ID NO: 142 | 338 aa | MW at 37830.4 kD |

GSSYPIWWSLAVGPQYSSLGSQPILCASIPGLVPKQLRFCRNYVEIMPSVAEGIKIGIQECQHQFRGR
RWNCTTVHDSLAIFGPVLDKATRESAFVHATASAGVAFAVTRSCAEGTAAICGCSSRHQGSPGKGWKW
GGCSEDIEFGGMVSREFADARENRPDARSAMNRHNNEAGRQAIASHMHLKCKCHGLSGSCEVKTCWWS
QPDFRAIGDFLKDKYDSASEMVVEKHRESRGWVETLRPRYTYFKVPTERDLVYYEASPNFCEPNPETG
SFGTRDRTCNVSSHGIDGCDLLCCGRGHNARAERRREKCRCVFHWCCYVSCQECTRVYDVHTCKLE

| | | |
|---|---|---|
| NOV5c,CG54611-01 | SEQ ID NO: 143 | 1116 bp |
| DNA Sequence | ORF Start: ATG at 31 | ORF Stop: TAG at 1087 |

TCCCGGCCCTCCGCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGCCCACAGTATTCCTCCCTGG
GCTCGCAGCCCATCCTGTGTGCCAGCATCCCGGGCCTGGTCCCCAAGCAGCTCCGCTTCTGCAGGAAC
TACGTGGAGATCATGCCCAGCGTGGCCGAGGGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGGCTGCAGCAGCCGCCACCAGCGCTCACCAGGCAAGGG
CTGGAAGTGGGGTGGCTGTAGCGAGGACATCGAGTTTGGTGGGATGGTGTCTCGGGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGGGCTGTCGGGCAGCTGCGAGGTGAAGACATG
CTGGTGGTCGCAACCCGACTTCCGCGCCATCGGTGACTTCCTCAAGGACAAGTACGACAGCGCCTCGG
AGATGGTGGTGGAGAAGCACCGGGAGTCCCGCGGCTGGGTGGAGACCCTGCGGCCGCGCTACACCTAC
TTCAAGGTGCCCACGGAGCGCGACCTGGTCTACTACGAGGCCTCGCCCAACTTCTGCGAGCCCAACCC
TGAGACGGGCTCCTTCGGCACGCGCGACCGCACCTGCAACCTCAGCTCGCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGGAGTGCACGCGCGTCTACGACGTGCACACCTGCAAGTA
GGCACCGGCCGCGGCTCCCCCTGGACGG

| | | | |
|---|---|---|---|
| NOV5c, CG54611-01 | | | |
| Protein Sequence | SEQ ID NO: 144 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| NOV5d, CG54611-02 | SEQ ID NO: 145 | 1060 bp |
| DNA Sequence | ORF Start: ATG at 2 | ORF Stop: TAG at 1058 |

GATGGCCCCACTCGGATACTTCTTACTCCTCTGCAGCCTGAAGCAGGCTCTGGGCAGCTACCCGATCT
GGTCGTCGCTGGCTGTTGGGCCACAGTATTCCTCCCTGGGCTCGCAGCCCATCCTGTGTGCCAGCATC
CCGGGCCTGGTCCCCAAGCAGCTCCGCTTCTGCAGGAACTACGTGGAGATCATGCCCAGCGTGGCCGA
GGGCATCAAGATTGGCATCCAGGAGTGCCAGCACCAGTTCCGCGGCCGCCGGTGGAACTGCACCACCG
TCCACGACAGCCTGGCCATCTTCGGGCCCGTGCTGGACAAAGCTACCAGGGAGTCGGCCTTTGTCCAC
GCCATTGCCTCAGCCGGTGTGGCCTTTGCAGTGACACGCTCATGTGCAGAAGGCACGGCCGCCATCTG
TGGCTGCAGCAGCCGCCACCAGGGCTCACCAGGCAAGGGCTGGAAGTGGGGTGGCTGTAGCGAGCACA
TCGAGTTTGGTGGGATGGTGTCTCGGGAGTTCGCCGACGCCCGGGAGAACCGGCCAGATCCCCGCTCA
GCCATGAACCGCCACAACAACGAGGCTGGGCGCCAGGCCATCGCCAGCCACATGCACCTCAAGTGCAA
GTGCCACGGGCTGTCGGGCAGCTGCGAGGTGAAGACATGCTGGTGGTCGCAACCCCACTTCCGCGCCA
TCGGTGACTTCCTCAAGGACAAGTACGACAGCCCCTCGGAGATGGTGGTGGAGAAGCACCGGGAGTCC
CGCGGCTGGGTGGAGACCCTGCGGCCGCGCTACACCTACTTCAAGGTGCCCACGGAGCGCGACCTGGT
CTACTACGAGGCCTCGCCCAACTTCTGCGAGCCCAACCCTGAGACGGGCTCCTTCGGCACGCGCGACC
GCACCTGCAACGTCAGCTCGCACGGCATCGACGGCTGCGACCTGCTGTGCTGCGGCCGCGGCCACAAC
GCCCGAGCGGAGCGGCGCCGGGAGAAGTGCCGCTGCGTGTTCCACTGGTGCTGCTACGTCAGCTCCCA
CGAGTGCACGCGCGTCTACGACGTCCACACCTGCAAGTAG

| | | | |
|---|---|---|---|
| NOV5d, CG54611-02 | | | |
| Protein Sequence | SEQ ID NO: 146 | 352 aa | MW at 39364.3 kD |

MAPLCYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRCWVETLRPRYTYFKVPTERDLV
YYEASPNFCEPNPETGSFGTRDRTCNVSSHGIDGCDLLCCCRGHNARAERRREKCRCVFHWCCYVSCQ
ECTRVYDVHTCK

| | | |
|---|---|---|
| NOV5e, CG54611-03 | SEQ ID NO: 147 | 1002 bp |
| DNA Sequence | ORF Start: at 1 | ORF Stop: end of sequence |

AGCTACCCCATCTGGTGGTCGCTGGCTGTTGGGCCACAGTATTCCTCCCTGGGCTCGCAGCCCATCCT
GTGTGCCAGCATCCCGGGCCTGGTCCCCAAGCAGCTCCGCTTCTGCAGGAACTACGTGGAGATCATGC
CCAGCGTGGCCGAGGGCATCAAGATTGGCATCCAGGAGTGCCAGCACCAGTTCCGCGGCCGCCGGTGG
AACTGCACCACCGTCCACGACAGCCTGGCCATCTTCGGGCCCGTGCTGGACAAAGCTACCAGGGAGTC
GGCCTTTGTCCACGCCATTGCCTCAGCCGGTGTGGCCTTTGCAGTGACACGCTCATGTCCAGAAGGCA
CGGCCGCCATCTGTCGCTGCAGCAGCCGCCACCAGGGCTCACCAGGCAAGGGCTGGAAGTGGGGTGGC
TGTAGCGAGGACATCGAGTTTGGTGGGATGGTGTCTCGGGAGTTCGCCGACGCCCGGGAGAACCGGTC
AGATGCCCGCTCAGCCATGAACCGCCACAACAACGAGGCTGGGCGCCACGCCATCGCCAGCCACATGC
ACCTCAAGTGCAAGTGCCACGGGCTGTCGGGCAGCTGCGAGGTGAAGACATGCTGGTGGTCGCAACCC
GACTTCCGCGCCATCGGTGACTTCCTCAAGGACAAGTACGACAGCGCCTCGGAGATGGTGGTCGAGAA
GCACCGGGAGTCCCGCGGCTGGGTGGAGACCCTGCGGCCGCGCTACACCTACTTCAAGGTGCCCACGG
AGCGCGACCTGGTCTACTACGAGGCCTCGCCCAACTTCTGCGAGCCCAACCCTCAGACGGGCTCCTTC
GGCACGCGCGACCGCACCTGCAACGTCAGCTCGCACGGCATCGACGGCTGCGACCTGCTGTGCTCCGG
CCGCGGCCACAACGCGCGAGCGGACCGGCGCCGGGAGAAGTGCCGCTGCGTGTTCCACTGGTGCTCCT
ACGTCAGCTGCCAGGAGTGCACGCGCGTCTACGACGTGCACACCTGCAAG

| | | | |
|---|---|---|---|
| NOV5e, CG54611-03 | | | |
| Protein Sequence | SEQ ID NO: 148 | 334 aa | MW at 37433.9 kD |

SYPIWWSLAVGPQYSSLGSQPILCASIPGLVPKQLRFCRNYVEIMPSVAEGIKIGIQECQHQFRGRRW
NCTTVHDSLAIFGPVLDKATRESAFVHAIASAGVAFAVTRSCAEGTAAICGCSSRHQGSPGKGWKWGG
CSEDIEFGGMVSREFADARENRSDARSAMNRHNNEAGRQAIASHMHLKCKCHGLSGSCEVKTCWWSQP
DFRAIGDFLKDKYDSASEMVVEKHRESRGWVETLRPRYTYFKVPTERDLVYYEASPNFCEPNPETGSF
GTRRTCNVSSHGIDGCDLLCCGRGHNARAERRREKCRCVFHWCCYVSCQECTRVYDVHTCK

| | | |
|---|---|---|
| NOV5f, CG54611-04 | SEQ ID NO: 149 | 849 bp |
| DNA Sequence | ORF Start: at 1 | ORF Stop: end of sequence |

AGCTACCCGATCTGGTGGTCGCTGGCTGTTGGGCCACAGTATTCCTCCCTGGGCTCGCAGCCCATCCT
GTGTGCCAGCATCCCGGGCCTGGTCCCCAAGCAGCTCCGCTTCTGCAGGAACTACGTGGAGATCATGC
CCAGCGTGGCCGAGGGCATCAAGATTGGCATCCAGGAGTGCCAGCACCAGTTCCGCGGCCGCCGGTGG
AACTGCACCACCGTCCACGACAGCCTGGGCATCTTCGGGCCCGTGCTGGACAAAGCTACCAGGGAGTC
GGCCTTTGTCCACGCCATTGCCTCAGCCGGTGTGGCCTTTGCAGTGACACGCTCATGTGCAGAAGGCA
CGGCCGCCATCTGTGGCTGCAGCAGCCGCCACCAGGGCTCACCACCCAAGGGCTGGAAGTGGGGTGGC
TGTAGCGAGGACATCCAGTTTGGTGGGATGGTGTCTCGGGAGTTCGCCGACGCCCGCGAGAACCGGCC
AGATGCCCGCTCAGCCATGAACCGCCACAACAACGAGGCTGGGCGCCAGGCCATCGCCAGCCACATGC
ACCTCAAGTGCAAGTGCCACGGGCTGTCGGGCAGCTGCGAGGTGAAGACATCCTGGTGGTCGCAACCC
GACTTCCGCGCCATCGGTGACTTCCTCAAGGACAAGTACGACAGCGCCTCGGAGATGGTGGTGGAGAA
GCACCGGGAGTCCCGCGGCTGGGTGGAGACCCTGCGGCCGCGCTACACCTACTTCAAGGTGCCCACGG
AGCGCGACCTGGTCTACTACGAGGCCTCGCCCAACTTCTGCGAGCCCAACCCTGAGACGGGCTCCTTC
GGCACGCGCGTCTACGACGTGCACACCTGCAAG

| | | | |
|---|---|---|---|
| NOV5f, CG54611-04 | | | |
| Protein Sequence | SEQ ID NO: 150 | 283 aa | MW at 31567.3 kD |

SYPIWWSLAVGPQYSSLGSQPILCASIPGLVPKQLRFCRNYVEIMPSVAEGIKIGIQECQHQFRGRRW
NCTTVHDSLAIFGPVLDKATRESAFVHAIASAGVAFAVTRSCAEGTAAICGCSSRHQGSPGKGWKWGG
CSEDIEFGGMVSREFADARENRPDARSAMNRHNNEAGRQAIASHMHLKCKCHGLSGSCEVKTCWWSQP
DFRAIGDFLKDKYDSASEMVVEKHRESRGWVETLRPRYTYFKVPTERDLVYYEASPNFCEPNPETGSF
GTRVYDVHTCK

| | | |
|---|---|---|
| NOV5g, CG54611-05 | SEQ ID NO: 151 | 1056 bp |
| DNA Sequence | ORF Start: ATG at 1 | ORF Stop: end of sequence |

ATGGCCCCACTCGGATACTTCTTACTCCTCTGCAGCCTGAAGCAGGCTCTGGGCAGCTACCCGATCTG
GTGGTCGCTGGCTGTTGGGCCACAGTATTCCTCCCTCGGCTCGCAGCCCATCCTGTGTGCCAGCATCC
CGGGCCTGGTCCCCAAGCAGCTCCCCTTCTGCAGGAACTACGTGGAGATCATGCCCAGCGTGGCCGAG
GGCATCAAGATTGGCATCCAGGAGTGCCAGCACCAGTTCCGCGGCCGCCGGTGGAACTGCACCACCGT
CCACGACAGCCTGGCCATCTTCGGGCCCGTGCTGGACAAAGCTACCAGGGAGTCGGCCTTTGTCCACG
CCATTGCCTCAGCCGGTGTGGCCTTTGCAGTGACACGCTCATGTCCAGAAGGCACGGCCGCCATCTGT
GGCTGCAGCAGCCGCCACCAGGCCTCACCAGGCAAGGGCTGGAAGTGGGGTGGCTGTAGCGAGGACAT
CGAGTTTGGTCGGATGGTGTCTCGGGAGTTCGCCGACGCCCGGGAGAACCGGCCAGATGCCCGCTCAG
CCATGAACCGCCACAACAACGAGGCTGGGCGCCAGGCCATCGCCAGCCACATGCACCTCAAGTGCAAG
TGCCACGGGCTGTCGGGCAGCTGCGAGGTGAAGACATGCTGGTGGTCGCAACCCGACTTCCGCGCCAT
CGGTGACTTCCTCAAGGACAAGTACGACAGCGCCTCGGAGATGGTGGTGGAGAAGCACCGGGAGTCCC
GCGGCTGGGTGGAGACCCTGCGGCCGCGCTACACCTACTTCAAGGTGCCCACGGAGCGCGACCTGGTC
TACTACGAGGCCTCGCCCAACTTCTGCGAGCCCAACCCTGAGACGGGCTCCTTCGGCACGCGCGACCG
CACCTGCAACGTCAGCTCGCACGGCATCGACGGCTGCGACCTCCTGTGCTGCGGCCGCGCCCACAACG
CGCGAGCGGAGCGCCGCCGGGAGAAGTGCCGCTGCGTGTTCCACTGGTGCTGCTACGTCAGCTGCCAG
GAGTGCACGCGCGTCTACGACGTGCACACCTGCAAG

| | | | |
|---|---|---|---|
| NOV5g, CG54611-05 | | | |
| Protein Sequence | SEQ ID NO: 152 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAICDFLKDKYDSASEMVVEKHRESRGWVETLRPRYTYFKVPTERDLV
YYEASPNFCEPNPETCSFGTRDRTCNVSSHGIDGCDLLCCGRGHNARAERRREKCRCVFHWCCYVSCQ
ECTRVYDVHTCK

| | | |
|---|---|---|
| NOV5h,CG54611-07 | SEQ ID NO: 153 | 1099 bp |
| DNA Sequence | ORF Start: at 2 | ORF Stop: TGA at 1088 |

CACCGGATCCACCATGGCCCCACTCGGCTACTTCTTACTCCTCTGCAGCCTGAAGCAGGCTCTGGGCA
GCTACCCGATCTGGTGCTCGCTGGCTGTTGGGCCACAGTATTCCTCCCTGGGCTCGCAGCCCATCCTC
TGTGCCAGCATCCCTGGCCTGGTCCCCAAGCAGCTCCGCTTCTGCAGGAACTACGTGGAGATCATGCC
CAGCGTGGCCGAGGGCATCAAGATTGGCATCCAGGAGTGCCAGCACCAGTTCCGCGGCCGCCGGTGCA
ACTGCACCACCCTCCACGACAGCCTGGCCATCTTCGCGCCCGTGCTGGACAAAGCTACCAGGGAGTCG
GCCTTTGTCCACGCCATTGCCTCAGCCGGTGTGGCCTTTGCAGTGACACGCTCATCTGCAGAAGGCAC
GGCCGCCATCTGTGGCTGCAGCAGCCGCCACCAGGGCTCACCAGGCAAGGCCTGGAAGTGGGGTGGCT
GTAGCGAGGACATCGAGTTTGGTGGGATGGTGTCTCGGGAGTTCGCCGACGCCCGGCAGAACCGGCCA
GATGCCCGCTCAGCCATGAACCGCCACAACAACGAGGCTGGGCGCCAGGCCATCGCCAGCCACATGCA
CCTCAACTGCAAGTGCCACGGGCTGTCGGGCAGCTGCGAGGTGAAGACATGCTGGTGGTCGCAACCCG
ACTTCCGCGCCATCGGTGACTTCCTCAAGGACAAGTACGACAGCGCCTCGGAGATGGTGGTGGAGAAC
CACCGGGAGTCCCGCGGCTGGGTGGAGACCCTGCGGCCGCGCTACACCTACTTCAAGGTGCCCACGGA
GCGCGACCTGGTCTACTACGAGGCCTCGCCCAACTTCTGCGAGCCCAACCCTGAGACGGGCTCCTTCG
GCACGCGCGACCGCACCTGCAACGTCAGCTCGCACGGCATCGACGGCTGCGACCTGCTGTGCTGCGGC
CGCGCCCACAACGCGCGAGCGGAGCGGCGCCGGGAGAAGTGCCGCTGCGTGTTCCACTGGTGCTGCTA
CGTCAGCTGCCAGGAGTGCACGCGCGTCTACGACGTGCACACCTGCAAGCACCATCACCACCATCACT
GACTCGAGCGG

| | | | |
|---|---|---|---|
| NOV5h, CG54611-07 | | | |
| Protein Sequence | SEQ ID NO: 154 | 362 aa | MW at 40533.5 kD |

TGSTMAPLGYFLLLCSLKQALGSYPTNWSLAVGPQYSSLGSQPILCASTPGLVPKQLRFCRNYVEIMP
SVAEGIKIGIQECQHQFRGRRWNCTTVHDSLAIFGPVLDKATRESAFVHAIASAGVAFAVTRSCAEGT
AAICGCSSRHQGSPGKGWKWGGCSEDIEFGGMVSREFADARENRPDARSAMNRHNNEAGRQAIASHMH
LKCKCHGLSGSCEVKTCWWSQPDFRAIGDFLKDKYDSASEMVVEKHRESRGWVETLRPRYTYFKVPTE
RDLVYYEASPNFCEPNPETGSFGTRDRTCNVSSHGIDGCDLLCCGRGHNARAERRREKCRCVFHWCCY
VSCQECTRVYDVHTCKHHHHHH

| | | |
|---|---|---|
| NOV5i, CG54611-08 | SEQ ID NO: 155 | 1071 bp |
| DNA Sequence | ORF Start: ATG at 10 | ORF Stop: at 1066 |

GGATCCACCATGGCCCCACTCGGATACTTCTTACTCCTCTGCAGCCTGAAGCAGGCTCTGGGCAGCTA
CCCGATCTGGTGGTCGCTGGCTGTTGGGCCACAGTATTCCTCCCTGGGCTCGCAGCCCATCCTGTGTG
CCAGCATCCCGGGCCTGGTCCCCAAGCAGCTCCGCTTCTGCAGGAACTACGTGGAGATCATGCCCAGC
GTGGCCGAGCGCATCAAGATTGGCATCCAGGAGTGCCAGCACCAGTTCCGCGGCCCCCGGTGGAACTG
CACCACCGTCCACGACAGCCTGGCCATCTTCGGGCCCGTGCTGGACAAAGCTACCAGGGAGTCGGCCT
TTGTCCACGCCATTGCCTCAGCCGGTGTGGCCTTTGCAGTGACACGCTCATGTGCAGAAGGCACGGCC
GCCATCTGTGGCTGCAGCAGCCGCCACCAGGGCTCACCAGGCAAGGGCTGGAAGTGGGGTGGCTGTAG
CGAGGACATCCAGTTTGGTGGGATGGTGTCTCGGGAGTTCGCCGACGCCCGGGAGAACCGGCCAGATG
CCCGCTCAGCCATGAACCGCCACAACAACGAGGCTGGGCGCCAGGCCATCGCCAGCCACATGCACCTC
AAGTGCAAGTGCCACGGGCTGTCGGGCAGCTGCGAGGTGAAGACATGCTGGTGGTCGCAACCCGACTT
CCGCGCCATCGGTGACTTCCTCAAGGACAAGTACGACAGCGCCTCGGAGATGGTGGTGGAGAAGCACC
GGGAGTCCCGCGGCTGGGTGGAGACCCTGCGGCCGCGCTACACCTACTTCAAGGTGCCCACGGAGCGC
GACCTGGTCTACTACGAGGCCTCGCCCAACTTCTGCGAGCCCAACCCTGAGACGGGCTCCTTCGGCAC
GCGCGACCGCACCTGCAACGTCAGCTCGCACGGCATCGACGGCTGCGACCTGCTGTGCTGCGGCCGCG
GCCACAACGCGCGAGCGGAGCGCCGCCGGGAGAAGTGCCGCTGCGTGTTCCACTGGTGCTGCTACGTC
AGCTGCCAGGAGTGCACGCGCGTCTACGACGTGCACACCTGCAAGCTCGAG

| | | | |
|---|---|---|---|
| NOV5i, CG54611-08 | | | |
| Protein Sequence | SEQ ID NO: 156 | 352 aa | MW at 39364.3 kD |

MAPLGYFLLLCSLKQALGSYPIWWSLAVGPQYSSLGSQPTLCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| NOV5j, CG54611-09 | SEQ ID NO: 157 | 2932 bp |
| DNA Sequence | ORF Start: ATG at 79 | ORF Stop: TAG at 1135 |

AGCTCCCAGGGCCCGGCCCCCCCCGGCGCTCACGCTCTCGGGGCCGACTCCCGGCCCTCCCCGCCCTC
CCGCGCGGCGATGGCCCCACTCGCATACTTCTTACTCCTCTGCAGCCTGAAGCAGCCTCTGGGCAGCT
ACCCCATCTGGTGGTCGCTGGCTGTTGGGCCACAGTATTCCTCCCTGGGCTCGCAGCCCATCCTGTGT
GCCAGCATCCCGGGCCTGGTCCCCAAGCAGCTCCGCTTCTGCAGGAACTACGTGGAGATCATGCCCAG
CGTGGCCGAGGGCATCAAGATTGGCATCCAGGAGTGCCAGCACCAGTTCCGCGGCCGCCGGTGGAACT
GCACCACCGTCCACGACAGCCTGGCCATCTTCGGGCCCGTGCTGGACAAAGCTACCAGGGAGTCGGCC
TTTGTCCACGCGATTGCCTCAGCCGGTGTGGCCTTTGCAGTGACACGCTCATGTCCAGAAGGCACGGC
CGCCATCTGTGGCTGCAGCACCCGCCACCAGGGCTCACCAGGCAAGGGCTGGAAGTGGGGTGGCTGTA
GCGAGGACATCGAGTTTGGTGGGATGGTGTCTCGGGAGTTCGCCCACGCCCGGGAGAACCGGCCAGAT
GCCCGCTCAGCCATGAACCGCCACAACAACGAGGCTGGGCGCCAGGCCATCGCCAGCCACATGCACCT
CAAGTGCAAGTGCCACGGGCTGTCGCGCAGCTGCGAGGTGAAGACATGCTGGTGGTCGCAACCCGACT
TCCGCGCCATCGGTGACTTCCTCAAGGACAAGTACGACAGCGCCTCGGAGATGGTGGTGGAGAAGCAC
CGGGAGTCCCGCGGCTGGGTGGAGACCCTGCGGCCGCGCTACACCTACTTCAAGGTGCCCACGGAGCG
CGACCTGGTCTACTACGAGGCCTCGCCCAACTTCTGCGAGCCCAACCCTGAGACGGGCTCCTTCGGCA
CGCGCGACCGCACCTGCAACGTCAGCTCGCACGGCATCGACGGCTGCGACCTGCTGTGCTGCGGCCGC
GGCCACAACGCGCGAGCGGAGCGGCGCCGGGAGAAGTGCCGCTGCGTGTTCCACTGGTGCTGCTACGT
CAGCTGCCAGGAGTGCACGCGCGTCTACGACGTGCACACCTGCAAGTAGGCACCGGCCGCGGCTCCCC
CTGGACGGGGCGGGCCCTGCCTGAGGGTGGGCTTTTCCCTGGGTGGAGCAGGACTCCCACCTAAACGG
GGCAGTACTCCTCCCTGGGGGCGGGACTCCTCCCTGGGGGTGGGGCTCCTACCTGGGGGCACAACTCC
TACCTGAAGGCAGGGCTCCTCCCTGGAGCCAGTGTCTCCTCTCTGGTGGCTCGGCTGCTCCTGAATGA
GGCGGAGCTCCAGGATGGGGAGGCGCTCTGCGTTGGCTTCTCCCTGGGGACGGGGCTCCCCTCGACAG
AGGCGGGGCTACAGATTCGGCGGGGCTTCTCTTGGGTGGGACAGGGCTTCTCCTGCGGGGGCGAGGCC
CCTCCCAGTAAGGGCGTCGCTCTGGGTGGGCGGGGCACTAGGTAGGCTTCTACCTGCAGGCCGGGCTC
CTCCTGAAGGAGGCGGGGCTCTAGGATGGGGCACGGCTCTGGGGTAGGCTGCTCCCTGAGGGCGGAGC
GCCTCCTTAGGAGTGGCGTTTTATGGTGGATGAGGCTTCTTCCTGGATGGCGCAGAGCTTCTCCTGAC
CAGGGCAAGGCCCCTTCCACGGGGGCTGTGGCTCTGGGTGGGCGTGGCCTGCATAGGCTCCTTCCTGT
GGGTGGGGCTTCTCTGGGACCAGGCTCCAATGGGGCGGGGCTTCTCTCCGCGGGTGCGACTCTTCCCT
GGGAACCGCCCTCCTGATTAAGGCGTGGCTTCTCCAGGAATCCCGGCTCCAGAGCAGGAAATTCAGCG
CACCAGCCACCTCATCCCCAACCCCCTGTAACGTTCCATCCACCCCTGCGTCGAGCTGGGAAGGTTCG
ATGAAGCGAGTCGGGTCCCCAACCCGTGCCCCTGGGATCCGAGGGCCCCTCTCCAAGCGCCTGGCTTT
GGAATGCTCCAGCCGCGCCGACGCCTGTGCCACCCCTTCCTCAGCCTGGGGTTTGACCACCCACCTGA
CCAGGGGCCCTACCTGGGGAAAGCCTGAAGGGCCTCCCAGCCCCCAACCCCAAGACCAAGCTTACTCC
TGGGAGAGGACAGGGACTTCGCAGAGGCAAGCGACCGAGGCCCTCCCAAAGAGGCCCGCCCTGCCCGG
GCTCCCACACCGTCAGGTACTCCTGCCAGGGAACTGGCCTGCTGCGCCCCAGGCCCCGCCCGTCTCTG
CTCTGCTCAGCTGCGCCCCCTTCTTTGCAGCTGCCCAGCCCCTCCTCCCTGCCCTCGGGTCTCCCCAC
CTGCACTCCATCCAGCTACAGGAGAGATAGAAGCCTCTCGTCCCGTCCCTCCCTTTCCTCCGCCTGTC
CACAGCCCCTTAAGGGAAAGGTAGGAAGAGAGGTCCAGCCCCCCAGGCTGCCCAGAGCTGCTGGTCTC
ATTTGGGGGCGTTCGGGAGGTTTGGGGGGCATCAACCCCCCGACTGTGCTGCTCGCGAAGGTCCCACA
GCCCTGAGATCGGCCGGCCCCCTTCCTGGCCCCTCATGGCGCGACTGCAGAAATGGTCCGCTTTCCTG
GAGCCAATGGCCCGGCCCCTCCTGACTCATCCGCCTGGCCCGGGAATGAATGGGGAGGCCGCTGAACC
CACCCGGCCCATATCCCTGGTTGCCTCATGGCCAGCGCCCCTCAGCCTCTGCCACTGTGAACCGGCTC
CCACCCTCAAGGTGCGGGGAGAAGAAGCGGCCAGGCGGGGCGCCCCAAGAGCCCAAAAGAGGGCACAC
CGCCATCCTCTGCCTCAAATTCTGCGTTTTTGGTTTTAATGTTATATCTGATGCTGCTATATCCACTG
TCCAACGG

| | | | |
|---|---|---|---|
| NOV5j, CG54611-09 | | | |
| Protein Sequence | SEQ ID NO: 158 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASACVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| NOV5k, CG54611-10 | SEQ ID NO: 159 | 1014 bp |
| DNA Sequence | ORF Start: at 7 | ORF Stop: at 1009 |

GGATCCAGCTACCCGATCTGGTGGTCGCTGGCTGTTGGGCCACAGTATTCCTCCCTGGGCTCGCAGCC
CATCCTGTGTGCCAGCATCCCGGGCCTGGTCCCCAAGCAGCTCCGCTTCTGCAGGAACTACGTGGAGA
TCATGCCCACCGTGGCCGAGGGCATCAAGATTGGCATCCAGGAGTGCCAGCACCAGTTCCGCGGCCGC
CGGTGGAACTGCACCACCGTCCACGACAGCCTGGCCATCTTCGGCCCCGTGCTGGACAAAGCTACCAG
GGAGTCGGCCTTTGTCCACGCCATTGCCTCAGCCGGTGTGGCCTTTGCAGTGACACGCTCATGTGCAG
AAGGCACGGCCGCCATCTGTGGCTGCAGCAGCCGCCACCAGGGCTCACCAGGCAAGGGCTGGAAGTGG
GGTGGCTGTAGCGAGGACATCGAGTTTGGTGGGATGGTGTCTCGCGAGTTCGCCGACGCCCGGGAGAA
CCGGCCAGATGCCCGCTCAGCCATGAACCGCCACAACAACGAGGCTGGGCGCCAGGCCATCGCCAGCC
ACATGCACCTCAAGTGCAAGTGCCACGGCCTGTCGGGCAGCTGCGAGGTGAAGACATGCTGGTGGTCG
CAACCCGACTTCCGCGCCATCGGTGACTTCCTCAAGGACAAGTACGACAGCGCCTCCGAGATGGTGGT
GGAGAAGCACCGGGAGTCCCGCGGCTGGGTGGAGACCCTGCGGCCGCGCTACACCTACTTCAAGGTGC
CCACGGAGCGCGACCTGGTCTACTACGAGGCCTCGCCCAACTTCTGCGAGCCCAACCCTGAGACGGGC
TCCTTCGGCACGCGCGACCGCACCTGCAACGTCAGCTCGCACGGCATCGACGGCTGCGACCTGCTGTG
CTGCGGCCGCGGCCACAACGCGCGAGCGGAGCGGCGCCCGGAGAAGTGCCCCTGCGTGTTCCACTGGT
GCTGCTACGTCAGCTGCCAGGAGTGCACGCGCGTCTACCACGTGCACACCTGCAAGCTCGAG

| | | | |
|---|---|---|---|
| NOV5k, CG54611-10 | | | |
| Protein Sequence | SEQ ID NO: 160 | 334 aa | MW at 37444.0 kD |

SYPIWWSLAVGPQYSSLGSQPILCASIPGLVPKQLRFCRNYVEIMPSVAEGIKIGIQECQHQFRGRRW
NCTTVHDSLAIFGPVLDKATRESAFVHAIASAGVAFAVTRSCAEGTAAICGCSSRHQGSPGKGWKWGG
CSEDIEFGGMVSREFADARENRPDARSAMNRHNNEAGRQAIASHMHLKCKCHGLSGSCEVKTCWWSQP
DFRAIGDFLKDKYDSASEMVVEKHRESRGWVETLRPRYTYFKVPTERDLVYYEASPNFCEPNPETGSF
GTRDRTCNVSSHGIDGCDLLCCGRGHNARAERRREKCRCVFHWCCYVSCQECTRVYDVHTCK

| | | |
|---|---|---|
| NOV5l, CG54611-11 | SEQ ID NO: 161 | 1081 bp |
| DNA Sequence | ORF Start: ATG at 14 | ORF Stop: TAG at 1070 |

CACCGGATCCACCATGGCCCCACTCGGATACTTCTTACTCCTCTGCAGCCTGAAGCAGGCTCTGGGCA
GCTACCCCATCTGGTGGTCGCTGGCTGTTGGGCCACAGTATTCCTCCCTGGGCTCGCAGCCCATCCTG
TGTGCCAGCATCCCGGGCCTGGTCCCCAAGCAGCTCCCCTTCTGCAGGAACTACGTGGAGATCATGCC
CAGCGTGGCCGAGGGCATCAAGATTGGCATCCAGGAGTGCCAGCACCAGTTCCGCGGCCGCCGGTGGA
ACTGCACCACCGTCCACGACAGCCTGGCCATCTTCGGGCCCGTGCTGGACAAAGCTACCAGGGAGTCG
GCCTTTGTCCACGCCATTGCCTCAGCCGGTGTGGCCTTTGCAGTGACACGCTCATGTGCAGAAGGCAC
GGCCGCCATCTGTGGCTGCAGCAGCCGCCACCAGGGCTCACCAGGCAAGGGCTGGAAGTGGGGTGGCT
GTAGCGAGGACATCGAGTTTGGTGGGATGGTGTCTCGGGAGTTCGCCCACGCCCGGGAGAACCGGCCA
GATGCCCGCTCAGCCATGAACCGCGACAACAACGAGGCTGGGCGCCAGGCCATCCCCAGCCACATGCA
CCTCAAGTGCAAGTGCCACGGGCTGTCGGGCAGCTGCGAGGTGAAGACATGCTGGTGGTCGCAACCCG
ACTTCCGCGCCATCGGTGACTTCCTCAAGGACAAGTACGACAGCGCCTCGGAGATGGTGGTGGAGAAG
CACCGGGAGTCCCGCGGCTGGGTGGAGACCCTGCGGCCGCGCTACACCTACTTCAAGGTGCCCACGGA
GCGCGACCTGGTCTACTACGAGGCCTCGCCCAACTTCTGCGAGCCCAACCCTGAGACGGGCTCCTTCG
GCACGCGCCACCGCACCTGCAACGTCAGCTCGCACGGCATCGACGCCTGCGATCTGCTGTGCTGCGGC
CGCGGCCACAACGCGCGAGCGGAGCGGCGCCGGGAGAAGTGCCGGTGCGTGTTCCACTGGTGCTGCTA
CGTCAGCTGCCAGGAGTGCACGCGCGTCTACGACGTGCACACCTGCAAGTAGCTCGAGGGC

| | | | |
|---|---|---|---|
| NOV5l, CG54611-11 | | | |
| Protein Sequence | SEQ ID NO: 162 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| NOV5m, CG54611-12 | SEQ ID NO: 163 | 947 bp |
| DNA Sequence | ORF Start: ATG at 5 | ORF Stop: TAG at 944 |

CTTGATGGCCCCACTCGGATACTTCTTACTCCTCTGCAGCCTGAAGCAGGCTCTGGGCAGCTACCCGA
TCTGGTGGTCGCTGGCTGTTGGCCCACAGTATTCCTCCCTGGGCTCGCAGCCCATCCTGTGTGCCAGC
ATCCCGGGCCTGGTCCCCAAGCAGCTCCGCTTCTGCAGGAACTACGTGGACATCATGCCCAGCGTGGC
CGAGGGCATCAAGATCGGCATCCAGGAGTGCCAGCACCAGTTCCGCGGCCGCCGGTGCAACTGCACCA
CCGTCCACGACAGCCTGGCCATCTTCGGGCCCGTGCTGCACAAAGCTACCAGGGAGTCGGCCTTTGTC
CACGCCATTGCCTCAGCCGGTGTGGCCTTTGCAGTGACACGCTCATCTGCAGAAGGCGCCGCCCCCAT
CTGTGGCTGCAGCAGCCGCCACCAGGGCTCACCAGGCAAGGGCTGGAAGTGGGGTGGCTGTACCGAGG
ACATCGAGTTTGGTGGGATGGTGTCTCGGGAGTTCGCCGACGCCCGGGAGAACCGGCCAGATGTCCGC
TCAGCCATGAACCGCCACAACAACGAGGCTGGGCGCCAGGACAAGTACGACAGCGCCTCGGAGATGGT
GGTGGAGAAGCACCGGGAGTCCCGCGGCTGGGTGGAGACCCTGCGGCCGCGCTACACCTACTTCAAGG
TGGCCACGGAGCGCGACCTGGTCTACTACGAGGCCTCGCCCAACTTCTGCGAGCCCAACCCTGAGACG
GGCTCCTTCGGCACGCGCGACCGCACCTGCAACGTCAGCTCGCACGGCATCGACGGCTGCGACCTGCT
GTGCTGCGGCCGCGGCCACAACGCGCGAGCGGAGCGGCGCCGGGAGAAGTGCCGCTGCGTGTTCCACT
GGTGCTGCTACGTCAGCTGCCAGGAGTGCACGCGCGTCTACGACGTGCACACCTGCAAGTAGG

| | | | |
|---|---|---|---|
| NOV5m, CG54611-12 | | | |
| Protein Sequence | SEQ ID NO: 164 | 313 aa | MW at 34988.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGAAAIC
GCSSRHQGSPGKGWKWGGCSEDIEFGGMVSREFADARENRPDVRSANNRHNNEAGRQDKYDSASEMVV
EKHRESRGWVETLRPRYTYFKVPTERDLVYYEASPNFCEPNPETGSFGTRDRTCNVSSHGIDGCDLLC
CGRGHNARAERRREKCRCVFHWCCYVSCQECTRVYDVHTCK

| | | |
|---|---|---|
| NOV5n,CG54611-13 | SEQ ID NO: 165 | 1194 bp |
| DNA Sequence | ORF Start: ATG at 28 | ORF Stop: TAG at 1165 |

GATGGCCCACTCGGATACTTCTTACTGATGGCCCCACTCGGATACTTCTTACTGATGGCCCCACTCGG
ATACTTCTTACTGATGGCCCCACTCGGATACTTCTTACTGATCGCCCCACTCGGATACTTCTTACTCC
TCTGCAGCCTGAAGCAGGCTCTGGGCAGCTACCCGATCTGGTGGTCGCTGGCTGTTGGGCCACAGTAT
TCCTCCCTGGGCTCGCAGCCCATCCTGTGTGCCAGCATCCCGGGCCTGGTCCCCAAGCAGCTCCGCTT
CTGCAGGAACTACGTGGAGATCATGCCCAGCGTGGCCGAGGGCATCAAGATTGGCATCCACGAGTGCC
AGCACCAGTTCCGCGGCCGCCGGTGGAACTGCACCACCGTCCACGACAGCCTGGCCATCTTCGGGCCC
GTGCTGGACAAAGCTACCAGGGAGTCGGCCTTTGTCCACGCCATTGCCTCAGCCGGTGTGGCCTTTGC
AGTGACACGCTCATGTGCAGAAGGCACGGCCGCCATCTGTGGCTGCAGCAGCCGCCACCAGGGCTCAC
CGGGCAAGGGCTGGAAGTGGGGTGGCTGTAGCGAGGACATCGAGTTTGGTGGGATGGTGTCTCGGGAG
TTCGCCGACGCCCGGGAGAACCGGCCAGATGCCCGCTCAGCCATGAACCGCCACAACAACGAGGCTGG
GCGCCAGGCCATCGCCAGCCACATGCACCTCAAGTGCAAGTGCCACGGGCTGTCGGGCAGCTGCGAGG
TGAAGACATGCTGGTGGTCGCAACCCGACTTCCGCGCCATCGGTGACTTCCTCAAGGACAAGTACGAC
AGCGCCTCGGAGATGGTGGTGGAGAAGCACCGGGAGTCCCGCGGCTGGGTGGAGACCCTGCGGCCGCG
CTACACCTACTTCAAGGTGCCCACGGACCGCGACCTGGTCTACTACGAGGCCTCGCCCAACTTCTGCG
AGCCCAACCCTGAGACGGGCTCTTTCGGCACGCGCGACCGCACCTGCAACGTCAGCTCGCACGGCATC
GACGGCTGCGACCTGCTGTGCTGCGGCCGCGGCCACAACGCGCGAGCGGAGCGGCGCCGGGAGAAGTG
CCGCTGCGTGTTCCACTGGTGCTGCTACGTCAGCTGCCAGGAGTGCACGCGCGTCTACGACGTGCACA
CCTGCAAGTAGGCACGTGCACACCTGCAAGTAGGCATC

| | | | |
|---|---|---|---|
| NOV5n, CG54611-13 | | | |
| Protein Sequence | SEQ ID NO: 166 | 379 aa | MW at 42383.1 kD |

MAPLGYFLLMAPLGYFLLMAPLGYFLLMAPLGYFLLLCSLKQALGSYPIWWSLAVCPQYSSLGSQPIL
CASIPGLVPKQLRFCRNYVEIMPSVAEGIKIGIQECQHQFRGRRWNCTTVHDSLAIFGPVLDKATRES
AFVHAIASAGVAFAVTRSCAEGTAAICGCSSRHQGSPGKGWKWGGCSEDIEFGGMVSREFADARENRP
DARSAMNRHNNEAGRQAIASHMHLKCKCHGLSGSCEVKTCWWSQPDFRAIGDFLKDKYDSASEMVVEK
HRESRGWVETLRPRYTYFKVPTERDLVYYEASPNFCEPNPETGSFGTRDRTCNVSSHGIDGCDLLCCG
RGHNARAERRREKCRCVFHWCCYVSCQECTRVYDVHTCK

| | | |
|---|---|---|
| NOV5o, CG54611-14 | SEQ ID NO: 167 | 1082 bp |
| DNA Sequence | ORF Start: ATG at 16 | ORF Stop: TAG at 1072 |

CCCTCTCGCGCGGCGATGGCCCCACTCGGATACTTCTTACTCCTCTGCAGCCTGAAGCAGGCTCTGGG
CAGCTACCCGATCTGGTGGTCGCTGGCTGTTGGGCCACAGTATTCCTCCCTGGGCTCGCAGCCCATCC
TGTGTGCCAGCATCCCGGGCCTGGTCCCCAAGCAGCTCCGCTTCTGCAGGAACTACGTGGAGATCATG
CCCAGCGTGGCCGAGGGCATCAAGATTGGCATCCAGGAGTGCCAGCACCAGTTCCGCGGCCGCCGGTG
GAACTGCACCACCGTCCACGACAGCCTCGCCATCTTCGGGCCCGTGCTGGACAAAGCTACCAGGGAGT
CGCCCTTTGTCCACGCCATTGCCTCAGCCGGTGTGGCCTTTGCAGTGACACGCTCATGTGCAGAACGC
ACGGCCGCCATCTGTGGCTGCAGCAGCCGCCACCAGGGCTCACCAGGCAAGGGCTGGAAGTGGGGTGG
CTGTAGCGAGGACATCGAGTTTGGTGGGATGGTGTCTCGGGAGTTCGCCGACGCCCGGGAGAACCGGC
CAGATGCCCGCTCAGCCATGAACCGCCACAACAACGAGGCTGGGCGCCAGGCCATCGCCACCCACATG
CACCTCAAGTGCAAGTGCCACGGGCTGTCGGGCAGCTGCGAGGTGAAGACATGCTGGTGGTCGCAACC
CGACTTCCCCGCCATCGGTGACTTCCTCAAGGACAACTACGACACCGCCTCGGACATGGTCGTGCAGA
AGCACCGGGAGTCCCGCGGCTGGGTGGAGACCCTGCGGCCGCGCTACACCTACTTCAAGGTGCCCACG
GAGCGCGACCTGGTCTACTACGAGGCCTCGCCCAACTTCTGCGAGCCCAACCCTGAGACGGGCTCCTT
CGGCACGCGCGACCGCACCTGCAACGTCAGCTCGCACGGCATCGACGGCTGCGACCTGCTGTGCTGCG
GCCGCGGCCACAACGCGCGAGCGGAGCGGCGCCGGGAGAAGTGCCGCTGCGTGTTCCACTGGTGCTGC
TACGTCAGCTGCCAGGAGTGCACGCGCGTCTACGACGTGCACACCTGCAAGTAGGCACCGGC

| | | | |
|---|---|---|---|
| NOV5o, CG54611-14 | | | |
| Protein Sequence | SEQ ID NO: 168 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSANNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| NOV5p, CG54611-15 | SEQ ID NO: 169 | 1076 bp |
| DNA Sequence | ORF Start: ATG at 2 | ORF Stop: TAG at 1058 |

GATGGCCCCACTCGGATACTTCTTACTCCTCTGCAGCCTGAAGCAGGCTCTGGGCAGCTACCCGATCT
GGTGGTCGCTGGCTGTTGGGCCACAGTATTCCTCCCTCGGCTCGCAGCCCATCCTGTGTGCCAGCATC
CCGGGCCTCGTCCCCAAGCAGCTCCGCTTCTGCAGGAACTACGTGGAGATCATGCCCAGCGTGGCCGA
GGGCATCAACATTGGCATCCAGGAGTGCCAGCACCAGTTCCGCGGCCGCCGGTGGAACTGCACCACCG
TCCACGACACCCTGGCCATCTTCGGGCCCGTGCTGGACAAAGCTACCAGGGAGTCGGCCTTTGTCCAC
GCCATTGCCTCAGCCGGTGTGGCCTTTGCAGTGACACGCTCATGTGCAGAACGCACGGCCGCCATCTG
TGGCTGCAGCAGCCGCCACCAGGGCTCACCAGGCAAGGGCTGGAAGTGGGGTGGCTGTAGCGAGGACA
TCGAGTTTGGTGGGATGGTGTCTCGGGAGTTCGCCGACGCCCGGGAGAACCGGCCAGATGCCCGCTCA
GCCATGAACCGCCACAACAACGAGGCTGGGCGCCAGGCCATCGCCAGCCACATGCACCTCAAGTGCAA
GTGCCACGGGCTGTCGGGCAGCTGCGAGGTGAAGACATGCTGGTGGTCGCAACCCGACTTCCGCGCCA
TCGGTGACTTCCTCAAGGACAAGTACGACAGCGCCTCGGAGATGGTGGTGGAGAAGCACCGGGAGTCC
CGCGGCTGGGTGGAGACCCTGCGGCCGCGCTACACCTACTTCAAGGTGCCCACGGAGCGCGACCTGGT
CTACTACGAGGCCTCGCCCAACTTCTGCGAGCCCAACCCTGAGACGGGCTCCTTCGGCACGCGCGACC
GCACCTGCAACGTCAGCTCGCACGGCATCGACGGCTGCGACCTGCTGTGCTGCGGCCGCGGCCACAAC
GCGCGAGCGGAGCGGCGCCGGGAGAAGTGCCGCTGCGTGTTCCACTGGTGCTGCTACGTCAGCTGCCA
GGAGTGCACCCGCGTCTACGACGTGCACACCTGCAAGTAGAAGGGCGAATTCCGCC

| | | | |
|---|---|---|---|
| NOV5p, CG54611-15 | | | |
| Protein Sequence | SEQ ID NO: 170 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDTEFGGMVSREFADARENRPDARSAMNRHNNEAGRQAIASHMHLKCK
CHGLSGSCEVKTCWWSQPDFRATGDFLKDKYDSASEMVVEKHRESRGWVETLRPRYTYFKVPTERDLV
YYEASPNFCEPNPETGSFGTRDRTCNVSSHGIDGCDLLCCGRGHNAPAERRREKCRCVFHWCCYVSCQ
ECTRVYDVHTCK

| | | |
|---|---|---|
| NOV5q, CG54611-16 | SEQ ID NO: 171 | 1194 bp |
| DNA Sequence | ORF Start: ATG at 28 | ORF Stop: TAG at 1165 |

GATGGCCCACTCGGATACTTCTTACTGATGGCCCCACTCGGATACTTCTTACTGATGGCCCCACTCGG
ATACTTCTTACTGATGGCCCCACTCGGATACTTCTTACTGATGGCCCCACTGGGATACTTCTTACTCC
TCTGCAGCCTGAAGCAGGCTCTGGGCAGCTACCCGATCTGGTGGTCGCTGGCTGTTGGGCCACAGTAT
TCCTCCCTGGGCTCGCAGCCCATCCTGTGTGCCAGCATCCCGGGCCTGGTCCCCAAGCAGCTCCGCTT
CTGCAGGAACTACGTGGAGATCATGCCCAGCGTGGCCGAGGGCATCAAGATTGGCATCCAGGAGTGCC
AGCACCAGTTCCGCGGCCGCCGGTGGAACTGCACCACCGTCCACGACAGCCTGGCCATCTTCGGGCCC
GTGCTGGACAAAGCTACCAGGGAGTCGGCCTTTGTCCACGCCATTGCCTCACCCGGTGTGGCCTTTGC
AGTGACACGCTCATGTGCAGAAGGCACGGCCGCCATCTGTGGCTGCAGCAGCCGCCACCAGGGCTCAC
CGGGCAAGGGCTGGAAGTGGGGTGGCTGTAGCGAGGACATCGAGTTTGGTGGGATGGTGTCTCGGGAG
TTCGCCGACCCCCGGGACAACCGGCCAGATGCCCGCTCAGCCATGAACCGCCACAACAACGAGGCTGG
GCGCCAGGCCATCGCCAGCCACATGCACCTCAAGTGCAAGTGCCACGGGCTGTCGGGCAGCTGCGAGC
TGAAGACATGCTGGTGGTCGCAACCCGACTTCCGCGCCATCGGTGACTTCCTCAAGGACAAGTACGAC
AGCGCCTCGGAGATGGTGGTGGAGAAGCACCGGGAGTCCCGCGGCTGGGTGGAGACCCTGCGGCCGCG
CTACACCTACTTCAAGGTGCCCACGGAGCGCGACCTGGTCTACTACCAGGCCTCGCCCAACTTCTGCG
AGCCCAACCCTGAGACGGGCTCTTTCGGCACGCGCGACCGCACCTGCAACGTCAGCTCGCACGGCATC
GACGGCTGCGACCTGCTGTGCTGCGGCCGCGGCCACAACGCGCGAGCGGAGCGGCGCCGGGAGAAGTG
CCGCTGCGTGTTCCACTGGTGCTGCTACGTCAGCTGCCAGGAGTGCACGCGCGTCTACGACGTGCACA
CCTGCAAGTAGGCACGTGCACACCTGCAAGTAGGCATC

| | | | |
|---|---|---|---|
| NOV5q, CG54611-16 | | | |
| Protein Sequence | SEQ ID NO: 172 | 379 aa | MW at 42383.1 kD |

MAPLGYFLLMAPLGYFLLMAPLGYFLLMAPLGYFLLLCSLKQALGSYPIWWSLAVGPQYSSLGSQPIL
CASIPGLVPKQLRFCRNYVEIMPSVAEGIKIGIQECQHQFRGRRWNCTTVHDSLAIFGPVLDKATRES
AFVHAIASAGVAFAVTRSCAEGTAAICGCSSRHQGSPGKGWKWGGCSEDIEFGGMVSREFADARENRP
DARSAMNRHNNEAGRQAIASHMHLKCKCHGLSGSCEVKTCWWSQPDFRAIGDFLKDKYDSASEMVVEK
HRESRGWVETLRPRYTYFKVPTERDLVYYEASPNFCEPNPETGSFGTRDRTCNVSSHGIDGCDLLCCG
RGHNARAERRREKCRCVFHWCCYVSCQECTRVYDVHTCK

| | | |
|---|---|---|
| NOV5r, CG54611-17 | SEQ ID NO: 173 | 1060 bp |
| DNA Sequence | ORF Start: ATG at 2 | ORF Stop: TAG at 1058 |

GATGGCCCCACTCGGATACTTCTTACTCCTCTGCAGCCTGAAGCAGGCTCTGGGCAGCTACCCGATCT
GGTGGTCGCTGGCTGTTGGGCCACAGTATTCCTCCCTGGGCTCGCAGCCCATCCTGTGTGCCAGCATC
CCGGGCCTGGTCCCCAAGCAGCTCCGCTTCTGCAGGAACTACGTGGAGATCATGCCCAGCGTGGCCGA
GGGCATCAAGATTGGCATCCAGGAGTGCCAGCACCAGTTCCGCGGCCGCCGGTGGAACTGCACCACCG
TCCACGACAGCCTGGCCATCTTCGGGCCCGTGCTGGACAAAGCTACCAGGGAGTCGGCCTTTGTCCAC
GCCATTGCCTCAGCCGGTGTGGCCTTTGCAGTGACACGCTCATGTGCAGAAGGCACGGCCGCCATCTG
TGGCTGCAGCAGCCGCCACCAGGGCTCACCAGGCAAGGGCTGGAAGTGGGGTGGCTGTAGCGAGGACA
TCGAGTTTGGTGGGATGGTGTCTCGGGAGTTCGCCGACGCCCGGGAGAACCGGCCAGATGCCCGCTCA
GCCATGAACCGCCACAACAACGAGGCTGGCCGCCAGGCCATCGCCAGCCACATGCACCTCAAGTCCAA
GTGCCACCGGCTGTCGGGCAGCTGCGAGGTGAAGACATGCTGGTGGTCGCAACCCGACTTCCGCGCCA
TCGGTGACTTCCTCAAGGACAAGTACGACAGCGCCTCGGAGATGGTGGTGGAGAAGCACCGGCAGTCC
CGCGGGTGGGTGGAGACCCTGCGGCCGCGCTACACCTACTTCAAGGTGCCCACGGAGCGCGACCTGGT
CTACTACGAGGCCTCGCCCAACTTCTGCGAGCCCAACCCTGAGACGGGCTCCTTCGGCACGCGCGACC
GCACCTGCAACGTCAGCTCGCACGGCATCGACGGCTGCGACCTGCTGTGCTGCGGCCGCGGCCACAAC
GCGCGAGCGGAGCGGCGCCGGGAGAAGTGCCGCTGCGTGTTCCACTGGTGCTGCTACGTCAGCTGCCA
GGAGTGCACGCGCGTCTACGACGTGCACACCTGCAAGTAG

| | | | |
|---|---|---|---|
| NOV5r, CG54611-17 | | | |
| Protein Sequence | SEQ ID NO: 174 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIOECOHOFRGRRWNCTTVHDSLAIFGPVLDKATRESAFVHAIASAGVAFAVTRSCAEGTAAIC
GCSSRHQGSPGKGWKWGGCSEDTEFGGMVSREFADARENRPDARSAMNRHNNEAGRQAIASHNHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGXNARAERRREKCRCVFHWCCYVSCQ
ECTRVYDVHTCK

| | | |
|---|---|---|
| NOV5s, CG54611-18 | SEQ ID NO: 175 | 1060 bp |
| DNA Sequence | ORF Start: ATG at 2 | ORF Stop: TAG at 1058 |

GATGGCCCCACTCGGATACTTCTTACTCCTCTGCAGCCTGAAGCAGGCTCTGGGCAGCTACCCGATCT
GCTGGTCGCTGGCTGTTGGGCCACAGTATTCCTCCCTGGGCTCGCAGCCCATCCTGTGTGCCAGCATC
CCGGGCCTGGTCCCCAAGCAGCTCCGCTTCTGCAGGAACTACGTGGAGATCATGCCCAGCGTGGCCGA
GGGCATCAAGATTGGCATCCAGGAGTGCCAGCACCAGTTCCGCGGCCGCCGGTGGAACTGCACCACCG
TCCACGACAGCCTGGCCATCTTCGGGCCCGTGCTGGACAAAGCTACCAGGGAGTCGGCCTTTGTCCAC
GCCATTGCCTCAGCCGGTGTGGCCTTTGCAGTGACACGCTCATGTGCAGAAGGCACGGCCGCCATCTG
TGGCTGCAGCAGCCGCCACCAGGGCTCACCAGGCAAGGGCTGGAAGTGGGGTGGCTGTAGCGAGGACA
TCGAGTTTGGTGGGATGGTGTCTCGGGAGTTCGCCGACGCCCGGGAGAACCGGCCAGATGCCCGCTCA
GCCATGAACCGCCACAACAACGAGGCTGGGCGCCAGGCCATCGCCAGCCACATGCACCTCAAGTGCAA
GTGCCACCGGCTGTCGGGCAGCTGCGAGGTGAAGACATGCTGGTGGTCGCAACCCGACTTCCGCGCCA
TCGGTGACTTCCTCAAGGACAAGTACGACAGCGCCTCGGAGATGGTGGTGGAGAAGCACCGGGAGTCC
CGCGGCTGGGTGGAGACCCTGCGGCCGCGCTACACCTACTTCAAGGTGCCCACGGAGCGCGACCTGGT
CTACTACGAGGCCTCGCCCAACTTCTGCGAGCCCAACCCTGAGACGGGCTCCTTCGGCACGCGCGACC
GCACCTGCAACGTCAGCTCGCACGGCATCGACGGCTGCGACCTGCTGTGCTGCGGCCGCGGCCACAAC
GCGCGAGCGGAGCGGCGCCGGGAGAAGTGCCGCTGCGTGTTCCACTGGTGCTGCTACGTCAGCTGCCA
GGAGTGCACGCGCGTCTACGACGTGCACACCTGCAAGTAG

| | | | |
|---|---|---|---|
| NOV5s, CG54611-18 | | | |
| Protein Sequence | SEQ ID NO: 176 | 352 aa | MW at 39364.3 kD |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNAEAERRREKCRCVFHWCCYVSCQ
ECTRVYDVHTCK

| | | |
|---|---|---|
| | SEQ ID NO: 177 | 1116 bp |
| NOV5t, SNP13378438 of | ORF Start: ATG at 31 | ORF Stop: TAG at 1087 |
| CG54611-01, DNA Sequence | SNP Pos: 149 | SNP Change: T to C |

TCCCGGCCCTCCGCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGGCCACAGTATTCCTCCCTGG
GCTCGCAGCCCACCCTGTGTGCCAGCATCCCGGGCCTGGTCCCCAAGCAGCTCCGCTTCTGCAGGAAC
TACGTGGAGATCATGCCCAGCGTGGCCGAGGGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGCCTGCAGCAGCCGCCACCAGGGCTCACCAGGCAAGGG
CTGGAAGTGGGGTGGCTGTAGCGAGGACATCGAGTTTGGTGGCATGGTGTCTCGGGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGCGCTGTCGGGCAGCTGCGAGGTGAAGACATG
CTGGTGGTCGCAACCCGACTTCCGCGCCATCGGTGACTTCCTCAAGGACAAGTACGACAGCGCCTCGG
AGATGGTGGTGGAGAAGCACCGGGAGTCCCGCGGCTGGGTGGAGACCCTGCGGCCGCGCTACACCTAC
TTCAAGGTGCCCACGGAGCGCGACCTGGTCTACTACGAGGCCTCGCCCAACTTCTGCGAGCCCAACCC
TGAGACGGGCTCCTTCGGCACGCGCGACCGCACCTGCAACGTCAGCTCGCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGCAGTGCACGCGCGTCTACGACGTGCACACCTGCAAGTA
GGCACCGGCCGCGGCTCCCCCTGGACCG

| | | | |
|---|---|---|---|
| NOV5t, SNP13378438 of | SEQ ID NO: 178 | | MW at 39352.3 kD |
| CG54611-01, Protein Sequence | SNP Pos: 40 | 352 aa | SNP Change: Ile to Thr |

MAPLGYFLLLCSLKQALGSYPIWWSLAVGPQYSSLGSQPTLCASIPCLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCGGRGHNARAERRREKCRCVFHWCCYVSCQ
ECTRVYDVHTCK

| | | |
|---|---|---|
| | SEQ ID NO: 179 | 1116 bp |
| NOV5u, SNP13378437 of | ORF Start: ATG at 31 | ORF Stop: TAG at 1087 |
| CG54611-01, DNA Sequence | SNP Pos: 160 | SNP Change: A to G |

TCCCGGCCCTCCGCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGGCCACAGTATTCCTCCCTGG
GCTCGCAGCCCATCCTGTGTGCCGGCATCCCGGGCCTGGTCCCCAAGCAGCTCCGCTTCTGCAGGAAC
TACGTGGACATCATGCCCAGCGTGGCCGAGGGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGGCTGCAGCAGCCGCCACCAGGGCTCACCAGGCAAGGG
CTGGAAGTGGGGTGGCTGTAGCGAGGACATCGAGTTTGGTGGGATGGTGTCTCGGGAGTTCGCCGACG
CCCGGGAGAACCGGCCAGATGCCCGCTCAGCCATGAACCCCCACAACAACGAGGCTGGGCGCCAGGCC
ATCGCCAGCCACATGCACCTCAAGTGCAAGTGCCACGGGCTGTCGGGCAGCTGCGAGGTGAAGACATG
CTGGTGGTCGCAACCCGACTTCCGCGCCATCGGTGACTTCCTCAAGGACAAGTACGACAGCGCCTCGG
AGATGGTGGTGGAGAAGCACCGGGAGTCCCGCGGCTGGGTGGAGACCCTGCGCCCGCGCTACACCTAC
TTCAAGGTGCCCACGGAGCGCGACCTGGTCTACTACGAGGCCTCCCCCAACTTCTGCCAGCCCAACCC
TGAGACGGGCTCCTTCGGCACGCGCGACCGCACCTGCAACGTCAGCTCGCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGGAGTGCACGCGCGTCTACGACGTGCACACCTGCAAGTA
GGCACCGGCCGCGGCTCCCCCTGGACGG

| | | | |
|---|---|---|---|
| NOV5u, SNP13378437 of | SEQ ID NO: 180 | | MW at 39334.3 kD |
| CG54611-01, Protein Sequence | SNP Pos: 44 | 352 aa | SNP Change: Ser to Gly |

MAPLGYFLLLCSLKQALGSYPIWWSLAVGPQYSSLGSQPILCAGIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSANNRHNNEAGRQAIASHMHLKCK
CHGLSGSCEVKTCWWSQPDFRAICDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| | SEQ ID NO: 181 | 1116 bp |
| NOV5v, SNP13381548 of | ORF Start: ATG at 31 | ORF Stop: TAG at 1087 |
| CG54611-01, DNA Sequence | SNP Pos: 430 | SNP Change: G to A |

TCCCGGCCCTCCGCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGGCCACAGTATTCCTCCCTGG
GCTCGCAGCCCATCCTGTGTGCCAGCATCCCGGGCCTGGTCCCCAAGCAGCTCCGCTTCTGCAGGAAC
TACGTGGAGATCATGCCCAGCGTGGCCGAGGGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCACCATCTGTGGCTGCAGCAGCCGCCACCAGGGCTCACCAGGCAAGGG
CTGGAAGTGGGGTGGCTGTAGCGAGGACATCGAGTTTGGTGGGATGGTGTCTCGCGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGGGCTGTCGGGCAGCTGCGAGGTGAAGACATG
CTGGTGGTCGCAACCCGACTTCCGCGCCATCGGTGACTTCCTCAAGGACAAGTACGACAGCGCCTCGG
AGATGGTGGTGGAGAAGCACCGGGAGTCCCGCGGCTGGGTGGAGACCCTGCGGCCGCGCTACACCTAC
TTCAAGGTGCCCACGGAGCGCGACCTGGTCTACTACGAGGCCTCGCCCAACTTCTGCGAGCCCAACCC
TGAGACGGGCTCCTTCGGCACGCGCGACCGCACCTGGAACGTCAGCTCGCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGGAGTGCACGCGCGTCTACGACGTGCACACCTGCAAGTA
GGCACCGGCCGCGGCTCCCCCTGGACGG

| | | | |
|---|---|---|---|
| NOV5v, SNP13381548 of | SEQ ID NO: 182 | | MW at 39394.3 kD |
| CG54611-01, Protein Sequence | SNP Pos: 134 | 352 aa | SNP Change: Ala to Thr |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTATIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| | SEQ ID NO: 183 | 1116 bp |
| NOV5w, SNP13381645 of | ORF Start: ATG at 31 | ORF Stop: TAG at 1087 |
| CG54611-01, DNA Sequence | SNP Pos: 664 | SNP Change: T to C |

TCCCGGCCCTCCCCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGGCCACAGTATTCCTCCCTGG
GCTCGCAGCCCATCCTGTGTGCCAGCATCCCGGGCCTGGTCCCCAAGCAGCTCCGCTTCTGCAGGAAC
TACGTGGAGATCATGCCCAGCGTGGCCGAGGGCATCAAGATTGGCATCCAGGAGTGCCAGCACCAGTT
CCGCGGCCGCCGGTCGAACTGCACCACCGTCCACGACAGCCTGGCCATCTTCGGGCCCGTCCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGGCTGCAGCAGCCGCCACCAGGGCTCACCAGGCAAGGG
CTGGAAGTCGGGTGGCTGTAGCGAGGACATCGAGTTTGCTGGGATGGTGTCTCGGGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGGGCTGTCGGGCAGCCGCGAGGTGAAGACATG
CTGGTGGTCGCAACCCGACTTCCGCGCCATCGGTGACTTCCTCAAGGACAAGTACGACAGCGCCTCGG
AGATGGTGGTGGAGAAGCACCGGGAGTCCCGCGGCTGGGTGGAGACCCTGCGGCCGCGCTACACCTAC
TTCAAGGTGCCCACGGAGCGCGACCTGGTCTACTACGAGGCCTCGCCCAACTTCTGCGAGCCCAACCC
TGAGACGGGCTCCTTCGGCACGCGCGACCGCACCTGCAACGTCAGCTCGCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGGAGTGCACGCGCGTCTACGACGTGCACACCTGCAAGTA
GGCACCGGCCGCGCCTCCCCCTGCACGG

| | | | |
|---|---|---|---|
| NOV5w, SNP13381645 of | SEQ ID NO: 184 | | MW at 39417.4 kD |
| CG54611-01, Protein Sequence | SNP Pos: 212 | 352 aa | SNP Change: Cys to Arg |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSANNRHNNEAGRQAIASHMHLKCK
CHGLSGSREVKTCWWSQPDFRAIGDFLKDKYDSASEMVVEKHRESRGWVETLRPRYTYFKVPTERDLV
YYEASPNFCEPNPETGSFCTRDRTCNVSSHGIDGCDLLCCGRGHNARAERRREKCRCVFHWCCYVSCQ
ECTRVYDVHTCK

| | | |
|---|---|---|
| | SEQ ID NO: 185 | 1116 bp |
| NOV5x, SNP13381646 of | ORF Start: ATG at 31 | ORF Stop: TAG at 1087 |
| CG54611-01, DNA Sequence | SNP Pos: 731 | SNP Change: A to G |

TCCCGGCCCTCCGCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGGCCACAGTATTCCTCCCTGC
GCTCGCAGCCCATCCTGTGTGCCAGCATCCCGGGCCTGGTCCCCAAGCAGCTCCGCTTCTGCAGGAAC
TACGTGGAGATCATGCCCAGCGTGGCCGAGGGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGGCTGCAGCAGCCGCCACCAGGGCTCACCAGGCAAGGG
CTGGAAGTGGGGTGGCTGTAGCGAGGACATCGAGTTTGGTGGCATGGTGTCTCGGGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGGGCTGTCGGGCAGCTGCGAGGTGAAGACATG
CTGGTGGTCGCAACCCGACTTCCGCGCCATCGGTGACTTCCTCAAGGACAGGTACGACAGCGCCTCGG
AGATGGTGGTGGAGAAGCACCGGCAGTCCCGCGGCTGGGTGGAGACCCTGCGGCCGCGCTACACCTAC
TTCAAGGTGCCCACGGAGCGCGACCTGGTCTACTACGAGGCCTCGCCCAACTTCTGCGAGCCCAACCC
TGACACGGGCTCCTTCGGCACGCGCGACCGCACCTGCAACGTCAGCTCGCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGGAGTGCACGCGCGTCTACGACGTGCACACCTGCAAGTA
GGCACCGGCCGCGGCTCCCCCTGGACGG

| | | | |
|---|---|---|---|
| NOV5x, SNP13381646 of SEQ ID NO: 186 | | MW at 39392.3 kD | |
| CG54611-01, Protein Sequence | SNP Pos: 234 | 352 aa | SNP Change: Lys to Arg |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSANNRHNNEAGRQAIASHMHLKCK
CHGLSGSCEVKTCWWSQPDFRAIGDFLKDRYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDVHTCK

| | | |
|---|---|---|
| | SEQ ID NO: 187 | 1116 bp |
| NOV6y, SNP13381647 of | ORF Start: ATG at 31 | ORF Stop: TAG at 1087 |
| CG54611-01, DNA Sequence | SNP Pos: 895 | SNP Change: T to C |

TCCCGGCCCTCCGCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGGCCACAGTATTCCTCCCTGG
GCTCGCAGCCCATCCTGTGTGCCAGCATCCCGGGCCTGGTCCCCAAGCAGCTCCGCTTCTGCAGGAAC
TACGTGGAGATCATGCCCAGCGTGGCCGAGGGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGGCTGCAGCAGCCGCCACCAGGGCTCACCAGGCAAGGG
CTGGAAGTGGGGTGGCTGTAGCCAGGACATCGAGTTTGGTGGGATGGTGTCTCGGGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGGGCTGTCGGGCAGCTGCGAGGTGAAGACATG
CTGGTGGTCGCAACCCGACTTCCGCGCCATCGGTGACTTCCTCAAGGACAAGTACGACAGCGCCTCGG
AGATGGTGGTGGAGAAGCACCGGGAGTCCCGCGGCTGGGTGGAGACCCTGCGGCCGCGCTACACCTAC
TTCAAGGTGCCCACGGAGCGCGACCTGGTCTACTACGAGGCCTCGCCCAACTTCTGCGAGCCCAACCC
TGAGACGGGCCCCTTCGGCACGCGCGACCGCACCTGCAACGTCAGCTCCCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGGAGTGCACGCCCGTCTACGACGTGCACACCTGCAAGTA
GGCACCGGCCGCGGCTCCCCCTGGACGG

| | | | |
|---|---|---|---|
| NOV6y, SNP13381647 of | SEQ ID NO: 188 | | MW at 39374.4 kD |
| CG54611-01, Protein Sequence | SNP Pos: 289 | 352 aa | SNP Change: Ser to Pro |

MAPLQYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGPFGTRDRTCNVSSHGIDGCDLLCCGRCHNARAERRREKCRCVFHWCCYVSCQ
ECTRVYDVHTCK

| | | |
|---|---|---|
| | SEQ ID NO: 189 | 1116 bp |
| NOV5z, SNP13381648 of | ORF Start: ATG at 31 | ORF Stop: TAG at 1087 |
| CG54611-01, DNA Sequence | SNP Pos: 961 | SNP Change: T to C |

TCCCGGCCCTCCGCGCCCTCTCGCGCGGCGATGGCCCCACTCCGATACTTCTTACTCCTCTGCAGCCT
GAAGCAGGCTCTGGGCAGCTACCCGATCTGGTGGTCGCTGGCTGTTGGGCCACAGTATTCCTCCCTGG
GCTCGCAGCCCATCCTGTGTGCCAGCATCCCGGGCCTGGTCCCCAAGCAGCTCCGCTTCTGCAGGAAC
TACGTGGAGATCATGCCCAGCGTGGCCGAGGGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGCACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGGCTGCAGCAGCCGCCACCAGGGCTCACCAGGCAAGGG
CTGGAAGTGGGGTGGCTGTAGCGAGGACATCGAGTTTGGTGGGATGGTGTCTCGGGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGGGCTGTCGGGCAGCTGCGAGGTGAAGACATG
CTGGTGGTCGCAACCCGACTTCCGCGCCATCGCTGACTTCCTCAACGACAAGTACGACAGCGCCTCGG
AGATGGTGGTGGAGAAGCACCGGGAGTCCCGCGGCTGGGTGGAGACCCTGCGGCCGCGCTACACCTAC
TTCAAGGTGCCCACGGAGCGCCACCTGGTCTACTACGAGGCCTCGCCCAACTTCTGCGAGCCCAACCC
TGAGACGGGCTCCTTCGGCACGCGCGACCGCACCTGCAACGTCAGCTCGCACGGCATCGACGGCTGCG
ACCTGCTGCGCTGCGGCCGCGGCCACAACGCGCGAGCGGAGCGGCGGCGGGAGAAGTGCCGCTGCGTG
TTCCACTGGTGCTGCTACGTCAGCTGCCAGGAGTGCACCCGCGTCTACGACGTGCACACCTGCAAGTA
GGCACCGGCCGCGGCTCCCCCTGGACGG

| | | | |
|---|---|---|---|
| NOV5z, SNP13381648 of | SEQ ID NO: 190 | | MW at 39417.4 kD |
| CG54611-01, Protein Sequence | SNP Pos: 311 | 352 aa | SNP Change: Cys to Arg |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHNHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLRCGRGHNAHAERRREKCRCVFHWCCYVSCQ
ECTRVYDVHTCK

| | | |
|---|---|---|
| | SEQ ID NO: 191 | 1116 bp |
| NOV5aa, SNP13381649 of | ORF Start: ATG at 31 | ORF Stop: TAG at 1087 |
| CG54611-01, DNA Sequence | SNP Pos: 1073 | SNP Change: T to C |

TCCCGGCCCTCCGCGCCCTCTCGCGCGGCGATGGCCCCACTCGGATACTTCTTACTCCTCTGCAGCCT
GAAGCAGGCTCTGGGCAGCTACCCGATCTGGTGGTCGCTGGCTGTTGGGCCACAGTATTCCTCCCTGG
GCTCGCAGCCCATCCTGTGTGCCAGCATCCCGGGCCTGGTCCCCAAGCAGCTCCGCTTCTGCAGGAAC
TACGTCGAGATCATGCCCAGCGTGGCCGAGCGCATCAAGATTGGCATCCAGGAGTGCCAGCACCAGTT
CCGCGGCCGCCGGTGGAACTGCACCACCGTCCACGACAGCCTGGCCATCTTCGGGCCCGTGCTGGACA
AAGCTACCAGGGAGTCGGCCTTTGTCCACGCCATTGCCTCAGCCGGTGTGGCCTTTGCAGTGACACGC
TCATGTGCAGAAGGCACGGCCGCCATCTGTGGCTGCAGCAGCCGCCACCAGGGCTCACCAGGCAAGGG
CTGGAAGTGGGGTGGCTGTAGCGAGGACATCGAGTTTGGTGGGATGGTGTCTCGGGAGTTCGCCGACG
CCCGGGAGAACCGGCCAGATGCCCGCTCAGCCATGAACCGCCACAACAACGAGGCTGGGCGCCAGGCC
ATCGCCAGCCACATGCACCTCAAGTGCAAGTGCCACGGGCTGTCGGGCAGCTGCGAGGTGAAGACATG
CTGGTGGTCGCAACCCGACTTCCGCGCCATCGGTGACTTCCTCAAGGACAAGTACGACAGCGCCTCGG
AGATGGTGGTGGAGAAGCACCGGGAGTCCCGCGGCTGGGTGGAGACCCTGCGGCCGCGCTACACCTAC
TTCAAGGTGCCCACGGAGCGCGACCTGGTCTACTACGAGGCCTCGCCCAACTTCTGCGAGCCCAACCC
TGAGACGGGCTCCTTCGGCACGCGCGACCGCACCTGCAACGTCAGCTCGCACGGCATCGACGGCTGCG
ACCTGCTGTGCTGCGGCCGCGGCCACAACGCGCGAGCGGAGCGGCGCCGGGAGAAGTGCCGCTGCGTG
TTCCACTGGTGCTCCTACGTCAGCTGCCAGGAGTGCACCCGCGTCTACGACGCGCACACCTGCAAGTA
GGCACCGGCCGCGCCTCCCCCTGGACGG

| | | | |
|---|---|---|---|
| NOV5aa, SNP13381649 of | SEQ ID NO: 192 | | MW at 39336.3 kD |
| CG54611-01, Protein Sequence | SNP Pos: 348 | 352 aa | SNP Change: Val to Ala |

MAPLGYFLLLCSLKQALGSYPIWWSLAVGPQYSSLGSQPILCASIPGLVPKQLRFCRNYVEIMPSVAE
GIKIGIQECQHQFRGRRWNCTTVHDSLAIFGPVLDKATRESAFVHAIASAGVAFAVTRSCAEGTAAIC
GCSSRHQGSPGKGWKWGGCSEDIEFGGMVSREFADARENRPDARSAMNRHNNEAGRQAIASHMHLKCK
CHGLSGSCEVKTCWWSQPDFRAIGDFLKDKYDSASEMVVEKHRESRGWVETLRPRYTYFKVPTERDLV
YYEASPNFCEPNPETGSFGTRDRTCNVSSHGIDGCDLLCCGRGHNARAERRREKCRCVFHWCCYVSCQ
ECTRVYDAHTCK

A ClustalW comparison of the above protein sequences yields the following sequence alignment shown in Table 5B.

TABLE 5B
Comparison of the NOV5 protein sequences.

| | |
|---|---|
| NOV5a | ---------------------------MAPLGYFLLLCSLKQALGSYPIWWSLAVGPQYS |
| NOV5b | -------------------------------------------GSSYPIWWSLAVGPQYS |
| NOV5c | ---------------------------MAPLGYFLLLCSLKQALGSYPIWWSLAVGPQYS |
| NOV5d | ---------------------------MAPLGYFLLLCSLKQALGSYPIWWSLAVGPQYS |
| NOV5e | ---------------------------------------------SYPIWWSLAVGPQYS |
| NOV5f | ---------------------------------------------SYPIWWSLAVGPQYS |
| NOV5g | ---------------------------MAPLGYFLLLCSLKQALGSYPIWWSLAVGPQYS |
| NOV5h | -----------------------TGSTMAPLGYFLLLCSLKQALGSYPIWWSLAVGPQYS |
| NOV5i | ---------------------------MAPLGYFLLLCSLKQALGSYPIWWSLAVGPQYS |
| NOV5j | ---------------------------MAPLGYFLLLCSLKQALGSYPIWWSLAVGPQYS |
| NOV5k | ---------------------------------------------SYPIWWSLAVGPQYS |
| NOV5l | ---------------------------MAPLGYFLLLCSLKQALGSYPIWWSLAVGPQYS |
| NOV5m | ---------------------------MAPLGYFLLLCSLKQALGSYPIWWSLAVGPQYS |
| NOV5n | MAPLGYFLLMAPLGYFLLMAPLGYFLLMAPLGYFLLLCSLKQALGSYPIWWSLAVCPQYS |
| NOV5o | ---------------------------MAPLGYFLLLCSLKQALGSYPIWWSLAVGPQYS |
| NOV5p | ---------------------------MAPLGYFLLLCSLKQALGSYPIWWSLAVGPQYS |
| NOV5q | MAPLGYFLLMAPLGYFLLMAPLGYFLLMAPLGYFLLLCSLKQALGSYPIWWSLAVCPQYS |
| NOV5r | ---------------------------MAPLGYFLLLCSLKQALGSYPIWWSLAVGPQYS |
| NOV5s | ---------------------------MAPLGYFLLLCSLKQALGSYPIWWSLAVGPQYS |
| NOV5a | SLGSQPILCASIPGLVPKQLRFCRNYVEIMPSVAEGIKIGIQECQHQFRGRRWNCTTVHD |
| NOV5b | SLGSQPILCASIPGLVPKQLRFCRNYVEIMPSVAEGIKIGIQECQHQFRGRRWNCTTVHD |
| NOV5c | SLGSQPILCASIPGLVPKQLRFCRNYVEIMPSVAEGIKIGIQECQHQFRGRRWNCTTVHD |
| NOV5d | SLGSQPILCASIPGLVPKQLRFCRNYVEIMPSVAEGIKIGIQECQHQFRGRRWNCTTVHD |
| NOV5e | SLGSQPILCASIPGLVPKQLRFCRNYVEIMPSVAEGIKIGIQECQHQFRGRRWNCTTVHD |
| NOV5f | SLGSQPILCASIPGLVPKQLRFCRNYVEIMPSVAEGIKIGIQECQHQFRGRRWNCTTVHD |
| NOV5g | SLGSQPILCASIPGLVPKQLRFCRNYVEIMPSVAEGIKIGIQECQHQFRGRRWNCTTVHD |
| NOV5h | SLGSQPILCASIPGLVPKQLRFCRNYVEIMPSVAEGIKIGIQECQHQFRGRRWNCTTVHD |
| NOV5i | SLGSQPILCASIPGLVPKQLRFCRNYVEIMPSVAEGIKIGIQECQHQFRGRRWNCTTVHD |
| NOV5j | SLGSQPILCASIPGLVPKQLRFCRNYVEIMPSVAEGIKIGIQECQHQFRGRRWNCTTVHD |
| NOV5k | SLGSQPILCASIPGLVPKQLRFCRNYVEIMPSVAEGIKIGIQECQHQFRGRRWNCTTVHD |
| NOV5l | SLGSQPILCASIPGLVPKQLRFCRNYVEIMPSVAEGIKIGIQECQHQFRGRRWNCTTVHD |
| NOV5m | SLGSQPILCASIPGLVPKQLRFCRNYVEIMPSVAEGIKIGIQECQHQFRGRRWNCTTVHD |
| NOV5n | SLGSQPILCASIPGLVPKQLRFCRNYVEIMPSVAEGIKIGIQECQHQFRGRRWNCTTVHD |
| NOV5o | SLGSQPILCASIPGLVPKQLRFCRNYVEIMPSVAEGIKIGIQECQHQFRGRRWNCTTVHD |
| NOV5p | SLGSQPILCASIPGLVPKQLRFCRNYVEIMPSVAEGIKIGIQECQHQFRGRRWNCTTVHD |
| NOV5q | SLGSQPILCASIPGLVPKQLRFCRNYVEIMPSVAEGIKIGIQECQHQFRGRRWNCTTVHD |
| NOV5r | SLGSQPILCASIPGLVPKQLRFCRNYVEIMPSVAEGIKIGIQECQHQFRGRRWNCTTVHD |
| NOV5s | SLGSQPILCASIPGLVPKQLRFCRNYVEIMPSVAEGIKIGIQECQHQFRGRRWNCTTVHD |
| NOV5a | SLAIFGPVLDKATRESAFVHAIASAGVAFAVTRSCAEGTAAICGCSSRHQGSPGKGWKWG |
| NOV5b | SLAIFGPVLDKATRESAFVHAIASAGVAFAVTRSCAEGTAAICGCSSRHQGSPGKGWKWG |
| NOV5c | SLAIFGPVLDKATRESAFVHAIASAGVAFAVTRSCAEGTAAICGCSSRHQGSPGKGWKWG |
| NOV5d | SLAIFGPVLDKATRESAFVHAIASAGVAFAVTRSCAEGTAAICGCSSRHQGSPGKGWKWG |
| NOV5e | SLAIFGPVLDKATRESAFVHAIASAGVAFAVTRSCAEGTAAICGCSSRHQGSPGKGWKWG |
| NOV5f | SLAIFGPVLDKATRESAFVHAIASAGVAFAVTRSCAEGTAAICGCSSRHQGSPGKGWKWG |
| NOV5g | SLAIFGPVLDKATRESAFVHAIASAGVAFAVTRSCAEGTAAICGCSSRHQGSPGKGWKWG |
| NOV5h | SLAIFGPVLDKATRESAFVHAIASAGVAFAVTRSCAEGTAAICGCSSRHQGSPGKGWKWG |
| NOV5i | SLAIFGPVLDKATRESAFVHAIASAGVAFAVTRSCAEGTAAICGCSSRHQGSPGKGWKWG |
| NOV5j | SLAIFGPVLDKATRESAFVHAIASAGVAFAVTRSCAEGTAAICGCSSRHQGSPGKGWKWG |
| NOV5k | SLAIFGPVLDKATRESAFVHAIASAGVAFAVTRSCAEGTAAICGCSSRHQGSPGKGWKWG |
| NOV5l | SLAIFGPVLDKATRESAFVHAIASAGVAFAVTRSCAEGTAAICGCSSRHQGSPGKGWKWG |
| NOV5m | SLAIFGPVLDKATRESAFVHAIASAGVAFAVTRSCAEGTAAICGCSSRHQGSPGKGWKWG |
| NOV5n | SLAIFGPVLDKATRESAFVHAIASAGVAFAVTRSCAEGTAAICGCSSRHQGSPGKGWKWG |
| NOV5o | SLAIFGPVLDKATRESAFVHAIASAGVAFAVTRSCAEGTAAICGCSSRHQGSPGKGWKWG |
| NOV5p | SLAIFGPVLDKATRESAFVHAIASAGVAFAVTRSCAEGTAAICGCSSRHQGSPGKGWKWG |
| NOV5q | SLAIFGPVLDKATRESAFVHAIASAGVAFAVTRSCAEGTAAICGCSSRHQGSPGKGWKWG |
| NOV5r | SLAIFGPVLDKATRESAFVHAIASAGVAFAVTRSCAEGTAAICGCSSRHQGSPGKGWKWG |
| NOV5s | SLAIFGPVLDKATRESAFVHAIASAGVAFAVTRSCAEGTAAICGCSSRHQGSPGKGWKWG |
| NOV5a | GCSEDIEFGGMVSREFADARENRPDARSAMNRHNNEAGRQAIASHMHLKCKCHGLSGSCE |
| NOV5b | GCSEDIEFGGMVSREFADARENRPDARSAMNRHNNEAGRQAIASHMHLKCKCHGLSGSCE |
| NOV5c | GCSEDIEFGGMVSREFADARENRPDARSAMNRHNNEAGRQAIASHMHLKCKCHGLSGSCE |
| NOV5d | GCSEDIEFGGMVSREFADARENRPDARSAMNRHNNEAGRQAIASHMHLKCKCHGLSGSCE |
| NOV5e | GCSEDIEFGGMVSREFADARENRPDARSAMNRHNNEAGRQAIASHMHLKCKCHGLSGSCE |
| NOV5f | GCSEDIEFGGMVSREFADARENRPDARSAMNRHNNEAGRQAIASHMHLKCKCHGLSGSCE |
| NOV5g | GCSEDIEFGGMVSREFADARENRPDARSAMNRHNNEAGRQAIASHMHLKCKCHGLSGSCE |
| NOV5h | GCSEDIEFGGMVSREFADARENRPDARSAMNRHNNEAGRQAIASHMHLKCKCHGLSGSCE |
| NOV5i | GCSEDIEFGGMVSREFADARENRPDARSAMNRHNNEAGRQAIASHMHLKCKCHGLSGSCE |
| NOV5j | GCSEDIEFGGMVSREFADARENRPDARSAMNRHNNEAGRQAIASHMHLKCKCHGLSGSCE |
| NOV5k | GCSEDIEFGGMVSREFADARENRPDARSAMNRHNNEAGRQAIASHMHLKCKCHGLSGSCE |
| NOV5l | GCSEDIEFGGMVSREFADARENRPDARSAMNRHNNEAGRQAIASHMHLKCKCHGLSGSCE |
| NOV5m | GCSEDIEFGGMVSREFADARENRPDARSAMNRHNNEAGRQ-------------------- |
| NOV5n | GCSEDIEFGGMVSREFADARENRPDARSAMNRHNNEAGRQAIASHMHLKCKCHGLSGSCE |
| NOV5o | GCSEDIEFGGMVSREFADARENRPDARSAMNRHNNEAGRQAIASHMHLKCKCHGLSGSCE |
| NOV5p | GCSEDIEFGGMVSREFADARENRPDARSAMNRHNNEAGRQAIASHMHLKCKCHGLSGSCE |
| NOV5q | GCSEDIEFGGMVSREFADARENRPDARSAMNRHNNEAGRQAIASHMHLKCKCHGLSGSCE |
| NOV5r | GCSEDIEFGGMVSREFADARENRPDARSAMNRHNNEAGRQAIASHMHLKCKCHGLSGSCE |
| NOV5s | GCSEDIEFGGMVSREFADARENRPDARSAMNRHNNEAGRQAIASHMHLKCKCHGLSGSCE |
| NOV5a | VKTCWWSQPDFRAIGDFLKDKYDSASEMVVEKHRESRGWVETLRPRYTYFKVPTERDLVY |
| NOV5b | VKTCWWSQPDFRAIGDFLKDKYDSASEMVVEKHRESRGWVETLRPRYTYFKVPTERDLVY |
| NOV5c | VKTCWWSQPDFRAIGDFLKDKYDSASEMVVEKHRESRGWVETLRPRYTYFKVPTERDLVY |
| NOV5d | VKTCWWSQPDFRAIGDFLKDKYDSASEMVVEKHRESRGWVETLRPRYTYFKVPTERDLVY |
| NOV5e | VKTCWWSQPDFRAIGDFLKDKYDSASEMVVEKHRESRGWVETLRPRYTYFKVPTERDLVY |
| NOV5f | VKTCWWSQPDFRAIGDFLKDKYDSASEMVVEKHRESRGWVETLRPRYTYFKVPTERDLVY |
| NOV5g | VKTCWWSQPDFRAIGDFLKDKYDSASEMVVEKHRESRGWVETLRPRYTYFKVPTERDLVY |
| NOV5h | VKTCWWSQPDFRAIGDFLKDKYDSASEMVVEKHRESRGWVETLRPRYTYFKVPTERDLVY |
| NOV5i | VKTCWWSQPDFRAIGDFLKDKYDSASEMVVEKHRESRGWVETLRPRYTYFKVPTERDLVY |
| NOV5j | VKTCWWSQPDFRAIGDFLKDKYDSASEMVVEKHRESRGWVETLRPRYTYFKVPTERDLVY |
| NOV5k | VKTCWWSQPDFRAIGDFLKDKYDSASEMVVEKHRESRGWVETLRPRYTYFKVPTERDLVY |
| NOV5l | VKTCWWSQPDFRAIGDFLKDKYDSASEMVVEKHRESRGWVETLRPRYTYFKVPTERDLVY |
| NOV5m | -------------------DKYDSASEMVVEKHRESRGWVETLRPRYTYFKVPTERDLVY |
| NOV5n | VKTCWWSQPDFRAIGDFLKDKYDSASEMVVEKHRESRGWVETLRPRYTYFKVPTERDLVY |
| NOV5o | VKTCWWSQPDFRAIGDFLKDKYDSASEMVVEKHRESRGWVETLRPRYTYFKVPTERDLVY |
| NOV5p | VKTCWWSQPDFRAIGDFLKDKYDSASEMVVEKHRESRGWVETLRPRYTYFKVPTERDLVY |
| NOV5q | VKTCWWSQPDFRAIGDFLKDKYDSASEMVVEKHRESRGWVETLRPRYTYFKVPTERDLVY |
| NOV5r | VKTCWWSQPDFRAIGDFLKDKYDSASEMVVEKHRESRGWVETLRPRYTYFKVPTERDLVY |
| NOV5s | VKTCWWSQPDFRAIGDFLKDKYDSASEMVVEKHRESRGWVETLRPRYTYFKVPTERDLVY |
| NOV5a | YEASPNFCEPNPETGSFGTRDRTCNVSSHGIDGCDLLCCGRGHNARAERRREKCRCVFHW |
| NOV5b | YEASPNFCEPNPETGSFGTRDRTCNVSSHGIDGCDLLCCGRGHNARAERRREKCRCVFHW |
| NOV5c | YEASPNFCEPNPETGSFGTRDRTCNVSSHGIDGCDLLCCGRGHNARAERRREKCRCVFHW |
| NOV5d | YEASPNFCEPNPETGSFGTRDRTCNVSSHGIDGCDLLCCGRGHNARAERRREKCRCVFHW |
| NOV5e | YEASPNFCEPNPETGSFGTRDRTCNVSSHGIDGCDLLCCGRGHNARAERRREKCRCVFHW |
| NOV5f | YEASPNFCEPNPETGSFG------------------------------------------ |
| NOV5g | YEASPNFCEPNPETGSFGTRDRTCNVSSHGIDGCDLLCCGRGHNARAERRREKCRCVFHW |
| NOV5h | YEASPNFCEPNPETGSFGTRDRTCNVSSHGIDGCDLLCCGRGHNARAERRREKCRCVFHW |
| NOV5i | YEASPNFCEPNPETGSFGTRDRTCNVSSHGIDGCDLLCCGRGHNARAERRREKCRCVFHW |
| NOV5j | YEASPNFCEPNPETGSFGTRDRTCNVSSHGIDGCDLLCCGRGHNARAERRREKCRCVFHW |
| NOV5k | YEASPNFCEPNPETGSFGTRDRTCNVSSHGIDGCDLLCCGRGHNARAERRREKCRCVFHW |
| NOV5l | YEASPNFCEPNPETGSFGTRDRTCNVSSHGIDGCDLLCCGRGHNARAERRREKCRCVFHW |
| NOV5m | YEASPNFCEPNPETGSFGTRDRTCNVSSHGIDGCDLLCCGRGHNARAERRREKCRCVFHW |
| NOV5n | YEASPNFCEPNPETGSFGTRDRTCNVSSHGIDGCDLLCCGRGHNARAERRREKCRCVFHW |
| NOV5o | YEASPNFCEPNPETGSFGTRDRTCNVSSHGIDGCDLLCCGRGHNARAERRREKCRCVFHW |
| NOV5p | YEASPNFCEPNPETGSFGTRDRTCNVSSHGIDGCDLLCCGRGHNARAERRREKCRCVFHW |
| NOV5q | YEASPNFCEPNPETGSFGTRDRTCNVSSHGIDGCDLLCCGRGHNARAERRREKCRCVFHW |
| NOV5r | YEASPNFCEPNPETGSFGTRDRTCNVSSHGIDGCDLLCCGRGHNARAERRREKCRCVFHW |
| NOV5s | YEASPNFCEPNPETGSFGTRDRTCNVSSHGIDGCDLLCCGRGHNARAERRREKCRCVFHW |
| NOV5a | CCYVSCQECTRVYDVHTCK------ |
| NOV5b | CCYVSCQECTRVYDVHTCKLE---- |
| NOV5c | CCYVSCQECTRVYDVHTCK------ |
| NOV5d | CCYVSCQECTRVYDVHTCK------ |
| NOV5e | CCYVSCQECTRVYDVHTCK------ |
| NOV5f | ---------TRVYDVHTCK------ |
| NOV5g | CCYVSCQECTRVYDVHTCK------ |
| NOV5h | CCYVSCQECTRVYDVHTCKHHHHHH |
| NOV5i | CCYVSCQECTRVYDVHTCK------ |
| NOV5j | CCYVSCQECTRVYDVHTCK------ |
| NOV5k | CCYVSCQECTRVYDVHTCK------ |
| NOV5l | CCYVSCQECTRVYDVHTCK------ |
| NOV5m | CCYVSCQECTRVYDVHTCK------ |
| NOV5n | CCYVSCQECTRVYDVHTCK------ |
| NOV5o | CCYVSCQECTRVYDVHTCK------ |
| NOV5p | CCYVSCQECTRVYDVHTCK------ |
| NOV5q | CCYVSCQECTRVYDVHTCK------ |
| NOV5r | CCYVSCQECTRVYDVHTCK------ |
| NOV5s | CCYVSCQECTRVYDVHTCK------ |
| NOV5a | (SEQ ID NO: 140) |
| NOV5b | (SEQ ID NO: 142) |
| NOV5c | (SEQ ID NO: 144) |
| NOV5d | (SEQ ID NO: 146) |
| NOV5e | (SEQ ID NO: 148) |
| NOV5f | (SEQ ID NO: 150) |
| NOV5g | (SEQ ID NO: 152) |
| NOV5h | (SEQ ID NO: 154) |
| NOV5i | (SEQ ID NO: 156) |
| NOV5j | (SEQ ID NO: 158) |
| NOV5k | (SEQ ID NO: 160) |
| NOV5l | (SEQ ID NO: 162) |
| NOV5m | (SEQ ID NO: 164) |
| NOV5n | (SEQ ID NO: 166) |
| NOV5o | (SEQ ID NO: 168) |
| NOV5p | (SEQ ID NO: 170) |
| NOV5q | (SEQ ID NO: 172) |
| NOV5r | (SEQ ID NO: 174) |
| NOV5s | (SEQ ID NO: 176) |

Further analysis of the NOV5a protein yielded the following properties shown in Table 5C.

TABLE 5C
Protein Sequence Properties NOVSa

| SignalP analysis: | Cleavage site between residues 19 and 20 | | | |
|---|---|---|---|---|
| PSORT II | PSG: a new signal peptide prediction method | | | |
| analysis: | N-region: length 0; pos.chg 0; neg.chg 0 | | | |
| | H-region: length 13; peak value 9.00 | | | |
| | PSG score: 4.60 | | | |
| | GvH: von Heijne's method for signal seq. recognition | | | |
| | GvH score (threshold: −2.1): 0.73 | | | |
| | possible cleavage site: between 18 and 19 | | | |
| | >>> Seems to have a cleavable signal peptide (1 to 18) | | | |
| | ALOM: Klein et al's method for TM region allocation | | | |
| | Init position for calculation: 19 | | | |
| | Tentative number of TMS(s) for the threshold 0.5: 1 | | | |
| | Number of TMS(s) for threshold 0.5: 0 | | | |
| | PERIPHERAL Likelihood = 4.61 (at 33) | | | |
| | ALOM score: −0.37 (number of TMSs: 0) | | | |
| | MTOP: Prediction of membrane topology (Hartmann et al.) | | | |
| | Center position for calculation: 9 | | | |
| | Charge difference: 0.0 C(1.0)-N(1.0) | | | |
| | N >= C: N-terminal side will be inside | | | |
| | MITDISC: discrimination of mitochondrial targeting seq | | | |
| | R content: | 2 | Hyd Moment(75): | 1.56 |
| | Hyd Moment(95): | 3.50 | G content: | 5 |
| | D/E content: | 1 | S/T content: | 7 |

Score: −4.38
Gavel: prediction of cleavage sites for mitochondrial preseq
R-2 motif at 67 CRN|YV
NUCDISC: discrimination of nuclear localization signals
pat4: none
pat7: none
bipartite: none
content of basic residues: 12.5%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
33.3%: extracellular, including cell wall
33.3%: mitochondrial
11.1%: Golgi
11.1%: vacuolar
11.1%: endoplasmic reticulum
>> prediction for CG54611-06 is exc (k = 9)

A search of the NOV5a protein against the Geneseq database, a proprietary database that contains sequences published in patents and patent publication, yielded several homologous proteins shown in Table 5D.

TABLE 5D
Geneseq Results for NOV5a

| | | | Identities/ | |
|---|---|---|---|---|
| | | | Similarities for | |
| Geneseq | Protein/Organism/Length | NOV5a Residues/ | the Matched | Expect |
| Identifier | [Patent #, Date] | Match Residues | Region | Value |
| ABG60222 | Human Wnt-like protein NOV1b - | 1 . . . 352 | 352/352 (100%) | 0.0 |
| | Homo sapiens, 352 aa. | 1 . . . 352 | 352/352 (100%) | |
| | [WO200224733-A2, 28 MAR. | | | |
| | 2002] | | | |
| ABG60221 | Human Wnt-like protein NOV1a - | 1 . . . 352 | 352/352 (100%) | 0.0 |
| | Homo sapiens, 352 aa. | 1 . . . 352 | 352/352 (100%) | |
| | [WO200224733-A2, 28 MAR. | | | |
| | 2002] | | | |
| AAU96847 | Human NOV1b protein variant - | 1 . . . 352 | 350/352 (99%) | 0.0 |
| | Homo sapiens, 352 aa. | 1 . . . 352 | 350/352 (99%) | |
| | [WO200224733-A2, 28 MAR. | | | |
| | 2002] | | | |
| AAU96846 | Human Wnt-like protein NOV1a | 1 . . . 352 | 348/352 (98%) | 0.0 |
| | variant - Homo sapiens, 352 aa. | 1 . . . 352 | 348/352 (98%) | |
| | [WO200224733-A2, 28 MAR. | | | |
| | 2002] | | | |
| AAY57596 | Murine Wnt-3a protein - Mus sp, | 1 . . . 352 | 338/352 (96%) | 0.0 |
| | 352 aa. [WO9957248-A1, 11 | 1 . . . 352 | 344/352 (97%) | |
| | NOV. 1999] | | | |

In a BLAST search of public sequence databases, the NOV5a protein was found to have homology to the proteins shown in the BLASTP data in Table 6E.

TABLE 5E
Public BLASTP Results for NOV5a

| | | | Identities/ | |
|---|---|---|---|---|
| Protein | | | Similarities for | |
| Accession | | NOV5a Residues/ | the Matched | Expect |
| Number | Protein/Organism/Length | Match Residues | Portion | Value |
| P56704 | Wnt-3a protein precursor - Homo | 1 . . . 352 | 352/352 (100%) | 0.0 |
| | sapiens (Human), 352 aa. | 1 . . . 352 | 352/352 (100%) | |
| P27467 | Wnt-3a protein precursor - Mus | 1 . . . 352 | 338/352 (96%) | 0.0 |
| | musculus (Mouse), 352 aa. | 1 . . . 352 | 344/352 (97%) | |
| P31285 | Wnt-3a protein precursor (XWnt- | 1 . . . 352 | 296/352 (84%) | 0.0 |
| | 3a) - Xenopus laevis (African | 1 . . . 352 | 321/352 (91%) | |
| | clawed frog), 352 aa. | | | |
| P56703 | Wnt-3 proto-oncogene protein | 4 . . . 352 | 297/350 (84%) | 0.0 |
| | precursor - Homo sapiens | 6 . . . 355 | 319/350 (90%) | |
| | (Human), 355 aa. | | | |
| P17553 | Wnt-3 proto-oncogene protein | 4 . . . 352 | 298/350 (85%) | 0.0 |
| | precursor - Mus musculus | 6 . . . 355 | 318/350 (90%) | |
| | (Mouse), 355 aa. | | | |

PFam analysis predicts that the NOV5a protein contains the domains shown in the Table 5F.

TABLE 5F
Domain Analysis of NOV5a

| | | Identities/ | |
|---|---|---|---|
| | | Similarities | |
| | NOV5a Match | for the | Expect |
| Pfam Domain | Region | Matched Region | Value |
| wnt | 41 . . . 352 | 184/352 (52%) | 2e−220 |
| | | 293/352 (83%) | |

### Example 6

The NOV6 clone was analyzed, and the nucleotide and encoded polypeptide sequences are shown in Table 6A.

TABLE 6A

NOV6 Sequence Analysis

| | | |
|---|---|---|
| NOV6a, CG92035-02 | SEQ ID NO: 193 | 795 bp |
| DNA Sequence | ORF Start: at 28 | ORF Stop: TGA at 784 |

GGTGTGGTGATGCTGCTGGTGCTGGTGGTGCTCATCCCCGTGCTGGTGAGCTCGGGCGGCCCGGAAGG
CCACTATGAGATGCTGGGCACCTGCCGCATGGTGTGCGACCCCTACCCCGCGCGGGGCCCCGGCGCCG
GCGCGCGGACCGACGGCGGCGACGCCCTGAGCGAGCAGAGCGGCGCGCCCCCGCCTTCCACGCTGGTG
CAGGGCCCCCAGGGGAAGCCGGGCCGCACCGGCAAGCCCGGCCCTCCGGGGCCTCCCGGGGACCCAGG
TCCTCCCGGCCCTGTGGGGCCGCCGGGGGAGAAGGGTGAGCCAGGCAAGCCCGGCCCTCCGGGGCTGC
CGGGCGCGGGGCGCAGCGGCGCCATCAGCACTGCCACCTACACCACGGTGCCGCGCGTGGCCTTCTAC
GCCGGCCTCAAGAACCCCCACGAGGGTTACGAGGTACTCAAGTTTGACGACGTGGTCACCAACCTAGG
CAACAACTACGACGCGGCCAGCGGCAAGTTAACGTGCAACATTCCCGGCACCTACTTTTTCACCTACC
ATGTCCTCATGCGCGGCGGCGACGGCACCAGTATGTGGGCAGACCTCTGCAAGAATGGCCAGGTGCGG
GCCAGTGCTATTCCCCAGGACGCGGACCAGAACTACGACTACGCCAGCAACAGCGTGATCCTGCACCT
GGACGCCGGCGACGAGGTCTTCATCAAGCTGGATGGAGGCAAAGCACACGGCGGCAACAGCAACAAAT
ACAGCACCTTCTCTGGCTTCATCATCTACTCCGACTGAGCTCCCCAC

| | | | |
|---|---|---|---|
| NOV6a, CG92035-02 | | | |
| Protein Sequence | SEQ ID NO: 194 | 252 aa | MW at 25749.4kD |

VLIPVLVSSGGPEGHYEMLGTCRMVCDPYPARGPGAGARTDGGDALSEQSGAPPPSTLVQGPQGKPGR
TGKPGPPGPPGDPGPPGPVGPPGEKGEPGKPGPPGLPGAGGSGAISTATYTTVPRVAFYAGLKNPHEG
YEVLKFDDVVTNLGNNYDAASGKLTCNIPGTYFFTYHVLMRGGDGTSMWADLCKNGQVRASAIAQDAD
QNYDYASNSVILHLDAGDEVFIKLDGGKAHGGNSNKYSTFSGFIIYSD

| | | |
|---|---|---|
| NOV6b, 214458541 | SEQ ID NO: 195 | 387 bp |
| DNA Sequence | ORF Start: at 1 | ORF Stop: end of sequence |

GGATCCGCCTTCTACGCCGGCCTCAAGAACCCCCACGAGGGTTACGAGGTACTCAAGTTTGACGACGT
GGTCACCAACCTAGGTAACAACTACGACGCGGCCAGCGGCGAGTTTACGTGCAACATTCCCGGCACCT
ACTTTTTCACCTACCATCTCCTCATGCGCGGCGGCGACGGCACCAGTATGTGGGCAGACCTCTGCAAG
AATGGCCAGGTGCGGGCCAGTGCTATTGCCCAGGACGCGGACCAGAACTACGACTACGCCAGCAGCAG
CGTGATCCTGCACCTGGACGCCGGTGACGAGGTCTTCATCAAGCTGGATGGAGGCAAAGCACACGGCG
GCAACAGCAACAAATACAGCACGTTCTCTGGCTTCATCATCCTCGAG

| | | | |
|---|---|---|---|
| NOV6b, 214458541 | | | |
| Protein Sequence | SEQ ID NO: 196 | 129 aa | MW at 13937.2kD |

GSAFYAGLKNPHEGYEVLKFDDVVTNLGNNYDAASGEFTCNIPGTYFFTYHVLMRGGDGTSMWADLCK
NGQVRASAIAQDADQNYDYASSSVILHLDAGDEVFIKLDGGKAHGGNSNKYSTFSGFIILE

| | | |
|---|---|---|
| NOV6c, 214458545 | SEQ ID NO: 197 | 1387 bp |
| DNA Sequence | ORF Start: at 1 | ORF Stop: end of sequence |

GGATCCGCCTTCTACGCCGGCCTCAAGAACCCCCACGAGGGTTACGAGGTACTCAAGTTTGACGACGT
GGTCACCAACCTAGGCAACAACTACGACGCGGCCAGCGGCAAGTTTACGTGCAACATTCCCGGCACCT
ACTTTTTCACCTACCATGTCCTCATGCGCGGCGGCGACGGCACCAGTATGTGGGCAGACCTCTGCAAG
AATGGCCAGGTGCGGGCCAGTGCTATTGCCCAGGACGCGGACCAGAACTACGACTACGCCAGCAACAG
CGTGATCATGCACCTGGACGCCGGCGACGAGGTCTTCATCAAGCTGGATGGAGGCAAAGCACACGGCG
GCAACAGCAACAAATACACCACGTTCTCTGGCTTCATCATCCTCGAG

| | | | |
|---|---|---|---|
| NOV6c, 214458545 | | | |
| Protein Sequence | SEQ ID NO: 198 | 129 aa | MW at 13981.3kD |

GSAFYAGLKNPHEGYEVLKFDDVVTNLGNNYDAASGKFTCNIPGTYFFTYHVLMRGGDGTSMWADLCK
NGQVRASAIAQDADQNYDYASNSVIMHLDAGDEVFIKLDGGKAHGGNSNKYSTFSGFIILE

| | | |
|---|---|---|
| NOV6d, 214458564 | SEQ ID NO: 199 | 387 bp |
| DNA Sequence | ORF Start: at 1 | ORF Stop: end of sequence |

GCATCCGCCTTCTACGCCGGCCTCAACAACCCCCACCAGGGTTCCCAGGTACTCAAGTTTGACGACGT
GGTCACCAACCTAGGCAACAACTACGACGCGGCCAGCGGCAACTTTACGTGCAACATTCCCGGCACCT
ACTTTTTCACCTACCATGTCCTCATGCGCGGCGGCGACGGCACCAGTATGTGGGCAGACCTCTGCAAG
AATGGCCAGGTGCGGGCCAGTGCTATTGCCCAGCACGCGGACCAGAACTACGACTACGCCAGCAACAG
CGTGATCCTGCACCTCGACGCCGGCGACGAGGTCTTCATCAAGCTGGATGGAGGCAAAGCACACGCCG
GCAACAGCAACAAATACAGCACGTTCTCTGGCTTCATCATCCTCGAG

| | | | |
|---|---|---|---|
| NOV6d, 214458564 | | | |
| Protein Sequence | SEQ ID NO: 200 | 129 aa | MW at 13887.2kD |

GSAFYAGLKNPHEGSEVLKFDDVVTNLGNNYDAASGKFTCNIPGTYFFTYHVLMRGGDGTSMWADLCK
NGQVRASAIAQDADQNYDYASNSVILHLDAGDEVFIKLDGGKAHGGNSNKYSTFSGFIILE

| | | |
|---|---|---|
| NOV6e, CG92035-01 | SEQ ID NO: 201 | 813 bp |
| DNA Sequence | ORF Start: ATG at 28 | ORF Stop: TGA at 802 |

GCGGCGGCGGCCGCCGCGGGTGTGGTGATGCTGCTGGTGCTGGTGGTGCTCATCCCCGTGCTGGTGAG
CTCGGGCGGCCCGGAAGGCCACTATGAGATGCTGGGCACCTGCCGCATGGTGTGCGACCCCTACCCCG
CGCGGGGCCCCGCCGCCGGCGCGCGGACCGACGGCGGCGACGCCCTGAGCGAGCAGAGCGGCGCGCCC
CCGCCTTCCACGCTGGTGCAGGGCCCCCAGGGGAAGCCGGGCCGCACCGCCAAGCCCGGCCCTCCGGG
GCCTCCCGGGGACCCAGGTCCTCCCGGCCCTGTGGGGCCGCCGGGGGAGAAGGGTGAGCCAGGCAAGC
CGGGCCCTCCGGGGCTGCCGGGCGCGGGGGGCAGCGGCGCCATCAGCACTGCCACCTACACCACGGTG
CCGCGCGTGGCCTTCTACGCCGGCCTCAAGAACCCCCACGAGGGTTACGAGGTACTCAAGTTTGACGA
CGTGGTCACCACCTAGGCAACAACTACGACGCGGCCAGCGGCAAGTTAACGTGCAACATTCCCGGCA
CCTACTTTTTCACCTACCATGTCCTCATGCGCGGCGGCGACGGCACCAGTATGTGGGCAGACCTCTGC
AAGAATGGCCAGGTGCGGGCCAGTGCTATTGCCCAGGACGCGGACCAGAACTACGACTACGCCAGCAA
CAGCGTGATCCTGCACCTCGACGCCGGCGACGAGGTCTTCATCAAGCTGGATGGAGGCAAAGCACACG
GCGGCAACAGCAACAAATACACCACGTTCTCTGGCTTCATCATCTACTCCGACTGAGCTCCCCAC

| | | | |
|---|---|---|---|
| NOV6e, CG92035-01 | | | |
| Protein Sequence | SEQ ID NO: 202 | 258 aa | MW at 26418.3kD |

MLLVLVVLIPVLVSSGGPEGHYEMLGTCRMVCDPYPARCPGAGARTDGGDALSEQSGAPPPSTLVQGP
QGKPGRTGKPGPPGPPGDPGPPGPVGPPGEKCEPGKPGPPGLPGAGGSCAISTATYTTVPRVAFYAGL
KNPHEGYEVLKFDDVVTNLGNNYDAASGKLTCNIPGTYFFTYHVLMRGGDGTSMWADLCKNGQVPASA
IAQDADQNYDYASNSVILHLDAGDEVFIKLDGGKAHGGNSNKYSTFSGFIIYSD

| | | |
|---|---|---|
| NOV6f, CG92035-03 | SEQ ID NO: 203 | 387 bp |
| DNA Sequence | ORF Start: at 7 | ORF Stop: at 382 |

GGATCCGCCTTCTACGCCGGCCTCAAGAACCCCCACGAGGGTTACGAGGTACTCAACTTTGACGACGT
GGTCACCAACCTAGGCAACAACTACGACGCCGCCAGCGGCAAGTTTACGTGCAACATTCCCGGCACCT
ACTTTTTCACCTACGATGTCCTCATGCGCGGCGGCGACGGCACCAGTATGTGGGCAGACCTCTGCAAG
AATGGCCAGGTGCGGGCCAGTGCTATTGCCCAGGACGCGGACCAGAACTACGACTACGCCAGCAACAG
CGTGATCCTGCACCTGGACGCCGGTGACGAGGTCTTCATCAAGCTGGATGGAGGCAAAGCACACGGCG
GCAACAGCAACAAATACAGCACGTTCTCTGGCTTCATCATCCTCGAG

| | | | |
|---|---|---|---|
| NOV6f, CG92035-03 | | | |
| Protein Sequence | SEQ ID NO: 204 | 125 aa | MW at 13576.9kD |

AFYAGLKNPHEGYEVLKFDDVVTNLGNNYDAASGKFTCNIPGTYFFTYHVLMRGGDGTSMWADLCKNG
QVRASAIAQDADQNYDYASNSVILHLDAGDEVFIKLDGGKAHGGNSNKYSTFSGFII

A ClustalW comparison of the above protein sequences yields the following sequence alignment shown in Table 6B.

TABLE 7B
Comparison of the NOV6 protein sequences.

| | |
|---|---|
| NOV6a | ------VLIPVLVSSGGPEGHYEMLGTCRMVCDPYPARGPGAGARTDGGDALSEQSGAPP |
| NOV6b | ------------------------------------------------------------ |
| NOV6c | ------------------------------------------------------------ |
| NOV6d | ------------------------------------------------------------ |
| NOV6e | MLLVLVVLIPVLVSSGGPEGHYEMLGTCRMVCDPYPARGPGAGARTDGGDALSEQSGAPP |
| NOV6f | ------------------------------------------------------------ |
| NOV6a | PSTLVQGPQGKPGRTGKPGPPGPPGDPGPPGPVGPPGEKGEPGKPGPPGLPGAGGSGAIS |
| NOV6b | ------------------------------------------------------------ |
| NOV6c | ------------------------------------------------------------ |
| NOV6d | ------------------------------------------------------------ |
| NOV6e | PSTLVQGPQGKPGRTGKPGPPGPPGDPGPPGPVGPPGEKGEPGKPGPPGLPGAGGSGAIS |
| NOV6f | ------------------------------------------------------------ |
| NOV6a | TATYTTVPRVAFYAGLKNPHEGYEVLKFDDVVTNLGNNYDAASGKLTCNIPGTYFFTYHV |
| NOV6b | --------GSAFYAGLKNPHEGYEVLKFDDVVTNLGNNYDAASGKLTCNIPGTYFFTYHV |
| N0V6c | --------GSAFYAGLKNPHEGYEVLKFDDVVTNLGNNYDAASGKLTCNIPGTYFFTYHV |
| NOV6d | --------GSAFYAGLKNPHEGYEVLKFDDVVTNLGNNYDAASGKLTCNIPGTYFFTYHV |
| NOV6e | TATYTTVPRVAFYAGLKNPHEGYEVLKFDDVVTNLGNNYDAASGKLTCNIPGTYFFTYHV |
| NOV6f | ----------AFYAGLKNPHEGYEVLKFDDVVTNLGNNYDAASGKLTCNIPGTYFFTYHV |
| NOV6a | LMRGGDGTSMWADLCKNGQVRASAIAQDADQNYDYASNSVILHLDAGDEVFIKLDGGKAH |
| NOV6b | LMRGGDGTSMWADLCKNGQVRASAIAQDADQNYDYASNSVILHLDAGDEVFIKLDGGKAH |
| NOV6c | LMRGGDGTSMWADLCKNGQVRASAIAQDADQNYDYASNSVILHLDAGDEVFIKLDGGKAH |
| NOV6d | LMRGGDGTSMWADLCKNGQVRASAIAQDADQNYDYASNSVILHLDAGDEVFIKLDGGKAH |
| NOV6e | LMRGGDGTSMWADLCKNGQVRASAIAQDADQNYDYASNSVILHLDAGDEVFIKLDGGKAH |
| NOV6f | LMRGGDGTSMWADLCKNGQVRASAIAQDADQNYDYASNSVILHLDAGDEVFIKLDGGKAH |
| NOV6a | GGNSNKYSTFSGFIIYSD |
| NOV6b | GGNSNKYSTFSGFIILE- |
| NOV6c | GGNSNKYSTFSGFIILE- |
| NOV6d | GGNSNKYSTFSGFIILE- |
| NOV6e | GGNSNKYSTFSGFIIYSD |
| NOV6f | GGNSNKYSTFSGFII--- |
| NOV6a | (SEQ ID NO: 194) |
| NOV6b | (SEQ ID NO: 196) |
| NOV6c | (SEQ ID NO: 198) |
| NOV6d | (SEQ ID NO: 200) |
| NOV6e | (SEQ ID NO: 202) |
| NOV6f | (SEQ ID NO: 204) |

Further analysis of the NOV6a protein yielded the following properties shown in Table 6C.

TABLE 6C
Protein Sequence Properties NOV6a

| SignalP analysis: | No Known Signal Sequence Predicted | | | |
|---|---|---|---|---|
| PSORT II | PSG: a new signal peptide prediction method | | | |
| analysis: | N-region: length 0; pos.chg 0; neg.chg 0 | | | |
| | H-region: length 12; peak value 8.85 | | | |
| | PSG score: 4.45 | | | |
| | GvH: von Heijne's method for signal seq. recognition | | | |
| | GvH score (threshold: −2.1): −5.49 | | | |
| | possible cleavage site: between 14 and 15 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 7.43 (at 160) | | | |
| | ALOM score: 7.43 (number of TMSs: 0) | | | |
| | MTOP: Prediction of membrane topology (Hartmann et al.) | | | |
| | Center position for calculation: 6 | | | |
| | Charge difference: −2.5 C(−1.5)-N(1.0) | | | |
| | N >= C: N-terminal side will be inside | | | |
| | MITDISC: discrimination of mitochondrial targeting seq | | | |
| | R content: | 0 | Hyd Moment (75): | 3.67 |
| | Hyd Moment (95): | 2.25 | G content: | 3 |
| | D/E content: | 2 | S/T content: | 2 |

```
Score: −8.66
Gavel: prediction of cleavage sites for mitochondrial preseq
cleavage site motif not found
NUCDISC: discrimination of nuclear localization signals
pat4: none
pat7: none
bipartite: none
content of basic residues: 7.1%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 70.6
COIL: Lupas's algorithm to detect coiled-coil regions
total: 0 residues
Final Results (k = 9/23):
43.5%: cytoplasmic
34.8%: nuclear
13.0%: mitochondrial
  4.3%: extracellular, including cell wall
  4.3%: vacuolar
>> prediction for CG92035-02 is cyt (k = 23)
```

A search of the NOV6a protein against the Geneseq database, a proprietary database that contains sequences published in patents and patent publication, yielded several homologous proteins shown in Table 6D.

TABLE 6D
Geneseq Results for NOV6a

| | | | Identities/ | |
|---|---|---|---|---|
| | | | Similarities for | |
| Geneseq | Protein/Organism/Length [Patent | NOV6a Residues/ | the Matched | Expect |
| Identifier | #, Date] | Match Residues | Region | Value |
| ABG79646 | Human novel secreted protein | 1 . . . 252 | 194/256 (75%) | e−114 |
| | SECP22, Incyte ID No. | 7 . . . 255 | 213/256 (82%) | |
| | 5960119CD1 - Homo sapiens, 255 | | | |
| | aa. [WO200262841-A2, 15 AUG. | | | |
| | 2002] | | | |
| AAG64212 | Murine HSP47 interacting protein, | 1 . . . 252 | 190/256 (74%) | e−111 |
| | #2 - Mus sp, 255 aa. | 7 . . . 255 | 209/256 (81%) | |
| | [JP2001145493-A, 29 MAY 2001] | | | |
| ABB53290 | Human polypeptide #30 - Homo | 1 . . . 252 | 188/256 (73%) | e−110 |
| | sapiens, 255 aa. [WO200181363- | 7 . . . 255 | 208/256 (80%) | |
| | A1, 01 NOV. 2001] | | | |
| AAU09865 | Novel human secreted protein #6 - | 1 . . . 252 | 176/282 (62%) | e−101 |
| | Homo sapiens, 287 aa. | 7 . . . 287 | 207/282 (72%) | |
| | [WO200179454-A1, 25 OCT. 2001] | | | |
| ABG69646 | Human secreted protein SCEP-26 - | 1 . . . 252 | 176/282 (62%) | e−101 |
| | Homo sapiens, 287 aa. | 7 . . . 287 | 207/282 (72%) | |
| | [WO200248337-A2, 20 JUN. 2002] | | | |

In a BLAST search of public sequence databases, the NOV6a protein was found to have homology to the proteins shown in the BLASTP data in Table 6E.

TABLE 6E
Public BLASTP Results for NOV6a

| | | | Identities/ | |
|---|---|---|---|---|
| Protein | | | Similarities for | |
| Accession | | NOV6a Residues/ | the Matched | Expect |
| Number | Protein/Organism/Length | Match Residues | Portion | Value |
| O75973 | C1q-related factor precursor - | 1 . . . 252 | 251/252 (99%) | e−153 |
| | Homo sapiens (Human), 258 aa. | 7 . . . 258 | 251/252 (99%) | |
| O88992 | C1q-related factor precursor - Mus | 1 . . . 252 | 246/252 (97%) | e−150 |
| | musculus (Mouse), 258 aa. | 7 . . . 258 | 250/252 (98%) | |
| Q9ESN4 | Gliacolin precursor (C1q-like | 1 . . . 252 | 190/256 (74%) | e−111 |
| | protein) - Mus musculus (Mouse), | 7 . . . 255 | 209/256 (81%) | |
| | 255 aa. | | | |
| Q7ZZ82 | SI: dZ63M2.2 (Novel protein similar | 1 . . . 245 | 177/247 (71%) | e−103 |
| | to human gliacolin) - Brachydanio | 7 . . . 250 | 202/247 (81%) | |
| | rerio (Zebrafish) (Danio rerio), 250 | | | |
| | aa (fragment). | | | |
| Q8CFRO | Similar to C1q-like - Mus musculus | 1 . . . 252 | 176/282 (62%) | e−100 |
| | (Mouse), 287 aa. | 7 . . . 287 | 207/282 (72%) | |

PFam analysis predicts that the NOV6a protein contains the domains shown in the Table 6F.

TABLE 6F
Domain Analysis of NOV6a

| | | Identities/ | |
|---|---|---|---|
| | | Similarities | |
| | NOV6a Match | for the | Expect |
| Pfam Domain | Region | Matched Region | Value |
| Collagen | 59 . . . 117 | 34/60 (57%) | 0.00022 |
| | | 42/60 (70%) | |
| C1q | 125 . . . 249 | 42/140 (30%) | 4.7e−27 |
| | | 91/140 (65%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof. The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corporation proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain -whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s:18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer

Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tm must be 10° C. greater than primer Tm; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1×TaqMan® Universal Master mix (Applied Biosystems; catalog No. 4324020), following the manufacturer's instructions. PCR amplification and analysis were done as described above.

### Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or p1 effusion) and * indicates established from metastasis.

### General_screening_panel_v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

### Panels 2D, 2.2, 2.3 and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDRI), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

### HASS Panel v 1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

### ARDAIS Panel v1.0 and v1.1

The ARDAIS Panel v1.0 and v1.1 included 2 controls and 22 test samples including: human lung adenocarcinomas, lung squamous cell carcinomas, and in some cases matched adjacent normal tissues (NAT) obtained from Ardais (Lexington, Mass.). Unmatched malignant and non-malignant RNA samples from lungs with gross histopathological assessment of tumor differentiation grade and stage and clinical state of the patient were obtained from Ardais.

### ARDAIS Prostate v1.0

ARDAIS Prostate v1.0 panel included 2 controls and 68 test samples of human prostate malignancies and in some cases matched adjacent normal tissues (NAT) obtained from Ardais (Lexington, Mass.). RNA from unmatched malignant and non-malignant prostate samples with gross histopathological assessment of tumor differentiation grade and stage and clinical state of the patient were also obtained from Ardais.

### ARDAIS Kidney v1.0

ARDAIS Kidney v1.0 panel included 2 control wells and 44 test samples of human renal cell carcinoma and in some cases matched adjacent normal tissue (NAT) obtained from Ardais (Lexington, Mass.). RNA from unmatched renal cell carcinoma and normal tissue with gross histopathological assessment of tumor differentiation grade and stage and clinical state of the patient were also obtained from Ardais.

### ARDAIS Breast v1.0

ARDAIS Breast v1.0 panel included 2 control wells and 71 test samples of human breast malignancies and in some cases matched adjacent normal tissue (NAT) obtained from Ardais (Lexington, Mass.). RNA from unmatched malignant and non-malignant breast samples with gross histopathological assessment of tumor differentiation grade and stage and clinical state of the patient were also obtained from Ardais.

### Panel 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

### Panels 4D, 4R, and 4.1D

Panels 4D, 4R, and 4. 1D included 2 control wells and 94 test samples of RNA (Panel 4R) or cDNA (Panels 4D and 4. 1D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDRI, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta ˜1-5 ng/ml, TNF alpha ˜5-10 ng/ml, iFN gamma ˜20-50 ng/ml, IL-4˜5-10 ng/ml, IL-9˜5-10 ng/ml, IL-13 5-10 ng/ml or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (Hyclone, Logan, Utah), 100 mM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 mg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of ˜2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+ lymphocytes were isolated by depleting mononuclear cells of CD8+, CD56+, CD14+ and CD19+ cells using CD8, CD56, CD14 and CD19 Miltenyi beads and positive selection. CD45RO Miltenyi beads were then used to separate the CD45+RO CD4+ lymphocytes from CD45+RA CD4+ lymphocytes. CD45+RA CD4+, CD45+RO CD4 + and CD8+ lymphocytes were cultured in culture media at 10⁶ cells/ml in culture plates precoated overnight with 0.5 mg/ml anti-CD28 (Pharmingen, San Diego, Calif.) and 3 Ig/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDRI). Tonsil cells were pelleted and resupended at 10⁶ cells/ml in culture media. Cells were activated using 5 μg/ml PWM (Sigma-Aldrich Corp., St. Louis, Mo.) or ˜10 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 10⁵-10⁶cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 4-5 days, the Th1, Th2 and Tr1 lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×10⁵ cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 ml buffer (Promega, Madison, Wis.) 5 μl DTT, 7 μt RNAsin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

### AI_comprehensive panel_v1.0

Autoimmunity (AI) comprehensive panel v1.0 included two controls and 89 cDNA test samples isolated from male (M) and female (F) surgical and postmortem human tissues that were obtained from the Backus Hospital and Clinomics (Frederick, Md.). Tissue samples included : normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

### Pulmonary and General Inflammation (PGI) Panel v1.0

Pulmonary and General inflammation (PGI) panel v1.0 included two controls and 39 test samples isolated as surgical or postmortem samples. Tissue samples include: five normal lung samples obtained from Maryland Brain and Tissue Bank, University of Maryland (Baltimore, Md.), International Bioresource systems, IBS (Tuscon, Ariz.), and Asterand (Detroit, Mich.), five normal adjacent intestine tissues (NAT) from Ardais (Lexington, Mass.), ulcerative colitis samples (UC) from Ardais (Lexington, Mass.); Crohns disease colon from NDRI, National Disease Research Interchange (Philadelphia, Pa.); emphysematous tissue samples from Ardais (Lexington, Mass.) and Genomic Collaborative Inc. (Cambridge, Mass.), asthmatic tissue from Maryland Brain and Tissue Bank, University of Maryland (Baltimore, Md.) and Genomic Collaborative Inc (Cambridge, Mass.) and fibrotic tissue from Ardais (Lexinton, Mass.) and Genomic Collaborative (Cambridge, Mass.).

### Cellular OA/RA Panel

Cellular OA.RA panel includes 2 control wells and 35 test samples comprised of cDNA generated from total RNA isolated from human cell lines or primary cells representative of the human joint and its inflammatory condition. Cell types included normal human osteoblasts (Nhost) from Clonetics (Cambrex, East Rutherford, N.J.), human chondrosarcoma SW1353 cells from ATCC (Manossas, Va.)), human fibroblast-like synoviocytes from Cell Applications, Inc. (San Diego, Calif.) and MH7A cell line (a rheumatoid fibroblast-like synoviocytes transformed with SV40 T antigen) from Riken Cell bank (Tsukuba Science City, Japan). These cell types were activated by incubating with various cytokines (IL-1 beta ˜1-10 ng/ml, TNF alpha ˜5-50 ng/ml, or prostaglandin E2 for Nhost cells) for 1, 6, 18 or 24 h. All these cells were starved for at least 5 h and cultured in their corresponding basal medium with ˜0.1 to 1% FBS.

### Minitissue OA/RA Panel

The OA/RA mini panel includes two control wells and 31 test samples comprised of cDNA generated from total RNA isolated from surgical and postmortem human tissues obtained from the University of Calgary (Alberta, Canada), NDRI (Philadelphia, Pa.), and Ardais Corporation (Lexington, Mass.). Joint tissue samples include synovium, bone and cartilage from osteoarthritic and rheumatoid arthritis patients undergoing reconstructive knee surgery, as well as, normal synovium samples (RNA and tissue). Visceral normal tissues were pooled from 2-5 different adults and included adrenal gland, heart, kidney, brain, colon, lung, stomach, small intestine, skeletal muscle, and ovary.

### AI.05 Chondrosarcoma

AI.05 chondrosarcoma plates included SW1353 cells (ATCC) subjected to serum starvation and treated for 6 and 18 h with cytokines that are known to induce MMP (1, 3 and 13) synthesis (e.g. IL1beta). These treatments included: IL-1beta (10 ng/ml), IL-1beta+TNF-alpha (50 ng/ml), IL-1beta+Oncostatin (50 ng/ml) and PMA (100 ng/ml). Supernatants were collected and analyzed for MMP 1, 3 and 13 production. RNA was prepared from these samples using standard procedures.

### Panels 5D and 51

Panel 5D and 5I included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18-40 years), otherwise healthy women with and without gestational diabetes undergoing routine (elective) Caesarean section. Uterine wall smooth muscle (UT), visceral (Vis) adipose, skeletal muscle (SK), placenta (PI) greater omentum adipose (GO Adipose) and subcutaneous (SubQ) adipose samples (less than 1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr. 2 1999: 143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures.

Panel 5I also contains pancreatic islets (Diabetes Research Institute at the University of Miami School of Medicine).

### Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (PI), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non- diabetic Caucasians; Patient 22, a treated diabetic Caucasian of normal weight; Patient 23, an overweight non-diabetic Caucasian; and Patients 26 and 27, obese, treated, diabetic Caucasians.

Total RNA was isolated from metabolic tissues including: hypothalamus, liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

### CNS Panels

CNS Panels CNSD.01, CNS Neurodegeneration V1.0 and CNS Neurodegeneration V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −80° C. in liquid nitrogen vapor.

### Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

### Panel CNS Neurodegeneration V1.0

The CNS Neurodegeneration V1.0 panel included: six Alzheimer's disease (AD) brains and eight normals which included no dementia and no Alzheimer's like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value for all wells across all runs). The scaled CT value is the result of the raw CT value plus the scaling factor.

### Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

A. NOV1 (CG104903-03 and CG104903-09 and CG104903-10): HMW Kininogen with 5′utr.

Expression of genes the CG104903-03, CG104903-09 and CG104903-10 was assessed using the primer-probe sets Ag3374 and Ag4269, described in Tables AA and AB. Results of the RTQ-PCR runs are shown in Tables AC and AD.

TABLE AA

Probe Name Ag3374

| | | | Start | SEQ ID |
|---|---|---|---|---|
| Primers | Sequences | Length | Position | No |
| Forward | 5′-gattgcaacgctgaagtttatg-3′ | 22 | 1097 | 205 |
| Probe | TET-5′-ctgtcaactgtcaaccactgggaatg-3′- | 26 | 1149 | 206 |
| | TAMRA | | | |
| Reverse | 5′-gaggccttttcatcagtgagat-3′ | 22 | 1175 | 207 |

TABLE AB

Probe Name Ag4269

| | | | Start | SEQ ID |
|---|---|---|---|---|
| Primers: | Sequences | Length | Position | No |
| Forward | 5′-acagagcatttggcaagct-3′ | 19 | 1610 | 208 |
| Probe | TET-5′-cagtactacaccttctgcacagacaca-3′- | 27 | 1639 | 209 |
| | TAMRA | | | |
| Reverse | 5′-gttggcccttctgtcttctc-3′ | 20 | 1667 | 210 |

TABLE AC
Ardais Kidney 1.0

| Tissue Name | A |
|---|---|
| Kidney cancer(10A8) | 0.0 |
| Kidney NAT(10A9) | 42.9 |
| Kidney cancer(10AA) | 0.0 |
| Kidney NAT(10AB) | 27.7 |
| Kidney cancer(10AC) | 0.0 |
| Kidney NAT(10AD) | 28.1 |
| Kidney cancer(10B6) | 0.2 |
| Kidney NAT(10B7) | 20.3 |
| Kidney cancer(10B8) | 2.0 |
| Kidney NAT(10B9) | 41.5 |
| Kidney cancer(10BC) | 0.2 |
| Kidney NAT(10BD) | 31.6 |
| Kidney cancer(10BE) | 0.4 |
| Kidney NAT(10BF) | 100.0 |
| Kidney cancer(10C2) | 0.1 |
| Kidney NAT(10C3) | 22.1 |
| Kidney cancer(10C4) | 0.1 |
| Kidney NAT(10C5) | 36.1 |
| Kidney cancer(10B4) | 0.0 |
| Kidney cancer(10C8) | 0.0 |
| Kidney cancer(10D0) | 0.0 |
| Kidney cancer(10C0) | 0.0 |
| Kidney cancer(10C6) | 0.0 |
| Kidney cancer(10C9) | 0.0 |
| Kidney cancer(10D1) | 0.0 |
| Kidney cancer(10CA) | 0.0 |
| Kidney cancer(10D2) | 0.0 |
| Kidney cancer(10CB) | 0.0 |
| Kidney cancer(10D4) | 0.1 |
| Kidney cancer(10CD) | 0.0 |
| Kidney cancer(10D5) | 1.0 |
| Kidney cancer(10CE) | 0.1 |
| Kidney cancer(10D6) | 0.0 |
| Kidney cancer(10CF) | 0.1 |
| Kidney cancer(10D8) | 0.0 |
| Kidney cancer(10CC) | 0.1 |
| Kidney cancer(10D3) | 0.0 |
| Kidney NAT(10D9) | 23.2 |
| Kidney NAT(10DB) | 10.5 |
| Kidney NAT(10DC) | 55.9 |
| Kidney NAT(10DD) | 50.0 |
| Kidney NAT(10DE) | 57.0 |
| Kidney NAT(10B1) | 8.8 |
| Kidney NAT(10DA) | 23.8 |

Column A - Rel. Exp. (%) Ag3374, Run 343519951

TABLE AD
Panel 4.1D

| Tissue Name | A |
|---|---|
| Secondary Th1 act | 0.4 |
| Secondary Th2 act | 0.4 |
| Secondary Tr1 act | 0.0 |
| Secondary Th1 rest | 0.0 |
| Secondary Th2 rest | 0.1 |
| Secondary Tr1 rest | 0.2 |
| Primary Th1 act | 0.3 |
| Primary Th2 act | 0.3 |
| Primary Tr1 act | 0.1 |
| Primary Th1 rest | 0.1 |
| Primary Th2 rest | 0.1 |
| Primary Tr1 rest | 0.2 |
| CD45RA CD4 lymphocyte act | 0.0 |
| CD45RO CD4 lymphocyte act | 0.5 |
| CD8 lymphocyte act | 0.4 |
| Secondary CD8 lymphocyte rest | 0.5 |
| Secondary CD8 lymphocyte act | 1.1 |
| CD4 lymphocyte none | 0.1 |
| 2ry Th1/Th2/Tr1 anti-CD95 CH11 | 0.0 |
| LAK cells rest | 0.1 |
| LAK cells IL-2 | 0.4 |
| LAK cells IL-2 + IL-12 | 0.2 |
| LAK cells IL-2 + IFN gamma | 0.3 |
| LAK cells IL-2 + IL-18 | 0.4 |
| LAK cells PMA/ionomycin | 0.5 |
| NK Cells IL-2 rest | 0.4 |
| Two Way MLR 3 day | 0.0 |
| Two Way MLR 5 day | 0.1 |
| Two Way MLR 7 day | 0.1 |
| PBMC rest | 0.0 |
| PBMC PWM | 0.5 |
| PBMC PHA-L | 0.4 |
| Ramos (B cell) none | 0.3 |
| Ramos (B cell) ionomycin | 0.0 |
| B lymphocytes PWM | 0.5 |
| B lymphocytes CD40L and IL-4 | 0.0 |
| EOL-1 dbcAMP | 0.1 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.1 |
| HUVEC TNF alpha + IFN gamma | 0.0 |
| HUVEC TNF alpha + IL4 | 0.0 |
| HUVEC IL-11 | 0.2 |
| Lung Microvascular EC none | 0.2 |
| Lung Microvascular EC TNFalpha + IL-1beta | 0.0 |
| Microvascular Dermal EC none | 0.1 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.1 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 26.4 |
| NCI-H292 none | 0.0 |
| NCI-H292 IL-4 | 0.0 |
| NCI-H292 IL-9 | 0.1 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.1 |
| Lung fibroblast TNF alpha + IL-1 beta | 0.3 |
| Lung fibroblast IL-4 | 0.0 |
| Lung fibroblast IL-9 | 0.1 |
| Lung fibroblast IL-13 | 0.1 |
| Lung fibroblast IFN gamma | 0.1 |
| Dermal fibroblast CCD1070 rest | 0.0 |
| Dermal fibroblast CCD1070 TNF alpha | 0.3 |
| Dermal fibroblast CCD1070 IL-1 beta | 0.0 |
| Dermal fibroblast IFN gamma | 0.6 |
| Dermal fibroblast IL-4 | 0.1 |
| Dermal Fibroblasts rest | 0.9 |
| Neutrophils TNFa + LPS | 0.5 |
| Neutrophils rest | 0.3 |
| Colon | 0.8 |
| Lung | 1.8 |
| Thymus | 8.8 |
| Kidney | 100.0 |

Column A - Rel. Exp. (%) Ag4269, Run 182243380

Ardais Kidney 1.0 Summary: Ag3374 Much higher expression of this gene was detected in normal kidney tissues (CT=19.71-23.21) adjacent to a tumor, while most renal tumor tissues had low to non-detectable levels of its expression. Thus, expression of this gene can be used as a marker of normal kidney. In addition, therapeutic modulation of this gene, expressed protein and/or use of antibodies or small molecule drugs targeting the gene or gene product are useful in the treatment of renal cancer.

Panel 4.1D Summary: Ag5115/Ag4269 Two experiments with two different probe and primer sets were in good agreement with highest expression of the CG104903-06 in the kidney (CTs=27). Moderate levels of expression of this gene was also seen in thymus, lung and colon. The probe and primer sets for Ag5115 are specific to CG104903-06. In a second experiment with Ag4269 low levels of expression of this gene was also seen in selected samples, including T cells, neutrophils, and activated dermal fibroblasts, indicating that the protien encoded by this gene play an important role in T cell development. In addition, moderate expression was seen in liver cirrhosis (CT=28.73). Thus, therapeutic modulation of this gene, expressed protein and/or use of antibodies or small molecule drugs targeting the gene or gene product are useful in modulation of liver function, identification and treatment of inflammatory or autoimnmune diseases which effect the liver including liver cirrhosis and fibrosis.

B. NOV2a (CG120844-02): IL-1 Beta-like

Expression of gene CG120844-02 was assessed using the primer-probe sets Ag1141, Ag6369, Ag6601, Ag6658 and Ag6701, described in Tables BA, BB, BC, BD and BE. Results of the RTQ-PCR runs are shown in Tables BF, BG and BH.

TABLE BA

Probe Name Ag1141

| | | | Start | SEQ ID |
|---|---|---|---|---|
| Primers | Sequences | Length | Position | No |
| Forward | 5′-tctcaagcagaaaacatgcc-3′ | 20 | 572 | 211 |
| Probe | TET-5′-aggcggccaggatataactgacttca-3′- | 26 | 613 | 212 |
| | TAMRA | | | |
| Reverse | 5′-ggaagacacaaattgcatgg-3′ | 20 | 639 | 213 |

TABLE BB

Probe Name Ag6369

| | | | Start | SEQ ID |
|---|---|---|---|---|
| Primers | Sequences | Length | Position | No |
| Forward | 5′-aactgaaagctctccacctcc-3′ | 21 | 321 | 214 |
| Probe | TET-5′-tgtggccttgggcctcaagg-3′-TAMRA | 20 | 370 | 215 |
| Reverse | 5′-cgcaggacaggtacagattctt-3′ | 22 | 392 | 216 |

TABLE BC

Probe Name Ag6601

| | | | Start | SEQ ID |
|---|---|---|---|---|
| Primers | Sequences | Length | Position | No |
| Forward | 5′-tccacctccaaggagaagaa-3′ | 20 | 333 | 217 |
| Probe | TET-5′-cccaaggccacaggtattttgtcatt-3′- | 26 | 356 | 218 |
| | TAMRA | | | |
| Reverse | 5′-acacgcaggacaggtacagat-3′ | 21 | 396 | 219 |

TABLE BD

Probe Name Ag6658

| | | | Start | SEQ ID |
|---|---|---|---|---|
| Primers | Sequences | Length | Position | No |
| Forward | 5′-tccacctccaaggagaagaa-3′ | 20 | 333 | 220 |
| Probe | TET-5′-cccaaggccacaggtattttgtcatt-3′- | 26 | 356 | 221 |
| | TAMRA | | | |
| Reverse | 5′-acacgcaggacaggtacagat-3′ | 21 | 396 | 222 |

TABLE BE

Probe Name Ag6701

| | | | Start | SEQ ID |
|---|---|---|---|---|
| Primers | Sequences | Length | Position | No |
| Forward | 5′-tccacctccaaggagaagaa-3′ | 20 | 333 | 223 |
| Probe | TET-5′-cccaaggccacaggtattttgtcatt-3′- | 26 | 356 | 224 |
| | TAMRA | | | |
| Reverse | 5′-acacgcaggacaggtacagat-3′ | 21 | 396 | 225 |

TABLE BF
AI_comprehensive panel_v1.0

| Tissue Name | A | B | C |
|---|---|---|---|
| 110967 COPD-F | 0.4 | 0.3 | 0.0 |
| 110980 COPD-F | 0.5 | 0.5 | 0.0 |
| 110968 COPD-M | 0.7 | 0.5 | 0.0 |
| 110977 COPD-M | 1.0 | 1.3 | 0.0 |
| 110989 Emphysema-F | 0.8 | 0.7 | 0.0 |
| 110992 Emphysema-F | 0.9 | 0.7 | 0.0 |
| 110993 Emphysema-F | 0.5 | 0.3 | 0.0 |
| 110994 Emphysema-F | 0.0 | 0.2 | 0.0 |
| 110995 Emphysema-F | 1.7 | 2.6 | 0.0 |
| 110996 Emphysema-F | 1.4 | 1.1 | 0.0 |
| 110997 Asthma-M | 2.9 | 1.0 | 0.0 |
| 111001 Asthma-F | 1.6 | 1.1 | 0.0 |
| 111002 Asthma-F | 0.4 | 1.3 | 0.0 |
| 111003 Atopic Asthma-F | 1.2 | 1.1 | 0.0 |
| 111004 Atopic Asthma-F | 1.4 | 1.0 | 0.0 |
| 111005 Atopic Asthma-F | 0.8 | 0.6 | 0.0 |
| 111006 Atopic Asthma-F | 0.1 | 0.1 | 0.0 |
| 111417 Allergy-M | 0.8 | 0.7 | 0.0 |
| 112347 Allergy-M | 0.1 | 0.0 | 0.0 |
| 112349 Normal Lung-F | 0.0 | 0.0 | 0.0 |
| 112357 Normal Lung-F | 1.5 | 1.5 | 0.0 |
| 112354 Normal Lung-M | 1.4 | 1.0 | 0.0 |
| 112374 Crohns-F | 0.4 | 0.4 | 18.9 |
| 112389 Match Control Crohns-F | 0.4 | 0.4 | 0.0 |
| 112375 Crohns-F | 0.7 | 0.4 | 0.0 |
| 112732 Match Control Crohns-F | 3.3 | 4.0 | 11.8 |
| 112725 Crohns-M | 0.8 | 0.2 | 0.0 |
| 112387 Match Control Crohns-M | 0.9 | 0.5 | 0.0 |
| 112378 Crohns-M | 0.2 | 0.0 | 0.0 |
| 112390 Match Control Crohns-M | 0.8 | 0.8 | 0.0 |
| 112726 Crohns-M | 1.3 | 1.6 | 0.0 |
| 112731 Match Control Crohns-M | 1.8 | 1.0 | 0.0 |
| 112380 Ulcer Col-F | 0.5 | 0.5 | 0.0 |
| 112734 Match Control Ulcer Col-F | 100.0 | 100.0 | 100.0 |
| 112384 Ulcer Col-F | 4.9 | 3.5 | 0.0 |
| 112737 Match Control Ulcer Col-F | 1.4 | 1.3 | 0.0 |
| 112386 Ulcer Col-F | 0.7 | 0.3 | 0.0 |
| 112738 Match Control Ulcer Col-F | 5.7 | 7.4 | 0.0 |
| 112381 Ulcer Col-M | 1.1 | 0.0 | 0.0 |
| 112735 Match Control Ulcer Col-M | 2.0 | 0.5 | 0.0 |
| 112382 Ulcer Col-M | 1.5 | 0.9 | 0.0 |
| 112394 Match Control Ulcer Col-M | 0.4 | 0.1 | 0.0 |
| 112383 Ulcer Col-M | 7.3 | 10.0 | 18.4 |
| 112736 Match Control Ulcer Col-M | 0.4 | 0.3 | 0.0 |
| 112423 Psoriasis-F | 17.2 | 7.2 | 0.0 |
| 112427 Match Control Psoriasis-F | 1.1 | 1.1 | 0.0 |
| 112418 Psoriasis-M | 1.1 | 0.7 | 0.0 |
| 112723 Match Control Psoriasis-M | 0.0 | 0.0 | 0.0 |
| 112419 Psoriasis-M | 1.4 | 1.3 | 0.0 |
| 112424 Match Control Psoriasis-M | 0.5 | 0.5 | 0.0 |
| 112420 Psoriasis-M | 1.9 | 1.7 | 0.0 |
| 112425 Match Control Psoriasis-M | 1.3 | 0.7 | 0.0 |
| 104689 (MF) OA Bone-Backus | 3.1 | 3.6 | 11.6 |
| 104690 (MF) Adj “Normal” | 1.0 | 1.0 | 0.0 |
| Bone-Backus | | | |
| 104691 (MF) OA Synovium-Backus | 0.8 | 0.5 | 0.0 |
| 104692 (BA) OA Cartilage-Backus | 0.0 | 0.0 | 0.0 |
| 104694 (BA) OA Bone-Backus | 1.6 | 1.3 | 0.0 |
| 104695 (BA) Adj “Normal” | 1.1 | 0.8 | 0.0 |
| Bone-Backus | | | |
| 104696 (BA) OA Synovium-Backus | 1.0 | 1.0 | 0.0 |
| 104700 (SS) OA Bone-Backus | 0.8 | 2.5 | 16.2 |
| 104701 (SS) Adj “Normal” | 1.6 | 1.9 | 0.0 |
| Bone-Backus | | | |
| 104702 (SS) OA Synovium-Backus | 0.9 | 1.1 | 0.0 |
| 117093 OA Cartilage Rep7 | 3.0 | 3.4 | 12.7 |
| 112672 OA Bone5 | 1.8 | 1.1 | 0.0 |
| 112673 OA Synovium5 | 1.6 | 0.6 | 0.0 |
| 112674 OA Synovial Fluid cells5 | 0.8 | 0.8 | 0.0 |
| 117100 OA Cartilage Rep14 | 0.2 | 0.4 | 0.0 |
| 112756 OA Bone9 | 4.8 | 1.1 | 0.0 |
| 112757 OA Synovium9 | 0.1 | 0.1 | 0.0 |
| 112758 OA Synovial Fluid Cells9 | 0.5 | 0.3 | 0.0 |
| 117125 RA Cartilage Rep2 | 1.6 | 1.0 | 0.0 |
| 113492 Bone2 RA | 12.7 | 6.7 | 0.0 |
| 113493 Synovium2 RA | 6.0 | 2.5 | 0.0 |
| 113494 Syn Fluid Cells RA | 8.1 | 5.1 | 0.0 |
| 113499 Cartilage4 RA | 16.4 | 5.6 | 0.0 |
| 113500 Bone4 RA | 18.0 | 5.8 | 0.0 |
| 113501 Synovium4 RA | 10.5 | 3.7 | 0.0 |
| 113502 Syn Fluid Cells4 RA | 8.5 | 3.8 | 0.0 |
| 113495 Cartilage3 RA | 7.1 | 5.4 | 0.0 |
| 113496 Bone3 RA | 9.7 | 5.4 | 0.0 |
| 113497 Synovium3 RA | 6.0 | 3.7 | 0.0 |
| 113498 Syn Fluid Cells3 RA | 12.3 | 7.5 | 10.8 |
| 117106 Normal Cartilage Rep20 | 0.3 | 0.1 | 0.0 |
| 113663 Bone3 Normal | 0.1 | 0.0 | 0.0 |
| 113664 Synovium3 Normal | 0.0 | 0.0 | 0.0 |
| 113665 Syn Fluid Cells3 Normal | 0.0 | 0.0 | 0.0 |
| 117107 Normal Cartilage Rep22 | 0.2 | 0.1 | 0.0 |
| 113667 Bone4 Normal | 2.7 | 1.7 | 0.0 |
| 113668 Synovium4 Normal | 3.0 | 1.6 | 0.0 |
| 113669 Syn Fluid Cells4 Normal | 2.9 | 2.5 | 0.0 |

Column A - Rel. Exp. (%) Ag1141, Run 211061879
Column B - Rel. Exp. (%) Ag6369, Run 275481216
Column C - Rel. Exp. (%) Ag6658, Run 275481272

TABLE BG
Cellular OA/RA

| Tissue Name | A |
|---|---|
| 158667 Nhost medium 1 h | 0.0 |
| 158670 Nhost + IL-1b (10 ng/ml), 1 h | 1.1 |
| 158673 Nhost + PGE2 (10-6M) 1 h | 0.0 |
| 158668 Nhost medium alone 6h | 0.0 |
| 158671 Nhost + IL-1b (10 ng/ml) 6 h | 13.7 |
| 158674 Nhost + PGE2 (10-6M) 6 h | 0.3 |
| 158669 Nhost medium alone 24 h | 0.0 |
| 158672 Nhost + IL-1b (10 ng/ml) 24 h | 0.4 |
| 158675 Nhost + PGE2 (10-6M) 24 h | 0.0 |
| 164327 SW1353 medium alone 1 h | 0.0 |
| 164328 SW1353 + IL-1b (1 ng/ml) 1 h | 1.2 |
| 164329 SW1353 + IL-1b (10 ng/ml) 1 h | 0.8 |
| 164330 SW1353 +TNF-a (10 ng/ml) 1 h | 0.4 |
| 164331 SW1353 +TNF-a (100 ng/ml) 1 h | 0.5 |
| 164332 SW1353 medium alone 6 h | 0.1 |
| 164333 SW1353 + IL-1b (1 ng/ml) 6 h | 4.6 |
| 164334 SW1353 + IL-1b (10 ng/ml) 6 h | 5.4 |
| 164335 SW1353 + TNF-a (10 ng/ml) 6 h | 0.9 |
| 164336 SW1353 + TNF-a (100 ng/ml) 6 h | 1.7 |
| 164337 SW1353 medium alone 18 h | 0.1 |
| 164338 SW1353 + IL-1b (1 ng/ml) 18 h | 11.0 |
| 164339 SW1353 + IL-1b (10 ng/ml) 18 h | 10.2 |
| 164340 SW1353 + TNF-a (10 ng/ml) 18 h | 1.9 |
| 164341 SW1353 + IL-1b (100 ng/ml) 18 h | 3.1 |
| 173326 HFLS-RA (cell application) medium alone 18 h | 0.0 |
| 173327 HFLS-RA (cell application) + TNF-a 18 h | 1.5 |
| 173331 MH7A (synoviocyte cell line) medium 1 h | 0.9 |
| 173332 MH7A (synoviocyte cell line) + IL1b 1 h | 18.7 |
| 173334 MH7A (synoviocyte cell line) TNFa 1 h | 100.0 |
| 173336 MH7A (synoviocyte cell line) medium alone 6 h | 1.5 |
| 173339 MH7A (synoviocyte cell line) + IL1b 6 h | 36.6 |
| 173341 MH7A (synoviocyte cell line) TNFa 6 h | 11.6 |
| 173342 MH7A (synoviocyte cell line) medium alone 18 h | 4.1 |
| 173344 MH7A (synoviocyte cell line) + IL1b 18 h | 19.9 |
| 173346 MH7A (synoviocyte cell line) TNF-a 18 h | 54.3 |

Column A - Rel. Exp. (%) Ag1141, Run 411804421

TABLE BH
General_screening_panel_v1.4

| Tissue Name | A | B |
|---|---|---|
| Adipose | 0.7 | 0.9 |
| Melanoma* Hs688(A).T | 0.0 | 0.0 |
| Melanoma* Hs688(B).T | 0.1 | 0.1 |
| Melanoma* M14 | 0.0 | 0.1 |
| Melanoma* LOXIMVI | 80.7 | 94.0 |
| Melanoma* SK-MEL-5 | 0.0 | 0.0 |
| Squamous cell carcinoma SCC-4 | 4.2 | 4.5 |
| Testis Pool | 0.1 | 0.1 |
| Prostate ca.* (bone met) PC-3 | 1.2 | 1.1 |
| Prostate Pool | 0.1 | 0.0 |
| Placenta | 0.1 | 0.1 |
| Uterus Pool | 0.0 | 0.0 |
| Ovarian ca. OVCAR-3 | 0.1 | 0.0 |
| Ovarian ca. SK-OV-3 | 0.3 | 0.3 |
| Ovarian ca. OVCAR-4 | 0.0 | 0.0 |
| Ovarian ca. OVCAR-5 | 0.1 | 0.1 |
| Ovarian ca. IGROV-1 | 0.0 | 0.0 |
| Ovarian ca. OVCAR-8 | 0.1 | 0.0 |
| Ovary | 0.0 | 0.0 |
| Breast ca. MCF-7 | 0.0 | 0.0 |
| Breast ca. MDA-MB-231 | 0.1 | 0.3 |
| Breast ca. BT 549 | 0.1 | 0.2 |
| Breast ca. T47D | 0.1 | 0.1 |
| Breast ca. MDA-N | 0.0 | 0.0 |
| Breast Pool | 0.1 | 0.1 |
| Trachea | 0.3 | 0.2 |
| Lung | 0.0 | 0.0 |
| Fetal Lung | 2.9 | 2.8 |
| Lung ca. NCI-N417 | 0.0 | 0.0 |
| Lung ca. LX-1 | 0.7 | 0.7 |
| Lung ca. NCI-H146 | 0.0 | 0.0 |
| Lung ca. SHP-77 | 0.0 | 0.0 |
| Lung ca. A549 | 0.0 | 0.0 |
| Lung ca. NCI-H526 | 0.0 | 0.0 |
| Lung ca. NCI-H23 | 0.1 | 0.2 |
| Lung ca. NCI-H460 | 0.5 | 0.3 |
| Lung ca. HOP-62 | 0.6 | 0.8 |
| Lung ca. NCI-H522 | 0.0 | 0.1 |
| Liver | 0.0 | 0.0 |
| Fetal Liver | 0.3 | 0.3 |
| Liver ca. HepG2 | 0.0 | 0.0 |
| Kidney Pool | 0.1 | 0.1 |
| Fetal Kidney | 0.1 | 0.1 |
| Renal ca. 786-0 | 0.1 | 0.1 |
| Renal ca. A498 | 0.1 | 0.0 |
| Renal ca. ACHN | 0.0 | 0.1 |
| Renal ca. UO-31 | 0.8 | 1.0 |
| Renal ca. TK-10 | 0.0 | 0.0 |
| Bladder | 0.3 | 0.3 |
| Gastric ca. (liver met.) NCI-N87 | 0.3 | 0.3 |
| Gastric ca. KATO III | 0.1 | 0.1 |
| Colon ca. SW-948 | 0.0 | 0.0 |
| Colon ca. SW480 | 0.1 | 0.2 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.1 | 0.1 |
| Colon cancer tissue | 8.2 | 9.4 |
| Colon ca. SW1116 | 0.0 | 0.0 |
| Colon ca. Colo-205 | 0.0 | 0.0 |
| Colon ca. SW-48 | 0.0 | 0.0 |
| Colon Pool | 0.1 | 0.0 |
| Small Intestine Pool | 0.0 | 0.1 |
| Stomach Pool | 2.0 | 1.5 |
| Bone Marrow Pool | 0.0 | 0.1 |
| Fetal Heart | 0.0 | 0.0 |
| Heart Pool | 0.0 | 0.0 |
| Lymph Node Pool | 0.0 | 0.0 |
| Fetal Skeletal Muscle | 0.0 | 0.0 |
| Skeletal Muscle Pool | 0.0 | 0.0 |
| Spleen Pool | 0.4 | 0.4 |
| Thymus Pool | 0.1 | 0.1 |
| CNS cancer (glio/astro) U87-MG | 100.0 | 100.0 |
| CNS cancer (glio/astro) U-118-MG | 0.3 | 0.3 |
| CNS cancer (neuro; met) SK-N-AS | 0.1 | 0.2 |
| CNS cancer (astro) SF-539 | 0.0 | 0.0 |
| CNS cancer (astro) SNB-75 | 0.1 | 0.1 |
| CNS cancer (glio) SNB-19 | 0.0 | 0.0 |
| CNS cancer (glio) SF-295 | 2.6 | 6.4 |
| Brain (Amygdala) Pool | 0.3 | 0.3 |
| Brain (cerebellum) | 0.1 | 0.1 |
| Brain (fetal) | 0.3 | 0.2 |
| Brain (Hippocampus) Pool | 1.0 | 0.9 |
| Cerebral Cortex Pool | 0.3 | 0.3 |
| Brain (Substantia nigra) Pool | 0.4 | 0.4 |
| Brain (Thalamus) Pool | 0.4 | 0.4 |
| Brain (whole) | 0.2 | 0.1 |
| Spinal Cord Pool | 0.4 | 0.4 |
| Adrenal Gland | 0.1 | 0.1 |
| Pituitary gland Pool | 0.0 | 0.0 |
| Salivary Gland | 0.1 | 0.1 |
| Thyroid (female) | 0.2 | 0.1 |
| Pancreatic ca. CAPAN2 | 0.1 | 0.1 |
| Pancreas Pool | 0.1 | 0.1 |

Column A - Rel. Exp. (%) Ag1141, Run 208030042
Column B - Rel. Exp. (%) Ag1141, Run 212141055

AI_comprehensive panel_v1.0 Summary: Ag1141/μg6369 Highest expression of this gene was detected in matched control sample for ulcerative colitis (CTs=27).

Significant expression of this gene was also seen in samples derived from normal and orthoarthitis/rheumatoid arthritis bone, cartilage, synovium and synovial fluid samples, from normal lung, COPD lung, emphysema, atopic asthma, asthma, Crohn's disease (normal matched control and diseased), ulcerative colitis(normal matched control and diseased), and psoriasis (normal matched control and diseased). Therefore, therapeutic modulation of this gene, encoded protein and/or use of antibodies or small molecule targeting this gene or gene product will ameliorate symptoms/conditions associated with autoimmune and inflammatory disorders including psoriasis, allergy, asthma, inflammatory bowel disease, rheumatoid arthritis and osteoarthritis.

Cellular OA/RA Summary: Ag1141 Highest expression of this gene was detected in TNF alpha treated MH7A (synoviocyte) cell line (CT=19.7). Expression of this gene was upregulated in activated normal osteoblast (Nhost), chondrocytes (SW1353 cell line) and synoviocyte cell lines. Therefore, modulation of this gene, encoded protein and/or use of antibodies or small molecule targeting this gene or gene product is useful in the treatment of inflammatory and autoimmune diseases such as osteoarthritis and rheumatoid arthritis.

General_screening_panel_v1.4 Summary: Ag1141 Highest expression of this gene was detected in a brain cancer U87-MG cell line (CT=20.9). High to moderate expression of this gene was also seen in number of cancer cell lines derived from pancreatic, gastric, colon, lung, liver, renal, breast, ovarian, prostate, squamous cell carcinoma, melanoma and brain cancers. Thus, expression levels of this gene is useful as marker to detect the presence of these cancers. Therapeutic modulation of this gene, encoded protein and/or use of small molecule targeting this gene or gene product is effective in the treatment of pancreatic, gastric, colon, lung, liver, renal, breast, ovarian, prostate, squamous cell carcinoma, melanoma and brain cancers.

Among tissues with metabolic or endocrine function, this gene was expressed at moderate levels in pancreas, adipose, adrenal gland, thyroid, pituitary gland, skeletal muscle, heart, liver and the gastrointestinal tract. Therefore, therapeutic modulation of this gene, encoded protein is useful in the treatment of endocrine/metabolically related diseases, such as obesity and diabetes.

This gene was expressed at moderate levels in all regions of the central nervous system examined, including amygdala, hippocampus, substantia nigra, thalamus, cerebellum, cerebral cortex, and spinal cord. Therapeutic modulation of this gene, encoded protein and/or use of antibodies or small molecule targeting this gene or gene product is useful in the treatment of central nervous system disorders such as Alzheimer's disease, Parkinson's disease, epilepsy, multiple sclerosis, schizophrenia and depression.

C. NOV3 (CG127616-01 and CG127616-02): FL_{—}104 aa (del 54-143aa) and Partial (del 1-4aa; 54-143aa) [Erythropoietin Precursor-like]

Expression of genes CG127616-01 and CG127616-02 was assessed using the primer-probe sets Ag4746 and Ag6361, described in Tables CA and CB. Results of the RTQ-PCR runs are shown in Tables CC and CD.

TABLE CA

Probe Name Ag4746

| | | | Start | SEQ ID |
|---|---|---|---|---|
| Primers | Sequences | Length | Position | No |
| Forward | 5′-gaatatcacgaaggaagcca-3′ | 20 | 155 | 226 |
| Probe | TET-5′-cctcagctgctccactccgaaca-3′- | 23 | 193 | 227 |
| | TAMRA | | | |
| Reverse | 5′-cggaaagtgtcagcagtgat-3′ | 20 | 216 | 228 |

TABLE CB

Probe Name Ag6361

| | | | Start | SEQ ID |
|---|---|---|---|---|
| Primers | Sequences | Length | Position | No |
| Forward | 5′-acaatcactgctgacactttcc-3′ | 22 | 213 | 229 |
| Probe | TET-5′-ttccgagtctactccaatttcctccg-3′- | 26 | 243 | 230 |
| | TAMRA | | | |
| Reverse | 5′-cctgtgtacagcttcagctttc-3′ | 22 | 271 | 231 |

TABLE CC
General_screening_panel_v1.4

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.6 |
| Testis Pool | 0.8 |
| Prostate ca.* (bone met) PC-3 | 0.6 |
| Prostate Pool | 1.0 |
| Placenta | 1.4 |
| Uterus Pool | 2.8 |
| Ovarian ca. OVCAR-3 | 1.3 |
| Ovarian ca. SK-OV-3 | 0.2 |
| Ovarian ca. OVCAR-4 | 0.0 |
| Ovarian ca. OVCAR-5 | 0.3 |
| Ovarian ca. IGROV-1 | 0.0 |
| Ovarian ca. OVCAR-8 | 1.5 |
| Ovary | 0.0 |
| Breast ca. MCF-7 | 0.0 |
| Breast ca. MDA-MB-231 | 3.7 |
| Breast ca. BT 549 | 3.1 |
| Breast ca. T47D | 0.4 |
| Breast ca. MDA-N | 0.0 |
| Breast Pool | 4.9 |
| Trachea | 0.0 |
| Lung | 1.0 |
| Fetal Lung | 0.2 |
| Lung ca. NCI-N417 | 0.0 |
| Lung ca. LX-1 | 1.1 |
| Lung ca. NCI-H146 | 1.1 |
| Lung ca. SHP-77 | 0.3 |
| Lung ca. A549 | 0.0 |
| Lung ca. NCI-H526 | 0.0 |
| Lung ca. NCI-H23 | 18.0 |
| Lung ca. NCI-H460 | 0.2 |
| Lung ca. HOP-62 | 0.0 |
| Lung ca. NCI-H522 | 0.0 |
| Liver | 0.0 |
| Fetal Liver | 18.8 |
| Liver ca. HepG2 | 100.0 |
| Kidney Pool | 4.9 |
| Fetal Kidney | 1.0 |
| Renal ca. 786-0 | 1.3 |
| Renal ca. A498 | 0.5 |
| Renal ca. ACHN | 0.4 |
| Renal ca. UO-31 | 0.0 |
| Renal ca. TK-10 | 27.4 |
| Bladder | 29.1 |
| Gastric ca. (liver met.) NCI-N87 | 1.2 |
| Gastric ca. KATO III | 0.0 |
| Colon ca. SW-948 | 0.5 |
| Colon ca. SW480 | 0.2 |
| Colon ca.* (SW480 met) SW620 | 0.0 |
| Colon ca. HT29 | 0.0 |
| Colon ca. HCT-116 | 2.4 |
| Colon ca. CaCo-2 | 33.2 |
| Colon cancer tissue | 0.0 |
| Colon ca. SW1116 | 0.0 |
| Colon ca. Colo-205 | 0.0 |
| Colon ca. SW-48 | 0.4 |
| Colon Pool | 12.3 |
| Small Intestine Pool | 0.2 |
| Stomach Pool | 5.8 |
| Bone Marrow Pool | 1.9 |
| Fetal Heart | 0.0 |
| Heart Pool | 0.7 |
| Lymph Node Pool | 14.5 |
| Fetal Skeletal Muscle | 0.0 |
| Skeletal Muscle Pool | 0.0 |
| Spleen Pool | 0.2 |
| Thymus Pool | 1.3 |
| CNS cancer (glio/astro) U87-MG | 1.6 |
| CNS cancer (glio/astro) U-118-MG | 0.0 |
| CNS cancer (neuro; met) SK-N-AS | 0.2 |
| CNS cancer (astro) SF-539 | 0.0 |
| CNS cancer (astro) SNB-75 | 0.2 |
| CNS cancer (glio) SNB-19 | 0.0 |
| CNS cancer (glio) SF-295 | 1.4 |
| Brain (Amygdala) Pool | 0.0 |
| Brain (cerebellum) | 0.1 |
| Brain (fetal) | 1.1 |
| Brain (Hippocampus) Pool | 0.4 |
| Cerebral Cortex Pool | 0.0 |
| Brain (Substantia nigra) Pool | 6.1 |
| Brain (Thalamus) Pool | 0.0 |
| Brain (whole) | 4.8 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 7.7 |

Column A - Rel. Exp. (%) Ag4746, Run 214145483

TABLE CD
general oncology screening panel_v_2.4

| Tissue Name | A |
|---|---|
| Colon cancer 1 | 0.0 |
| Colon NAT 1 | 0.0 |
| Colon cancer 2 | 0.0 |
| Colon NAT 2 | 0.0 |
| Colon cancer 3 | 0.0 |
| Colon NAT 3 | 0.0 |
| Colon malignant cancer 4 | 0.0 |
| Colon NAT 4 | 0.0 |
| Lung cancer 1 | 0.0 |
| Lung NAT 1 | 0.0 |
| Lung cancer 2 | 0.0 |
| Lung NAT 2 | 0.0 |
| Squamous cell carcinoma 3 | 1.7 |
| Lung NAT 3 | 0.0 |
| Metastatic melanoma 1 | 1.0 |
| Melanoma 2 | 0.0 |
| Melanoma 3 | 0.0 |
| Metastatic melanoma 4 | 0.6 |
| Metastatic melanoma 5 | 0.0 |
| Bladder cancer 1 | 0.0 |
| Bladder NAT 1 | 0.8 |
| Bladder cancer 2 | 0.0 |
| Bladder NAT 2 | 0.0 |
| Bladder NAT 3 | 0.0 |
| Bladder NAT 4 | 0.0 |
| Prostate adenocarcinoma 1 | 5.8 |
| Prostate adenocarcinoma 2 | 0.6 |
| Prostate adenocarcinoma 3 | 0.0 |
| Prostate adenocarcinoma 4 | 0.0 |
| Prostate NAT 5 | 0.0 |
| Prostate adenocarcinoma 6 | 0.0 |
| Prostate adenocarcinoma 7 | 0.0 |
| Prostate adenocarcinoma 8 | 0.0 |
| Prostate adenocarcinoma 9 | 0.0 |
| Prostate NAT 10 | 0.0 |
| Kidney cancer 1 | 71.2 |
| Kidney NAT 1 | 0.0 |
| Kidney cancer 2 | 0.0 |
| Kidney NAT 2 | 0.9 |
| Kidney cancer 3 | 1.1 |
| Kidney NAT 3 | 0.0 |
| Kidney cancer 4 | 100.0 |
| Kidney NAT 4 | 0.0 |

Column A - Rel. Exp. (%) Ag4746, Run 259807104

General_screening_panel_v1.4 Summary: Ag4746 Highest expression of this gene was seen in a liver cancer cell line (CT=30.3). Moderate levels of expression were also seen in colon, renal and lung cancer cell lines, with low but significant expression detectable in lymph node and fetal liver. The transcript for this gene encodes a putative variant of erythropoietin (Epo), which is produced in the kidney and liver of normal adults. Human erythropoietin (Epo) is an acidic glycoprotein hormone that mediates the production of red blood cells, promotes erythroid differentiation, initiates hemoglobin synthesis. In addition, Epo has been shown to be a potent growth factor for the development of red blood cells from hematopoetic stem cells. Thus, the expression in hematopoietic tissues in this panel is consistent with the characterization of this novel protein as a novel variant of Epo. Modulation of the expression or function of this gene product is useful in the treatment of hematopoietic disorders.

general oncology screening panel_v_{—}2.4 Summary: Ag4746 Moderate expression of this gene was seen in two samples derived from kidney cancers (CTs=32). The expression of this gene is useful as a marker for kidney cancer.

D. NOV4 (CG54455-03 and CG54455-07): FGF-10X

Expression of genes CG54455-03 and CG54455-07 was assessed using the primer-probe sets Ag4346, Ag4347 and Ag7772, described in Tables DA, DB and DC. Results of the RTQ-PCR runs are shown in Tables DD, DE, DF, DG, DH, DI, DJ and DK.

TABLE DA

Probe Name Ag4346

| | | | Start | SEQ ID |
|---|---|---|---|---|
| Primers | Sequences | Length | Position | No |
| Forward | 5′-cgtggtcatcaaagcagtgt-3′ | 20 | 249 | 232 |
| Probe | TET-5′-ctcaggcttctacgtggccatgaac-3′- | 25 | 270 | 233 |
| | TAMRA | | | |
| Reverse | 5′-tgcagtccacggtgtagagt-3′ | 20 | 321 | 234 |

TABLE DB

Probe Name Ag4347

| | | | Start | SEQ ID |
|---|---|---|---|---|
| Primers | Sequences | Length | Position | No |
| Forward | 5′-tggagatccgctctgtacacg-3′ | 21 | 221 | 235 |
| Probe | TET-5′-cctgaggacactgctttgatgaccacg-3′- | 27 | 249 | 236 |
| | TAMRA | | | |
| Reverse | 5′-cggttcatggccacgtaga-3′ | 19 | 278 | 237 |

TABLE DC

Probe Name Ag7772

| | | | | |
|---|---|---|---|---|
| | | | Start | SEQ ID |
| Primers | Sequences | Length | Position | No |

| | | | | |
|---|---|---|---|---|
| Forward | 5′-tggagatccgctctgtacac-3′ | 20 | 221 | 238 |
| Probe | TET-5′-tcatcaaagcagtgtcctcaggcttc-3′- | 26 | 254 | 239 |
| | TAMRA | | | |
| Reverse | 5′-tgcagtccacggtgtagagt-3′ | 20 | 321 | 240 |

TABLE DD
AI_comprehensive panel_v1.0

| Tissue Name | A |
|---|---|
| 110967 COPD-F | 10.0 |
| 110980 COPD-F | 19.2 |
| 110968 COPD-M | 13.3 |
| 110977 COPD-M | 59.5 |
| 110989 Emphysema-F | 16.4 |
| 110992 Emphysema-F | 14.3 |
| 110993 Emphysema-F | 6.2 |
| 110994 Emphysema-F | 4.1 |
| 110995 Emphysema-F | 19.3 |
| 110996 Emphysema-F | 9.3 |
| 110997 Asthma-M | 1.7 |
| 111001 Asthma-F | 4.5 |
| 111002 Asthma-F | 15.7 |
| 111003 Atopic Asthma-F | 14.0 |
| 111004 Atopic Asthma-F | 21.5 |
| 111005 Atopic Asthma-F | 6.4 |
| 111006 Atopic Asthma-F | 5.1 |
| 111417 Allergy-M | 18.7 |
| 112347 Allergy-M | 4.8 |
| 112349 Normal Lung-F | 2.7 |
| 112357 Normal Lung-F | 100.0 |
| 112354 Normal Lung-M | 8.3 |
| 112374 Crohns-F | 19.9 |
| 112389 Match Control Crohns-F | 33.9 |
| 112375 Crohns-F | 6.7 |
| 112732 Match Control Crohns-F | 31.4 |
| 112725 Crohns-M | 9.1 |
| 112387 Match Control Crohns-M | 2.2 |
| 112378 Crohns-M | 4.3 |
| 112390 Match Control Crohns-M | 28.7 |
| 112726 Crohns-M | 1.4 |
| 112731 Match Control Crohns-M | 15.3 |
| 112380 Ulcer Col-F | 4.4 |
| 112734 Match Control Ulcer Col-F | 18.8 |
| 112384 Ulcer Col-F | 7.0 |
| 112737 Match Control Ulcer Col-F | 1.2 |
| 112386 Ulcer Col-F | 19.6 |
| 112738 Match Control Ulcer Col-F | 1.0 |
| 112381 Ulcer Col-M | 0.0 |
| 112735 Match Control Ulcer Col-M | 0.0 |
| 112382 Ulcer Col-M | 7.1 |
| 112394 Match Control Ulcer Col-M | 11.9 |
| 112383 Ulcer Col-M | 8.4 |
| 112736 Match Control Ulcer Col-M | 29.1 |
| 112423 Psoriasis-F | 1.0 |
| 112427 Match Control Psoriasis-F | 60.3 |
| 112418 Psoriasis-M | 12.1 |
| 112723 Match Control Psoriasis-M | 0.8 |
| 112419 Psoriasis-M | 4.9 |
| 112424 Match Control Psoriasis-M | 15.7 |
| 112420 Psoriasis-M | 15.2 |
| 112425 Match Control Psoriasis-M | 30.1 |
| 104689 (MF) OA Bone-Backus | 21.5 |
| 104690 (MF) Adj “Normal” Bone-Backus | 10.9 |
| 104691 (MF) OA Synovium-Backus | 11.4 |
| 104692 (BA) OA Cartilage-Backus | 27.4 |
| 104694 (BA) OA Bone-Backus | 5.4 |
| 104695 (BA) Adj “Normal” Bone-Backus | 36.1 |
| 104696 (BA) OA Synovium-Backus | 2.0 |
| 104700 (SS) OA Bone-Backus | 4.7 |
| 104701 (SS) Adj “Normal” Bone-Backus | 15.0 |
| 104702 (SS) OA Synovium-Backus | 10.6 |
| 117093 OA Cartilage Rep7 | 7.2 |
| 112672 OA Bone5 | 2.5 |
| 112673 OA Synovium5 | 3.4 |
| 112674 OA Synovial Fluid cells5 | 9.9 |
| 117100 OA Cartilage Rep14 | 2.7 |
| 112756 OA Bone9 | 18.7 |
| 112757 OA Synovium9 | 4.3 |
| 112758 OA Synovial Fluid Cells9 | 4.7 |
| 117125 RA Cartilage Rep2 | 3.1 |
| 113492 Bone2 RA | 5.8 |
| 113493 Synovium2 RA | 1.8 |
| 113494 Syn Fluid Cells RA | 8.0 |
| 113499 Cartilage4 RA | 2.6 |
| 113500 Bone4 RA | 10.4 |
| 113501 Synovium4 RA | 5.6 |
| 113502 Syn Fluid Cells4 RA | 5.3 |
| 113495 Cartilage3 RA | 5.9 |
| 113496 Bone3 RA | 3.5 |
| 113497 Synovium3 RA | 0.4 |
| 113498 Syn Fluid Cells3 RA | 1.6 |
| 117106 Normal Cartilage Rep20 | 4.2 |
| 113663 Bone3 Normal | 0.0 |
| 113664 Synovium3 Normal | 0.0 |
| 113665 Syn Fluid Cells3 Normal | 3.6 |
| 117107 Normal Cartilage Rep22 | 3.3 |
| 113667 Bone4 Normal | 10.7 |
| 113668 Synovium4 Normal | 16.4 |
| 113669 Syn Fluid Cells4 Normal | 4.2 |

Column A - Rel. Exp. (%) Ag4347, Run 278182082

TABLE DE
Cellular OA/RA

| Tissue Name | A |
|---|---|
| 158667 Nhost medium 1 h | 8.5 |
| 158670 Nhost + IL-1b (10 ng/ml), 1 h | 3.5 |
| 158673 Nhost + PGE2 (10-6M) 1 h | 18.2 |
| 158668 Nhost medium alone 6 h | 26.6 |
| 158671 Nhost + IL-1b (10 ng/ml) 6 h | 29.3 |
| 158674 Nhost + PGE2 (10-6M) 6 h | 2.3 |
| 158669 Nhost medium alone 24 h | 0.0 |
| 158672 Nhost + IL-1b (10 ng/ml) 24 h | 33.7 |
| 158675 Nhost + PGE2 (10-6M) 24 h | 41.5 |
| 164327 SW1353 medium alone 1 h | 66.0 |
| 164328 SW1353 + IL-1b (1 ng/ml) 1 h | 22.5 |
| 164329 SW1353 + IL-1b (10 ng/ml) 1 h | 26.8 |
| 164330 SW1353 + TNF-a (10 ng/ml) 1 h | 20.4 |
| 164331 SW1353 + TNF-a (100 ng/ml) 1 h | 51.1 |
| 164332 SW1353 medium alone 6 h | 42.6 |
| 164333 SW1353 + IL-1b (1 ng/ml) 6 h | 41.5 |
| 164334 SW1353 + IL-1b (10 ng/ml) 6 h | 88.3 |
| 164335 SW1353 + TNF-a (10 ng/ml) 6 h | 34.4 |
| 164336 SW1353 + TNF-a (100 ng/ml) 6 h | 24.5 |
| 164337 SW1353 medium alone 18 h | 49.7 |
| 164338 SW1353 + IL-1b (1 ng/ml) 18 h | 38.2 |
| 164339 SW1353 + IL-1b (10 ng/ml) 18 h | 34.9 |
| 164340 SW1353 + TNF-a (10 ng/ml) 18 h | 19.9 |
| 164341 SW1353 + IL-1b (100 ng/ml) 18 h | 55.1 |
| 173326 HFLS-RA (cell application) medium alone 18 h | 12.2 |
| 173327 HFLS-RA (cell application) + TNF-a 18 h | 36.9 |
| 173331 MH7A (synoviocyte cell line) medium 1 h | 36.6 |
| 173332 MH7A (synoviocyte cell line) + IL1b 1 h | 15.3 |
| 173334 MH7A (synoviocyte cell line) TNFa 1 h | 0.0 |
| 173336 MH7A (synoviocyte cell line) medium alone 6 h | 100.0 |
| 173339 MH7A (synoviocyte cell line) + IL1b 6 h | 77.9 |
| 173341 MH7A (synoviocyte cell line) TNFa 6 h | 7.6 |
| 173342 MH7A (synoviocyte cell line) medium alone 18 h | 15.1 |
| 173344 MH7A (synoviocyte cell line) + IL1b 18 h | 30.8 |
| 173346 MH7A (synoviocyte cell line) TNF-a 18 h | 25.2 |

Column A - Rel. Exp. (%) Ag4346, Run 406013342

TABLE DF
General_screening_panel_v1.4

| Tissue Name | A |
|---|---|
| Adipose | 0.0 |
| Melanoma* Hs688(A).T | 0.0 |
| Melanoma* Hs688(B).T | 3.2 |
| Melanoma* M14 | 5.6 |
| Melanoma* LOXIMVI | 3.7 |
| Melanoma* SK-MEL-5 | 2.3 |
| Squamous cell carcinoma SCC-4 | 0.0 |
| Testis Pool | 20.4 |
| Prostate ca.* (bone met) PC-3 | 5.9 |
| Prostate Pool | 2.1 |
| Placenta | 18.9 |
| Uterus Pool | 2.5 |
| Ovarian ca. OVCAR-3 | 8.6 |
| Ovarian ca. SK-OV-3 | 100.0 |
| Ovarian ca. OVCAR-4 | 7.4 |
| Ovarian ca. OVCAR-5 | 41.8 |
| Ovarian ca. IGROV-1 | 13.3 |
| Ovarian ca. OVCAR-8 | 27.4 |
| Ovary | 27.4 |
| Breast ca. MCF-7 | 0.0 |
| Breast ca. MDA-MB-231 | 17.1 |
| Breast ca. BT 549 | 10.0 |
| Breast ca. T47D | 83.5 |
| Breast ca. MDA-N | 10.2 |
| Breast Pool | 4.5 |
| Trachea | 14.4 |
| Lung | 0.0 |
| Fetal Lung | 0.0 |
| Lung ca. NCI-N417 | 2.5 |
| Lung ca. LX-1 | 15.7 |
| Lung ca. NCI-H146 | 0.0 |
| Lung ca. SHP-77 | 14.0 |
| Lung ca. A549 | 6.8 |
| Lung ca. NCI-H526 | 4.4 |
| Lung ca. NCI-H23 | 17.1 |
| Lung ca. NCI-H460 | 0.0 |
| Lung ca. HOP-62 | 8.8 |
| Lung ca. NCI-H522 | 12.9 |
| Liver | 1.2 |
| Fetal Liver | 0.0 |
| Liver ca. HepG2 | 5.1 |
| Kidney Pool | 13.6 |
| Fetal Kidney | 8.6 |
| Renal ca. 786-0 | 11.5 |
| Renal ca. A498 | 3.5 |
| Renal ca. ACHN | 8.2 |
| Renal ca. UO-31 | 0.0 |
| Renal ca. TK-10 | 15.2 |
| Bladder | 4.3 |
| Gastric ca. (liver met.) NCI-N87 | 8.7 |
| Gastric ca. KATO III | 3.7 |
| Colon ca. SW-948 | 1.7 |
| Colon ca. SW480 | 11.1 |
| Colon ca.* (SW480 met) SW620 | 12.6 |
| Colon ca. HT29 | 4.7 |
| Colon ca. HCT-116 | 40.1 |
| Colon ca. CaCo-2 | 3.6 |
| Colon cancer tissue | 0.0 |
| Colon ca. SW1116 | 29.7 |
| Colon ca. Colo-205 | 0.0 |
| Colon ca. SW-48 | 0.0 |
| Colon Pool | 3.9 |
| Small Intestine Pool | 10.5 |
| Stomach Pool | 3.7 |
| Bone Marrow Pool | 18.2 |
| Fetal Heart | 0.0 |
| Heart Pool | 1.1 |
| Lymph Node Pool | 17.4 |
| Fetal Skeletal Muscle | 0.0 |
| Skeletal Muscle Pool | 9.3 |
| Spleen Pool | 5.1 |
| Thymus Pool | 9.9 |
| CNS cancer (glio/astro) U87-MG | 14.6 |
| CNS cancer (glio/astro) U-118-MG | 32.3 |
| CNS cancer (neuro; met) SK-N-AS | 15.7 |
| CNS cancer (astro) SF-539 | 3.6 |
| CNS cancer (astro) SNB-75 | 8.7 |
| CNS cancer (glio) SNB-19 | 3.9 |
| CNS cancer (glio) SF-295 | 54.3 |
| Brain (Amygdala) Pool | 70.7 |
| Brain (cerebellum) | 26.1 |
| Brain (fetal) | 40.3 |
| Brain (Hippocampus) Pool | 34.4 |
| Cerebral Cortex Pool | 52.5 |
| Brain (Substantia nigra) Pool | 53.6 |
| Brain (Thalamus) Pool | 57.8 |
| Brain (whole) | 31.9 |
| Spinal Cord Pool | 34.9 |
| Adrenal Gland | 1.3 |
| Pituitary gland Pool | 2.1 |
| Salivary Gland | 7.4 |
| Thyroid (female) | 0.0 |
| Pancreatic ca. CAPAN2 | 1.8 |
| Pancreas Pool | 0.0 |

Column A - Rel. Exp. (%) Ag4347, Run 222523511

TABLE DG
Mini tissue OA/RA

| Tissue Name | A |
|---|---|
| 161315 OA PT 5 Cartilage | 2.4 |
| 161291 OA PT 7 Cartilage | 0.4 |
| 161303 OA PT 8 Cartilage | 0.8 |
| 161287 OA PT 10 Cartilage | 0.4 |
| 173546 RA Cartilage PT 85 | 3.4 |
| 161316 OA PT 5 Synovium | 0.4 |
| 161290 OA PT 7 Synovium | 1.3 |
| 161304 OA PT 8 Synovium | 0.5 |
| 161289 OA PT 10 Synovium | 2.7 |
| 161237 RA PT 1 Synovium | 1.8 |
| 173547 RA PT 85 Synovium | 5.0 |
| 173553 RA Synovium Ardais RNA 1 | 6.8 |
| 173554 RA Synovium Ardais RNA 2 | 1.8 |
| 173543 Normal Synovium 1 NDRI | 5.6 |
| 173545 Normal Synovium 3 NDRI | 2.1 |
| 173555 Normal Synovium Ardais RNA 1 | 20.6 |
| 161314 OA PT 5 Bone | 2.4 |
| 161292 OA PT 7 Bone | 2.0 |
| 161302 OA PT 8 Bone | 1.1 |
| 161288 OA PT 10 Bone | 3.6 |
| 173548 RA PT 85 Bone | 0.6 |
| 150168 Adrenal gland | 4.2 |
| 150171 Brain (whole) | 100.0 |
| 154975 Colon | 4.4 |
| 154947 Heart | 0.0 |
| 155689 Kidney pool | 8.4 |
| 150176 Lung* | 2.9 |
| 150178 Ovary* | 7.9 |
| 144488 Skeletal muscle pool* | 0.0 |
| 154967 Small intestine | 8.0 |
| 154970 Stomach* | 0.6 |

Column A - Rel. Exp. (%) Ag4346, Run 406107070

TABLE DH
PGI1.0

| Tissue Name | A |
|---|---|
| 162191 Normal Lung 1 (IBS) | 0.5 |
| 162570 Normal Lung 4 (Aastrand) | 0.0 |
| 160468 MD lung | 0.0 |
| 156629 MD Lung 13 | 0.0 |
| 162571 Normal Lung 3 (Aastrand) | 0.4 |
| 162186 Fibrosis Lung 1 (Genomic Collaborative) | 100.0 |
| 162187 Fibrosis Lung 2 (Genomic Collaborative) | 47.6 |
| 151281 Fibrosis lung 11(Ardais) | 3.5 |
| 162190 Asthma Lung 4 (Genomic Collaborative) | 33.2 |
| 160467 Asthma Lung 13 (MD) | 0.0 |
| 137027 Emphysema Lung 1 (Ardais) | 0.0 |
| 137028 Emphysema Lung 2 (Ardais) | 3.7 |
| 137040 Emphysema Lung 3 (Ardais) | 2.8 |
| 137041 Emphysema Lung 4 (Ardais) | 0.0 |
| 137043 Emphysema Lung 5 (Ardais) | 0.5 |
| 142817 Emphysema Lung 6 (Ardais) | 1.9 |
| 142818 Emphysema Lung 7 (Ardais) | 2.6 |
| 142819 Emphysema Lung 8 (Ardais) | 5.9 |
| 142820 Emphysema Lung 9 (Ardais) | 2.0 |
| 142821 Emphysema Lung 10 (Ardais) | 1.1 |
| 162185 Emphysema Lung 12 (Ardais) | 0.0 |
| 162184 Emphysema Lung 13 (Ardais) | 1.1 |
| 162183 Emphysema Lung 14 (Ardais) | 6.6 |
| 162188 Emphysema Lung 15 (Genomic Collaborative) | 82.4 |
| 162177 NAT UC Colon 1(Ardais) | 0.4 |
| 162176 UC Colon 1(Ardais) | 0.0 |
| 162179 NAT UC Colon 2(Ardais) | 0.0 |
| 162178 UC Colon 2(Ardais) | 0.0 |
| 162181 NAT UC Colon 3(Ardais) | 0.9 |
| 162180 UC Colon 3(Ardais) | 0.2 |
| 162182 NAT UC Colon 4 (Ardais) | 3.1 |
| 137042 UC Colon 1108 | 0.0 |
| 137029 UC Colon 8215 | 0.6 |
| 137031 UC Colon 8217 | 0.6 |
| 137036 UC Colon 1137 | 1.1 |
| 137038 UC Colon 1491 | 0.8 |
| 137039 UC Colon 1546 | 0.7 |
| 162594 NAT Crohn's 47751 (NDRI) | 0.0 |
| 162593 Crohn's 47751 (NDRI) | 0.0 |

Column A - Rel. Exp. (%) Ag4346, Run 416198456

TABLE DI
Panel 3D

| Tissue Name | A |
|---|---|
| 94905 Daoy Medulloblastoma/Cerebellum | 0.0 |
| 94906 TE671 Medulloblastom/Cerebellum | 0.0 |
| 94907 D283 Med Medulloblastoma/Cerebellum | 7.7 |
| 94908 PFSK-1 Primitive | 3.7 |
| Neuroectodermal/Cerebellum | |
| 94909 XF-498 CNS | 0.0 |
| 94910 SNB-78 CNS/glioma | 27.9 |
| 94911 SF-268 CNS/glioblastoma | 0.0 |
| 94912 T98G Glioblastoma | 0.0 |
| 96776 SK-N-SH Neuroblastoma (metastasis) | 3.5 |
| 94913 SF-295 CNS/glioblastoma | 19.3 |
| 94914 Cerebellum | 15.1 |
| 96777 Cerebellum | 14.9 |
| 94916 NCI-H292 Mucoepidermoid | 14.1 |
| lung carcinoma | |
| 94917 DMS-114 Small cell lung cancer | 63.7 |
| 94918 DMS-79 Small cell lung | 100.0 |
| cancer/neuroendocrine | |
| 94919 NCI-H146 Small cell lung | 7.4 |
| cancer/neuroendocrine | |
| 94920 NCI-H526 Small cell lung | 1.2 |
| cancer/neuroendocrine | |
| 94921 NCI-N417 Small cell lung | 0.0 |
| cancer/neuroendocrine | |
| 94923 NCI-H82 Small cell lung | 0.0 |
| cancer/neuroendocrine | |
| 94924 NCI-H157 Squamous cell lung | 0.0 |
| cancer (metastasis) | |
| 94925 NCI-H1155 Large cell lung | 4.5 |
| cancer/neuroendocrine | |
| 94926 NCI-H1299 Large cell lung | 0.0 |
| cancer/neuroendocrine | |
| 94927 NCI-H727 Lung carcinoid | 0.0 |
| 94928 NCI-UMC-11 Lung carcinoid | 0.0 |
| 94929 LX-1 Small cell lung cancer | 0.0 |
| 94930 Colo-205 Colon cancer | 0.0 |
| 94931 KM12 Colon cancer | 0.0 |
| 94932 KM20L2 Colon cancer | 0.0 |
| 94933 NCI-H716 Colon cancer | 28.3 |
| 94935 SW-48 Colon adenocarcinoma | 7.2 |
| 94936 SW1116 Colon adenocarcinoma | 11.0 |
| 94937 LS 174T Colon adenocarcinoma | 13.5 |
| 94938 SW-948 Colon adenocarcinoma | 0.0 |
| 94939 SW-480 Colon adenocarcinoma | 0.0 |
| 94940 NCI-SNU-5 Gastric carcinoma | 10.5 |
| KATO III- Gastric carcinoma | 29.1 |
| 94943 NCI-SNU-16 Gastric carcinoma | 0.0 |
| 94944 NCI-SNU-1 Gastric carcinoma | 7.5 |
| 94946 RF-1 Gastric adenocarcinoma | 0.0 |
| 94947 RF-48 Gastric adenocarcinoma | 0.0 |
| 96778 MKN-45 Gastric carcinoma | 23.2 |
| 94949 NCI-N87 Gastric carcinoma | 0.0 |
| 94951 OVCAR-5 Ovarian carcinoma | 0.0 |
| 94952 RL95-2 Uterine carcinoma | 0.0 |
| 94953 HelaS3 Cervical adenocarcinoma | 0.0 |
| 94954 Ca Ski Cervical epidermoid | 0.0 |
| carcinoma (metastasis | |
| 94955 ES-2 Ovarian clear cell carcinoma | 0.0 |
| 94957 Ramos Stimulated with | 0.0 |
| PMA/ionomycin 6 h | |
| 94958 Ramos Stimulated with | 0.0 |
| PMA/ionomycin 14 h | |
| 94962 MEG-01 Chronic myelogenous | 0.0 |
| leukemia (megokaryoblast) | |
| 94963 Raji Burkitt's lymphoma | 7.1 |
| 94964 Daudi Burkitt's lymphoma | 5.7 |
| 94965 U266 B-cell plasmacytoma/myeloma | 0.0 |
| 94968 CA46 Burkitt's lymphoma | 6.7 |
| 94970 RL non-Hodgkin's B-cell lymphoma | 0.0 |
| 94972 JM1 pre-B-cell lymphoma/leukemia | 0.0 |
| 94973 Jurkat T cell leukemia | 0.0 |
| 94974 TF-1 Erythroleukemia | 0.0 |
| 94975 HUT 78 T-cell lymphoma | 0.0 |
| 94977 U937 Histiocytic lymphoma | 0.0 |
| 94980 KU-812 Myelogenous leukemia | 14.0 |
| 769-P- Clear cell renal carcinoma | 13.8 |
| 94983 Caki-2 Clear cell renal carcinoma | 0.0 |
| 94984 SW 839 Clear cell renal carcinoma | 19.6 |
| 94986 G401 Wilms' tumor | 7.8 |
| 94987 Hs766T Pancreatic carcinoma | 6.8 |
| (LN metastasis) | |
| 94988 CAPAN-1 Pancreatic | 0.0 |
| adenocarcinoma (liver metastasis) | |
| 94989 SU86.86 Pancreatic carcinoma | 0.0 |
| (liver metastasis) | |
| 94990 BxPC-3 Pancreatic adenocarcinoma | 0.0 |
| 94991 HPAC Pancreatic adenocarcinoma | 0.0 |
| 94992 MIA PaCa-2 Pancreatic carcinoma | 0.0 |
| 94993 CFPAC-1 Pancreatic ductal | 20.4 |
| adenocarcinoma | |
| 94994 PANC-1 Pancreatic epithelioid | 0.0 |
| ductal carcinoma | |
| 94996 T24 Bladder carcinoma | 19.9 |
| (transitional cell | |
| 5637- Bladder carcinoma | 0.0 |
| 94998 HT-1197 Bladder carcinoma | 0.0 |
| 94999 UM-UC-3 Bladder carcinoma | 0.0 |
| (transitional cell) | |
| 95000 A204 Rhabdomyosarcoma | 14.4 |
| 95001 HT-1080 Fibrosarcoma | 0.0 |
| 95002 MG-63 Osteosarcoma (bone) | 0.0 |
| 95003 SK-LMS-1 Leiomyosarcoma (vulva) | 8.2 |
| 95004 SJRH30 Rhabdomyosarcoma | 0.0 |
| (met to bone marrow) | |
| 95005 A431 Epidermoid carcinoma | 0.0 |
| 95007 WM266-4 Melanoma | 5.2 |
| DU 145- Prostate carcinoma (brain metastasis) | 0.0 |
| 95012 MDA-MB-468 Breast adenocarcinoma | 0.0 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| 95017 CAL 27 Squamous cell | 6.7 |
| carcinoma of tongue | |

Column A - Rel. Exp. (%) Ag4347, Run 190952231

TABLE DJ
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1 anti-CD95 CH11 | 3.3 |
| LAK cells rest | 0.7 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 2.3 |
| PBMC rest | 0.0 |
| PBMC PWM | 1.3 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 1.3 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.9 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.7 |
| CCD1106 (Keratinocytes) TNFalpha + IL-1beta | 0.0 |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 0.5 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.6 |
| Dermal fibroblast IFN gamma | 0.0 |
| Dermal fibroblast IL-4 | 0.6 |
| Dermal Fibroblasts rest | 0.0 |
| Neutrophils TNFa + LPS | 1.1 |
| Neutrophils rest | 0.6 |
| Colon | 0.5 |
| Lung | 2.2 |
| Thymus | 12.2 |
| Kidney | 100.0 |

Column A - Rel. Exp. (%) Ag4346, Run 190944493

TABLE DK
general oncology screening panel_v_2.4

| Tissue Name | A |
|---|---|
| Colon cancer 1 | 58.6 |
| Colon NAT 1 | 0.0 |
| Colon cancer 2 | 0.0 |
| Colon NAT 2 | 0.0 |
| Colon cancer 3 | 0.0 |
| Colon NAT 3 | 29.1 |
| Colon malignant cancer 4 | 21.8 |
| Colon NAT 4 | 0.0 |
| Lung cancer 1 | 0.0 |
| Lung NAT 1 | 0.0 |
| Lung cancer 2 | 0.0 |
| Lung NAT 2 | 0.0 |
| Squamous cell carcinoma 3 | 0.0 |
| Lung NAT 3 | 0.0 |
| Metastatic melanoma 1 | 79.0 |
| Melanoma 2 | 69.7 |
| Melanoma 3 | 19.5 |
| Metastatic melanoma 4 | 100.0 |
| Metastatic melanoma 5 | 49.3 |
| Bladder cancer 1 | 0.0 |
| Bladder NAT 1 | 0.0 |
| Bladder cancer 2 | 0.0 |
| Bladder NAT 2 | 0.0 |
| Bladder NAT 3 | 0.0 |
| Bladder NAT 4 | 25.0 |
| Prostate adenocarcinoma 1 | 23.2 |
| Prostate adenocarcinoma 2 | 0.0 |
| Prostate adenocarcinoma 3 | 44.8 |
| Prostate adenocarcinoma 4 | 0.0 |
| Prostate NAT 5 | 25.5 |
| Prostate adenocarcinoma 6 | 10.1 |
| Prostate adenocarcinoma 7 | 0.0 |
| Prostate adenocarcinoma 8 | 0.0 |
| Prostate adenocarcinoma 9 | 10.6 |
| Prostate NAT 10 | 0.0 |
| Kidney cancer 1 | 24.7 |
| Kidney NAT 1 | 24.3 |
| Kidney cancer 2 | 75.3 |
| Kidney NAT 2 | 28.1 |
| Kidney cancer 3 | 7.4 |
| Kidney NAT 3 | 23.7 |
| Kidney cancer 4 | 0.0 |
| Kidney NAT 4 | 28.1 |

Column A - Rel. Exp. (%) Ag4347, Run 260280470

AI_comprehensive panel_v1.0 Summary: Ag4347 Highest expression of this gene was detected in normal lung (CT=30.8). This gene showed a wide spread low expression in this panel. Moderate to low levels of expression of this gene were detected in samples derived from normal and orthoarthitis/rheumatoid arthritis bone, cartilage, and synovium samples, from normal lung, COPD lung, emphysema, atopic asthma, asthma, allergy, Crohn's disease (normal matched control and diseased), ulcerative colitis (normal matched control and diseased), and psoriasis (normal matched control and diseased). Therefore, therapeutic modulation of this gene, encoded protein and/or use of antibodies or small molecule targeting this gene or gene product is useful in the treatment of autoimmune and inflammatory disorders including psoriasis, allergy, asthma, inflammatory bowel disease, rheumatoid arthritis and osteoarthritis

Cellular OA/RA Summary: Ag4346 Highest expression of this gene was detected in MH7A (synoviocyte) cell line (CT=33.5). Low expression of this gene was also detected in untreated and activated SW1353 (chondrocyte) cell lines, and activated MH7A cells. Therefore, modulation of this gene or encoded protein will be useful in the treatment of orthoarthritis and rheumatoid arthritis.

General_screening_panel_v1.4 Summary: Ag4347 Highest expression of this gene was detected in ovarian cancer SK-OV-3 cell line (CT=32). Low expression of this gene was detected in number of cancer cell lines derived from ovarian, breast, and colon cancers. Therefore, therapeutic modulaion of this gene, expressed protein and/or use of antibodies or small molecule drug targeting this gene or gene product is useful in the treatment of ovarian, breast and colon cancers.

Low levels of expression of this gene were seen in all the regions of the central nervous system examined, including amygdala, hippocampus, substantia nigra, thalamus, cerebellum, cerebral cortex, and spinal cord. Expression analysis of this gene using CuraChip 1.2 (see example 2) showed that this gene was down-regulated in the temporal cortex of Alzheimer's patient but was up-regulated in patients who were found to have serious Alzheimer disease-like pathology with no associated dementia relative to the control patients. Therefore, therapeutic modulation of this gene, expressed protein and/or use of antibodies or small molecule drug targeting this gene or gene product is useful in the treatment of central nervous system disorders such as Alzheimer's disease, Parkinson's disease, epilepsy, multiple sclerosis, schizophrenia and depression.

Mini tissue OA/RA Summary: Ag4346 Highest expression of this gene was detected in brain (CT=29.9). Significant expression of this gene was also seen in normal synovium, kidney, colon, ovary and small intestine. Expression of this gene was slightly downregulated in synovium from OA and RA patients. Therefore, modulation of this gene and/or encoded protein is useful in the treatment of orthoarthritis and rheumatoid arthritis.

PGI1.0 Summary: Ag4346 Highest expression of this gene was detected in lung fibrosis sample (CT=30.8). Significant levels of expression of this gene was also detected in emphyzema and asthma lung. Therefore, therapeutic modulaion of this gene, encoded protein and/or use of expressed protein, antibodies or small molecule drug targeting this gene or gene product is useful in the treatment of emphyzema, asthma and lung fibrosis.

Panel 3D Summary: Ag4347 Low expression of this gene was detected mainly in a small cell lung cancer DMS-79 and DMS-114 cell lines (CTs=33-34). Therefore, therapeutic modulation of this gene, encoded protein, and/or use of small molecule drug targeting this gene or gene product is useful in the treatment of small cell lung cancer.

Panel 4.1D Summary: Ag4346 Moderate levels of expression of this gene was detected mainly in kidney sample (CT=31.8). Therefore, therapeutic modulation of this gene is useful in the treatment of kidney related diseases including lupus erythematosus and glomerulonephritis.

general oncology screening panel_v_{—}2.4 Summary: Ag4347 Low expression of this gene was detected in a colon cancer, a metastatic melanoma and a kidney cancer samples. Therefore, therapeutic modulation of this gene, encoded protein,and/or use of antibodies or small molecule drug targeting this gene or gene product is useful in the treatment of these cancers.

E. NOV5a (CG54611-06): WNT-3A Protein Precursor

Expression of gene CG54611-06 was assessed using the primer-probe sets Ag2445 and Ag7111, described in Tables EA and EB. Results of the RTQ-PCR runs are shown in Tables EC and ED.

TABLE EA

Probe Name Ag2445

| | | | Start | SEQ ID |
|---|---|---|---|---|
| Primers | Sequences | Length | Position | No |
| Forward | 5′-gccccactcggatacttct-3′ | 19 | 22 | 241 |
| Probe | TET-5′-tactcctctgcagcctgaagcaggct-3′- | 26 | 41 | 242 |
| | TAMRA | | | |
| Reverse | 5′-ggaatactgtggcccaaca-3′ | 19 | 99 | 243 |

TABLE EB

Probe Name Ag7111

| | | | Start | SEQ ID |
|---|---|---|---|---|
| Primers | Sequences | Length | Position | No |
| Forward | 5′-cgtgctggacaaagctacc-3′ | 19 | 318 | 244 |
| Probe | TET-5′-agtcggcctttgtccacgccatt-3′- | 23 | 341 | 245 |
| | TAMRA | | | |
| Reverse | 5′-gtcactgcaaaggccaca-3′ | 18 | 375 | 246 |

TABLE EC
Ardais Panel 1.1

| Tissue Name | A |
|---|---|
| 136803 Lung cancer(368) | 0.0 |
| 136804 Lung cancer(369) | 5.1 |
| 136805 Lung NAT(36A) | 100.0 |
| 136787 lung cancer(356) | 0.0 |
| 136788 lung NAT(357) | 62.9 |
| 136806 Lung cancer(36B) | 0.6 |
| 136807 Lung NAT(36C) | 73.2 |
| 136810 Lung NAT(36F) | 17.6 |
| 136789 lung cancer(358) | 1.4 |
| 136802 Lung cancer(365) | 0.5 |
| 136811 Lung cancer(370) | 0.5 |
| 136791 Lung cancer(35A) | 4.5 |
| 136794 lung NAT(35D) | 96.6 |
| 136815 Lung cancer(374) | 8.1 |
| 136816 Lung NAT(375) | 31.6 |
| 136813 Lung cancer(372) | 17.6 |
| 136814 Lung NAT(373) | 31.9 |
| 136795 Lung cancer(35E) | 29.9 |
| 136797 Lung cancer(360) | 5.9 |
| 136799 Lung cancer(362) | 30.6 |
| 136800 Lung NAT(363) | 17.7 |

Column A - Rel. Exp. (%) Ag2445, Run 306368467

TABLE ED
General_screening_panel_v1.7

| Tissue Name | A | B |
|---|---|---|
| Adipose | 1.6 | 0.1 |
| HUVEC | 0.0 | 0.0 |
| Melanoma* Hs688(A).T | 0.0 | 0.0 |
| Melanoma* Hs688(B).T | 56.3 | 54.0 |
| Melanoma (met) SK-MEL-5 | 0.0 | 0.0 |
| Testis | 0.8 | 0.4 |
| Prostate ca. (bone met) PC-3 | 0.0 | 0.0 |
| Prostate ca. DU145 | 1.7 | 1.6 |
| Prostate pool | 4.0 | 6.0 |
| Uterus pool | 0.0 | 0.4 |
| Ovarian ca. OVCAR-3 | 0.0 | 0.0 |
| Ovarian ca. (ascites) SK-OV-3 | 0.0 | 0.0 |
| Ovarian ca. OVCAR-4 | 0.0 | 3.8 |
| Ovarian ca. OVCAR-5 | 0.2 | 1.0 |
| Ovarian ca. IGROV-1 | 0.0 | 18.8 |
| Ovarian ca. OVCAR-8 | 0.0 | 0.0 |
| Ovary | 0.0 | 0.0 |
| Breast ca. MCF-7 | 1.5 | 1.8 |
| Breast ca. MDA-MB-231 | 0.4 | 0.6 |
| Breast ca. BT 549 | 0.0 | 0.0 |
| Breast ca. T47D | 0.0 | 0.0 |
| Breast pool | 0.0 | |
| Trachea | 23.5 | 34.6 |
| Lung | 100.0 | 97.3 |
| Fetal Lung | 1.1 | 1.2 |
| Lung ca. NCI-N417 | 0.0 | 0.5 |
| Lung ca. LX-1 | 0.0 | 0.0 |
| Lung ca. NCI-H146 | 0.0 | 0.0 |
| Lung ca. SHP-77 | 0.0 | 0.0 |
| Lung ca. NCI-H23 | 0.0 | 0.6 |
| Lung ca. NCI-H460 | 0.4 | 0.0 |
| Lung ca. HOP-62 | 0.5 | 0.0 |
| Lung ca. NCI-H522 | 0.2 | 1.3 |
| Lung ca. DMS-114 | 0.4 | 0.4 |
| Liver | 0.0 | 0.0 |
| Fetal Liver | 0.0 | 0.0 |
| Kidney pool | 1.1 | 1.5 |
| Fetal Kidney | 0.9 | 5.7 |
| Renal ca. 786-0 | 13.7 | 27.9 |
| Renal ca. A498 | 0.0 | 0.6 |
| Renal ca. ACHN | 0.0 | 0.0 |
| Renal ca. UO-31 | 0.0 | 0.0 |
| Renal ca. TK-10 | 0.0 | 0.0 |
| Bladder | 0.0 | 0.0 |
| Gastric ca. (liver met.) NCI-N87 | 0.0 | 0.0 |
| Stomach | 0.0 | 0.0 |
| Colon ca. SW-948 | 0.0 | 0.0 |
| Colon ca. SW480 | 0.0 | 0.0 |
| Colon ca. (SW480 met) SW620 | 0.0 | 0.0 |
| Colon ca. HT29 | 0.0 | 0.0 |
| Colon ca. HCT-116 | 75.8 | 100.0 |
| Colon cancer tissue | 0.0 | 0.0 |
| Colon ca. SW1116 | 0.0 | 0.0 |
| Colon ca. Colo-205 | 0.0 | 0.0 |
| Colon ca. SW-48 | 0.0 | 0.0 |
| Colon | 0.0 | 0.0 |
| Small Intestine | 0.0 | 0.0 |
| Fetal Heart | 0.0 | 0.0 |
| Heart | 0.0 | 0.0 |
| Lymph Node pool 2 | 0.7 | 0.2 |
| Fetal Skeletal Muscle | 0.0 | 0.0 |
| Skeletal Muscle pool | 0.0 | 0.0 |
| Skeletal Muscle | 0.0 | 0.0 |
| Spleen | 0.0 | 0.0 |
| Thymus | 0.6 | 0.0 |
| CNS cancer (glio/astro) SF-268 | 0.0 | 0.0 |
| CNS cancer (glio/astro) T98G | 0.0 | 0.0 |
| CNS cancer (neuro; met) SK-N-AS | 0.0 | 0.0 |
| CNS cancer (astro) SF-539 | 0.5 | 0.2 |
| CNS cancer (astro) SNB-75 | 0.0 | 1.9 |
| CNS cancer (glio) SNB-19 | 0.0 | 0.0 |
| CNS cancer (glio) SF-295 | 0.0 | 0.0 |
| Brain (Amygdala) | 0.0 | 0.0 |
| Brain (Cerebellum) | 0.0 | 0.0 |
| Brain (Fetal) | 0.0 | 0.0 |
| Brain (Hippocampus) | 0.0 | 0.0 |
| Cerebral Cortex pool | 0.0 | 0.0 |
| Brain (Substantia nigra) | 0.0 | 0.0 |
| Brain (Thalamus) | 0.0 | 0.0 |
| Brain (Whole) | 0.0 | 0.0 |
| Spinal Cord | 0.0 | 0.0 |
| Adrenal Gland | 0.0 | 0.0 |
| Pituitary Gland | 0.0 | 0.0 |
| Salivary Gland | 6.5 | 4.9 |
| Thyroid | 0.0 | 0.5 |
| Pancreatic ca. PANC-1 | 0.0 | 2.0 |
| Pancreas pool | 0.0 | 0.0 |

Column A - Rel. Exp. (%) Ag2445, Run 405874582
Column B - Rel. Exp. (%) Ag7111, Run 318037265

Ardais Panel 1.1 Summary: Ag2445 Highest expression of this gene was detected in a matched control sample for lung cancer (CT=28.5). The gene expression was down-regulated in coresponding cancer tissues. This gene encodes wingless-type MMTV integration site family, member 3A (WNT3A). Therapeutic modulation of this gene, expressed protein and/or use of antibodies or small molecule drugs targeting the gene or gene product are useful in the treatment of lung cancer.

General_screening_panel_v1.7 Summary: Ag2445 and Ag7111 Results from two experiments using the two different probe and primer sets that respond to the AL391534_C gene are in very good agreement. Moderate expression was detected in normal lung (CT=28.7, 29.8) but not in any of the 9 lung cancer lines examined. It is consistant with data from patient tissues, see Ardais Panel 1.1. Thus, therapeutic modulation of this gene, expressed protein and/or use of antibodies or small molecule drugs targeting the gene or gene product is useful in the treatment of lung cancer.

Moderate expression was also detected in trachea (CT=30.26, 31.89), one (out of 9) colon cancer line (CT=28.9, 30.2) and one (out of 3) Melanoma cell line (CT=29.6, 30.6).

F. NOV6a (CG92035-02): C1Q-Related Factor Precursor

Expression of gene CG92035-02 was assessed using the primer-probe sets Ag3767, Ag4935 and Ag4902, described in Tables FA, FB and FC. Results of the RTQ-PCR runs are shown in Tables FD and FE.

TABLE FA

Probe Name Ag3767

| | | | Start | SEQ ID |
|---|---|---|---|---|
| Primers | Sequences | Length | Position | No |
| Forward | 5′-gcggaccagaactacgactac-3′ | 21 | 634 | 247 |
| Probe | TET-5′-agcaacagcgtgatcctgcacct-3′- | 23 | 658 | 248 |
| | TAMRA | | | |
| Reverse | 5′-tccatccagcttgatgaaga-3′ | 20 | 698 | 249 |

TABLE FB

Probe Name Ag4935

| | | | Start | SEQ ID |
|---|---|---|---|---|
| Primers | Sequences | Length | Position | No |
| Forward | 5′-gcggaccagaactacgactac-3′ | 21 | 634 | 250 |
| Probe | TET-5′-agcaacagcgtgatcctgcacct-3′- | 23 | 658 | 251 |
| | TAMRA | | | |
| Reverse | 5′-tccatccagcttgatgaaga-3′ | 20 | 698 | 252 |

TABLE FC

Probe Name Ag4902

| | | | Start | SEQ ID |
|---|---|---|---|---|
| Primers | Sequences | Length | Position | No |
| Forward | 5′-gcggaccagaactacgactac-3′ | 21 | 634 | 253 |
| Probe | TET-5′-agcaacagcgtgatcctgcacct-3′- | 23 | 658 | 254 |
| | TAMRA | | | |
| Reverse | 5′-tccatccagcttgatgaaga-3′ | 20 | 698 | 255 |

TABLE FD
General_screening_panel_v1.4

| Tissue Name | A |
|---|---|
| Adipose | 0.2 |
| Melanoma* Hs688(A).T | 0.1 |
| Melanoma* Hs688(B).T | 0.1 |
| Melanoma* M14 | 0.6 |
| Melanoma* LOXIMVI | 0.0 |
| Melanoma* SK-MEL-5 | 2.0 |
| Squamous cell carcinoma SCC-4 | 0.1 |
| Testis Pool | 0.1 |
| Prostate ca.* (bone met) PC-3 | 1.8 |
| Prostate Pool | 0.3 |
| Placenta | 0.0 |
| Uterus Pool | 0.2 |
| Ovarian ca. OVCAR-3 | 0.2 |
| Ovarian ca. SK-OV-3 | 14.2 |
| Ovarian ca. OVCAR-4 | 0.1 |
| Ovarian ca. OVCAR-5 | 2.2 |
| Ovarian ca. IGROV-1 | 4.0 |
| Ovarian ca. OVCAR-8 | 8.9 |
| Ovary | 0.1 |
| Breast ca. MCF-7 | 1.7 |
| Breast ca. MDA-MB-231 | 0.7 |
| Breast ca. BT 549 | 7.9 |
| Breast ca. T47D | 10.0 |
| Breast ca. MDA-N | 0.1 |
| Breast Pool | 0.1 |
| Trachea | 0.2 |
| Lung | 0.0 |
| Fetal Lung | 0.4 |
| Lung ca. NCI-N417 | 1.1 |
| Lung ca. LX-1 | 0.0 |
| Lung ca. NCI-H146 | 4.9 |
| Lung ca. SHP-77 | 4.2 |
| Lung ca. A549 | 2.1 |
| Lung ca. NCI-H526 | 0.3 |
| Lung ca. NCI-H23 | 1.7 |
| Lung ca. NCI-H460 | 0.2 |
| Lung ca. HOP-62 | 3.1 |
| Lung ca. NCI-H522 | 1.8 |
| Liver | 0.0 |
| Fetal Liver | 0.1 |
| Liver ca. HepG2 | 0.0 |
| Kidney Pool | 1.0 |
| Fetal Kidney | 0.4 |
| Renal ca. 786-0 | 34.6 |
| Renal ca. A498 | 100.0 |
| Renal ca. ACHN | 4.3 |
| Renal ca. UO-31 | 0.2 |
| Renal ca. TK-10 | 0.6 |
| Bladder | 0.3 |
| Gastric ca. (liver met.) NCI-N87 | 0.1 |
| Gastric ca. KATO III | 0.0 |
| Colon ca. SW-948 | 0.0 |
| Colon ca. SW480 | 0.5 |
| Colon ca.* (SW480 met) SW620 | 0.0 |
| Colon ca. HT29 | 0.0 |
| Colon ca. HCT-116 | 0.5 |
| Colon ca. CaCo-2 | 0.6 |
| Colon cancer tissue | 0.0 |
| Colon ca. SW1116 | 1.3 |
| Colon ca. Colo-205 | 0.0 |
| Colon ca. SW-48 | 0.0 |
| Colon Pool | 0.1 |
| Small Intestine Pool | 0.3 |
| Stomach Pool | 0.2 |
| Bone Marrow Pool | 0.2 |
| Fetal Heart | 1.9 |
| Heart Pool | 0.3 |
| Lymph Node Pool | 0.4 |
| Fetal Skeletal Muscle | 0.2 |
| Skeletal Muscle Pool | 0.0 |
| Spleen Pool | 0.0 |
| Thymus Pool | 0.1 |
| CNS cancer (glio/astro) U87-MG | 1.2 |
| CNS cancer (glio/astro) U-118-MG | 3.3 |
| CNS cancer (neuro; met) SK-N-AS | 2.0 |
| CNS cancer (astro) SF-539 | 1.0 |
| CNS cancer (astro) SNB-75 | 11.0 |
| CNS cancer (glio) SNB-19 | 3.3 |
| CNS cancer (glio) SF-295 | 5.0 |
| Brain (Amygdala) Pool | 1.1 |
| Brain (cerebellum) | 1.8 |
| Brain (fetal) | 1.9 |
| Brain (Hippocampus) Pool | 1.8 |
| Cerebral Cortex Pool | 2.0 |
| Brain (Substantia nigra) Pool | 2.4 |
| Brain (Thalamus) Pool | 2.3 |
| Brain (whole) | 2.5 |
| Spinal Cord Pool | 4.0 |
| Adrenal Gland | 0.2 |
| Pituitary gland Pool | 0.0 |
| Salivary Gland | 0.0 |
| Thyroid (female) | 0.1 |
| Pancreatic ca. CAPAN2 | 0.1 |
| Pancreas Pool | 0.3 |

Column A - Rel. Exp. (%) Ag3767, Run 218981839

TABLE FE
Panel 4.1D

| Tissue Name | A | B |
|---|---|---|
| Secondary Th1 act | 0.0 | 0.5 |
| Secondary Th2 act | 4.0 | 1.4 |
| Secondary Tr1 act | 4.5 | 0.5 |
| Secondary Th1 rest | 0.0 | 0.6 |
| Secondary Th2 rest | 2.9 | 2.5 |
| Secondary Tr1 rest | 2.9 | 0.0 |
| Primary Th1 act | 2.3 | 0.3 |
| Primary Th2 act | 0.0 | 0.3 |
| Primary Tr1 act | 3.5 | 0.6 |
| Primary Th1 rest | 0.7 | 0.6 |
| Primary Th2 rest | 0.0 | 0.3 |
| Primary Tr1 rest | 3.0 | 2.9 |
| CD45RA CD4 lymphocyte act | 9.1 | 3.6 |
| CD45RO CD4 lymphocyte act | 1.2 | 0.0 |
| CD8 lymphocyte act | 0.6 | 0.0 |
| Secondary CD8 lymphocyte rest | 0.6 | 0.3 |
| Secondary CD8 lymphocyte act | 1.5 | 1.5 |
| CD4 lymphocyte none | 0.8 | 0.0 |
| 2ry Th1/Th2/Tr1 anti-CD95 CH11 | 1.7 | 0.0 |
| LAK cells rest | 6.7 | 1.2 |
| LAK cells JL-2 | 2.8 | 1.6 |
| LAK cells IL-2 + IL-12 | 2.4 | 0.0 |
| LAK cells IL-2 + IFN gamma | 3.1 | 0.3 |
| LAK cells IL-2 + IL-18 | 8.7 | 1.3 |
| LAK cells PMA/ionomycin | 3.3 | 2.7 |
| NK Cells IL-2 rest | 25.5 | 12.4 |
| Two Way MLR 3 day | 0.7 | 0.0 |
| Two Way MLR 5 day | 0.8 | 0.7 |
| Two Way MLR 7 day | 0.7 | 0.0 |
| PBMC rest | 0.0 | 0.0 |
| PBMC PWM | 0.0 | 0.0 |
| PBMC PHA-L | 14.9 | 9.2 |
| Ramos (B cell) none | 0.0 | 0.0 |
| Ramos (B cell) ionomycin | 0.0 | 0.0 |
| B lymphocytes PWM | 0.0 | 1.1 |
| B lymphocytes CD40L and IL-4 | 0.0 | 0.4 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.4 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.0 | 0.9 |
| HUVEC none | 1.7 | 0.0 |
| HUVEC starved | 1.8 | 1.8 |
| HUVEC IL-1beta | 3.3 | 2.3 |
| HUVEC IFN gamma | 2.9 | 4.0 |
| HUVEC TNF alpha + IFN gamma | 1.4 | 1.2 |
| HUVEC TNF alpha + IL4 | 0.7 | 1.1 |
| HUVEC IL-11 | 3.2 | 1.8 |
| Lung Microvascular EC none | 5.6 | 2.0 |
| Lung Microvascular EC TNFalpha + IL-1beta | 5.8 | 2.9 |
| Microvascular Dermal EC none | 1.5 | 0.8 |
| Microsvasular Dermal EC TNFalpha + IL-1beta | 0.0 | 1.1 |
| Bronchial epithelium TNFalpha + IL1beta | 1.8 | 1.9 |
| Small airway epithelium none | 0.0 | 0.0 |
| Small airway epithelium TNFalpha + IL-1beta | 1.7 | 0.2 |
| Coronery artery SMC rest | 2.7 | 0.8 |
| Coronery artery SMC TNFalpha + IL-1beta | 0.0 | 0.9 |
| Astrocytes rest | 100.0 | 43.8 |
| Astrocytes TNFalpha + IL-1beta | 53.2 | 15.8 |
| KU-812 (Basophil) rest | 0.0 | 1.4 |
| KU-812 (Basophil) PMA/ionomycin | 0.0 | 1.1 |
| CCD1106 (Keratinocytes) none | 23.5 | 8.5 |
| CCD1106 (Keratinocytes) TNFalpha + IL-1beta | 8.5 | 5.4 |
| Liver cirrhosis | 6.8 | 4.2 |
| NCI-H292 none | 22.2 | 13.7 |
| NCI-H292 IL-4 | 49.7 | 19.8 |
| NCI-H292 IL-9 | 79.0 | 43.5 |
| NCI-H292 IL-13 | 50.0 | 40.6 |
| NCI-H292 IFN gamma | 66.9 | 31.2 |
| HPAEC none | 6.7 | 1.5 |
| HPAEC TNF alpha + IL-1 beta | 5.0 | 1.6 |
| Lung fibroblast none | 62.0 | 36.9 |
| Lung fibroblast TNF alpha + IL-1 beta | 42.3 | 31.4 |
| Lung fibroblast IL-4 | 35.1 | 35.1 |
| Lung fibroblast IL-9 | 59.9 | 26.1 |
| Lung fibroblast IL-13 | 48.0 | 20.0 |
| Lung fibroblast IFN gamma | 88.9 | 44.4 |
| Dermal fibroblast CCD1070 rest | 14.5 | 6.0 |
| Dermal fibroblast CCD1070 TNF alpha | 16.8 | 6.3 |
| Dermal fibroblast CCD1070 IL-1 beta | 10.8 | 6.6 |
| Dermal fibroblast IFN gamma | 21.5 | 8.8 |
| Dermal fibroblast IL-4 | 19.2 | 17.1 |
| Dermal Fibroblasts rest | 13.5 | 7.9 |
| Neutrophils TNFa + LPS | 0.0 | 0.6 |
| Neutrophils rest | 0.0 | 1.5 |
| Colon | 24.8 | 7.5 |
| Lung | 5.4 | 7.4 |
| Thymus | 3.0 | 22.1 |
| Kidney | 44.8 | 100.0 |

Column A - Rel. Exp. (%) Ag3767, Run 170069112
Column B - Rel. Exp. (%) Ag4935, Run 223598631

General_screening_panel_v1.4 Summary: Ag3767 High expression of this gene was detected in 2 renal cancer A498 and 786-0 cell lines (CTs=26-28). Moderate expression of this gene was also seen in number of cancer cell lines derived from melanoma, brain, breast, ovary, lung and prostate cancers. Therefore, expression of this gene can be used as diagnostic marker to detect the presence of these cancers. Therapeutic modulation of this gene, expressed protein and/or use of antibodies or small molecule drugs targeting the gene or gene product is useful in the treatment of kidney, melanoma, brain, breast, ovary, lung and prostate cancers.

This gene was expressed at moderate to low levels in all regions of the central nervous system examined, including amygdala, hippocampus, substantia nigra, thalamus, cerebellum, cerebral cortex, and spinal cord. Therapeutic modulation of this gene, expressed protein and/or use of antibodies or small molecule drugs targeting the gene or gene product is useful in the treatment of central nervous system disorders such as Alzheimer's disease, Parkinson's disease, epilepsy, multiple sclerosis, schizophrenia and depression.

This gene was expressed at much higher level in fetal (CT=32.3) when compared to adult heart(CT=35). This observation indicates that the protein product may enhance heart growth or development in the fetus and thus act in a regenerative capacity in the adult. Therapeutic modulation of this gene, expressed protein and/or use of antibodies or small molecule drugs targeting the gene or gene product is useful in treatment of heart related diseases.

Panel 4.1D Summary: Ag3767/μg4935 Highest expression of this gene was detected in resting astrocytes and kidney (CTs=30-32). In addition, moderate to low expression of this gene was also seen in keratinocytes, resting and activated mucoepidermoid NCI-H292, resting and activated lung and dermal fibroblasts. Therefore, therapeutic modulation of this gene or its protein product through the use of antibodies or small molecule drug is useful in the threatment of psoriasis, asthma, allergies, chronic obstructive pulmonary disease, emphysema and kidney related diseases including lupus erythematosus.

### Example D

### Expression of CG54455-06 in stable CHO-K1 cells

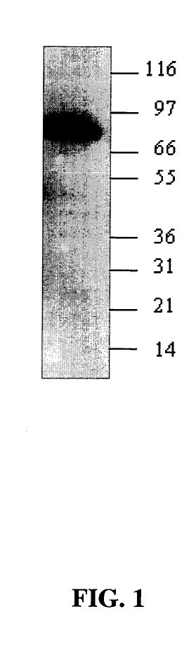

A 456 bp long Bg1II-XhoI fragment containing the CG54455-06 (mature form of CG54455-01) sequence was subcloned into BamHI-XhoI digested pEE14.4FL2_MSA to generate plasmid 3337. The resulting plasmid 3337 was transfected into CHO-K1 cells using the LipofectaminePlus reagent following the manufacturer's instructions (Invitrogen/Gibco) and stable clones were selected based on resistance against MSX. The culture media was DMEM, 10% FBS, 1× nonessential amino acids. The expression and secretion levels of the clone were assessed by Western blot analysis using HRP conjugated V5 antibody. The V5 epitope is fused to the gene of interest at the Cter, in the pEE14.4Sec vector. FIG. 1 shows that CG54455 is expressed, and a 94 kDa protein is secreted by the CHO-K1 cells.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequence selected from the group consisting of SEQ ID NO:2n, wherein n is an integer between 1 and 102.

2. An isolated polypeptide comprising an amino acid sequence selected from the group consisting of SEQ ID NO:2n, wherein n is an integer between 1 and 102.

3. An isolated polypeptide comprising an amino acid sequence which is at least 95% identical to an amino acid sequence selected from the group consisting of SEQ ID NO:2n, wherein n is an integer between 1 and 102.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 102.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease, wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide; whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 102, or a biologically active fragment thereof.

20. An isolated nucleic acid molecule comprising a nucleic acid sequence selected from the group consisting of SEQ ID NO:2n−1, wherein n is an integer between 1 and 102.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 102.

23. An isolated nucleic acid molecule encoding the mature form of a polypeptide having an amino acid sequence selected from the group consisting of SEQ ID NO:2n, wherein n is an integer between 1 and 102.

24. A composition comprising an isolated nucleic acid molecule, said molecule comprising a nucleic acid sequence selected from the group consisting of SEQ ID NO: 2n−1, wherein n is an integer between 1 and 102, and a carrier.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 102, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule, thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease; wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 102.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 102.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.

46. An isolated polypeptide comprising an amino acid sequence at least 95% similar to SEQ ID NO: 4, wherein said amino acid sequence comprises at least one amino acid substitution, wherein said substitution is at the amino acid position selected from the group consisting of 19, 68, 82, 103, 115, 136, 310, 320, 325, 331, 370 and 581 when numbered in accordance with SEQ ID NO: 4.

47. An isolated nucleic acid molecule comprising an nucleic acid sequence at least 95% similar to SEQ ID NO: 3, wherein said nucleic acid sequence comprises at least one nucleic acid substitution, wherein said substitution is at the nucleic acid position selected from the group consisting of 105, 253, 295, 356, 392, 456, 978, 1008, 1023, 1041, 1157 and 1791 when numbered in accordance with SEQ ID NO: 3.

48. An isolated polypeptide comprising an amino acid sequence at least 95% similar to SEQ ID NO: 78, wherein said amino acid sequence comprises at least one amino acid substitution, wherein said substitution is at the amino acid position selected from the group consisting of 77, 91 125, 174, 189 and 203 when numbered in accordance with SEQ ID NO: 78.

49. An isolated nucleic acid molecule comprising an nucleic acid sequence at least 95% similar to SEQ ID NO: 77, wherein said nucleic acid sequence comprises at least one nucleic acid substitution, wherein said substitution is at the nucleic acid position selected from the group consisting of 231, 272, 374, 522, 566 and 608 when numbered in accordance with SEQ ID NO: 77.

50. An isolated polypeptide comprising an amino acid sequence at least 95% similar to SEQ ID NO: 116, wherein said amino acid sequence comprises at least one amino acid substitution, wherein said substitution is at amino acid position 120 when numbered in accordance with SEQ ID NO: 116.

51. An isolated nucleic acid molecule comprising an nucleic acid sequence at least 95% similar to SEQ ID NO: 115, wherein said nucleic acid sequence comprises at least one nucleic acid substitution, wherein said substitution is at nucleic acid position 358 when numbered in accordance with SEQ ID NO: 115.

52. An isolated polypeptide comprising an amino acid sequence at least 95% similar to SEQ ID NO: 32, wherein said amino acid sequence comprises at least one amino acid substitution, wherein said substitution is at the amino acid position selected from the group consisting of 40, 44, 134, 212, 234, 289, 311 and 348 when numbered in accordance with SEQ ID NO: 32.

53. An isolated nucleic acid molecule comprising an nucleic acid sequence at least 95% similar to SEQ ID NO: 31, wherein said nucleic acid sequence comprises at least one nucleic acid substitution, wherein said substitution is at the nucleic acid position selected from the group consisting of 149, 160, 430, 664, 731, 895, 961 and 1073 when numbered in accordance with SEQ ID NO: 31.
